(19) United States
(12) Patent Application Publication
(10) Pub. No.: US 2026/0226437 A1
(43) Pub. Date: Aug. 6, 2026

(54) FANZORS ARE RNA-GUIDED NUCLEASES ENCODED IN EUKARYOTIC GENOMES

(71) Applicant: Massachusetts Institute of Technology, Cambridge, MA (US)

(72) Inventors: Omar Abudayyeh, Cambridge, MA (US); Jonathan Gootenberg, Cambridge, MA (US); Justin Lim, Boston, MA (US); Kaiyi Jiang, Cambridge, MA (US)

(73) Assignee: Massachusetts Institute of Technology, Cambridge, MA (US)

(21) Appl. No.: 19/466,914

(22) Filed: Feb. 2, 2026

Related U.S. Application Data
(63) Continuation of application No. 18/406,066, filed on Jan. 5, 2024, now abandoned.
(60) Provisional application No. 63/450,947, filed on Mar. 8, 2023.
(60) Provisional application No. 63/507,968, filed on Jun. 13, 2023.
(60) Provisional application No. 63/510,866, filed on Jun. 28, 2023.
(60) Provisional application No. 63/578,625, filed on Aug. 24, 2023.

(51) Int. Cl. C12N 9/22; C12N 15/11; C12N 15/90

(52) U.S. Cl. CPC C12N 9/22; C12N 15/11; C12N 15/902; C12N 2310/20

(57) ABSTRACT

The invention relates to compositions and methods for targeting polynucleotides with eukaryotic RNA-guided nucleases. In particular, programmable RNA-guided DNA endonucleases termed Fanzors, can be harnessed for genome editing.

### RELATED APPLICATIONS

This application is a continuation of U.S. patent application Ser. No. 18/406,066, filed Jan. 5, 2024, which claims priority to U.S. Provisional Application No. 63/450,947, filed Mar. 8, 2023; U.S. Provisional Application No. 63/507,968, filed Jun. 13, 2023; U.S. Provisional Application No. 63/510,866, filed Jun. 28, 2023; and U.S. Provisional Application No. 63/578,625, filed Aug. 24, 2023. The entire contents of each of these applications are incorporated herein by reference.

### FEDERALLY SPONSORED RESEARCH

This invention was made with government support under EB031957 awarded by National Institutes of Health. The government has certain rights in the invention.

### REFERENCE TO AN ELECTRONIC SEQUENCE LISTING

The contents of the electronic sequence listing (M065670531US06-SEQ-HCL.xml; Size: 7,280,350 bytes; and Date of Creation: Jan. 28, 2026) are herein incorporated by reference in their entirety.

### FIELD OF THE INVENTION

The present invention relates generally to methods of using programmable RNA-guided DNA endonucleases for genome-editing.

### BACKGROUND OF THE INVENTION

Prokaryotic and eukaryotic genomes are replete with diverse transposons, a broad class of mobile genetic elements (MGE). Transposons of the highly abundant IS200/605 family encode a pair of genes: TnpA, which codes for a DDE class transposase responsible for single-strand ‘peel and paste’ transposition, and TnpB, which has an unknown role in the transposition mechanism (Kapitonov et al. 2015; He et al. 2013). TnpB contains a RuvC-like nuclease domain (RNase H fold) that is specifically related to the homologous nuclease domain of the type V CRISPR effector Cas12 (Zetsche et al. 2015; Fonfara et al. 2016), specifically the Cas12f systems (Harrington et al. 2018), suggesting a direct evolutionary path from TnpB to Cas12 (Karevelis et al. 2021; Bao and Jurka 2013; Altae-Tran et al. 2021). This relationship is supported by phylogenetic analysis of the RuvC-like domains, which indicates independent origins of Cas12s of different type V subtypes from distinct groups of TnpBs. Bioinformatic analysis demonstrated that, along with IscB, IsrB, and IshB nucleases, TnpBs are components of obligate mobile element-guided activity (OMEGA) systems, which encode the guide ωRNA nearby the nuclease gene, often overlapping the coding region. Biochemical and cellular validation demonstrated ωRNA-TnpB complex forms an RNA-guided DNA endonuclease system (Karevelis et al. 2021; Altae-Tran et al. 2021).

RuvC-containing proteins are not limited to prokaryotic systems: a set of TnpB homologs, Fanzors, are present in eukaryotes (Bao and Jurka 2013). Mirroring the diversity of TnpBs in bacteria and archaea, Fanzor nucleases have been identified in diverse eukaryotic lineages, including metazoans, fungi, algae, amorphea, and double-stranded (ds) DNA viruses. Identified Fanzors fall into two major groups: 1) Fanzor1 nucleases are associated with eukaryotic transposons, including Mariners, IS4-like elements, Sola, Helitron, and MuDr, and occur predominantly in diverse eukaryotes; 2) Fanzor2 nucleases are found in IS607-like transposons and are present in large dsDNA viral genomes. Despite the similarities between TnpB and Fanzors, Fanzors have not been surveyed comprehensively throughout eukaryotic diversity, and they have not been demonstrated to be active nucleases in either biochemical or cellular contexts.

### SUMMARY OF THE INVENTION

The present disclosure reports a comprehensive census of RNA-guided nucleases in eukaryotic and viral genomes, discovering a broad class of nucleases termed Fanzors. Fanzor diversity was used herein to perform phylogenetic analysis revealing their evolution from prokaryotic origins and to validate activity through biochemical and cellular experiments, demonstrating the programmable RNA-guided endonuclease activity of the Fanzor. The invention relates, in one aspect, to the discovery that Fanzors comprise programmable RNA-guided endonuclease activity that can be harnessed for genome editing in human cells, highlighting the utility of the widespread eukaryotic RNA-guided nucleases for biotechnology applications. The invention relates, in some aspects, to the discovery that Fanzor programmable RNA-guided endonuclease activity can be harnessed for genome editing in any type of organism (e.g., eukaryotic, prokaryotic, and/or fungi).

Accordingly, aspects of the present disclosure provide compositions non-naturally occurring, engineered composition comprising: (a) a Fanzor polypeptide comprising an RuvC domain; and (b) a fRNA molecule comprising a scaffold and a reprogrammable target spacer sequence, wherein the fRNA molecule is capable of forming a complex with the Fanzor polypeptide and directing the Fanzor polypeptide to a target polynucleotide sequence.

In some embodiments, the RuvC domain further comprises a RuvC-I subdomain, a RuvC-II subdomain, and a RuvC-III subdomain, wherein the RuvC-II subdomain is a rearranged RuvC-II subdomain.

In some embodiments, the Fanzor polypeptide comprises about 200 to about 2212 amino acids.

In some embodiments, the reprogrammable target spacer sequence comprises about 12 to about 22 nucleotides.

In some embodiments, the scaffold comprises about 21 to about 1487 nucleotides.

In some embodiments, the complex binds a target adjacent motif (TAM) sequence 5′ of the target polynucleotide sequence. In some embodiments, the TAM sequence comprises GGG.

In some embodiments, the TAM sequence comprises TTTT. In some embodiments, the TAM sequence comprises TAT. In some embodiments, the TAM sequence comprises TTG. In some embodiments, the TAM sequence comprises TTTA. In some embodiments, the TAM sequence comprises TA. In some embodiments, the TAM sequence comprises TTA. In some embodiments, the TAM sequence comprises TGAC.

In some embodiments, the target polynucleotide is DNA.

In some embodiments, the Fanzor polypeptide is selected from a sequence listed in Table 1. In some embodiments, the Fanzor polypeptide shares at least 50%, at least 55%, at least 60%, at least 65%, at least 70%, at least 75%, at least 80%, at least 85%, at least 90%, at least 95%, at least 99%, or 100% sequence identity with a Fanzor polypeptide listed in Table 1.

In some embodiments, the Fanzor polypeptide is selected from a sequence listed in Table 4. In some embodiments, the Fanzor polypeptide shares at least 50%, at least 55%, at least 60%, at least 65%, at least 70%, at least 75%, at least 80%, at least 85%, at least 90%, at least 95%, at least 99%, or 100% sequence identity with a Fanzor polypeptide listed in Table 4.

In some embodiments, (a) the Fanzor polypeptide is a Fanzor polypeptide; and (b) the fRNA molecule is an fRNA molecule. In some embodiments, the Fanzor polypeptide is a Fanzor 1 polypeptide. In some embodiments, the Fanzor polypeptide is a Fanzor 2 polypeptide. In some embodiments, the Fanzor polypeptide further comprises a nuclear localization signal (NLS).

In some embodiments, the Fanzor polypeptide further comprises a helix-turn-helix (HTH) domain.

Further aspects of the present disclosure relate to compositions comprising one or more vectors comprising (a) a nucleic acid sequence encoding a Fanzor polypeptide comprising an RuvC domain; and (b) a nucleic acid sequence encoding a fRNA molecule comprising a scaffold and a reprogrammable target spacer sequence, wherein the fRNA molecule is capable of forming a complex with the Fanzor polypeptide and directing the Fanzor polypeptide to a target polynucleotide sequence. In some embodiments, (a) and (b) are comprised by one vector. In some embodiments, (a) and (b) are comprised by more than one vector.

In some embodiments, the composition further comprises one or more of a donor template comprising a donor sequence, optionally for use in homology-directed repair (HDR), a linear insert sequence, optionally for use in non-homologous end joining-based insertion, a reverse transcriptase, optionally for use in prime editing, a recombinase, optionally for use for integration, a transposase, optionally for use for integration, an integrase, optionally for use for integration, a deaminase, optionally for use of base-editing, a transcriptional activator, optionally for use of targeted gene activation, a transcriptional repressor, optionally for use of targeted gene repression, and/or a transposon, optionally for RNA guided transposition.

In some embodiments, the linear insert sequence comprises DNA. In some embodiments, the linear insert sequence comprises RNA. In some embodiments, the linear insert sequence comprises mRNA. In some embodiments, the linear insert is comprised by a viral vector, optionally wherein the viral vector is Adeno-associated viral (AAV) vector, a virus, optionally wherein the virus is an Adenovirus, a lentivirus, a herpes simplex virus; and/or a lipid nanoparticle.

In some embodiments, the integration comprises programmable addition via site-specific targeting elements (PASTE).

In some embodiments, the transposon is a eukaryotic transposon, optionally wherein the eukaryotic transposon is CMC, Copia, ERV, Gypsy, hAT, helitron, Zator, Sola, LINE, Tc1-Mariner, Novosib, Crypton, or EnSpm.

Further aspects of the present disclosure relate to engineered cells comprising (a) a Fanzor polypeptide comprising an RuvC domain; and (b) a fRNA molecule comprising a scaffold and a reprogrammable target spacer sequence, wherein the fRNA molecule is capable of forming a complex with the Fanzor polypeptide and directing the Fanzor polypeptide to a target polynucleotide sequence.

In some embodiments, the engineered cell is a mammalian cell. In some embodiments, the mammalian cell is a human cell. In some embodiments, the engineered cell is a non-mammalian, animal cell. In some embodiments, the engineered cell is a plant cell. In some embodiments, the engineered cell is a bacterial cell. In some embodiments, the engineered cell is a fungal cell. In some embodiments, the engineered cell is a yeast cell.

In some embodiments, the engineered cell further comprises one or more of a donor template comprising a donor sequence, optionally for use in homology-directed repair (HDR), a linear insert sequence, optionally for use in non-homologous end joining-based insertion, a reverse transcriptase, optionally for use in prime editing, a recombinase, optionally for use for integration, a transposase, optionally for use for integration, an integrase, optionally for use for integration, a deaminase, optionally for use of base-editing, a transcriptional activator, optionally for use of targeted gene activation, a transcriptional repressor, optionally for use of targeted gene repression, and/or a transposon, optionally for RNA guided transposition.

In some embodiments, the linear insert sequence comprises DNA. In some embodiments, the linear insert sequence comprises RNA. In some embodiments, the linear insert sequence comprises mRNA. In some embodiments, the linear insert is comprised by a viral vector, optionally wherein the viral vector is Adeno-associated viral (AAV) vector, a virus, optionally wherein the virus is an Adenovirus, a lentivirus, a herpes simplex virus; and/or a lipid nanoparticle.

In some embodiments, the integration comprises programmable addition via site-specific targeting elements (PASTE).

In some embodiments, the transposon is a eukaryotic transposon, optionally wherein the eukaryotic transposon is CMC, Copia, ERV, Gypsy, hAT, helitron, Zator, Sola, LINE, Tc1-Mariner, Novosib, Crypton, or EnSpm.

Further aspects of the present disclosure relate to methods of modifying a target polynucleotide sequence in a cell, comprising delivering to the cell (a) a nucleic acid encoding a Fanzor polypeptide comprising an RuvC domain; and (b) a nucleic acid encoding a fRNA molecule comprising a scaffold and a reprogrammable target spacer sequence, wherein the fRNA molecule is capable of forming a complex with the Fanzor polypeptide and directing the Fanzor polypeptide to a target polynucleotide sequence.

In some embodiments, the modifying comprises cleavage of the target polynucleotide sequence. In some embodiments, the cleavage occurs within the target polynucleotide near the 3′ end of the target polynucleotide sequence. In some embodiments, the cleavage occurs about −6 to about +3 nucleotides relative to the 3′ end of the target polynucleotide sequence.

In some embodiments, the cleavage occurs with the TAM sequence. In some embodiments, the target polynucleotide sequence is DNA.

In some embodiments, one or more mutations comprising substitutions, deletions, and insertions are introduced into the target polynucleotide sequence.

In some embodiments, (a) and (b) are delivered to the cell together. In some embodiments, (a) and (b) are delivered to the cell separately. In some embodiments, the delivering to a cell occurs (a) in vivo; (b) ex vivo; or (c) in vitro.

In some embodiments, the cell is a mammalian cell. In some embodiments, the mammalian cell is a human cell. In some embodiments, the cell is a non-mammalian, animal cell. In some embodiments, the cell is a eukaryotic cell. In some embodiments, the cell is a prokaryotic cell. In some embodiments, the cell is a plant cell. In some embodiments, the cell is a bacterial cell. In some embodiments, the cell is a fungal cell. In some embodiments, the cell is a yeast cell. In some embodiments the cell is a rodent cell. In some embodiments, the cell is a primate cell.

Further aspects of the present disclosure relate to compositions comprising a stabilized Fanzor polypeptide comprising an RuvC domain, comprising one or more mutations relative to wildtype Fanzor polypeptide wherein the mutations stabilize the Fanzor polypeptide. Further aspects of the present disclosure relate to methods of modifying a target polynucleotide sequence in a cell, comprising (a) delivering to the cell a stabilized Fanzor polypeptide comprising an RuvC domain and further comprising one or more mutations relative to a wildtype Fanzor polypeptide wherein the mutations stabilize the Fanzor polypeptide; and (b) separately delivering to the cell a fRNA molecule.

Further aspects of the present disclosure relate to method of modifying a target polynucleotide sequence in a mammal in vivo, comprising delivering to the mammal (a) a nucleic acid encoding a Fanzor polypeptide comprising an RuvC domain; and (b) a nucleic acid encoding a fRNA molecule comprising a scaffold and a reprogrammable target spacer sequence, wherein the fRNA molecule is capable of forming a complex with the Fanzor polypeptide and directing the Fanzor polypeptide to a target polynucleotide sequence.

Further aspects of the present disclosure relate to methods of modifying a target polynucleotide sequence in a mammal in vivo or in a mammalian cell ex vivo, comprising delivering to the mammal or the mammalian cell a composition of the present disclosure. In some embodiments, the mammal is a human, a primate, or a rodent, optionally a mouse; or the mammalian cell is a human cell, a primate cell, or a rodent cell, optionally a mouse cell. Further aspects of the present disclosure relate to method of modifying a target polynucleotide sequence in a plant in vivo, comprising delivering to the plant (a) a nucleic acid encoding a Fanzor polypeptide comprising an RuvC domain; and (b) a nucleic acid encoding a fRNA molecule comprising a scaffold and a reprogrammable target spacer sequence, wherein the fRNA molecule is capable of forming a complex with the Fanzor polypeptide and directing the Fanzor polypeptide to a target polynucleotide sequence.

Further aspects of the present disclosure relate to methods of modifying a target polynucleotide sequence in a plant in vivo, comprising delivering to the plant a composition of the present disclosure.

Further aspects of the present disclosure relate to method of modifying a target polynucleotide sequence in a fungi in vivo, comprising delivering to the fungi (a) a nucleic acid encoding a Fanzor polypeptide comprising an RuvC domain; and (b) a nucleic acid encoding a fRNA molecule comprising a scaffold and a reprogrammable target spacer sequence, wherein the fRNA molecule is capable of forming a complex with the Fanzor polypeptide and directing the Fanzor polypeptide to a target polynucleotide sequence.

Further aspects of the present disclosure relate to methods of modifying a target polynucleotide sequence in a fungi in vivo, comprising delivering to the fungi a composition of the present disclosure.

Further aspects of the present disclosure relate to method of modifying a target polynucleotide sequence in a virus, comprising delivering to the virus (a) a nucleic acid encoding a Fanzor polypeptide comprising an RuvC domain; and (b) a nucleic acid encoding a fRNA molecule comprising a scaffold and a reprogrammable target spacer sequence, wherein the fRNA molecule is capable of forming a complex with the Fanzor polypeptide and directing the Fanzor polypeptide to a target polynucleotide sequence.

Further aspects of the present disclosure relate to methods of modifying a target polynucleotide sequence in a virus, comprising delivering to the virus a composition of the present disclosure.

Further aspects of the present disclosure relate to method of modifying a target polynucleotide sequence in a bacteria, comprising delivering to the bacteria (a) a nucleic acid encoding a Fanzor polypeptide comprising an RuvC domain; and (b) a nucleic acid encoding a fRNA molecule comprising a scaffold and a reprogrammable target spacer sequence, wherein the fRNA molecule is capable of forming a complex with the Fanzor polypeptide and directing the Fanzor polypeptide to a target polynucleotide sequence.

Further aspects of the present disclosure relate to methods of modifying a target polynucleotide sequence in a bacteria, comprising delivering to the bacteria a composition of the present disclosure.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### BRIEF DESCRIPTION OF DRAWINGS

The following drawings form part of the present specification and are included to further demonstrate certain aspects of the present disclosure, which can be better understood by reference to one or more of these drawings in combination with the detailed description of specific embodiments presented herein. The figures are illustrative only and are not required for enablement of the invention disclosed herein.

FIGS. 1A-1F show Fanzor 2 protein associates with its non-coding RNA FIG. 1A shows phylogenetic tree of all Fanzor proteins as well as TnpB and IscB proteins. FIG. 1B shows phylogenetic tree of only Fanzor proteins with their host genome of origin shown as a ring. FIG. 1C shows schematic of the Acanthamoeba Polyphaga mimivirus (“IsvMimi Fanzor2” also referred to herein as “ApmHNuc”) system, including the Fanzor2 ORF, associated TnpA, the non-coding RNA region, and the left and right inverted repeat elements (ILR and IRR). FIG. 1D shows conservation of the three Fanzor2 loci in the Isvmimi genome, showing high conservation of the Fanzor2 protein coding regions and the nearby non-coding RNA genome. FIG. 1E shows a schematic of the method used for identifying the Isvmimi non-coding RNA. The Isvmimi protein is co-purified with its non-coding RNA, allowing for isolation of the non-coding RNA species and identification by sequencing. FIG. 1F shows RNA sequencing coverage of the Isvmimi-1 non-coding RNA region showing robust expression of the non-coding RNA and its guide sequence extending into and slightly past the IRR element. FIG. 1G shows secondary structure of the observed non-coding RNA species from FIG. 1F showing significant folding of the non-coding RNA.

FIGS. 2A-2A shows Fanzor 2 ribonucleoproteins can be programmed to cleave DNA targets in vitro. FIG. 2A shows a schematic of Isvmimi Fanzor 2 RNP purification. Isvmimi Fanzor 2 and guide are co-expressed in bacteria and harvested from collected pellet. Recombinant protein and RNA are purified via affinity tag purification and isolated via FPLC to determine RNP-containing fractions. FIG. 2B shows in vitro cleavage by Isvmimi Fanzor 2 showing dependence on targeting guide, Isvmimi Fanzor 2 protein, and magnesium. In vitro cleavage was performed with purified RNP containing either a targeting or non-targeting guide and incubated at 37C with a 7N TAM library target. FIG. 2C shows sequencing of the TAM library to determine depleted sequences revealed a distinct population of depleted TAMs (pink) compared to a non-targeting guide. FIG. 2D shows sequence motif of TAM preference computed from depleted TAMs, showing an AT-rich tam preference. FIG. 2E shows validation of the Isvmimi TAM preference via in vitro cleavage on top-depleted TAMs. In vitro cleavage of validated TAMs was performed as in FIG. 2B, with incubation with DNA target, magnesium containing buffer, and RNP containing a targeting guide. FIG. 2F shows cleavage sites of Isvmimi Fanzor 2 as mapped by Sanger sequencing show cleavage in the TAM region with multiple cut sites. Cleavage was mapped via gel extraction of cleaved bands after in vitro cleavage and Sanger sequencing with corresponding primers. Multiple cleavage positions are evident from multiple A sites added via polymerase run off. FIG. 2G shows next generation sequencing mapping of the TAM cleavage by Isvmimi Fanzor 2 via ligation. Cleavage products from in vitro cleavage reactions were prepared for sequencing via ligation of sequencing adaptors and PCR prior to sequencing on an Illumina Miseq. Reads were aligned to the TAM target to map cleavage locations.

FIGS. 3A-3F show TnpB systems with a rearranged glutamate are also active nucleases. FIG. 3A shows phylogenetic tree of Fanzor proteins, showing that Fanzor systems have a rearranged glutamate site in the RuvC catalytic domain. FIG. 3B shows Isvmimi Fanzor2 collateral activity is measured using a ssDNA fluorescent reporter, showing lack of collateral for this enzyme. FIG. 3C shows predicted AlphaFold-2 structure of Isvmimi Fanzor2, showing that despite having a rearranged glutamate in the RuvC catalytic domain, that the catalytic aspartates and glutamates still form an active site. FIG. 3D shows expression of the non-coding RNA for Thermoplasma volcanium (Istvo5) TnpB, revealing a specific non-coding RNA species that associates with the Istvo5 TnpB protein. FIG. 3E shows cleavage of the TAM library plasmid by Istvo5 TnpB, showing significant cleavage activity at 37 and 20 degrees Celsius. FIG. 3F shows DNA Cleavage of Isvmimi Fanzor2 truncated to the 65th start codon position, full length protein, catalytically dead protein (aspartate to alanine mutation), protein mutated to have a canonical glutamate in the catalytic RuvC domain, and Isvmimi full length protein. Cleavage is compared to a condition with no Fanzor protein.

FIGS. 4A-4E show Fanzor 1 proteins are active programmable nucleases. FIG. 4A shows Fanzors projected onto the eukaryotic tree of life, showing that Fanzors are present in all four kingdoms of life. FIG. 4B shows RNA sequencing of the non-coding RNA region from Fanzor1 from Chlamydomonas reinhardtii (Cre Fanzor1). Robust expression of a non-coding RNA is seen. FIG. 4C shows secondary structure of Cre Fanzor1's non-coding RNA, showing significant folding of the guide RNA. FIG. 4D shows TAM library DNA Cleavage by Cre Fanzor1, revealing RNA guided DNA targeting. FIG. 4E shows sequence motif of TAM preference computed from depleted TAMs.

FIGS. 5A-5A show Fanzor nucleases can be programmed to target DNA in mammalian cells for genome editing FIG. 5A shows secondary structures of modified guide RNA for Isvmimi Fanzor 2 engineered for expression off of Polymerase III promoters. Guide RNAs are modified to remove poly U tracts that would lead to premature termination. FIG. 5B shows schematic of delivery and testing of Isvmimi Fanzor 2 in mammalian cells.

FIGS. 6A-6H show Fanzor nucleases associate with their non-coding RNA. FIG. 6A shows a phylogenetic tree of representative Fanzor and TnpB proteins with the host genome kingdom and Fanzor family designation. For TnpBs, Fanzor family designation corresponds to the Fanzor family that the TnpB is most similar too by sequence alignment. Fanzor and TnpB orthologs experimentally studied in this work are labeled. FIG. 6B shows a phylogenetic tree of only Fanzor proteins with the phyla of their host species and predicted associated transposons marked as rings. Family and kingdom correspond to those in FIG. 6A. FIG. 6C shows a comparison of predicted ncRNA lengths at the 5′ end of MGE of IscB, TnpB and Fanzor systems (****, p<0.0001, one way ANOVA). FIG. 6D shows a comparison of predicted ncRNA lengths at the 3′ end of MGE of IscB, TnpB and Fanzor systems (****, p<0.0001, one way ANOVA). FIG. 6E shows a schematic of the Acanthamoeba Polyphaga mimivirus (ApmHNuc Fanzor) system, including the Fanzor ORF, associated IS607 TnpA, the non-coding RNA region, and the left and right inverted repeat elements (ILR and IRR). FIG. 6F shows conservation of the three Fanzor loci in the Acanthamoeba Polyphaga mimivirus genome, showing high conservation of the Fanzor protein-coding regions and the nearby non-coding RNA. FIG. 6G shows secondary structure of the observed non-coding RNA species from FIG. 6F, showing significant folding of the non-coding RNA. FIG. 6H shows conserved secondary structure of ApmHNuc Fanzor's non-coding RNA with its most similar Fanzor systems.

FIGS. 7A-7H show Fanzor ribonucleoproteins can be programmed to cleave DNA targets in vitro. FIG. 7A shows a schematic of the method used for identifying the ApmHNuc associated non-coding RNA. The ApmHNuc protein is co-purified with its non-coding RNA, allowing for the isolation of the non-coding RNA species and identification by small RNA sequencing. FIG. 7B shows RNA sequencing coverage of the ApmHNuc-1 non-coding RNA region showing robust expression of the non-coding RNA and its guide sequence extending past the IRR element. FIG. 7C shows scatter plots of the fold change of individual TAM sequences in a 7N library plasmid relative to input plasmid library distribution with either ApmHNuc RNP with a targeting fRNA or a non-targeting fRNA. FIG. 7D shows sequence motif of TAM preference computed from depleted TAMs, showing an NGGG-rich tam preference. FIG. 7E shows biochemical validation of individual ApmHNuc TAM sequences including 4 preferred TAMs (TGGG, AGGG, CGGG, and GGGG) as well as 3 non-TAM sequences and 1 non-targeting sequence. ApmHNuc RNP is incubated with DNA targets containing each of these sequences and cleavage is visualized by gel electrophoresis. FIG. 7F shows ApmHNuc RNP purified with either targeting (T) or non-targeting (NT) fRNA as well as two catalytic dead ApmHNuc mutants (D324A and E467A) are tested on either a plasmid containing the correct target spacer DNA sequences or a scrambled DNA sequence containing the 5′ TAM TGGG. EDTA is added in lane 5 to quench the cleavage by chelating ions inside the reaction. FIG. 7G shows Sanger sequencing traces of ApmHNuc RNP cleavage on the 5′ CGGG TAM target, showing cleavage downstream of the guide target. FIG. 7H shows next-generation sequencing mapping of the TAM cleavage by ApmHNuc Fanzor via NEB adaptor ligation. Cleavage products from in vitro cleavage reactions were prepared for sequencing via ligation of sequencing adaptors and PCR prior to next-generation sequencing. Reads were aligned to the TAM target to map cleavage locations. Two separate reactions were ran in parallel with and without addition of ApmHNuc RNP. The cleavage products were amplified in both 5′ and 3′ directions with F denoting 3′ direction and R denoting the 5′ direction.

FIGS. 8A-8I show TnpB systems with rearranged glutamates are also active nucleases. A FIG. 8A shows alignment of the split RuvC domains of Fanzor and TnpB nucleases showing the rearranged glutamic acid inside RuvC-II versus the canonical glutamic acid. FIG. 8B shows phylogenetic tree of TnpB and Fanzor proteins, showing which TnpBs and Fanzor nucleases have a rearranged glutamic acid site. FIG. 8C shows predicted AlphaFold-2 structure of ApmHNuc, TvoTnpB, Isdra2TnpB, and Uncas12f, showing that despite having a rearranged glutamate in the RuvC catalytic domain, the catalytic aspartates and glutamates still form an active catalytic triad (red residues). FIG. 8D shows schematic of the Thermoplasma volcanium GSS1TnpB (TvoTnpB) system, including the alternatively rearranged TnpB, associated IS605 TnpA, and the left and right end elements (LE and RE). FIG. 8E shows expression of the non-coding RNA for TvoTnpB, revealing a specific non-coding RNA species that associates with the TvoTnpB protein extending from the ORF to outside the RE element similar to Isdra2TnpB. FIG. 8F shows sequence logo motif of TAM preference by TvoTnpB. FIG. 8G shows biochemical validation of individual TAM preference by TvoTnpB showing that the cleavage by TvoTnpB is TAM (NTGAC) specific. TvoTnpB RNP is incubated with targets containing different 5′ TAMs and cleavage is visualized by gel electrophoresis. FIG. 8H shows next-generation sequencing mapping of the TAM cleavage by TvoTnpB via adaptor ligation. Reads were aligned to the TAM target to map cleavage locations. Two separate reactions were ran in parallel with and without addition of TvoTnpB RNP. The cleavage products were amplified in both 5′ and 3′ directions with F denoting 3′ direction and R denoting the 5′ direction. FIG. 8I shows ApmHNuc, TvoTnpB, and Isdra2TnpB DNA collateral cleavage activity are measured using an ssDNA fluorescent reporter, showing a lack of collateral activity for nucleases with the rearranged glutamic acid in RuvC-II. DNase I is used as a positive nuclease control for collateral cleavage activity.

FIGS. 9A-9G show Fanzor are widespread in the eukaryotic genome and associates with their fRNA. FIG. 9A shows Fanzor systems projected onto the eukaryotic tree of life. Nodes and tips of the tree are marked with circles if there are Fanzor in the corresponding taxonomic group. Circle sizes are proportional to the Fanzor copy number and shown by family. FIG. 9B shows phylogenetic tree of Fanzor sequences for which splicing prediction was available. The outer ring shows intron density of the corresponding Fanzor nucleases. FIG. 9C shows schematic of the Chlamydomonas reinhardtii Fanzor system, including the 5′ asymmetrical terminal inverted repeats (ATIR), 3′ ATIR, 5′ target site duplications (TSD), 3′ TSD, and the mRNA and coding sequences for Cre-1 Fanzor. FIG. 9D shows small RNA sequencing of Chlamydomonas reinhardtii showing expression of noncoding RNA at the 3′ end of the CreHNuc that extends beyond the ATIR into the TSD. FIG. 9E shows alignment of all 6 copies of Cre Fanzor inside the annotated part of Chlamydomonas reinhardtii genome, showing highly conserved 3′ ends of the Cre Fanzor proteins along with its fRNA and variable 5′ end composition of the proteins. FIG. 9F shows secondary structure of CreHNuc-1 Fanzor′ non-coding RNA from 4D-E, showing significant folding of the guide RNA. FIG. 9G shows conserved secondary structure of CreHNuc-1 Fanzor's non-coding RNA and its most similar Fanzor systems.

FIGS. 10A-10F show Fanzor nucleases encode natural nuclear localization signals (NLS) and have mammalian genome editing activity. FIG. 10A shows protein schematic of ApmHNuc Fanzor showing the core catalytic triads of split RuvC domain and the predicted N-terminal nuclear localization signal (NLS). The N-terminal NLS like element is shown and the catalytic triad is shown as the space filling residues inside the RuvC domain on the AF2 predicted ApmHNuc structure. FIG. 10B shows phylogenetic tree of Fanzor proteins showing which sequences have predicted NLS elements within 15 residues of their N-terminal or C-terminal ends. The phyla and families of the sequences are also marked as rings. FIG. 10C shows confocal images of a regular sfGFP, the predicted ApmHNuc NLS fused to sfGFP on either the N-terminal or C-terminal end, and sfGFP fused directly to the N-terminal of ApmHNuc transfected into HEK293FT cells and stained with SYTO Red nuclear stain. Images include the nuclear stain, GFP signal, and a merged image. FIG. 10D shows an ApmHNuc mammalian expression vector and fRNA expression plasmid are co-transfected into HEK293FT cells targeting a luciferase reporter where a Cypridina luciferase (Cluc) is driven by a constitutive promoter and a Gaussia luciferase (Gluc) is placed out of frame from the native start codon. ApmHNuc with a targeting guide against the reporter shows a significantly higher normalized luciferase signal than a non-targeting guide (***, p<0.001, two-sided t-test). FIG. 10E shows indel frequency on the luciferase reporter is measured by next-generation sequencing. The targeting guide with either wild type ApmHNuc fRNA scaffold or T to C mutant scaffold to boost expression is compared against a non-targeting guide. Both scaffolds show a significant increase in indel frequency compared to the non-targeting guide (***, p<0.001, **, p<0.01, one-way ANOVA). FIG. 10F shows representative indel alleles from the targeting guide condition on the luciferase reporter, showing deletions centered around the 3′ end of the guide target.

FIGS. 11A-11D show genomic characteristics of Fanzor family members. FIG. 11A shows a histogram of the copy number of individual Fanzor members inside their respective genomes. FIG. 11B shows frequency of predicted associated transposons nearby Fanzor (within +/−10 kb) per transposon family type. FIG. 11C shows frequency of the top occurring nearby protein domains within 5 genes upstream or downstream of the Fanzor MGE. FIG. 11D shows phylogenetic tree of Fanzor with the positions of the known Fanzor proteins marked. Phylum and Fanzor family information are also marked as rings.

FIGS. 12A-12C show purification of ApmHNuc. FIG. 12A shows protein gel showing flow through and eluant of AmpHNuc products during gravity flow strep-bead purifications prior to loading of FPLC. The square denotes the desired protein product. FIG. 12B shows FPLC traces of ApmHNuc purified with its fRNA and protein gels showing each fraction's protein products with the desired protein product that was pooled labeled with squares. FIG. 12C FPLC traces of AmpHNuc purified without its fRNA and protein gels showing no RNP product in all observed fractions.

FIGS. 13A-13D show characterization of ApmHNuc nuclease activity. FIG. 13A shows alignment of ApmHNuc Ruvc domain with Isdra2TnpB RuvC domain to nominate the catalytic RuvC-I aspartic acid (D324) and the RuvC-II glutamic acid (E467A). FIG. 13B shows FPLC traces of ApmHNuc E467A mutant purified with its fRNA and protein gels showing each fraction's protein products with the desired protein product that was pooled shown with a square. FIG. 13C shows FPLC traces of ApmHNuc D324A mutant purified with its fRNA and protein gels showing each fraction's protein products with the desired protein product that was pooled shown with a square. FIG. 13D shows native TBE gel showing nuclease activity of AmpHNuc at temperatures from 10 to 65 degrees Celsius. Reactions were carried out by incubating wild-type ApmHNuc RNP on a plasmid with the TGGG TAM 5′ adjacent to the 21 nt spacer target. Cleavage was visualized by gel electrophoresis.

FIGS. 14A-14C show purification of Isdra2TnpB and TvoTnpB. FIG. 14A shows protein gel showing flow through and eluant fractions of Isdra2TnpB and TvoTnpB products during gravity flow strep-bead purifications. The desired protein product is shown via a square. FIG. 14B shows FPLC traces of TvoTnpB purified with its ωRNA and protein gels showing each fraction's protein products with the desired protein product that was pooled shown with a square. FIG. 14C shows FPLC traces of Isdra2TnpB purified without its ωRNA and protein gels showing each fraction's protein products with the desired protein product that was pooled shown with a square.

FIGS. 15A-15C show biochemical characterization of TvoTnpB. FIG. 15A shows TvoTnpB DNA cleavage of a 21 nt target containing a 5′ ATGAC TAM at temperatures ranging from 30 degrees Celsius to 90 degrees Celsius, showing optimal cleavage reaction temperature near 60 degrees for TvoTnpB. FIG. 15B shows Sanger sequencing traces of TvoTnpB cleavage on a 5′ CTGAC TAM target, showing cleavage at the end of the target. FIG. 15C shows fluorescent signal from RNase alert reporter detection of RNA collateral cleavage activity from RNase A, TvoTnpB, Isdra2TnpB, and ApmHNuc incubated with their target DNA sequences for 1 hour. The signal is normalized to a no DNA target condition.

FIGS. 16A-16C show intron characterization of Fanzor systems. FIG. 16A shows a comparison of the number of predicted introns in Fanzor genes and the mean number of introns per gene in the host genome. Number of introns was defined as the number of exons minus one and calculated from the annotations for the genome provided by GenBank. Correlation and significance values are shown as an inset. FIG. 16B shows a comparison of the mean number of introns in Fanzor genes in a genome and the mean number of introns per gene in the host genome. Correlation and significance values are shown as an inset. FIG. 16C shows standard deviation of the number of introns per Fanzor genes in clusters of 70% sequence identity and 95% alignment coverage. Only sequences with available splicing predictions were clustered and only clusters of two or more sequences are shown.

FIGS. 17A-17D show characterization of the CreHNuc fRNAs. FIG. 17A shows small RNA sequencing traces mapped onto all 6 copies of full CreHNuc systems in the Cre genome. FIG. 17B shows alignment of the 26 full or partial copies of CreHNuc MGEs inside the Cre genome at their 3′ end. FIG. 17C shows FPLC traces of CreHNuc purified either with or without its fRNA, showing the RNP complex is only stable with the correct fRNA present. The CreHNuc peak in the FPLC trace is labeled. FIG. 17D shows protein gel showing elution fractions of the CreHNuc with the desired protein product that was pooled labeled with a square.

FIG. 18 shows ApmHNuc nuclear localization signal characterization. Probability distribution of potential NLS elements across the ApmHNuc protein sequence as predicted by NLStradamus. The default cutoff at 0.6 is used to call significant NLS like elements, revealing one N-terminal NLS and one internal NLS.

FIGS. 19A-A1 show evolution of Fanzor nucleases and their association with non-coding fRNAs. FIG. 19A shows phylogenetic tree of representative Fanzor and TnpB proteins. From the inner ring outward, the rings show protein system, Fanzor family designation, host superkingdom, phyla of their host species predicted associated transposons, and protein length. Several Fanzor and TnpB proteins studied in this work are marked around the tree. Splits with bootstrap support less than 0.7 out of 1 were collapsed and the tree was rooted at the midpoint. FIG. 19B shows Fanzor systems projected onto the evolutionary tree of eukaryotes (Rees et al. 2017). Nodes and tips of the tree are marked with circles if there are Fanzors in the corresponding taxonomic group. Circle sizes are proportional to the Fanzor copy number and shown by family. FIG. 19C shows comparison of protein lengths (aa) between Fanzor nucleases and TnpB nucleases (****, p<0.0001, two side t-test). FIG. 19D shows intron density of Fanzor genes grouped by assigned families. Statistical tests measured each family's intron density distribution against the rest of the families via a two-sided Student's t-test with multiple hypothesis correction (****, p<0.0001; ***, p<0.001). FIG. 19E shows intron density of Fanzors grouped by taxonomic kingdom. Statistical tests measured each kingdom's intron density distribution against the rest of the kingdoms via a two-sided Student's t-test with multiple hypothesis correction (****, p<0.0001). FIG. 19F shows comparison of predicted flanking non-coding conservation lengths at the 5′ end and 3′ end of the MGEs of IscB, TnpB and Fanzor systems (****, p<0.0001, one way ANOVA). FIG. 19G Schematic of the Acanthamoeba Polyphaga mimivirus (ApmFNuc) system, including the Fanzor ORF, associated IS607 TnpA, the non-coding RNA region, and the left and right inverted repeat elements (ILR and IRR). The WED, RuvC, and REC domain is annotated based on structural similarity with the Isdra2 TnpB structure (Nakagawa et al. 2023). FIG. 19H shows conservation of the three Fanzor loci in the Acanthamoeba Polyphaga mimivirus genome, showing high conservation of the Fanzor protein-coding regions and the nearby non-coding regions. FIG. 19I shows putative RNA secondary structure of the conserved 3′ non-coding region from FIG. 19H, showing strong folding and structural elements of this putative non-coding RNA.

FIGS. 20A-20G shows viral Fanzor ribonucleoproteins can be programmed to cleave DNA targets in vitro. FIG. 20A shows a schematic of the method used for identifying the ApmFNuc associated non-coding RNA. The ApmFNuc protein is co-purified with its non-coding RNA, allowing for the isolation of the non-coding RNA species and identification by small RNA sequencing. FIG. 20B shows RNA sequencing coverage of the ApmFNuc-1 non-coding RNA region showing robust expression of the non-coding RNA and its guide sequence extending past the IRR element. FIG. 20C shows scatter plots of the fold change of individual TAM sequences in a 7N library plasmid relative to input plasmid library distribution with either ApmFNuc RNP with a targeting fRNA or a non-targeting fRNA. FIG. 20D shows sequence motif of TAM preference computed from depleted TAMs, showing an NGGG-rich tam preference. FIG. 20E shows biochemical validation of individual ApmFNuc TAM sequences including 4 preferred TAMs (TGGG, AGGG, CGGG, and GGGG) as well as 3 non-TAM sequences and 1 non-targeting sequence. ApmFNuc RNP is incubated with DNA targets containing each of these sequences and cleavage is visualized by gel electrophoresis on 6% TBE gel. FIG. 20F shows Sanger sequencing traces of ApmFNuc RNP cleavage on the 5′ CGGG TAM target, showing cleavage downstream of the guide target. FIG. 20G shows next-generation sequencing mapping of the TAM cleavage by ApmFNuc via NEB adaptor ligation. Cleavage products from in vitro cleavage reactions were prepared for sequencing via ligation of sequencing adaptors and PCR prior to next-generation sequencing. Reads were aligned to the TAM target to map cleavage locations. Two separate reactions were ran in parallel with and without addition of ApmFNuc RNP. The cleavage products were amplified in both 5′ and 3′ directions with F denoting 3′ direction and R denoting the 5′ direction.

FIGS. 21A-21R shows eukaryotic Fanzor orthologs are widespread across eukaryotic kingdoms, associate with fRNAs, and are RNA-guided nucleases. FIG. 21A shows locus schematics of four eukaryotic Fanzor systems from Mercenaria mercenaria, Dreseinna polymorpha, Batillaria attramentaria, and Klebsormidium nitens. WED, REC, and RuvC domains are identified by sequence and structural alignment with Isdra2 TnpB (Nakagawa et al. 2023). FIG. 21B shows a schematic of screening for fRNA expression, TAM, activity, and cleavage locations via cell-free transcription/translation. FIG. 21C shows small RNA sequencing of the MmFNuc locus showing expression of a non-coding RNA species extending outside the ORF. FIG. 21D shows small RNA sequencing of the DpFNuc locus showing expression of a non-coding RNA species extending outside the ORF. FIG. 21E shows small RNA sequencing of the BaFNuc locus showing expression of a non-coding RNA species extending outside the ORF. FIG. 21F shows small RNA sequencing of the KnFNuc locus showing expression of a non-coding RNA species extending outside the ORF. FIG. 21G shows Weblogo visualization of the TAM sequence preference of MmFNuc identified by adaptor ligation assay on a 7N TAM library incubated with MmFNuc protein and fRNA. FIG. 21H shows Weblogo visualization of the TAM sequence preference of DpFNuc identified by adaptor ligation assay on a 7N TAM library incubated with DpFNuc protein and fRNA. FIG. 21I shows Weblogo visualization of the TAM sequence preference of BaFNuc identified by adaptor ligation assay on a 7N TAM library incubated with BaFNuc protein and fRNA. FIG. 21J shows Weblogo visualization of the TAM sequence preference of KnFNuc identified by adaptor ligation assay on a 7N TAM library incubated with KnFNuc protein and fRNA. FIG. 21K shows validation of MmFNuc cleavage by incubating the MmFNuc RNP with its correct TTTA TAM, four mutated TAMs, and a non-targeted plasmid. FIG. 21L shows validation of DpFNuc cleavage by incubating the DpFNuc RNP with its correct TTTA TAM, four mutated TAMs, and a non-targeted plasmid. FIG. 21M shows validation of BaFNuc cleavage by incubating the BaFNuc RNP with its correct TTTA TAM, four mutated TAMs, and a non-targeted plasmid. FIG. 21N shows validation of KnFNuc cleavage by incubating the KnFNuc RNP with its correct TTTA TAM, four mutated TAMs, and a non-targeted plasmid. FIGS. 21O-21R shows next-generation sequencing mapping of the cleavage positions by MmFNuc, DpFNuc, and BaFNuc via NEB adaptor ligation of cleaved DNA targets that were incubated with the respective RNP complexes. Cleavage products from in vitro cleavage reactions were prepared for sequencing via ligation of sequencing adaptors and PCR prior to next-generation sequencing. Reactions were performed with and without addition of each Fanzor RNP. The cleavage products were amplified in both 5′ and 3′ directions with F denoting 3′ direction (top panel) and R denoting the 5′ direction (bottom panel).

FIGS. 22A-22H shows re-arranged RuvC catalytic residues enable Fanzor TnpB on-target cleavage without collateral activity. FIG. 22A shows alignment of the RuvC domains of Fanzor and TnpB nucleases (TnpB2) showing the alternative glutamate in RuvC-II versus the canonical glutamate that is typically observed in TnpB nucleases (TnpB1). FIG. 22B shows a phylogenetic tree of TnpB and Fanzor proteins, showing TnpBs and Fanzor nucleases with rearranged catalytic sites. FIG. 22C shows predicted AlphaFold-2 structure of ApmFNuc and TvTnpB compared with the solved structures of Isdra2TnpB, and Uncas12f, showing that despite having a rearranged glutamate in the RuvC catalytic domain, the catalytic aspartates and glutamates form a putative active catalytic triad. Domains identified are highlighted and the disordered N-terminal region is shown. FIG. 22D shows ApmFNuc RNP purified with either targeting (T) or non-targeting (NT) fRNAs as well as two catalytic dead ApmFNuc mutants (D324A and E467A) are tested on either a plasmid containing the correct target spacer DNA sequences or a scrambled DNA sequence containing the 5′ TAM TGGG. EDTA is added in lane 5 to quench the cleavage reaction. FIG. 22E shows a schematic of the Thermoplasma volcanium GSS1TnpB (TvTnpB) system, including the TnpB with a rearranged catalytic site, associated IS605 TnpA, and the left and right end elements (LE and RE). FIG. 22F shows a sequence logo of the TAM for TvTnpB. FIG. 22G shows biochemical validation of individual TAM preference by TvTnpB showing that the cleavage by TvTnpB is TAM (NTGAC) specific. TvTnpB RNP Is incubated with targets containing different 5′ TAMs and cleavage is visualized by gel electrophoresis. FIG. 22H shows ApmFNuc, TvTnpB, MmFNuc, DpFNuc, BaFNuc and Isdra2TnpB DNA collateral cleavage activity are measured using an ssDNA fluorescent reporter, showing a lack of collateral activity for nucleases with the rearranged glutamic acid in RuvC-II. DNase I is used as a positive nuclease control for collateral cleavage activity.

FIGS. 23A-23J show Fanzor nucleases contain nuclear localization signals (NLS) and have mammalian genome editing activity. FIG. 23A shows a schematic of ApmFNuc showing the split RuvC domain and the predicted N-terminal nuclear localization signal (NLS). NLS and the catalytic triad are shown as space filling residues inside the RuvC domain on the AF2 predicted ApmFNuc structure. FIG. 23B shows confocal images of unmodified super-folder GFP (sfGFP), the predicted ApmFNuc NLS fused to sfGFP on either the N-terminal or C-terminal end, and sfGFP fused directly to the N-terminus of ApmFNuc transfected into HEK293FT cells and stained with SYTO Red nuclear stain. Images display the nuclear stain, GFP signal, and a merged image. Scale bar, 10 μm. FIG. 23C shows a quantitative analysis of 22 predicted Fanzor NLS sequences. Putative NLS sequences are fused to the N-terminus of sfGFP and the nuclear to cytoplasmic ratio of GFP fluorescence is quantitated (n=3, *, p<0.01; one-way ANOVA with false-discovery rate correction). FIG. 23D shows a schematic of Fanzor nucleases adapted for genome editing in mammalian cells. FIG. 23E shows the indel formation rates generated by MmFNuc across 7 selected endogenous loci. For each locus, two fRNA guide sequences were tested and a non-targeting guide is used as a negative control. FIG. 23F shows the indel formation rates generated by DpFNuc across 7 selected endogenous loci. For each locus, two fRNA guide sequences were tested and a non-targeting guide is used as a negative control. FIG. 23G shows insertion and deletion rates at each base inside the quantification window generated by MmFNuc at the CXCR4 genomic locus. FIG. 23H shows insertion and deletion rates at each base inside the quantification window generated by DpFNuc at the GRIN2b genomic locus. FIG. 23I shows representative indel reads formed by MmFNuc at the CXCR4 genomic locus. FIG. 23J shows representative indel reads formed by DpFNuc at the GRIN2b genomic locus.

FIGS. 24A-24D show genomic characteristics of Fanzor family members. FIG. 24A shows a histogram of the copy number of individual Fanzor members inside their respective genomes. FIG. 24B shows a phylogenetic tree of Fanzors and TnpBs with the domain predictions of nearby proteins marked as a ring (the nearest 5 genes downstream and upstream). Previously discovered Fanzors are marked in the outer ring (Bao et al. 2013). FIG. 24C shows alignment of FanzorI proteins with closely related TnpBs. FIG. 24D shows alignment of Fanzor 2 proteins with closely related TnpBs.

FIGS. 25A-25D show Fanzor intron characterization. FIG. 25A shows a phylogenetic tree of Fanzors and TnpBs with rings to show the host superkingdom, phylum, and intron density of the Fanzor proteins. FIG. 25B shows a scatterplot of the intron density of the Fanzor proteins along with the mean intron density of their host genomes. Fanzor proteins are shown according to their family designations. FIG. 25C shows a scatterplot of the mean intron densities of the Fanzor proteins in a genome along with the mean intron density of their host genomes. FIG. 25D shows a histogram of the standard deviation of intron densities within 70% similarity clusters of Fanzor proteins.

FIGS. 26A-26G show locus characteristics of Fanzor family members. FIG. 26A shows the frequency of predicted associated transposons nearby Fanzor (within +/−10 kb) per transposon family type. FIG. 26B shows the frequency of the top occurring nearby protein domains within 5 genes upstream or downstream of the Fanzor MGE. FIG. 26C shows locus schematics of different Fanzor1 nucleases and their associated transposons. IRL marks the left inverted repeat and IRR marks the right inverted repeat. FIG. 26D shows locus schematics of different Fanzor2 nucleases and their associated transposons. FIG. 26E shows a comparison of predicted flanking non-coding conservation lengths at the 5′ end of the MGEs of IscB, TnpB, and each Fanzor family. FIG. 26F shows a comparison of predicting flanking non-coding conservation lengths at the 3′ end of the MGEs of IscB, TnpB, and each Fanzor family. FIG. 26G shows the conserved secondary structure of fRNAs between the different copies of the ApmFNuc family. The area corresponds to conserved sequence not present in the mature fRNA, potentially removed by RNase processing (cut site designated by the triangle). FIGS. 27A-27C show purification of ApmFNuc RNPs. FIG. 27A shows a protein gel of flowthrough and eluent of ApmFNuc products during gravity flow strep-bead purifications prior to loading of FPLC. Square denotes the desired protein product. FIG. 27B shows FPLC traces of ApmFNuc purified with its fRNA and protein gels showing each fraction's protein products with the desired protein product that was pooled labeled with squares. FIG. 27C shows FPLC traces of ApmFNuc purified without its fRNA and protein gels showing no RNP product in all observed fractions.

FIGS. 28A-28B shows characterization of eukaryotic Fanzor nucleases. FIG. 28A shows alignment and domain annotation of three eukaryotic Fanzor nucleases (DpFNuc, MmFNuc, and BaFNuc). RE and LE elements are determined by conservation dropoff between alignments of different copies in the genome. FIG. 28B shows secondary structure prediction of fRNAs associated with DpFNuc, MmFNuc, and BaFNuc determined by small RNA sequencing of the locus. Shaded regions denotes stem loops and multi-stem loops region in the fRNAs. FIGS. 29A-29I shows characterization of Cr-1FNuc and its fRNA. FIG. 29A shows a schematic of the Chlamydomonas reinhardtii Fanzor1 system (Cr-1FNuc), including the 5′ asymmetrical terminal inverted repeats (ATIR), 3′ ATIR, 5′ target site duplications (TSD), 3′ TSD, and the mRNA and coding sequences for Cr-1FNuc. The mRNA track shows the processed mRNA transcripts relative to the genome and the CDS track shows the ORF coding sequences relative to the genome. FIG. 29B shows alignment of all six copies of Fanzor systems inside the annotated parts of the C. reinhardtii genome showing highly conserved 3′ ends of the CrFNuc proteins along with their fRNAs and variable 5′ end compositions of the proteins. The track shows the processed mRNA transcripts relative to the genome and the track shows the ORF coding sequences relative to the genome. FIG. 29C shows small RNA sequencing traces mapped onto all 6 copies of RuvC-containing Fanzor systems in the C. reinhardtii genome. FIG. 29D shows small RNA sequencing of the Chlamydomonsa reinhardtii organism showing expression of a noncoding RNA species at the 3′ end of the Cr-1FNuc locus that extends beyond the ATIR into the TSD. FIG. 29E shows secondary structure of Cr-1FNuc non-coding RNA from FIG. 21J, showing significant folding of the fRNA. FIG. 29F shows conserved secondary structure of the six CrFNuc fRNA copies in the genome. FIG. 29G shows alignment of the 26 full or partial copies of Fanzor MGEs inside the C. reinhardtii genome at their 3′ ends. FIG. 29H shows FPLC traces of Cr-1FNuc purified either with or without its fRNA, showing that the RNP complex is only stable when the correct fRNA is expressed and present. The Cr-1FNuc peak in the FPLC trace is labeled. FIG. 29I shows a protein gel of elution fractions of the Cr-1FNuc with the desired protein product that was pooled labeled with a square.

FIGS. 30A-30G show further characterization of ApmFNuc nuclease activity. FIG. 30A shows predicted AlphaFold-2 structures of MmFNuc, DpFNuc, and BaFNuc showing that despite having a rearranged glutamate in the RuvC catalytic domain, the catalytic aspartates and glutamates form a putative active catalytic triad (resides). FIG. 30B shows alignment of ApmFNuc RuvC domain with Isdra2TnpB RuvC domain to nominate the catalytic RuvC-I aspartic acid (D324) and the RuvC-II glutamic acid (E467A). FIG. 30C shows FPLC traces of ApmFNuc E467A mutant purified with its fRNA and protein gels showing each fraction's protein products with the desired protein product that was pooled shown with a square. FIG. 30D shows FPLC traces of ApmFNuc D324A mutant purified with its fRNA and protein gels showing each fraction's protein products with the desired protein product that was pooled shown with a red square. FIG. 30E shows native TBE gel of nuclease activity of ApmFNuc at temperatures from 10 to 65 degrees Celsius. Reactions were carried out by incubating wild-type ApmFNuc RNP on a plasmid with the TGGG TAM 5′ adjacent to the 21 nt spacer target. Cleavage was visualized by gel electrophoresis. FIG. 30F shows a native TBE gel showing nuclease activity of ApmFNuc with different cations supplemented into the cleavage buffer. Reactions were carried out by incubating wild-type ApmFNuc RNP on a plasmid with the TGGG TAM 5′ adjacent to the 21 nt spacer target. Cleavage was visualized by gel electrophoresis. FIG. 30G shows a native TBE gel showing nuclease activity of ApmFNuc with different NaCl salt concentrations supplemented into the cleavage reaction buffer. Reactions were carried out by incubating wild-type ApmFNuc RNP on a plasmid with the TGGG TAM 5′ adjacent to the 21 nt spacer target. Cleavage was visualized by gel electrophoresis.

FIGS. 31A-31C show purification of Isdra2TnpB and TbTnpB. FIG. 31A shows a protein gel showing flowthrough and eluent fractions of Isdra2TnpB and TbTnpB products during gravity flow strep-bead purifications. The desired protein product is shown via a square. FIG. 31B shows FPLC traces of TvTnpB purified with its ωRNA and protein gels showing each fraction's protein products with the desired protein product that was pooled shown with a square. FIG. 31C shows FPLC traces of Isdra2TnpB purified without its ωRNA and protein gels showing each fraction's protein products with the desired protein product that was pooled shown with a square.

FIGS. 32A-32F show characterization of TvTnpB and collateral activity comparisons. FIG. 32A shows expression of the non-coding RNA for TvTnpB, revealing a specific non-coding RNA species that associates with the TvTnpB protein extending from the ORF to outside the RE element similar to Isdra2TnpB. FIG. 32B shows TvTnpB DNA cleavage of a 21 nt target containing a 5′ ATGAC TAM at temperatures ranging from 30 degrees Celsius to 90 degrees Celsius, showing optimal cleavage reaction temperature near 50 degrees for TvTnpB. FIG. 32C shows next-generation sequencing mapping of the TAMP cleavage by TvTnpB via adaptor ligation. Reads were aligned to the TAM target to map cleavage locations. Two separate reactions were ran in parallel with and without addition of TvTnpB RNP. The cleavage products were amplified in both 5′ and 3′ directions with F denoting 3′ direction and R denoting the 5′ direction. FIG. 32D shows Sanger sequencing traces of TvTnpB cleavage on a 5′ CTGAC TAM target, showing cleavage at the end of the target. FIG. 32E shows on target cleavage activity of TvTnpB, Isdra2TnpB, MmFNuc, BaFNuc, DpFNuc, and ApmFNuc. Nucleases were incubated with plasmids containing their preferred TAM site and on-target guide RNA sequences for 1 hour of cleavage and subsequently visualized on a native TBE gel for comparison of on-target cleavage activity. FIG. 32F shows fluorescent signal from RNase alert reporter detection of RNA collateral cleavage activity from RNase A, TvTnpB, Isdra2TnpB, MmFNuc, BaFNuc, DpFNuc, and ApmFNuc incubated with their target DNA sequences for 1 hour. The signal is normalized to a no DNA target condition.

FIGS. 33A-33E show characterization of Fanzor nuclear localization signals. FIG. 33A shows a probability distribution of potential NLS elements across the ApmFNuc protein sequence as predicted by NLStradamus (Nguyen Ba et al. 2009). The default cutoff at 0.6 is used to call significant NLS like elements, revealing one N-terminal NLS and one internal NLS. FIG. 33B shows a phylogenetic tree of Fanzor nucleases and TnpB orthologs, with rings marking the host phyla and family designations of the Fanzor orthologs and which proteins were predicted to have an NLS sequences. FIG. 33C shows a bar plot depicting NLS predictions rates on a set of known human cytosolic proteins (negative control), a set of known NLS containing proteins (positive control), and all Fanzor nucleases. FIG. 33D shows per family breakdown of NLS containing Fanzor predictions for Fanzor families 1-5. FIG. 33E shows confocal images of 22 different Fanzor nuclease N-terminal NLS predictions fused to sfGFP and transfected into HEK293FT cells for visualization of nuclear localization of the sfGFP. DAPI is sued to stain the nucleus and images are shown with the GFP and DAPI channel signals merged. Scale bar, 20 μm.

FIGS. 34A-34D show a schematic of engineered fRNA scaffolds for mammalian genome editing. fRNA secondary structures are predicted by viennaRNA fold for FIG. 34A ApmFNuc, FIG. 34B BaFNuc, FIG. 34C DpFNuc, and FIG. 34D MmFNuc. Mutated residues are labeled and the arrows pointing to each base denote the nucleic acid mutations introduced at the specific position.

FIGS. 35A-35F show characterization of Fanzor nuclease plasmid reporter editing in HEK293FT cells. FIG. 35A shows an ApmFNuc mammalian expression vector and its fRNA U6 expression plasmid are co-transfected into HEK293FT cells targeting a luciferase plasmid reporter. Different mutations on the wild-type fRNA scaffold are introduced as shown in FIGS. 34A-34D to eliminate poly-U stretches in the fRNA. Indel frequency is measured by next-generation sequencing with targeted primers on the plasmid reporter. FIG. 35B shows representative indel alleles from the M2+M5 scaffold targeting guide condition on the luceriferase reporter, showing deletions centered around the 3′ end of the guide target. FIG. 35C show indel frequency on the luciferase plasmid reporter for BaFNuc, MmFNuc, and DpFNuc with different engineered fRNA scaffolds. FIG. 35D shows representative indel alleles for MmFNuc with the M1 fRNA scaffold targeting the luciferase reporter plasmid, showing deletions centered around the 3′ end of the guide target. FIG. 35E shows quantification of insertion, deletion, and combined indel frequencies generated on the plasmid reporter by DpFNuc with the (M1+M3) scaffold targeting guide condition. Rates are shown per base throughout the quantification window of the amplicon. FIG. 35F shows quantification of insertion, deletion and combined indel frequencies generated on the plasmid reporter by MmFNuc with the targeting guide condition. Rates are shown per base throughout the quantification window of the amplicon.

FIGS. 36A-36C show characterization of KnFNuc Fanzor1 nuclease genomic editing in HEK293FT cells. FIG. 36A shows a KnFNuc mammalian expression vector and its fRNA U6 expression plasmid are cotransfected into HEK293FT cells targeting 6 different genomic targets. Indel frequency is measured by next-generation sequencing with targeted primers on the target. FIG. 36B shows quantification of insertion and deletion frequencies generated on the DYNC1H1 genomic target by KnFNuc. Rates are shown per base throughout the quantification window of the amplicon. FIG. 36C shows representative indel alleles showing deletions and insertions centered around the 3′ end of the guide target.

### DETAILED DESCRIPTION

RNA-programmed nucleases serve diverse functions in prokaryotic systems, yet their prevalence and role in eukaryotic genomes are unclear. Searching for putative RNA-guided nucleases in genomes of diverse eukaryotes and their viruses, the present disclosure identifies numerous predicted nucleases homologous to the prokaryotic family of RNA-guided TnpB nucleases. Reconstruction of the evolutionary trajectory of these nucleases, which are referred to herein as Fanzor(s), uncovers at least two potential routes for their diversification. Surprisingly, biochemical and cellular evidence described herein shows that Fanzor families, which include the previously discovered Fanzor systems, employ non-coding RNAs encoded adjacent to the nuclease for RNA-guided cleavage of double-stranded DNA. Fanzor nucleases contain a re-arranged catalytic site inside the split RuvC domain, similar to a distinct subset of TnpB ancestors, yet lack collateral cleavage activity. In their adaptation and spread in eukaryotic lineages, Fanzor nucleases acquired N-terminal nuclear localization signals necessary for nuclear translocation, and Fanzor ORFs acquired introns, suggesting extensive spread and evolution within eukaryotes and their viruses. The present disclosure provides that Fanzor systems can be harnessed for genome editing in human cells, highlighting the potential of these widespread eukaryotic RNA-guided nucleases for biotechnology applications.

RNA-guided nucleases are prominent in prokaryotes, with roles in both adaptive immunity, such as CRISPR systems, and putative RNA-guided transposition or mobility, such as OMEGA systems (Karevelis et al. 2021; Altae-Tran et al. 2021). It is shown herein that the previously uncharacterized eukaryotic homologs of the OMEGA effector TnpB, previously termed Fanzors, are RNA-guided, programmable DNA nucleases. Additionally, the metagenomic analysis described herein permitted discovery of thousands of additional RuvC-containing nucleases in eukaryotes and their viruses, which are collectively referred to herein Fanzor systems (Table 1 and Table 4). As used herein, the term “Fanzor nuclease(s)” is interchangeable with “Fanzor polypeptide(s)” and “Fanzor protein(s)”.

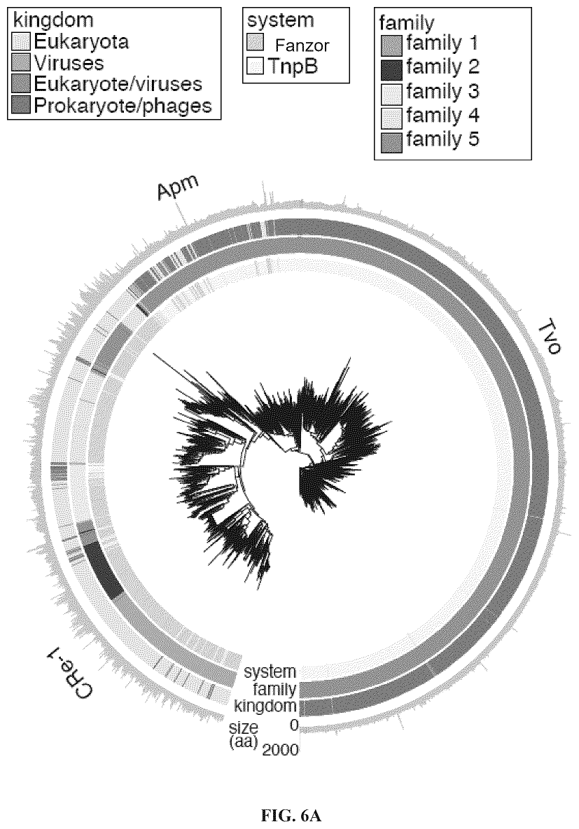

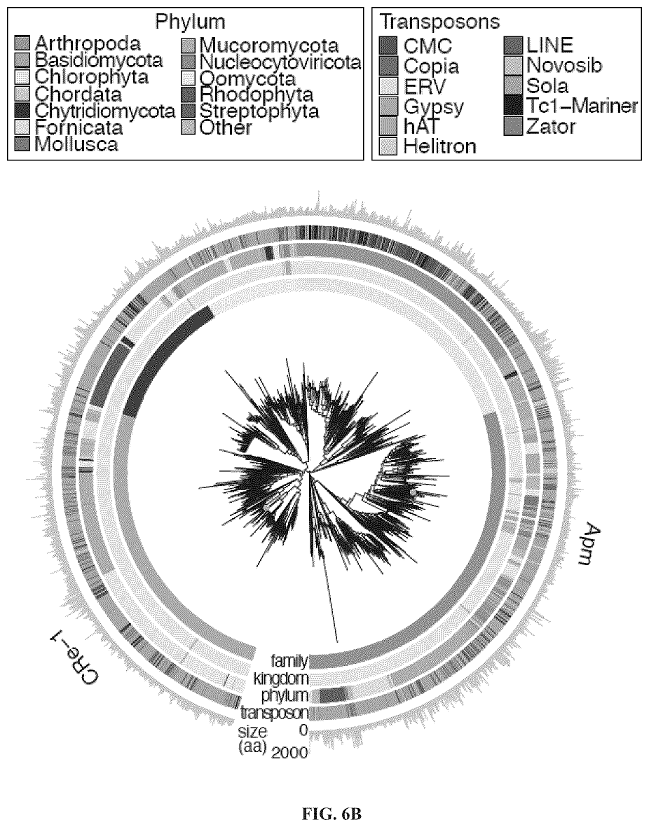

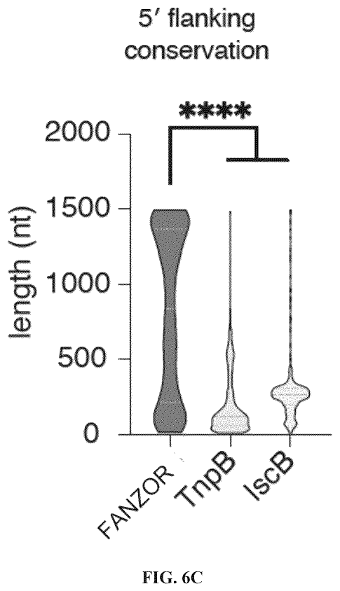

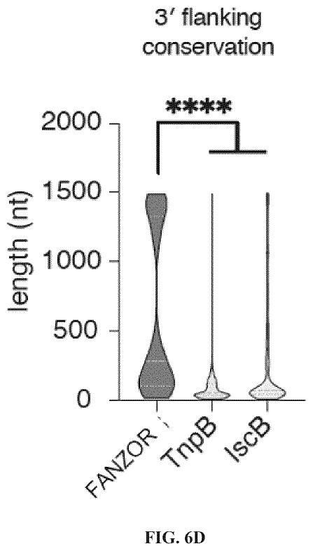

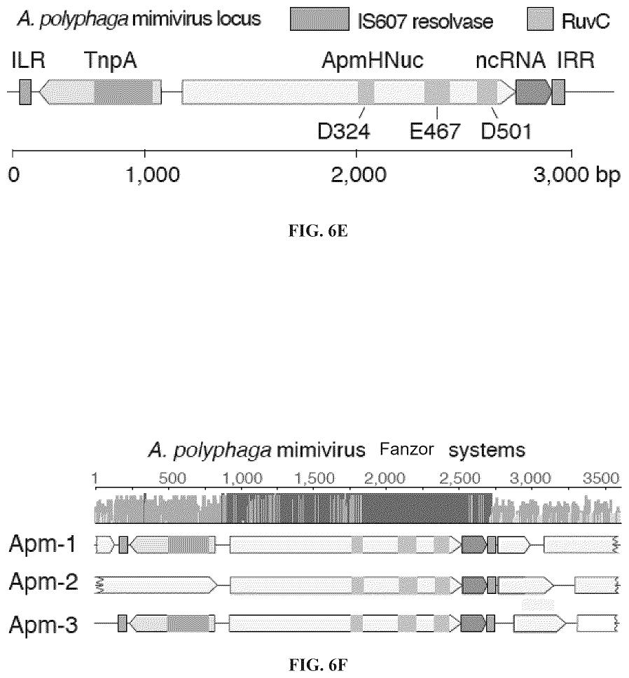

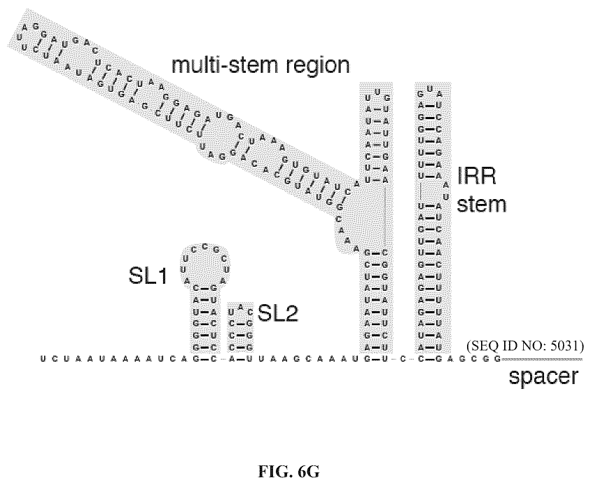

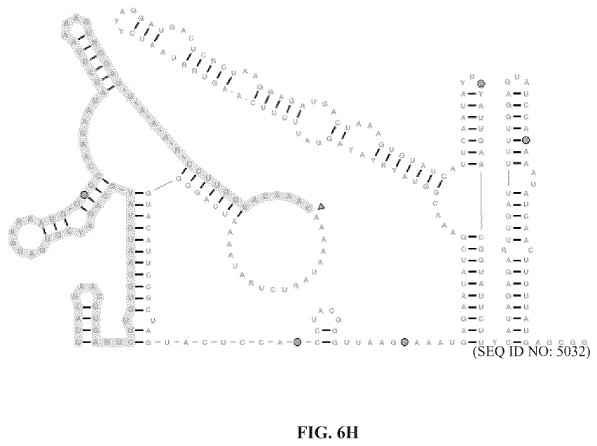

The phylogenetic analysis shown herein confirmed that the two previously identified families of Fanzors (Fanzors1 and Fanzor2) are distantly related. The Fanzor1 family, as well as diverse other Fanzor families, are present in numerous eukaryotes, including animals, plants, fungi and diverse protists whereas the Fanzor2 family is more narrowly represented in giant viruses of the family Mimiviridae. These two subsets of Fanzor systems most likely entered eukaryotes via distinct mechanisms in separate events. From evolutionary distances of different Fanzor families (FIG. 6A-6B), it is apparent that Fanzor systems in families 1-4, containing Fanzor1 proteins, likely evolved from an endosymbiotic pathway, with ancestral TnpB proteins driving multiple seeding events in different common ancestors, and that family 5 Fanzor systems, containing Fanzor2 proteins, likely originated from phagocytosis of TnpB-containing bacteria by amoeba and subsequent spread via amoeba-trophic giant viruses (Boyer et al. 2009). Notably, during their evolution in eukaryotic genomes, Fanzor nucleases acquired introns at densities that not significantly lower than mean intron densities in their host genes, similar to nuclear genes acquired from endosymbiotic organelles (Basu et al. 2008; Csuros et al. 2011). Additionally, many of these nucleases acquired N-terminal NLS, enabling nuclear invasion for genomic access. These independent evolutionary pathways likely contributed to the wide range of observed intron densities, NLS signals, N-terminal domains, and associated transposon systems across Fanzor diversity.

Fanzor nuclease association with transposases reported herein suggests a role for their RNA-guided nuclease activity in transposition. This role could be performed through a variety of mechanisms, including 1) precise excision of the transposon from the genome via self-homing, 2) passive homing of the transposon to new alleles via leveraging nuclease-induced DSBs and DNA repair mechanisms, such as homologous recombination, and 3) active homing of the transposon via RNA guided DNA binding or cleavage for direct targeting of transposase activity. The latter mechanism would be analogous to the CRISPR-associated Tn7-like transposons (CASTs) that undergo RNA-guided transposition mediated by CRISPR effectors that were captured by these transposons on multiple occasions, in conjunction with transposase components (Strecker et al. 2019; Klompe et al. 2019). Furthermore, given that Fanzor-containing transposons harbor associated genes with diverse functions, and different groups of Fanzor contain different N-terminal domains, Fanzor might perform additional functions that remain to be investigated.

The biochemical characterization of the Fanzor nucleases of the present disclosure revealed both similarities with the homologous TnpB and CRISPR-Cas12 nucleases and several important distinctions. Similar to TnpB and Cas12, Fanzor nucleases generate double-stranded breaks through a single RuvC domain and cleave the target DNA near the 3′ end of the target. However, unlike TnpB and Cas12 enzymes, which have strong collateral activity against free DNA and RNA species nearby, Fanzor proteins have a rearranged glutamic acid and do not have collateral activity. Accordingly, TnpB systems with similarly mutated and rearranged catalytic sites also do not display collateral activity, despite having targeted double-stranded DNA cleavage activity. As opposed to the more T rich sequence constraints of TnpB and Cas12 nucleases, the Fanzor TAM preference is diverse, with GC rich preference for Fanzor2 like nucleases. Importantly, the TAM preference seems to align with the insertion site sequence supporting the role of Fanzor systems in transposition. Finally, the fRNA of Fanzor overlaps with the transposon IRR, much like TnpB's ωRNA, but it extends farther downstream of the Fanzor ORF, in contrast to the ωRNAs that ends within the 3′ regions of the TnpB ORF as the noncoding region is significantly longer in the Fanzor MGE. Thus, although the Fanzor nucleases originated from TnpB systems, the properties of these eukaryotic RNA-guided nucleases are surprisingly and notably different from those of the prokaryotic ones.

It is demonstrated herein that Fanzor nucleases can be applied for genome editing with detectable cleavage and indel generation activity in human cells. While the Fanzor nucleases are compact (~500 amino acids), which could facilitate delivery, and their eukaryotic origins might help to reduce the immunogenicity of these nucleases in humans, additional engineering is needed to improve the activity of these systems in human cells, as has been accomplished for other miniature nucleases like Cas12f systems. See, e.g., Bigelyte et al. 2021; Wu et al. 2021; Xu et al. 2021; Kim et al. 2021. The broad distribution of Fanzor nucleases among diverse eukaryotic lineages and associated viruses suggests many more currently unknown RNA-guided systems could exist in eukaryotes, serving as a rich resource for future characterization and development of new biotechnologies.

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an,” and “the” include both singular and plural referents unless the context clearly dictates otherwise. Thus, for example, reference to “a cell” includes a plurality of such cells.

As used herein, the term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, the term “about” or “approximately” refers to a measurable value such as a parameter, an amount, a temporal duration, and the like, are meant to encompass variations of and from the specified value, such as variations of +/−10% or less, +/−5% or less, +/−1% or less, +/−0.5% or less, and +/−0.1% or less of and from the specified value, insofar such variations are appropriate to perform in the disclosure. It is to be understood that the value to which the modifier “about” or “approximately” refers is itself also specifically, and preferably, disclosed.

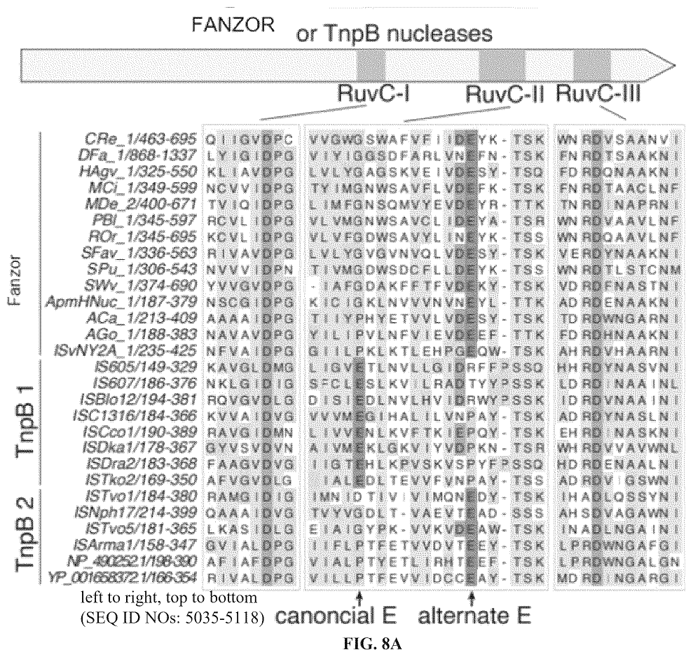

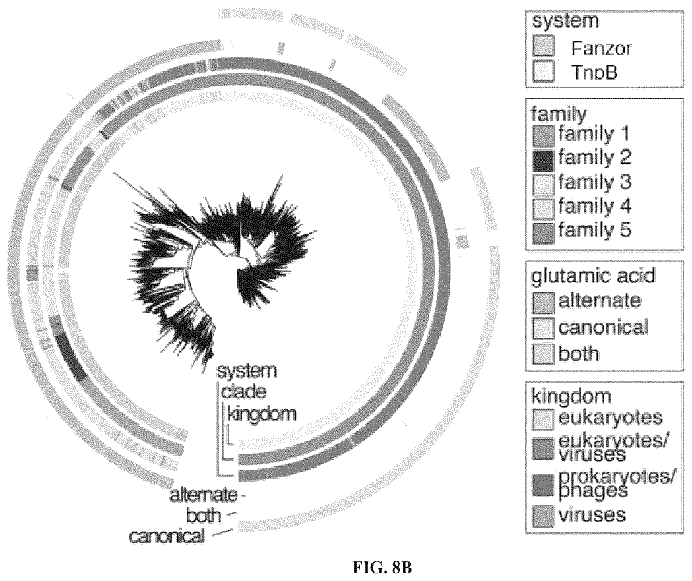

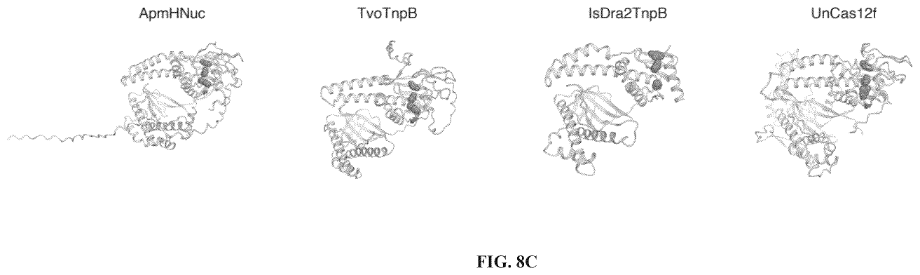

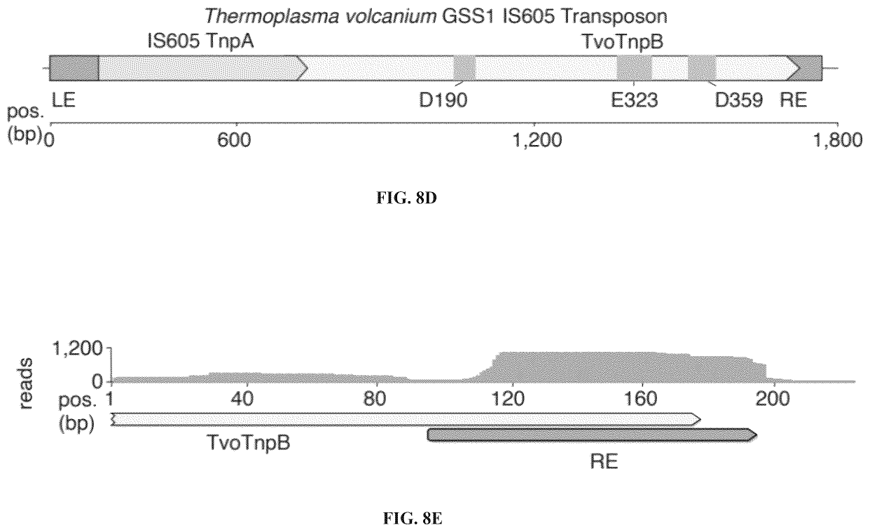

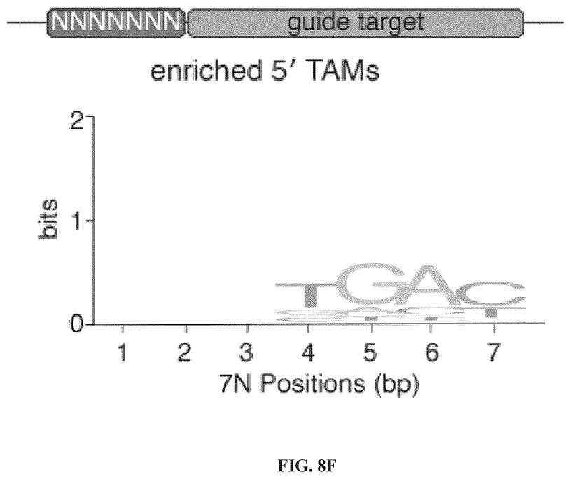

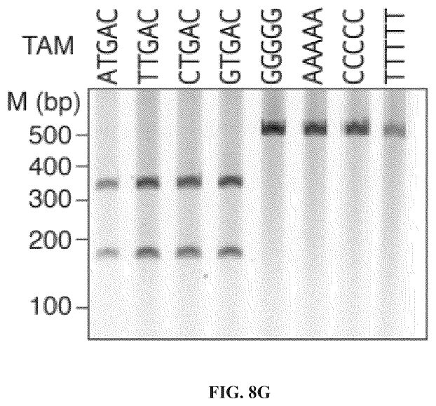

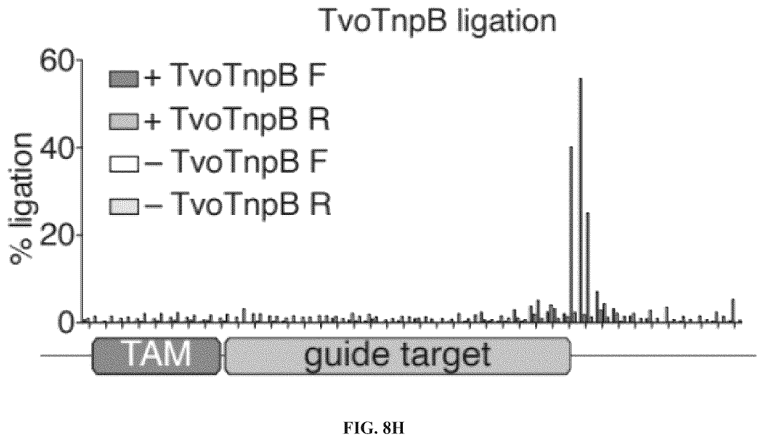

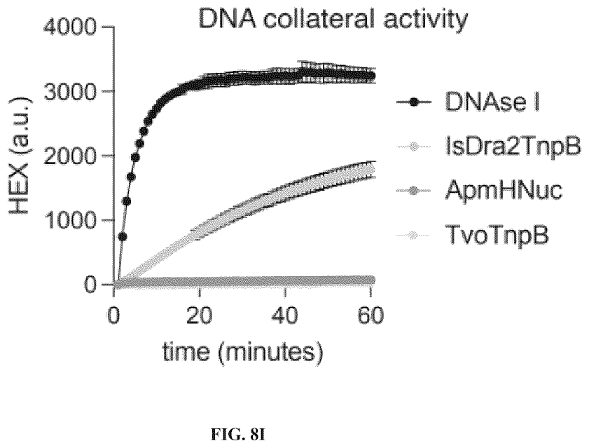

In some aspects, the present disclosure relates to non-naturally occurring, engineered compositions comprising a Fanzor polypeptide encoding a Fanzor nuclease. Fanzor polypeptides comprise a single RuvC domain. The single RuvC domain is further comprised of three subdomains: a RuvC-I subdomain, a RuvC-II subdomain, and a RuvC-III subdomain. In some embodiments, the RuvC-II subdomain of a Fanzor polypeptide is a rearranged RuvC-II subdomain. As used herein, a “rearranged RuvC-II subdomain” refers to a domain within a RuvC-containing nuclease (e.g., a Fanzor nuclease) further comprising a loss of the canonical glutamic acid in the RuvC-II subdomain and an alternative conserved glutamate approximately 45 residues away. As described herein, all Fanzor members and the rearranged TnpB orthologs, contained an alternative conserved glutamate approximately 45 residues away (FIG. 8A-8B). In some embodiments, the glutamic acid in the “rearranged RuvC-II subdomain” substitutes the role of canonical one in the wildtype RuvC-II subdomain, to allow for effective cleavage activity. In some embodiments, a Fanzor comprising a rearranged catalytic site (e.g., a rearranged RuvC-II subdomain) results in reduced collateral cleavage activity of the enzyme. As used herein, “collateral cleavage activity” or “collateral activity” are used interchangeably to describe nuclease activity (e.g., cleavage) of non-targeted DNA(s) and/or RNA(s). In some embodiments, a Fanzor nuclease lacks collateral DNA cleavage activity (e.g., lacks nuclease activity of non-targeted DNA). In some embodiments, a Fanzor nuclease lacks collateral RNA cleavage activity (e.g., lacks nuclease activity of non-targeted RNA). In some embodiments, a Fanzor nuclease lacks collateral DNA and RNA cleavage activity (e.g., lacks nuclease activity of non-targeted DNA and RNA). The presence or absence of collateral cleavage activity can be measured (e.g., profiled), for example, by co-incubating the Fanzor nuclease and fRNA complexes with their cognate targets along with either ssRNA or ssDNA cleavage reporters, single-stranded nucleic acid substrates functionalized with a quencher and fluorophore that become fluorescent upon nucleolytic cleavage. Other techniques known in the art for measuring collateral cleavage activity are also contemplated for use herein.

In some embodiments, a Fanzor polypeptide comprises an amino acid sequence identified by any one of the sequences provided herein (see e.g., Table 1, SEQ ID NOs: 1, 95-5029, and Table 4, SEQ ID NOs: 1-3, 5-7, and 9-16, or having an amino acid sequence at least at least 50%, at least 55%, at least 60%, at least 65%, at least 70%, at least 75%, at least 80%, at least 85%, at least 90%, at least 95%, at least 99%, or 100% sequence identity (including all values in between) with a Fanzor polypeptide listed in Table 1 or Table 4 (SEQ ID NOs: 1-3, 5-7, 9-16 and 95-5029).

As used herein, the term “percent identity” refers to a relationship between two nucleic acid sequences or two amino acid sequences, as determined by sequence comparison (alignment). In some embodiments, identity is determined across the entire length of a sequence. In some embodiments, identity is determined over a region of a sequence.

Identity of sequences can be readily calculated by those having ordinary skill in the art. In some embodiments, the percent identity of two sequences is determined using the algorithm of Karlin and Altschul 1990 Proc. Natl. Acad. Sci. U.S.A. 87:2264-68, modified as in Karlin and Altschul 1993 Proc. Natl. Acad. Sci. U.S.A. 90:5873-77. This algorithm is incorporated into the NBLAST® and XBLAST® programs (version 2.0) of Altschul et al. 1990 J. Mol. Biol. 215:403-10. BLAST® protein searches can be performed, for example, with the XBLAST program, score=50, wordlength=3 to obtain amino acid sequences homologous to the protein molecules of the invention. Where gaps exist between two sequences, Gapped BLAST® can be utilized, for example, as described in Altschul et al. 1997 Nucleic Acids Res. 25 (17): 3389-3402. When utilizing BLAST® and Gapped BLAST® programs, the default parameters of the respective programs (e.g., XBLAST® and NBLAST®) can be used, or the parameters can be adjusted appropriately as would be understood by one of ordinary skill in the art.

In some embodiments, a Fanzor polypeptide comprises about 200 to about 2212 amino acids (including all values in between). In some embodiments, a Fanzor polypeptide comprises about 200 amino acids. In some embodiments, a Fanzor polypeptide comprises about 500 amino acids. In some embodiments, a Fanzor polypeptide comprises about 1000 amino acids. In some embodiments, a Fanzor polypeptide comprises about 1500 amino acids. In some embodiments, a Fanzor polypeptide comprises about 2000 amino acids. In some embodiments, a Fanzor polypeptide comprises about 2212 amino acids.

In some embodiments, loci surrounding a nucleotide sequence encoding a Fanzor nuclease comprises a conserved non-coding sequence. In some embodiments, the conserved non-coding sequence extends at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, or at least 200 base pairs (including all values in between) past the end of a Fanzor open reading frame (ORF).

In some embodiments, directed evolution may be used to design modified Fanzor proteins capable of genome editing. In some embodiments, the directed evolution is performed using phage-assisted continuous evolution (PACE). In some embodiments, the directed evolution is performed using phage-assisted non-continuous evolution (PANCE). PACE technology has been described, for example, in International PCT Application, PCT/US 2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. Pat. No. 9,023,594, issued May 5, 2015; U.S. Pat. No. 9,771,574, issued Sep. 26, 2017; U.S. Pat. No. 9,394,537, issued Jul. 19, 2016; International PCT Application, PCT/US2015/012022, filed Jan. 20, 2015, published as WO 2015/134121 on Sep. 11, 2015; U.S. Pat. No. 10,179,911, issued Jan. 15, 2019; U.S. Pat. No. 10,179,911, issued Jan. 15, 2019; International PCT Application, PCT/US2016/027795, filed Apr. 15, 2016, published as WO 2016/168631 on Oct. 20, 2016, and International Patent Publication WO 2019/023680, published Jan. 31, 2019, the entire contents of each of which are incorporated herein by reference. In some embodiments, directed evolution is implemented using a protein folding neural network, e.g., based on a published approach or on software such as AlphaFold2. In some embodiments, the Fanzor proteins obtained by methods of directed evolution are physically synthesized.

In some embodiments, the modified Fanzor protein has improved editing efficiency relative to a control Fanzor protein. In some embodiments, the improved editing efficiency is detected in mammalian cells. In some embodiments, the improved editing efficiency can be measured by an indel formation rate. In some embodiments, the indel formation rate is at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%, including all values in between. In some embodiments, the modified Fanzor protein comprises one or more mutations of amino acid residues in the catalytic core (e.g., the catalytic RuvC domains) and/or of amino acid residues that contact the polynucleotide target relative to the wild type Fanzor protein. Non-limiting examples of mutations include one or more amino acid residues in a modified Fanzor protein mutated to arginine, lysine, and/or histidine relative to a wild type Fanzor protein. In some embodiments, the modified Fanzor protein comprises a mutation to arginine relative to the wild type Fanzor protein. In other embodiments, the modified Fanzor protein comprises one or more mutations to arginine relative to the wild type Fanzor protein. In some embodiments, the modified Fanzor protein comprises a mutation to lysine relative to the wild type Fanzor protein. In other embodiments, the modified Fanzor protein comprises one or more mutations to lysine relative to the wild type Fanzor protein. In some embodiments, the modified Fanzor protein comprises a mutation to histidine relative to the wild type Fanzor protein. In other embodiments, the modified Fanzor protein comprises one or more mutations to histidine relative to the wild type Fanzor protein. In some embodiments, the modified Fanzor protein contains one or more mutations to arginine, lysine, and/or histidine relative to the wild type Fanzor protein.

In some embodiments, the conserved non-coding sequence encodes a nuclease-associated RNA. In some embodiments, the nuclease-associated RNA is a Fanzor (“fRNA”) molecule. In some embodiments, the fRNA molecule is capable of directing binding and cleavage activity (e.g., guiding) of a Fanzor nuclease to a specific sequence (e.g., a target polypeptide sequence). In some embodiments, a fRNA is a guide RNA or gRNA. In some embodiments, the fRNA molecule comprises a scaffold. In some embodiments, the scaffold comprises about 21 to about 1487 nucleotides (including all values in between). In some embodiments, the scaffold comprises about 21 nucleotides. In some embodiments, the scaffold comprises about 50 nucleotides. In some embodiments, the scaffold comprises about 100 nucleotides. In some embodiments, the scaffold comprises about 150 nucleotides. In some embodiments, the scaffold comprises about 200 nucleotides. In some embodiments, the scaffold comprises about 250 nucleotides. In some embodiments, the scaffold comprises about 300 nucleotides. In some embodiments, the scaffold comprises about 350 nucleotides. In some embodiments, the scaffold comprises about 400 nucleotides. In some embodiments, the scaffold comprises about 450 nucleotides. In some embodiments, the scaffold comprises about 500 nucleotides. In some embodiments, the scaffold comprises about 550 nucleotides. In some embodiments, the scaffold comprises about 600 nucleotides. In some embodiments, the scaffold comprises about 650 nucleotides. In some embodiments, the scaffold comprises about 700 nucleotides. In some embodiments, the scaffold comprises about 750 nucleotides. In some embodiments, the scaffold comprises about 800 nucleotides. In some embodiments, the scaffold comprises about 850 nucleotides. In some embodiments, the scaffold comprises about 900 nucleotides. In some embodiments, the scaffold comprises about 950 nucleotides. In some embodiments, the scaffold comprises about 1000 nucleotides. In some embodiments, the scaffold comprises about 1050 nucleotides. In some embodiments, the scaffold comprises about 1150 nucleotides. In some embodiments, the scaffold comprises about 1200 nucleotides. In some embodiments, the scaffold comprises about 1250 nucleotides. In some embodiments, the scaffold comprises about 1300 nucleotides. In some embodiments, the scaffold comprises about 1350 nucleotides. In some embodiments, the scaffold comprises about 1400 nucleotides. In some embodiments, the scaffold comprises about 1487 nucleotides.

In some embodiments, the fRNA molecule comprises a reprogrammable target spacer sequence. In some embodiments, the reprogrammable target spacer sequence comprises about 12 to about 22 nucleotides (including all values in between). In some embodiments, the reprogrammable target spacer sequence comprises about 12 nucleotides. In some embodiments, the reprogrammable target spacer sequence comprises about 13 nucleotides. In some embodiments, the reprogrammable target spacer sequence comprises about 14 nucleotides. In some embodiments, the reprogrammable target spacer sequence comprises about 15 nucleotides. In some embodiments, the reprogrammable target spacer sequence comprises about 16 nucleotides. In some embodiments, the reprogrammable target spacer sequence comprises about 17 nucleotides. In some embodiments, the reprogrammable target spacer sequence comprises about 18 nucleotides. In some embodiments, the reprogrammable target spacer sequence comprises about 19 nucleotides. In some embodiments, the reprogrammable target spacer sequence comprises about 20 nucleotides. In some embodiments, the reprogrammable target spacer sequence comprises about 21 nucleotides. In some embodiments, the reprogrammable target spacer sequence comprises about 22 nucleotides.

In some embodiments, the fRNA molecule comprises a scaffold and a reprogrammable target spacer sequence. In some embodiments, the fRNA molecule comprises a scaffold about 21 to about 1487 nucleotides and a reprogrammable target spacer sequence comprises about 12 to about 22 nucleotides.

In some embodiments, the fRNA molecule is capable of forming a complex with the Fanzor polypeptide (e.g. a “Fanzor complex”) and directing the Fanzor polypeptide to a target polynucleotide sequence. The target polynucleotide of a complex (e.g., a Fanzor complex) can be any polynucleotide endogenous or exogenous to the eukaryotic cell. For example, the target polynucleotide can be a polynucleotide residing in the nucleus of the eukaryotic cell. The target polynucleotide can be a sequence coding a gene product (e.g., a protein) or a non-coding sequence (e.g., a regulatory polynucleotide or a junk DNA). In some embodiments, the complex (e.g., a Fanzor complex) binds a target adjacent motif (TAM) sequence (e.g., a short sequence recognized by the complex). In some embodiments, the complex (e.g., a Fanzor complex) binds a TAM sequence 5′ of the target polynucleotide sequence. In some embodiments, the TAM sequence comprises GGG. In some embodiments, the TAM sequence comprises TTTT. In some embodiments, the TAM sequence comprises TAT. In some embodiments, the TAM sequence comprises TTG. In some embodiments, the TAM sequence comprises TTTA. In some embodiments, the TAM sequence comprises TA. In some embodiments, the TAM sequence comprises TTA. In some embodiments, the TAM sequence comprises TGAC. A person of skill in the art would be able to identify further TAM sequences for use with a given Fanzor polypeptide. It is also contemplated herein that TAM interacting domain may be engineered by techniques known in the art to allow programming of specificity, improvement of target site recognition fidelity, and increased the versatility of the Fanzor nuclease genome engineering platform described herein. It is further contemplated that Fanzor nuclease may be engineered to alter their TAM specificity.

Examples of target polynucleotide sequences include, but are not limited to, a sequence associated with a signaling biochemical pathway, e.g., a signaling biochemical pathway-associated gene or polynucleotide. Further non limiting examples of target polynucleotide sequences include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non-disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown, and may be at a normal or abnormal level.

In some embodiments, a Fanzor polypeptide in a Fanzor polypeptide. In some embodiments, the Fanzor polypeptide is a Fanzor 1 polypeptide. In some embodiments, the Fanzor polypeptide is a Fanzor 2 polypeptide. In some embodiments, the RNA molecule associated with a Fanzor polypeptide is a fRNA. In some embodiments, a fRNA molecule is a fRNA molecule.

As described herein, in some embodiments, a Fanzor polypeptide may comprise additional domains other than the RuvC domain. In some embodiments, a Fanzor polypeptide comprises a nuclear localization signal (NLS). In some embodiments, a Fanzor polypeptide comprises a helix-turn-helix (HTH) domain.

In some embodiments, one or more vectors may comprise a nucleic acid sequence encoding a polypeptide described herein (e.g., a Fanzor polypeptide). As such, aspects of the present disclosure relate to one or more vectors for the expression of (a) a nucleic acid sequence encoding a Fanzor polypeptide; and (b) a nucleic acid sequence encoding a fRNA molecule comprising a scaffold and a reprogrammable target spacer sequence. In some embodiments, a vector may comprise both (a) a nucleic acid sequence encoding a Fanzor polypeptide; and (b) a nucleic acid sequence encoding a fRNA molecule. In some embodiments, a vector may comprise a nucleic acid sequence encoding a Fanzor polypeptide; and a second vector may comprise a nucleic acid sequence encoding a fRNA molecule.

The term “vector” or “expression vector” or “construct” means any molecular vehicle, such as a plasmid, phage, transposon, recombinant viral genome, cosmid, chromosome, artificial chromosome, virus, viral particle, viral vector (e.g., lentiviral vector or AAV vector), virion, etc. which can transfer gene sequences (e.g., a nucleic acid encoding a Fanzor polypeptide and/or a nucleic acid sequence encoding a fRNA molecule) into a cell or between cells.

In some embodiments, the vector may be maintained in high levels in a cell using a selection method such as involving an antibiotic resistance gene. In some embodiments, the vector may comprise a partitioning sequence which ensures stable inheritance of the vector. In some embodiments, the vector is a high copy number vector. In some embodiments, the vector becomes integrated into the chromosome of a cell.

Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory elements) in a manner that allows for expression of the nucleotide sequence (e.g. in an in vitro transcription/translation system or in a host cell when the vector is introduced into the host cell). With regards to recombination and cloning methods, mention is made of U.S. patent application Ser. No. 10/815,730, published Sep. 2, 2004 as US 2004-0171156 A1, the contents of which are herein incorporated by reference in their entirety.

The vectors can include the regulatory elements, (e.g., promoters). The vectors can comprise Fanzor nuclease encoding sequences, and/or fRNA(s). In a single vector there can be a promoter for a Fanzor nuclease encoding sequence and an fRNA. In multiple vectors, there can be a first vector comprising a promoter for a Fanzor nuclease encoding sequence and a second vector comprising a promoter for a fRNA. A non-limiting example of a suitable vector is AAV, and a non-limiting example of a suitable promoter is a U6 promoter. Accordingly, from the knowledge in the art and the teachings in this disclosure the skilled person can readily make and use vectors), e.g., a single vector, expressing multiple RNAs or guides under the control or operatively or functionally linked to one or more promoters—especially as to the numbers of RNAs or guides discussed herein, without any undue experimentation.

The Fanzor nuclease encoding sequences and/or fRNA, can be functionally or operatively linked to regulatory elements. In some embodiments, the regulatory elements drive expression of the Fanzor nuclease and the fRNA. Promoters can be constitutive promoters and/or conditional promoters and/or inducible promoters and/or tissue specific promoters. Exemplary promoters include RNA polymerases, pol I, pol II, pol III, T7, U6, HI, retroviral Rous sarcoma virus (RSV) LTR promoter, the cytomegalovirus (CMV) promoter, the SV40 promoter, the dihydrofolate reductase promoter, the β-actin promoter, the phosphoglycerol kinase (PGK) promoter, the EFla promoter, the U6 promoter, and the pCAG promoter. An advantageous promoter is the pCAG promoter. Other promoters known in the art are also contemplated for use herein.

In addition to a Fanzor polypeptide and a nucleic acid sequence encoding an fRNA molecule, compositions of the present disclosure may comprise additional components useful for gene-editing. As non-limiting examples, compositions of the present disclosure may comprise one or more of a donor template (e.g. exogenous template) comprising a donor sequence, a linear insert sequence, a reverse transcriptase, a recombinase, a transposase, an integrase, a deaminase, a transcriptional activator, a transcriptional repressor, and/or a transposon. In some embodiments, a composition of the present disclosure comprises a donor template (e.g., exogenous template) comprising a donor sequence. In some embodiments, the donor template comprising a donor sequence is optionally for use in homology-directed repair (HDR). In some embodiments, compositions optionally for use in homology-directed repair further comprises introducing specific sequences or genes at targeted genomic locations. Reference is made to PCT Publication No. WO2008/021207, the entire contents of which is incorporated herein by reference. In some embodiments, a composition of the present disclosure comprises a linear insert sequence. A linear insert sequence as described herein comprises, for example, DNA, RNA, or mRNA. In some embodiments, a linear insert sequence is DNA. In some embodiments, a linear insert sequence is RNA. In some embodiments, a linear insert sequence is mRNA. In some embodiments, a linear insert sequence is comprised by a viral vector, optionally wherein the viral vector is Adeno-associated viral (AAV) vector, a virus, optionally wherein the virus is an Adenovirus, a lentivirus, a herpes simplex virus; and/or a lipid nanoparticle (LNP). In some embodiments, a LNP comprises one or more components of the compositions of the present disclosure. In some embodiments, the linear insert sequence is optionally for use in non-homologous end joining-based insertion. Reference is made to US Patent Publication No. US2022/0000933A1, the entire contents of which is incorporated herein by reference. In some embodiments, a composition of the present disclosure comprises a reverse transcriptase. In some embodiments, a reverse transcriptase is optionally for use in prime editing. Reference is made to U.S. Pat. No. 11,447,770, the entire contents of which is incorporated herein by reference. In some embodiments, a composition of the present disclosure comprises a recombinase, optionally for use for integration. Reference is made to U.S. Pat. No. 11,572,556, the entire contents of which is incorporated herein by reference. In some embodiments, a composition of the present disclosure comprises a transposase, optionally for use for integration. In some embodiments, the transposase naturally occurs with Fanzor systems. In some embodiments, the transposase is any one of Table 1. Non-limiting examples of transposes include Ty3, Novosib, Copia, CMC, Tc1_Mariner, hAT, Helitron, LINE, Zator, ERV, Sola, Crypton, EnSpm, IS607, Gin, and piggybac. Reference is made to PCT Publication No. WO2021030756A1, the entire contents of which is incorporated herein by reference. In some embodiments, a composition of the present disclosure comprises an integrase, optionally for use for integration. Reference is made to PCT Application No. PCT/2023/070031 and U.S. application Ser. No. 18/048,238, the entire contents of each which is incorporated herein by reference. In some embodiments, compositions optionally for use for integration further comprises programmable addition via site-specific targeting elements (PASTE). Reference is made to U.S. Pat. No. 11,572,556, the entire contents of which is incorporated herein by reference. In some embodiments, a composition of the present disclosure comprises a deaminase, optionally for use of base-editing.

In some embodiments, compositions optionally for the use of base-editing are capable of acting on single-stranded DNA. In some embodiments, compositions optionally for the use of base-editing are capable of acting on double-stranded DNA. In some embodiments, compositions optionally for the use of base-editing are capable of acting on RNA. In some embodiments, the deaminase is a cytidine deaminase. In some embodiments, compositions optionally for use of base-editing further comprises changing cytosine to thymine. In some embodiments, compositions optionally for use of base-editing further comprises changing cytosine to thymine without double-stranded breaks. In some embodiments, the deaminase is an adenine deaminase. In some embodiments, compositions optionally for use of base-editing further comprises changing adenine to guanine. In some embodiments, compositions optionally for use of base-editing further comprises changing adenine to guanine without double-stranded breaks.

In some embodiments, a composition of the present disclosure comprises a transcriptional activator, optionally for use of targeted gene activation. In some embodiments, compositions optionally for the use of targeted gene activation recruit transcriptional domains. Non-limiting examples of transcriptional domains include the transactivation domain of a zinc-finger protein, transcription activator-like effector, the Herpes simplex viral protein 16 (VP16), multiple tandem copies of VP16, such as VP64 or VP160, p65, and HSF1. Other t In some embodiments, a composition of the present disclosure comprises a transcriptional repressor, optionally for use of targeted gene repression. Non-limiting examples of transcriptional repressors include Kruppel-associated box (KRAB), Sin3 interaction domain (SID), Enhancer of Zeste Homolog2 (EZH2), histone deacetylases, and TET1. In some embodiments, the transcriptional repressor is a methyltransferase. In some embodiments, the methyltransferase is DNMT3A. In some embodiments, the methyltransferase is an enzyme that enhances the activity of DNMT3A. In some embodiments, the methyltransferase is DNMT3L. In some embodiments, the transcriptional repressor is a histone modifier. Non-limiting examples of histone modifiers include p300, LSD1, and heterochromatin protein 1 (HP1).

In some embodiments, a composition of the present disclosure comprises an epigenetic modification domain, optionally for use of epigenetic editing. In some embodiments, the epigenetic editing further comprises modifying histone modifications. In some embodiments, the epigenetic editing further comprises modifying DNA methylation patterns. In some embodiments, the epigenetic editing upregulates gene expression. In some embodiments, the epigenetic editing downregulates gene expression. Non-limiting examples of epigenetic modification domains include histone acetyltransferase p300, histone demethylase (LSD1), histone methyltransferases, such as DOT1L and PRDM9, and DNA methyltransferase DNMT3A.

In some embodiments, a composition of the present disclosure comprises a transposon, optionally for RNA guided transposition. Non-limiting examples of eukaryotic transposons include CMC, Copia, ERV, Gypsy, hAT, helitron, Zator, Sola, LINE, Tc1-Mariner,Novosib, Crypton, and EnSpm. Other eukaryotic transposons known in the art are contemplated for use herein. Reference is also made to PCT Publication No. WO2022/087494 and PCT Publication No.WO2022/159892, the entire contents of each, which is incorporated herein by reference. Compositions of the present disclosure further comprising other components known in the art for use in gene-editing are also contemplated herein. Further aspects of the disclosure comprise engineered cells comprising the Fanzor polypeptides and fRNA molecules described herein. In some embodiments, engineered cells comprise mammalian cells. Non-limiting examples of engineered cells include human cells, and any non-human eukaryote or animal or mammal as herein discussed, e.g., rodent, mouse, rat, rabbit, dog, livestock, or non-human mammal or primate. In some embodiments, the engineered cell is a rodent cell. In some embodiments, the engineered cell is a human cell. Other mammalian cell types are contemplated for use herein. In some embodiments, engineered cells of the disclosure may be isolated from human cells or tissues, plants and/or seeds, or non-human animals. It is contemplated herein that in some embodiments, host cells and/or cell lines are generated from the engineered cells of the disclosure comprising Fanzor nucleases and fRNAs described herein. It is further contemplated that host cells and/or cell lines modified by the Fanzor nucleases and fRNAs described herein include isolated stem cells and progeny thereof.

Further aspects of the disclosure provide methods of modifying a target polynucleotide sequence in a cell comprising delivering to the cell the Fanzor polypeptides and fRNA molecules described herein. In some embodiments, delivery of the Fanzor polypeptides and fRNA molecules form a complex (e.g., a Fanzor complex) for modifying a target DNA or RNA (single or double stranded, linear or supercoiled). The Fanzor complex of the invention have a wide variety of utility including modifying (e.g., deleting, inserting, translocating, inactivating, activating) a target DNA or RNA in a multiplicity of cell types. As such, the nucleic acid-targeting complex of the invention has a broad spectrum of applications in, e.g., gene therapy, drug screening, disease diagnosis, and prognosis. An exemplary nucleic acid-targeting complex comprises a DNA or RNA-targeting effector protein complexed with a co-RNA or guide RNA (gRNA) hybridized to a target polynucleotide sequence within the target locus of interest.

In some embodiments, modifying a target polynucleotide sequence comprises cleavage (e.g., a single or a double strand break) of the target polynucleotide sequence. In some embodiments, the target polynucleotide sequence is DNA. In some embodiments, one or more mutations comprising substitutions, deletions, and insertions are introduced into the target polynucleotide sequence. In some embodiments, the one or more mutations introduces frameshift mutations. In some embodiments, the cleavage creates a single-stranded break. In some embodiments, the single-stranded break reduces off-target effects. In some embodiments, the single-stranded breaks are used in pairs to create staggered double-stranded breaks. In some embodiments, the one or more mutations introduces a point mutation. In some embodiments, the one or more mutations are introduced without double-stranded breaks. In some embodiments, the one or more mutations are introduced without donor DNA. In some embodiments, the cleavage occurs proximal to the 3′ end of the target polynucleotide sequence. In some embodiments, the cleavage occurs in a specific location relative to the 3′ end of the target polynucleotide sequence. In some embodiments, a Fanzor nuclease modifies a target polynucleotide sequence by cleaving between about −6 to about +3 nucleotides relative to the 3′ end of the target polynucleotide sequence. In some embodiments, a Fanzor nuclease modifies a target polynucleotide sequence by cleaving −6 nucleotides relative to the 3′ end of the target polynucleotide sequence. In some embodiments, a Fanzor nuclease modifies a target polynucleotide sequence by cleaving −5 nucleotides relative to the 3′ end of the target polynucleotide sequence. In some embodiments, a Fanzor nuclease modifies a target polynucleotide sequence by cleaving −4 nucleotides relative to the 3′ end of the target polynucleotide sequence. In some embodiments, a Fanzor nuclease modifies a target polynucleotide sequence by cleaving −3 nucleotides relative to the 3′ end of the target polynucleotide sequence. In some embodiments, a Fanzor nuclease modifies a target polynucleotide sequence by cleaving −2 nucleotides relative to the 3′ end of the target polynucleotide sequence. In some embodiments, a Fanzor nuclease modifies a target polynucleotide sequence by cleaving −1 nucleotides relative to the 3′ end of the target polynucleotide sequence. In some embodiments, a Fanzor nuclease modifies a target polynucleotide sequence by cleaving 0 nucleotides relative to the 3′ end of the target polynucleotide sequence (e.g., cleaving at the 3′ end of the target polynucleotide sequence). In some embodiments, a Fanzor nuclease modifies a target polynucleotide sequence by cleaving +1 nucleotides relative to the 3′ end of the target polynucleotide sequence. In some embodiments, a Fanzor nuclease modifies a target polynucleotide sequence by cleaving +2 nucleotides relative to the 3′ end of the target polynucleotide sequence. In some embodiments, a Fanzor nuclease modifies a target polynucleotide sequence by cleaving +3 nucleotides relative to the 3′ end of the target polynucleotide sequence.

In some embodiments, the Fanzor nuclease modifies a target polynucleotide sequence by cleaving within the TAM sequence.

The methods of according to the invention as described herein comprehend modifying a target polynucleotide sequence, comprising contacting a sample that comprises the target polynucleotide sequence with the composition, vectors, polynucleotides comprising Fanzor nucleases and fRNA molecules described herein wherein contacting results in modification of a target polynucleotide sequence or modification of the amount or expression of a gene and/or gene product. In some embodiments, the expression of the targeted gene and/or gene product is increased by the method relative to an unmodified control. In some embodiments, the expression of the targeted gene and/or gene product is increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, p at least 90%, at least 95%, 100% relative to an unmodified control. In some embodiments, the expression of the targeted gene and/or gene product is increased at least 1.5-fold, at least 2-fold, at least 2.5-fold, at least 3-fold, at least 3.5-fold, at least 3.5-fold, at least 4-fold, at least 4.5-fold, at least 5-fold, at least 10-fold, at least 10-fold, at least 15-fold, at least 20-fold, at least 25-fold, at least 50-fold, at least 100-fold relative to an unmodified control. In some embodiments, the expression of the targeted gene and/or gene product is reduced by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100% relative to an unmodified control. In some embodiments, the expression of the targeted gene and/or gene product is reduced at least 1.5-fold, at least 2-fold, at least 2.5-fold, at least 3-fold, at least 3.5-fold, at least 3.5-fold, at least 4-fold, at least 4.5-fold, at least 5-fold, at least 10-fold, at least 10-fold, at least 15-fold, at least 20-fold, at least 25-fold, at least 50-fold, at least 100-fold relative to an unmodified control. In some embodiments, the expression of the targeted gene and/or gene product is reduced by the method. In some embodiments, expression of the targeted gene may be completely eliminated, or may be considered eliminated as remnant expression levels of the targeted gene fall below the detection limit of methods known in the art that are used to quantify, detect, or monitor expression levels of genes.

The compositions and methods according to the invention as described herein comprehend inducing one or more nucleotide modifications in a eukaryotic cell (e.g., in a target polynucleotide sequence within a cell). In some embodiments, one or more modifications in a eukaryotic cell occurs in vitro, i.e. in an isolated eukaryotic cell, including but not limited to, a human cell) as herein discussed comprising delivering to cell a vector as herein discussed. In other embodiments, one or more modifications in a eukaryotic cell occurs in vivo. The mutation(s) can include the introduction, deletion, or substitution of one or more nucleotides at each target sequence of cell(s) via the guide RNA(s) or fRNA(s). The mutations can include the introduction, deletion, or substitution of a range of nucleotides (e.g., at each target sequence of said cell(s) via the guide(s) RNA(s) or fRNA(s). The mutations can include the introduction, deletion, or substitution of 1-100 nucleotides at each target sequence of said cell(s) via the guide RNA(s) or fRNA(s). The mutations can include the introduction, deletion, or substitution of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides at each target sequence of said cell(s) via the guide RNA(s) or fRNA(s). The mutations can include removing, adding, or rearranging large chromosomal segments at each target sequence of said cell(s) via the guide RNA(s) or fRNA(s). In some embodiments, the fRNA includes a primer binding site. In some embodiments the primer binding site (PBS) binds to exposed DNA. In some embodiments, the primer binding site binds to exposed DNA generated by Fanzor cleavage. In some embodiments, the fRNA further includes a reverse transcriptase (RT) region. In some embodiments, the RT region is complementary to the genome. In some embodiments, the mutation is introduced between the RT and PBS sites.

The nucleic acid molecule encoding a Fanzor nuclease may be codon optimized for expression in a particular host species. A codon optimized sequence includes a sequence optimized for expression in a different eukaryote relative to the eukaryote of origin for a Fanzor nuclease. As a non-limiting example, the nucleic acid molecule encoding a Fanzor nuclease from Chlamydomonas reinhardtii may be codon-optimized for expression in humans, or for another eukaryote, animal or mammal as herein. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g. 1, 2, 3, 4, 5, 10, 15, 20, 25, 50, or more, or all codons) in a sequence encoding a Fanzor nuclease correspond to the most frequently used codon for a particular amino acid. Other methods of codon optimization known in the art are contemplated for use herein.

The methods of modifying a target polynucleotide sequence in a cell according to the invention as described herein may comprise a Fanzor nuclease and a fRNA to be delivered together (e.g., by the same vector) or delivered separately (e.g. as separate vectors). A Fanzor nuclease of the present disclosure may be unstable without co-delivery of the fRNA molecule (e.g., when a Fanzor nuclease and the fRNA molecule are delivered by separate vectors). In some embodiments, the Fanzor nuclease is stable in the presence of the fRNA molecule. In some embodiments, the Fanzor nuclease is stable in the absence of the fRNA molecule. In some embodiments, the Fanzor polypeptide encoding the Fanzor nuclease (e.g., the Fanzor nuclease encoding sequence) is modified to increase stability. In some embodiments, the modifications include, but are not limited to, one or more mutations relative to the wildtype Fanzor polypeptide wherein the one or more mutations result in a Fanzor polypeptide that has increased stability in the absence of the fRNA relative to an unmodified Fanzor polypeptide. An exemplary modification is the fusion of a stabilizing domain to a Fanzor polypeptide to increase stability. Non-limiting examples of stabilizing domains that can be fused with a Fanzor nuclease of the present disclosure include a small ubiquitin-like modifier (SUMO) tag, glutathione-S-transferase (GST) tag, and/or superfolder green fluorescent protein (sfGFP). Other modifications known in the art for increasing the stabilization of a polypeptide, and/or of a nuclease, are contemplated herein.

The compositions described herein may be used in various nucleic acids-targeting applications, altering or modifying synthesis of a gene product, such as a protein, nucleic acids cleavage, nucleic acids editing, nucleic acids splicing; trafficking of target nucleic acids, tracing of target nucleic acids, isolation of target nucleic acids, visualization of target nucleic acids, etc. Aspects of the invention also encompass methods and uses of the compositions and systems described herein in genome engineering, e.g. for altering or manipulating the expression of one or more genes or the one or more gene products, in prokaryotic or eukaryotic cells, in vitro, in vivo or ex vivo. In some examples, the target polynucleotides are target sequences within genomic DNA, including nuclear genomic DNA, mitochondrial DNA, or chloroplast DNA. In some embodiments, the target sequence is a viral polynucleotide. In some embodiments, the viral polynucleotide is integrated within a host genome. Aspects of the invention also encompass methods and uses of the compositions and systems described herein for multiplexed editing. In some embodiments, the multiplexed editing targets 2, 3, 4, 5, 6, 7, 8, 9, 10 or more sites. In some embodiments, the target polynucleotide is a gene related to disease resistance or pest control. In some embodiments, the genome engineering is directed towards modifying crop traits. Non-limiting examples of crop trait modifications include improved yield, improved taste, and improved nutritional value. In some embodiments, the genome engineering is directed towards bioenergy production. In some embodiments, the genome engineering is directed towards modifying organisms to optimize the production of biofuels. Non-limiting examples of organisms that can be modified to optimize the production of biofuels include algae, bacteria, yeast, microalgae, sugarcane, corn, switchgrass, miscanthus, sorghum, soybean, canola, jatropha, Trichoderma, Aspergillus, and macroalgae. In some embodiments, the genome engineering is directed towards bioremediation. In some embodiments, the genome engineering is directed towards modifying microbes to degrade environmental pollutants. Non-limiting examples or microbes that can be modified to degrade environmental pollutants include Brevibacterium epidermis EZ-K02, Microbacterium oleivorans, Irpex lacteus, Bacillus subtilis HUK15, Anaeromyxobacter sp Fw109-5, Bacillus, Corprothermobacter, Rhodobacter, Pseudomonas, Achromobacter, Desfulitobacter, Desulfosporosinus, T78, Methanobacterium, Methanosaeta, Proteobacteria, Firmicutes, Naegleria, Vorticella, Arabidopsis, Asarum, Populus, Koribacter, Acidomicrobium, Bradyrhizobiu, Burkholderia, Solibacter, Singulisphaera, Desulfomonile, Rhodococcus, Bordatella, Chromobacter, Variovorax, Thiobacillus sp., Pseudoxanthomonas sp., Alcanivorax sp., Acinetobacter venetianus RAG-1, Dehalococcoides mccartyi, Actinobacter, Mycobacterium, Pseudomonas aeruginosa, Penicillium oxalicum, Sphingomonas sp. GY2B, Miscanthus sinesis, Rhizobiales, Burkholderiales, Actinomycetales, Pseudomonas putida, Pseudomonas putida KT2440, Rhodococcus aetherivorans BCP1, Rhodococcus opacus R7, and Pseudomonas stutzeri 5190. Aspects of the invention also encompass methods and uses of the compositions and systems described herein in chromosome imaging, e.g. for visualizing specific sequences within live cells. In some examples, chromosome imaging is performed by fluorescently-tagging the compositions described herein.

The compositions described herein may be used to create genetically modified animal models or to create functional genomic screens. In some embodiments, the genetically modified animal models can be used for disease research. In some embodiments, the functional genomic screens can be used to identify genes involved in specific biological processes. In some embodiments, the functional genomic screens can be used to identify polynucleotide sequences related to disease pathogens. In some embodiments, the polynucleotide sequences are DNA. In some embodiments, the polynucleotide sequences are RNA. Any disease or disorder that may be detected using any of the composition or methods described herein (e.g., Fanzor systems) are contemplated for detection herein.

In some aspects, the invention provides methods comprising delivering one or more polynucleotides, such as or one or more vectors as described herein, one or more transcripts thereof, and/or one or proteins transcribed therefrom, to a host cell. In some aspects, the invention further provides cells produced by such methods, and organisms (such as animals, plants, seeds, or fungi) comprising or produced from such cells. In some embodiments, a base editor as described herein in combination with (and optionally complexed with) a guide sequence is delivered to a cell.

Exemplary delivery strategies are known in the art, and described herein, which include vector-based strategies. In some embodiments, the method of delivery provided comprises nucleofection, microinjection, biolistics, virosomes, liposomes, immunoliposomes, polycation or lipid: nucleic acid conjugates, naked DNA, artificial virions, and agent-enhanced uptake of DNA. Exemplary methods of delivery of nucleic acids include lipofection, nucleofection, electoporation, stable genome integration (e.g., piggybac), microinjection, biolistics, virosomes, liposomes, immunoliposomes, polycation or lipid: nucleic acid conjugates, naked DNA, artificial virions, and agent-enhanced uptake of DNA. Lipofection is described in e.g., U.S. Pat. Nos.5,049,386, 4,946,787; and 4,897,355) and lipofection reagents are sold commercially (e.g., Transfectam™, Lipofectin™ and SF Cell Line 4D-Nucleofector X Kit™ (Lonza)). Cationic and neutral lipids that are suitable for efficient receptor-recognition lipofection of polynucleotides include those of Feigner, WO 91/17424; WO 91/16024. Other methods of delivery known in the art are contemplated for use with Fanzor system described herein.

Delivery may be to cells (e.g. in vitro or ex vivo administration) or target tissues (e.g. in vivo administration). Delivery methods known in the art are contemplated for use herein. As a non-limiting example, the compositions and methods of the present invention may be delivered via ex vivo administration to non-limiting cell types such as B cells, T cells, tumor infiltrating lymphocytes (TIL), CARTs, and/or stem cells (e.g., bone marrow stem cells) for the treatment of various diseases. Other cell types compatible with ex vivo administration known in the art are also contemplated for use with the compositions and methods disclosed herein. The compositions and methods of the present invention may be delivered via in vivo administration to target tissues and/or cells of target tissues using, as non-limiting examples, AAV or other programmable tissue-specific lipid nanoparticles (LNPs). Other methods of in vivo administration known in the art are also contemplated for use with the compositions and methods disclosed herein.

Delivery may be achieved through the use of RNP complexes. Examples of target polynucleotides include a sequence associated with a signaling biochemical pathway, e.g., a signaling biochemical pathway-associated gene or polynucleotide. Examples of target polynucleotides include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non-disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown, and may be at a normal or abnormal level. Examples of target polynucleotides include a viral associated gene or polynucleotide. A “viral-associated” gene or polynucleotide refers to any gene or polynucleotide of viral origin integrated within a host genome. It may be a gene that is involved in the replication, transcription, translation, or assembly of a virus. It may be a gene that is highly conserved among viruses. For example, in some embodiments, a method is provided that comprises administering to a subject having a viral disease an effective amount of the Fanzor editing system described herein that introduces a deactivating mutation into a viral-associated gene.

The “disease-associated” gene or polynucleotide can be associated with a monogenetic disorder selected from the group consisting of: Adenosine Deaminase (ADA) Deficiency; Alpha-1 Antitrypsin Deficiency; Cystic Fibrosis; Duchenne Muscular Dystrophy; Galactosemia; Hemochromatosis; Huntington's Disease; Maple Syrup Urine Disease; Marfan Syndrome; Neurofibromatosis Type 1; Pachyonychia Congenita; Phenylkeotnuria; Severe Combined Immunodeficiency; Sickle Cell Disease; Smith-Lemli-Opitz Syndrome; and Tay-Sachs Disease. In other embodiments, the disease-associated gene can be associated with a polygenic disorder selected from the group consisting of: heart disease; high blood pressure; Alzheimer's disease; arthritis; diabetes; cancer; and obesity. The compositions described herein may be administered to a subject in need thereof in a therapeutically effective amount to treat and/or prevent a disease or disorder the subject is suffering from. Any disease or disorder that may be treated and/or prevented using any of the composition or methods described herein (e.g., Fanzor systems) are contemplated for treatment herein. Any disease is conceivably treatable by such methods so long as delivery to the appropriate cells is feasible. The person having ordinary skill in the art will be able to choose and/or select a Fanzor delivery methodology to suit the intended purpose and the intended target cells.

For example, in some embodiments, a method is provided that comprises administering to a subject having such a disease, e.g., a cancer associated with a point mutation as described above, an effective amount of the Fanzor editing system described herein that corrects the point mutation or introduces a deactivating mutation into a disease-associated gene as mediated by homology-directed repair in the presence of a donor DNA molecule comprising desired genetic change. In some embodiments, a method is provided that comprises administering to a subject having such a disease, e.g., a cancer associated with a point mutation as described above, an effective amount of the Fanzor editing system described herein that corrects the point mutation or introduces a deactivating mutation into a disease-associated gene. In some embodiments, the disease is a proliferative disease. In some embodiments, the disease is a genetic disease. In some embodiments, the disease is a neoplastic disease. In some embodiments, the disease is a metabolic disease. In some embodiments, the disease is a lysosomal storage disease. Other diseases that can be treated by correcting a point mutation or introducing a deactivating mutation into a disease-associated gene will be known to those of skill in the art, and the disclosure is not limited in this respect.

The instant disclosure provides methods for the treatment of additional diseases or disorders, e.g., diseases or disorders that are associated or caused by a point mutation that can be corrected by Fanzor-mediated gene editing. Some such diseases are described herein, and additional suitable diseases that can be treated with the strategies and fusion proteins provided herein will be apparent to those of skill in the art based on the instant disclosure. Exemplary suitable diseases and disorders are listed below. It will be understood that the numbering of the specific positions or residues in the respective sequences depends on the particular protein and numbering scheme used. Numbering might be different, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment and determination of homologous residues. Exemplary suitable diseases and disorders include, without limitation: 2-methyl-3-hydroxybutyric aciduria; 3 beta-Hydroxysteroid dehydrogenase deficiency; 3-Methylglutaconic aciduria; 3-Oxo-5 alpha-steroid delta 4-dehydrogenase deficiency; 46,XY sex reversal, type 1, 3, and 5; 5-Oxoprolinase deficiency; 6-pyruvoyl-tetrahydropterin synthase deficiency; Aarskog syndrome; Aase syndrome; Achondrogenesis type 2; Achromatopsia 2 and 7; Acquired long QT syndrome; Acrocallosal syndrome, Schinzel type; Acrocapitofemoral dysplasia; Acrodysostosis 2, with or without hormone resistance; Acroerythrokeratoderma; Acromicric dysplasia; Acth-independent macronodular adrenal hyperplasia 2; Activated PI3K-delta syndrome; Acute intermittent porphyria; deficiency of Acyl-CoA dehydrogenase family, member 9; Adams-Oliver syndrome 5 and 6; Adenine phosphoribosyltransferase deficiency; Adenylate kinase deficiency; hemolytic anemia due to Adenylosuccinate lyase deficiency; Adolescent nephronophthisis; Renal-hepatic-pancreatic dysplasia; Meckel syndrome type 7; Adrenoleukodystrophy; Adult junctional epidermolysis bullosa; Epidermolysis bullosa, junctional, localisata variant; Adult neuronal ceroid lipofuscinosis; Adult neuronal ceroid lipofuscinosis; Adult onset ataxia with oculomotor apraxia; ADULT syndrome; Afibrinogenemia and congenital Afibrinogenemia; autosomal recessive Agammaglobulinemia 2; Age-related macular degeneration 3, 6, 11, and 12; Aicardi Goutieres syndromes 1, 4, and 5; Chilbain lupus 1; Alagille syndromes 1 and 2; Alexander disease; Alkaptonuria; Allan-Herndon-Dudley syndrome; Alopecia universalis congenital; Alpers encephalopathy; Alpha-1-antitrypsin deficiency; autosomal dominant, autosomal recessive, and X-linked recessive Alport syndromes; Alzheimer disease, familial, 3, with spastic paraparesis and apraxia; Alzheimer disease, types, 1, 3, and 4; hypocalcification type and hypomaturation type, IIA1 Amelogenesis imperfecta; Aminoacylase 1 deficiency; Amish infantile epilepsy syndrome; Amyloidogenic transthyretin amyloidosis; Amyloid Cardiomyopathy, Transthyretin-related; Cardiomyopathy; Amyotrophic lateral sclerosis types 1, 6, 15 (with or without frontotemporal dementia), 22 (with or without frontotemporal dementia), and 10; Frontotemporal dementia with TDP43 inclusions, TARDBP-related; Andermann syndrome; Andersen Tawil syndrome; Congenital long QT syndrome; Anemia, nonspherocytic hemolytic, due to G6PD deficiency; Angelman syndrome; Severe neonatal-onset encephalopathy with microcephaly; susceptibility to Autism, X-linked 3; Angiopathy, hereditary, with nephropathy, aneurysms, and muscle cramps; Angiotensin i-converting enzyme, benign serum increase; Aniridia, cerebellar ataxia, and mental retardation; Anonychia; Antithrombin III deficiency; Antley-Bixler syndrome with genital anomalies and disordered steroidogenesis; Aortic aneurysm, familial thoracic 4, 6, and 9; Thoracic aortic aneurysms and aortic dissections; Multisystemic smooth muscle dysfunction syndrome; Moyamoya disease 5; Aplastic anemia; Apparent mineralocorticoid excess; Arginase deficiency; Argininosuccinate lyase deficiency; Aromatase deficiency; Arrhythmogenic right ventricular cardiomyopathy types 5, 8, and 10; Primary familial hypertrophic cardiomyopathy; Arthrogryposis multiplex congenita, distal, X-linked; Arthrogryposis renal dysfunction cholestasis syndrome; Arthrogryposis, renal dysfunction, and cholestasis 2; Asparagine synthetase deficiency; Abnormality of neuronal migration; Ataxia with vitamin E deficiency; Ataxia, sensory, autosomal dominant; Ataxia-telangiectasia syndrome; Hereditary cancer-predisposing syndrome; Atransferrinemia; Atrial fibrillation, familial, 11, 12, 13, and 16; Atrial septal defects 2, 4, and 7 (with or without atrioventricular conduction defects); Atrial standstill 2; Atrioventricular septal defect 4; Atrophia bulborum hereditaria; ATR-X syndrome; Auriculocondylar syndrome 2; Autoimmune disease, multisystem, infantile-onset; Autoimmune lymphoproliferative syndrome, type 1a; Autosomal dominant hypohidrotic ectodermal dysplasia; Autosomal dominant progressive external ophthalmoplegia with mitochondrial DNA deletions 1 and 3; Autosomal dominant torsion dystonia 4; Autosomal recessive centronuclear myopathy; Autosomal recessive congenital ichthyosis 1, 2, 3, 4A, and 4B; Autosomal recessive cutis laxa type IA and 1B; Autosomal recessive hypohidrotic ectodermal dysplasia syndrome; Ectodermal dysplasia 11b; hypohidrotic/hair/tooth type, autosomal recessive; Autosomal recessive hypophosphatemic bone disease; Axenfeld-Rieger syndrome type 3; Bainbridge-Ropers syndrome; Bannayan-Riley-Ruvalcaba syndrome; PTEN hamartoma tumor syndrome; Baraitser-Winter syndromes 1 and 2; Barakat syndrome; Bardet-Biedl syndromes 1, 11, 16, and 19; Bare lymphocyte syndrome type 2, complementation group E; Bartter syndrome antenatal type 2; Bartter syndrome types 3, 3 with hypocalciuria, and 4; Basal ganglia calcification, idiopathic, 4; Beaded hair; Benign familial hematuria; Benign familial neonatal seizures 1 and 2; Seizures, benign familial neonatal, 1, and/or myokymia; Seizures, Early infantile epileptic encephalopathy 7; Benign familial neonatal-infantile seizures; Benign hereditary chorea; Benign scapuloperoneal muscular dystrophy with cardiomyopathy; Bernard-Soulier syndrome, types A1 and A2 (autosomal dominant); Bestrophinopathy, autosomal recessive; beta Thalassemia; Bethlem myopathy and Bethlem myopathy 2; Bietti crystalline corneoretinal dystrophy; Bile acid synthesis defect, congenital, 2; Biotinidase deficiency; Birk Barel mental retardation dysmorphism syndrome; Blepharophimosis, ptosis, and epicanthus inversus; Bloom syndrome; Borjeson-Forssman-Lehmann syndrome; Boucher Neuhauser syndrome; Brachydactyly types A1 and A2; Brachydactyly with hypertension; Brain small vessel disease with hemorrhage; Branched-chain ketoacid dehydrogenase kinase deficiency; Branchiootic syndromes 2 and 3; Breast cancer, early-onset; Breast-ovarian cancer, familial 1, 2, and 4; Brittle cornea syndrome 2; Brody myopathy; Bronchiectasis with or without elevated sweat chloride 3; Brown-Vialetto-Van laere syndrome and Brown-Vialetto-Van Laere syndrome 2; Brugada syndrome; Brugada syndrome 1; Ventricular fibrillation; Paroxysmal familial ventricular fibrillation; Brugada syndrome and Brugada syndrome 4; Long QT syndrome; Sudden cardiac death; Bull eye macular dystrophy; Stargardt disease 4; Cone-rod dystrophy 12; Bullous ichthyosiform erythroderma; Burn-Mckeown syndrome; Candidiasis, familial, 2, 5, 6, and 8; Carbohydrate-deficient glycoprotein syndrome type I and II; Carbonic anhydrase VA deficiency, hyperammonemia due to; Carcinoma of colon; Cardiac arrhythmia; Long QT syndrome, LQT1 subtype; Cardioencephalomyopathy, fatal infantile, due to cytochrome c oxidase deficiency; Cardiofaciocutaneous syndrome; Cardiomyopathy; Danon disease; Hypertrophic cardiomyopathy; Left ventricular noncompaction cardiomyopathy; Carnevale syndrome; Carney complex, type 1; Carnitine acylcarnitine translocase deficiency; Carnitine palmitoyltransferase I, II, II (late onset), and II (infantile) deficiency; Cataract 1, 4, autosomal dominant, autosomal dominant, multiple types, with microcornea, coppock-like, juvenile, with microcornea and glucosuria, and nuclear diffuse nonprogressive; Catecholaminergic polymorphic ventricular tachycardia; Caudal regression syndrome; Cd8 deficiency, familial; Central core disease; Centromeric instability of chromosomes 1,9 and 16 and immunodeficiency; Cerebellar ataxia infantile with progressive external ophthalmoplegi and Cerebellar ataxia, mental retardation, and dysequilibrium syndrome 2; Cerebral amyloid angiopathy, APP-related; Cerebral autosomal dominant and recessive arteriopathy with subcortical infarcts and leukoencephalopathy; Cerebral cavernous malformations 2; Cerebrooculofacioskeletal syndrome 2; Cerebro-oculo-facio-skeletal syndrome; Cerebroretinal microangiopathy with calcifications and cysts; Ceroid lipofuscinosis neuronal 2, 6, 7, and 10; Ch\xc3\xa9diak-Higashi syndrome, Chediak-Higashi syndrome, adult type; Charcot-Marie-Tooth disease types 1B, 2B2, 2C, 2F, 2I, 2U (axonal), 1C (demyelinating), dominant intermediate C, recessive intermediate A, 2A2, 4C, 4D, 4H, IF, IVF, and X; Scapuloperoneal spinal muscular atrophy; Distal spinal muscular atrophy, congenital nonprogressive; Spinal muscular atrophy, distal, autosomal recessive, 5; CHARGE association; Childhood hypophosphatasia; Adult hypophosphatasia; Cholecystitis; Progressive familial intrahepatic cholestasis 3; Cholestasis, intrahepatic, of pregnancy 3; Cholestanol storage disease; Cholesterol monooxygenase (side-chain cleaving) deficiency; Chondrodysplasia Blomstrand type; Chondrodysplasia punctata 1, X-linked recessive and 2 X-linked dominant; CHOPS syndrome; Chronic granulomatous disease, autosomal recessive cytochrome b-positive, types 1 and 2; Chudley-Mccullough syndrome; Ciliary dyskinesia, primary, 7, 11, 15, 20 and 22; Citrullinemia type I; Citrullinemia type I and II; Cleidocranial dysostosis; C-like syndrome; Cockayne syndrome type A; Coenzyme Q10 deficiency, primary 1, 4, and 7; Coffin Siris/Intellectual Disability; Coffin-Lowry syndrome; Cohen syndrome; Cold-induced sweating syndrome 1; COLE-CARPENTER SYNDROME 2; Combined cellular and humoral immune defects with granulomas; Combined d-2— and 1-2-hydroxyglutaric aciduria; Combined malonic and methylmalonic aciduria; Combined oxidative phosphorylation deficiencies 1, 3, 4, 12, 15, and 25; Combined partial and complete 17-alpha-hydroxylase/17,20-lyase deficiency; Common variable immunodeficiency 9; Complement component 4, partial deficiency of, due to dysfunctional c1 inhibitor; Complement factor B deficiency; Cone monochromatism; Cone-rod dystrophy 2 and 6; Cone-rod dystrophy amelogenesis imperfecta; Congenital adrenal hyperplasia and Congenital adrenal hypoplasia, X-linked; Congenital amegakaryocytic thrombocytopenia; Congenital aniridia; Congenital central hypoventilation; Hirschsprung disease 3; Congenital contractural arachnodactyly; Congenital contractures of the limbs and face, hypotonia, and developmental delay; Congenital disorder of glycosylation types 1B, 1D, 1G, 1H, 1J, 1K, IN, 1P, 2C, 2J, 2K, IIm; Congenital dyserythropoietic anemia, type I and II; Congenital ectodermal dysplasia of face; Congenital erythropoietic porphyria; Congenital generalized lipodystrophy type 2; Congenital heart disease, multiple types, 2; Congenital heart disease; Interrupted aortic arch; Congenital lipomatous overgrowth, vascular malformations, and epidermal nevi; Non-small cell lung cancer; Neoplasm of ovary; Cardiac conduction defect, nonspecific; Congenital microvillous atrophy; Congenital muscular dystrophy; Congenital muscular dystrophy due to partial LAMA2 deficiency; Congenital muscular dystrophy-dystroglycanopathy with brain and eye anomalies, types A2, A7, A8, A11, and A14; Congenital muscular dystrophy-dystroglycanopathy with mental retardation, types B2, B3, B5, and B15; Congenital muscular dystrophy-dystroglycanopathy without mental retardation, type B5; Congenital muscular hypertrophy-cerebral syndrome; Congenital myasthenic syndrome, acetazolamide-responsive; Congenital myopathy with fiber type disproportion; Congenital ocular coloboma; Congenital stationary night blindness, type 1A, 1B, 1C, 1E, 1F, and 2A; Coproporphyria; Cornea plana 2; Corneal dystrophy, Fuchs endothelial, 4; Corneal endothelial dystrophy type 2; Corneal fragility keratoglobus, blue sclerae and joint hypermobility; Cornelia de Lange syndromes 1 and 5; Coronary artery disease, autosomal dominant 2; Coronary heart disease; Hyperalphalipoproteinemia 2; Cortical dysplasia, complex, with other brain malformations 5 and 6; Cortical malformations, occipital; Corticosteroid-binding globulin deficiency; Corticosterone methyloxidase type 2 deficiency; Costello syndrome; Cowden syndrome 1; Coxa plana; Craniodiaphyseal dysplasia, autosomal dominant; Craniosynostosis 1 and 4; Craniosynostosis and dental anomalies; Creatine deficiency, X-linked; Crouzon syndrome; Cryptophthalmos syndrome; Cryptorchidism, unilateral or bilateral; Cushing symphalangism; Cutaneous malignant melanoma 1; Cutis laxa with osteodystrophy and with severe pulmonary, gastrointestinal, and urinary abnormalities; Cyanosis, transient neonatal and atypical nephropathic; Cystic fibrosis; Cystinuria; Cytochrome c oxidase i deficiency; Cytochrome-c oxidase deficiency; D-2-hydroxyglutaric aciduria 2; Darier disease, segmental; Deafness with labyrinthine aplasia microtia and microdontia (LAMM); Deafness, autosomal dominant 3a, 4, 12, 13, 15, autosomal dominant nonsyndromic sensorineural 17, 20, and 65; Deafness, autosomal recessive 1A, 2, 3, 6, 8, 9, 12, 15, 16, 18b, 22, 28, 31, 44, 49, 63, 77, 86, and 89; Deafness, cochlear, with myopia and intellectual impairment, without vestibular involvement, autosomal dominant, X-linked 2; Deficiency of 2-methylbutyryl-CoA dehydrogenase; Deficiency of 3-hydroxyacyl-CoA dehydrogenase; Deficiency of alpha-mannosidase; Deficiency of aromatic-L-amino-acid decarboxylase; Deficiency of bisphosphoglycerate mutase; Deficiency of butyryl-CoA dehydrogenase; Deficiency of ferroxidase; Deficiency of galactokinase; Deficiency of guanidinoacetate methyltransferase; Deficiency of hyaluronoglucosaminidase; Deficiency of ribose-5-phosphate isomerase; Deficiency of steroid 11-beta-monooxygenase; Deficiency of UDPglucose-hexose-1-phosphate uridylyltransferase; Deficiency of xanthine oxidase; Dejerine-Sottas disease; Charcot-Marie-Tooth disease, types ID and IVF; Dejerine-Sottas syndrome, autosomal dominant; Dendritic cell, monocyte, B lymphocyte, and natural killer lymphocyte deficiency; Desbuquois dysplasia 2; Desbuquois syndrome; DFNA 2 Nonsyndromic Hearing Loss; Diabetes mellitus and insipidus with optic atrophy and deafness; Diabetes mellitus, type 2, and insulin-dependent, 20; Diamond-Blackfan anemia 1, 5, 8, and 10; Diarrhea 3 (secretory sodium, congenital, syndromic) and 5 (with tufting enteropathy, congenital); Dicarboxylic aminoaciduria; Diffuse palmoplantar keratoderma, Bothnian type; Digitorenocerebral syndrome; Dihydropteridine reductase deficiency; Dilated cardiomyopathy 1A, 1AA, 1C, 1G, 1BB, 1DD, 1FF, 1HH, 1I, 1KK, IN, 1S, 1Y, and 3B; Left ventricular noncompaction 3; Disordered steroidogenesis due to cytochrome p450 oxidoreductase deficiency; Distal arthrogryposis type 2B; Distal hereditary motor neuronopathy type 2B; Distal myopathy Markesbery-Griggs type; Distal spinal muscular atrophy, X-linked 3; Distichiasis-lymphedema syndrome; Dominant dystrophic epidermolysis bullosa with absence of skin; Dominant hereditary optic atrophy; Donnai Barrow syndrome; Dopamine beta hydroxylase deficiency; Dopamine receptor d2, reduced brain density of; Dowling-degos disease 4; Doyne honeycomb retinal dystrophy; Malattia leventinese; Duane syndrome type 2; Dubin-Johnson syndrome; Duchenne muscular dystrophy; Becker muscular dystrophy; Dysfibrinogenemia; Dyskeratosis congenita autosomal dominant and autosomal dominant, 3; Dyskeratosis congenita, autosomal recessive, 1, 3, 4, and 5; Dyskeratosis congenita X-linked; Dyskinesia, familial, with facial myokymia; Dysplasminogenemia; Dystonia 2 (torsion, autosomal recessive), 3 (torsion, X-linked), 5 (Dopa-responsive type), 10, 12, 16, 25, 26 (Myoclonic); Seizures, benign familial infantile, 2; Early infantile epileptic encephalopathy 2, 4, 7, 9, 10, 11, 13, and 14; Atypical Rett syndrome; Early T cell progenitor acute lymphoblastic leukemia; Ectodermal dysplasia skin fragility syndrome; Ectodermal dysplasia-syndactyly syndrome 1; Ectopia lentis, isolated autosomal recessive and dominant; Ectrodactyly, ectodermal dysplasia, and cleft lip/palate syndrome 3; Ehlers-Danlos syndrome type 7 (autosomal recessive), classic type, type 2 (progeroid), hydroxylysine-deficient, type 4, type 4 variant, and due to tenascin-X deficiency; Eichsfeld type congenital muscular dystrophy; Endocrine-cerebroosteodysplasia; Enhanced s-cone syndrome; Enlarged vestibular aqueduct syndrome; Enterokinase deficiency; Epidermodysplasia verruciformis; Epidermolysa bullosa simplex and limb girdle muscular dystrophy, simplex with mottled pigmentation, simplex with pyloric atresia, simplex, autosomal recessive, and with pyloric atresia; Epidermolytic palmoplantar keratoderma; Familial febrile seizures 8; Epilepsy, childhood absence 2, 12 (idiopathic generalized, susceptibility to) 5 (nocturnal frontal lobe), nocturnal frontal lobe type 1, partial, with variable foci, progressive myoclonic 3, and X-linked, with variable learning disabilities and behavior disorders; Epileptic encephalopathy, childhood-onset, early infantile, 1, 19, 23, 25, 30, and 32; Epiphyseal dysplasia, multiple, with myopia and conductive deafness; Episodic ataxia type 2; Episodic pain syndrome, familial, 3; Epstein syndrome; Fechtner syndrome; Erythropoietic protoporphyria; Estrogen resistance; Exudative vitreoretinopathy 6; Fabry disease and Fabry disease, cardiac variant; Factor H, VII, X, v and factor viii, combined deficiency of 2, xiii, a subunit, deficiency; Familial adenomatous polyposis 1 and 3; Familial amyloid nephropathy with urticaria and deafness; Familial cold urticarial; Familial aplasia of the vermis; Familial benign pemphigus; Familial cancer of breast; Breast cancer, susceptibility to; Osteosarcoma; Pancreatic cancer 3; Familial cardiomyopathy; Familial cold autoinflammatory syndrome 2; Familial colorectal cancer; Familial exudative vitreoretinopathy, X-linked; Familial hemiplegic migraine types 1 and 2; Familial hypercholesterolemia; Familial hypertrophic cardiomyopathy 1, 2, 3, 4, 7, 10, 23 and 24; Familial hypokalemia-hypomagnesemia; Familial hypoplastic, glomerulocystic kidney; Familial infantile myasthenia; Familial juvenile gout; Familial Mediterranean fever and Familial mediterranean fever, autosomal dominant; Familial porencephaly; Familial porphyria cutanea tarda; Familial pulmonary capillary hemangiomatosis; Familial renal glucosuria; Familial renal hypouricemia; Familial restrictive cardiomyopathy 1; Familial type 1 and 3 hyperlipoproteinemia; Fanconi anemia, complementation group E, I, N, and O; Fanconi-Bickel syndrome; Favism, susceptibility to; Febrile seizures, familial, 11; Feingold syndrome 1; Fetal hemoglobin quantitative trait locus 1; FG syndrome and FG syndrome 4; Fibrosis of extraocular muscles, congenital, 1, 2, 3a (with or without extraocular involvement), 3b; Fish-eye disease; Fleck corneal dystrophy; Floating-Harbor syndrome; Focal epilepsy with speech disorder with or without mental retardation; Focal segmental glomerulosclerosis 5; Forebrain defects; Frank Ter Haar syndrome; Borrone Di Rocco Crovato syndrome; Frasier syndrome; Wilms tumor 1; Freeman-Sheldon syndrome; Frontometaphyseal dysplasia land 3; Frontotemporal dementia; Frontotemporal dementia and/or amyotrophic lateral sclerosis 3 and 4; Frontotemporal Dementia Chromosome 3-Linked and Frontotemporal dementia ubiquitin-positive; Fructose-biphosphatase deficiency; Fuhrmann syndrome; Gamma-aminobutyric acid transaminase deficiency; Gamstorp-Wohlfart syndrome; Gaucher disease type 1 and Subacute neuronopathic; Gaze palsy, familial horizontal, with progressive scoliosis; Generalized dominant dystrophic epidermolysis bullosa; Generalized epilepsy with febrile seizures plus 3, type 1, type 2; Epileptic encephalopathy Lennox-Gastaut type; Giant axonal neuropathy; Glanzmann thrombasthenia; Glaucoma 1, open angle, e, F, and G; Glaucoma 3, primary congenital, d; Glaucoma, congenital and Glaucoma, congenital, Coloboma; Glaucoma, primary open angle, juvenile-onset; Glioma susceptibility 1; Glucose transporter type 1 deficiency syndrome; Glucose-6-phosphate transport defect; GLUT1 deficiency syndrome 2; Epilepsy, idiopathic generalized, susceptibility to, 12; Glutamate formiminotransferase deficiency; Glutaric acidemia IIA and IIB; Glutaric aciduria, type 1; Gluthathione synthetase deficiency; Glycogen storage disease 0 (muscle), II (adult form), IXa2, IXc, type 1A; type II, type IV, IV (combined hepatic and myopathic), type V, and type VI; Goldmann-Favre syndrome; Gordon syndrome; Gorlin syndrome; Holoprosencephaly sequence; Holoprosencephaly 7; Granulomatous disease, chronic, X-linked, variant; Granulosa cell tumor of the ovary; Gray platelet syndrome; Griscelli syndrome type 3; Groenouw corneal dystrophy type I; Growth and mental retardation, mandibulofacial dysostosis, microcephaly, and cleft palate; Growth hormone deficiency with pituitary anomalies; Growth hormone insensitivity with immunodeficiency; GTP cyclohydrolase I deficiency; Hajdu-Cheney syndrome; Hand foot uterus syndrome; Hearing impairment; Hemangioma, capillary infantile; Hematologic neoplasm; Hemochromatosis type 1, 2B, and 3; Microvascular complications of diabetes 7; Transferrin serum level quantitative trait locus 2; Hemoglobin H disease, nondeletional; Hemolytic anemia, nonspherocytic, due to glucose phosphate isomerase deficiency; Hemophagocytic lymphohistiocytosis, familial, 2; Hemophagocytic lymphohistiocytosis, familial, 3; Heparin cofactor II deficiency; Hereditary acrodermatitis enteropathica; Hereditary breast and ovarian cancer syndrome; Ataxia-telangiectasia-like disorder; Hereditary diffuse gastric cancer; Hereditary diffuse leukoencephalopathy with spheroids; Hereditary factors II, IX, VIII deficiency disease; Hereditary hemorrhagic telangiectasia type 2; Hereditary insensitivity to pain with anhidrosis; Hereditary lymphedema type I; Hereditary motor and sensory neuropathy with optic atrophy; Hereditary myopathy with early respiratory failure; Hereditary neuralgic amyotrophy; Hereditary Nonpolyposis Colorectal Neoplasms; Lynch syndrome I and II; Hereditary pancreatitis; Pancreatitis, chronic, susceptibility to; Hereditary sensory and autonomic neuropathy type IIB amd IIA; Hereditary sideroblastic anemia; Hermansky-Pudlak syndrome 1, 3, 4, and 6; Heterotaxy, visceral, 2, 4, and 6, autosomal; Heterotaxy, visceral, X-linked; Heterotopia; Histiocytic medullary reticulosis; Histiocytosis-lymphadenopathy plus syndrome; Holocarboxylase synthetase deficiency; Holoprosencephaly 2, 3,7, and 9; Holt-Oram syndrome; Homocysteinemia due to MTHFR deficiency, CBS deficiency, and Homocystinuria, pyridoxine-responsive; Homocystinuria-Megaloblastic anemia due to defect in cobalamin metabolism, cblE complementation type; Howel-Evans syndrome; Hurler syndrome; Hutchinson-Gilford syndrome; Hydrocephalus; Hyperammonemia, type III; Hypercholesterolaemia and Hypercholesterolemia, autosomal recessive; Hyperekplexia 2 and Hyperekplexia hereditary; Hyperferritinemia cataract syndrome; Hyperglycinuria; Hyperimmunoglobulin D with periodic fever; Mevalonic aciduria; Hyperimmunoglobulin E syndrome; Hyperinsulinemic hypoglycemia familial 3, 4, and 5; Hyperinsulinism-hyperammonemia syndrome; Hyperlysinemia; Hypermanganesemia with dystonia, polycythemia and cirrhosis; Hyperornithinemia-hyperammonemia-homocitrullinuria syndrome; Hyperparathyroidism 1 and 2; Hyperparathyroidism, neonatal severe; Hyperphenylalaninemia, bh4-deficient, a, due to partial pts deficiency, BH4-deficient, D, and non-pku; Hyperphosphatasia with mental retardation syndrome 2, 3, and 4; Hypertrichotic osteochondrodysplasia; Hypobetalipoproteinemia, familial, associated with apob32; Hypocalcemia, autosomal dominant 1; Hypocalciuric hypercalcemia, familial, types 1 and 3; Hypochondrogenesis; Hypochromic microcytic anemia with iron overload; Hypoglycemia with deficiency of glycogen synthetase in the liver; Hypogonadotropic hypogonadism 11 with or without anosmia; Hypohidrotic ectodermal dysplasia with immune deficiency; Hypohidrotic X-linked ectodermal dysplasia; Hypokalemic periodic paralysis 1 and 2; Hypomagnesemia 1, intestinal; Hypomagnesemia, seizures, and mental retardation; Hypomyelinating leukodystrophy 7; Hypoplastic left heart syndrome; Atrioventricular septal defect and common atrioventricular junction; Hypospadias 1 and 2, X-linked; Hypothyroidism, congenital, nongoitrous, 1; Hypotrichosis 8 and 12; Hypotrichosis-lymphedema-telangiectasia syndrome; I blood group system; Ichthyosis bullosa of Siemens; Ichthyosis exfoliativa; Ichthyosis prematurity syndrome; Idiopathic basal ganglia calcification 5; Idiopathic fibrosing alveolitis, chronic form; Dyskeratosis congenita, autosomal dominant, 2 and 5; Idiopathic hypercalcemia of infancy; Immune dysfunction with T-cell inactivation due to calcium entry defect 2; Immunodeficiency 15, 16, 19, 30, 31C, 38, 40, 8, due to defect in cd3-zeta, with hyper IgM type 1 and 2, and X-Linked, with magnesium defect, Epstein-Barr virus infection, and neoplasia; Immunodeficiency-centromeric instability-facial anomalies syndrome 2; Inclusion body myopathy 2 and 3; Nonaka myopathy; Infantile convulsions and paroxysmal choreoathetosis, familial; Infantile cortical hyperostosis; Infantile GM1 gangliosidosis; Infantile hypophosphatasia; Infantile nephronophthisis; Infantile nystagmus, X-linked; Infantile Parkinsonism-dystonia; Infertility associated with multi-tailed spermatozoa and excessive DNA; Insulin resistance; Insulin-resistant diabetes mellitus and acanthosis nigricans; Insulin-dependent diabetes mellitus secretory diarrhea syndrome; Interstitial nephritis, karyomegalic; Intrauterine growth retardation, metaphyseal dysplasia, adrenal hypoplasia congenita, and genital anomalies; Iodotyrosyl coupling defect; IRAK4 deficiency; Iridogoniodysgenesis dominant type and type 1; Iron accumulation in brain; Ischiopatellar dysplasia; Islet cell hyperplasia; Isolated 17,20-lyase deficiency; Isolated lutropin deficiency; Isovaleryl-CoA dehydrogenase deficiency; Jankovic Rivera syndrome; Jervell and Lange-Nielsen syndrome 2; Joubert syndrome 1, 6, 7, 9/15 (digenic), 14, 16, and 17, and Orofaciodigital syndrome xiv; Junctional epidermolysis bullosa gravis of Herlitz; Juvenile GM>1<gangliosidosis; Juvenile polyposis syndrome; Juvenile polyposis/hereditary hemorrhagic telangiectasia syndrome; Juvenile retinoschisis; Kabuki make-up syndrome; Kallmann syndrome 1, 2, and 6; Delayed puberty; Kanzaki disease; Karak syndrome; Kartagener syndrome; Kenny-Caffey syndrome type 2; Keppen-Lubinsky syndrome; Keratoconus 1; Keratosis follicularis; Keratosis palmoplantaris striata 1; Kindler syndrome; L-2-hydroxyglutaric aciduria; Larsen syndrome, dominant type; Lattice corneal dystrophy Type III; Leber amaurosis; Zellweger syndrome; Peroxisome biogenesis disorders; Zellweger syndrome spectrum; Leber congenital amaurosis 11, 12, 13, 16, 4, 7, and 9; Leber optic atrophy; Aminoglycoside-induced deafness; Deafness, nonsyndromic sensorineural, mitochondrial; Left ventricular noncompaction 5; Left-right axis malformations; Leigh disease; Mitochondrial short-chain Enoyl-CoA Hydratase 1 deficiency; Leigh syndrome due to mitochondrial complex I deficiency; Leiner disease; Leri Weill dyschondrosteosis; Lethal congenital contracture syndrome 6; Leukocyte adhesion deficiency type I and III; Leukodystrophy, Hypomyelinating, 11 and 6; Leukoencephalopathy with ataxia, with Brainstem and Spinal Cord Involvement and Lactate Elevation, with vanishing white matter, and progressive, with ovarian failure; Leukonychia totalis; Lewy body dementia; Lichtenstein-Knorr Syndrome; Li-Fraumeni syndrome 1; Lig4 syndrome; Limb-girdle muscular dystrophy, type 1B, 2A, 2B, 2D, C1, C5, C9, C14; Congenital muscular dystrophy-dystroglycanopathy with brain and eye anomalies, type A14 and B14; Lipase deficiency combined; Lipid proteinosis; Lipodystrophy, familial partial, type 2 and 3; Lissencephaly 1, 2 (X-linked), 3, 6 (with microcephaly), X-linked; Subcortical laminar heterotopia, X-linked; Liver failure acute infantile; Loeys-Dietz syndrome 1, 2, 3; Long QT syndrome 1, 2, 2/9, 2/5, (digenic), 3, 5 and 5, acquired, susceptibility to; Lung cancer; Lymphedema, hereditary, id; Lymphedema, primary, with myelodysplasia; Lymphoproliferative syndrome 1, 1 (X-linked), and 2; Lysosomal acid lipase deficiency; Macrocephaly, macrosomia, facial dysmorphism syndrome; Macular dystrophy, vitelliform, adult-onset; Malignant hyperthermia susceptibility type 1; Malignant lymphoma, non-Hodgkin; Malignant melanoma; Malignant tumor of prostate; Mandibuloacral dysostosis; Mandibuloacral dysplasia with type A or B lipodystrophy, atypical; Mandibulofacial dysostosis, Treacher Collins type, autosomal recessive; Mannose-binding protein deficiency; Maple syrup urine disease type 1A and type 3; Marden Walker like syndrome; Marfan syndrome; Marinesco-Sj\xc3\xb6gren syndrome; Martsolf syndrome; Maturity-onset diabetes of the young, type 1, type 2, type 11, type 3, and type 9; May-Hegglin anomaly; MYH9 related disorders; Sebastian syndrome; McCune-Albright syndrome; Somatotroph adenoma; Sex cord-stromal tumor; Cushing syndrome; McKusick Kaufman syndrome; McLeod neuroacanthocytosis syndrome; Meckel-Gruber syndrome; Medium-chain acyl-coenzyme A dehydrogenase deficiency; Medulloblastoma; Megalencephalic leukoencephalopathy with subcortical cysts land 2a; Megalencephaly cutis marmorata telangiectatica congenital; PIK3CA Related Overgrowth Spectrum; Megalencephaly-polymicrogyria-polydactyly-hydrocephalus syndrome 2; Megaloblastic anemia, thiamine-responsive, with diabetes mellitus and sensorineural deafness; Meier-Gorlin syndromes land 4; Melnick-Needles syndrome; Meningioma; Mental retardation, X-linked, 3, 21, 30, and 72; Mental retardation and microcephaly with pontine and cerebellar hypoplasia; Mental retardation X-linked syndromic 5; Mental retardation, anterior maxillary protrusion, and strabismus; Mental retardation, autosomal dominant 12, 13, 15, 24, 3, 30, 4, 5, 6, and 9; Mental retardation, autosomal recessive 15, 44, 46, and 5; Mental retardation, stereotypic movements, epilepsy, and/or cerebral malformations; Mental retardation, syndromic, Claes-Jensen type, X-linked; Mental retardation, X-linked, nonspecific, syndromic, Hedera type, and syndromic, wu type; Merosin deficient congenital muscular dystrophy; Metachromatic leukodystrophy juvenile, late infantile, and adult types; Metachromatic leukodystrophy; Metatrophic dysplasia; Methemoglobinemia types 1 and 2; Methionine adenosyltransferase deficiency, autosomal dominant; Methylmalonic acidemia with homocystinuria; Methylmalonic aciduria cblB type; Methylmalonic aciduria due to methylmalonyl-CoA mutase deficiency; METHYLMALONIC ACIDURIA, mut (0) TYPE; Microcephalic osteodysplastic primordial dwarfism type 2; Microcephaly with or without chorioretinopathy, lymphedema, or mental retardation; Microcephaly, hiatal hernia and nephrotic syndrome; Microcephaly; Hypoplasia of the corpus callosum; Spastic paraplegia 50, autosomal recessive; Global developmental delay; CNS hypomyelination; Brain atrophy; Microcephaly, normal intelligence and immunodeficiency; Microcephaly-capillary malformation syndrome; Microcytic anemia; Microphthalmia syndromic 5, 7, and 9; Microphthalmia, isolated 3, 5, 6, 8, and with coloboma 6; Microspherophakia; Migraine, familial basilar; Miller syndrome; Minicore myopathy with external ophthalmoplegia; Myopathy, congenital with cores; Mitchell-Riley syndrome; mitochondrial 3-hydroxy-3-methylglutaryl-CoA synthase deficiency; Mitochondrial complex I, II, III, III (nuclear type 2, 4, or 8) deficiency; Mitochondrial DNA depletion syndrome 11, 12 (cardiomyopathie type), 2, 4B (MNGIE type), 8B (MNGIE type); Mitochondrial DNA-depletion syndrome 3 and 7, hepatocerebral types, and 13 (encephalomyopathic type); Mitochondrial phosphate carrier and pyruvate carrier deficiency; Mitochondrial trifunctional protein deficiency; Long-chain 3-hydroxyacyl-CoA dehydrogenase deficiency; Miyoshi muscular dystrophy 1; Myopathy, distal, with anterior tibial onset; Mohr-Tranebjaerg syndrome; Molybdenum cofactor deficiency, complementation group A; Mowat-Wilson syndrome; Mucolipidosis III Gamma; Mucopolysaccharidosis type VI, type VI (severe), and type VII; Mucopolysaccharidosis, MPS—I-H/S, MPS-II, MPS-III-A, MPS—III—B, MPS—III—C, MPS-IV-A, MPS—IV-B; Retinitis Pigmentosa 73; Gangliosidosis GM1 type1 (with cardiac involvement) 3; Multicentric osteolysis nephropathy; Multicentric osteolysis, nodulosis and arthropathy; Multiple congenital anomalies; Atrial septal defect 2; Multiple congenital anomalies-hypotonia-seizures syndrome 3; Multiple Cutaneous and Mucosal Venous Malformations; Multiple endocrine neoplasia, types land 4; Multiple epiphyseal dysplasia 5 or Dominant; Multiple gastrointestinal atresias; Multiple pterygium syndrome Escobar type; Multiple sulfatase deficiency; Multiple synostoses syndrome 3; Muscle AMP guanine oxidase deficiency; Muscle eye brain disease; Muscular dystrophy, congenital, megaconial type; Myasthenia, familial infantile, 1; Myasthenic Syndrome, Congenital, 11, associated with acetylcholine receptor deficiency; Myasthenic Syndrome, Congenital, 17, 2A (slow-channel), 4B (fast-channel), and without tubular aggregates; Myeloperoxidase deficiency; MYH-associated polyposis; Endometrial carcinoma; Myocardial infarction 1; Myoclonic dystonia; Myoclonic-Atonic Epilepsy; Myoclonus with epilepsy with ragged red fibers; Myofibrillar myopathy 1 and ZASP-related; Myoglobinuria, acute recurrent, autosomal recessive; Myoneural gastrointestinal encephalopathy syndrome; Cerebellar ataxia infantile with progressive external ophthalmoplegia; Mitochondrial DNA depletion syndrome 4B, MNGIE type; Myopathy, centronuclear, 1, congenital, with excess of muscle spindles, distal, 1, lactic acidosis, and sideroblastic anemia 1, mitochondrial progressive with congenital cataract, hearing loss, and developmental delay, and tubular aggregate, 2; Myopia 6; Myosclerosis, autosomal recessive; Myotonia congenital; Congenital myotonia, autosomal dominant and recessive forms; Nail-patella syndrome; Nance-Horan syndrome; Nanophthalmos 2; Navajo neurohepatopathy; Nemaline myopathy 3 and 9; Neonatal hypotonia; Intellectual disability; Seizures; Delayed speech and language development; Mental retardation, autosomal dominant 31; Neonatal intrahepatic cholestasis caused by citrin deficiency; Nephrogenic diabetes insipidus, Nephrogenic diabetes insipidus, X-linked; Nephrolithiasis/osteoporosis, hypophosphatemic, 2; Nephronophthisis 13, 15 and 4; Infertility; Cerebello-oculo-renal syndrome (nephronophthisis, oculomotor apraxia and cerebellar abnormalities); Nephrotic syndrome, type 3, type 5, with or without ocular abnormalities, type 7, and type 9; Nestor-Guillermo progeria syndrome; Neu-Laxova syndrome 1; Neurodegeneration with brain iron accumulation 4 and 6; Neuroferritinopathy; Neurofibromatosis, type land type 2; Neurofibrosarcoma; Neurohypophyseal diabetes insipidus; Neuropathy, Hereditary Sensory, Type IC; Neutral 1 amino acid transport defect; Neutral lipid storage disease with myopathy; Neutrophil immunodeficiency syndrome; Nicolaides-Baraitser syndrome; Niemann-Pick disease type C1, C2, type A, and type C1, adult form; Non-ketotic hyperglycinemia; Noonan syndrome 1 and 4, LEOPARD syndrome 1; Noonan syndrome-like disorder with or without juvenile myelomonocytic leukemia; Normokalemic periodic paralysis, potassium-sensitive; Norum disease; Epilepsy, Hearing Loss, And Mental Retardation Syndrome; Mental Retardation, X-Linked 102 and syndromic 13; Obesity; Ocular albinism, type I; Oculocutaneous albinism type 1B, type 3, and type 4; Oculodentodigital dysplasia; Odontohypophosphatasia; Odontotrichomelic syndrome; Oguchi disease; Oligodontia-colorectal cancer syndrome; Opitz G/BBB syndrome; Optic atrophy 9; Oral-facial-digital syndrome; Ornithine aminotransferase deficiency; Orofacial cleft 11 and 7, Cleft lip/palate-ectodermal dysplasia syndrome; Orstavik Lindemann Solberg syndrome; Osteoarthritis with mild chondrodysplasia; Osteochondritis dissecans; Osteogenesis imperfecta type 12, type 5, type 7, type 8, type I, type III, with normal sclerae, dominant form, recessive perinatal lethal; Osteopathia striata with cranial sclerosis; Osteopetrosis autosomal dominant type 1 and 2, recessive 4, recessive 1, recessive 6; Osteoporosis with pseudoglioma; Oto-palato-digital syndrome, types I and II; Ovarian dysgenesis 1; Ovarioleukodystrophy; Pachyonychia congenita 4 and type 2; Paget disease of bone, familial; Pallister-Hall syndrome; Palmoplantar keratoderma, nonepidermolytic, focal or diffuse; Pancreatic agenesis and congenital heart disease; Papillon-Lef\xc3\xa8vre syndrome; Paragangliomas 3; Paramyotonia congenita of von Eulenburg; Parathyroid carcinoma; Parkinson disease 14, 15, 19 (juvenile-onset), 2, 20 (early-onset), 6, (autosomal recessive early-onset, and 9; Partial albinism; Partial hypoxanthine-guanine phosphoribosyltransferase deficiency; Patterned dystrophy of retinal pigment epithelium; PC-K6a; Pelizaeus-Merzbacher disease; Pendred syndrome; Peripheral demyelinating neuropathy, central dysmyelination; Hirschsprung disease; Permanent neonatal diabetes mellitus; Diabetes mellitus, permanent neonatal, with neurologic features; Neonatal insulin-dependent diabetes mellitus; Maturity-onset diabetes of the young, type 2; Peroxisome biogenesis disorder 14B, 2A, 4A, 5B, 6A, 7A, and 7B; Perrault syndrome 4; Perry syndrome; Persistent hyperinsulinemic hypoglycemia of infancy; familial hyperinsulinism; Phenotypes; Phenylketonuria; Pheochromocytoma; Hereditary Paraganglioma-Pheochromocytoma Syndromes; Paragangliomas 1; Carcinoid tumor of intestine; Cowden syndrome 3; Phosphoglycerate dehydrogenase deficiency; Phosphoglycerate kinase 1 deficiency; Photosensitive trichothiodystrophy; Phytanic acid storage disease; Pick disease; Pierson syndrome; Pigmentary retinal dystrophy; Pigmented nodular adrenocortical disease, primary, 1; Pilomatrixoma; Pitt-Hopkins syndrome; Pituitary dependent hypercortisolism; Pituitary hormone deficiency, combined 1, 2, 3, and 4; Plasminogen activator inhibitor type 1 deficiency; Plasminogen deficiency, type I; Platelet-type bleeding disorder 15 and 8; Poikiloderma, hereditary fibrosing, with tendon contractures, myopathy, and pulmonary fibrosis; Polycystic kidney disease 2, adult type, and infantile type; Polycystic lipomembranous osteodysplasia with sclerosing leukoencephalopathy; Polyglucosan body myopathy 1 with or without immunodeficiency; Polymicrogyria, asymmetric, bilateral frontoparietal; Polyneuropathy, hearing loss, ataxia, retinitis pigmentosa, and cataract; Pontocerebellar hypoplasia type 4; Popliteal pterygium syndrome; Porencephaly 2; Porokeratosis 8, disseminated superficial actinic type; Porphobilinogen synthase deficiency; Porphyria cutanea tarda; Posterior column ataxia with retinitis pigmentosa; Posterior polar cataract type 2; Prader-Willi-like syndrome; Premature ovarian failure 4, 5, 7, and 9; Primary autosomal recessive microcephaly 10, 2, 3, and 5; Primary ciliary dyskinesia 24; Primary dilated cardiomyopathy; Left ventricular noncompaction 6; 4, Left ventricular noncompaction 10; Paroxysmal atrial fibrillation; Primary hyperoxaluria, type I, type, and type III; Primary hypertrophic osteoarthropathy, autosomal recessive 2; Primary hypomagnesemia; Primary open angle glaucoma juvenile onset 1; Primary pulmonary hypertension; Primrose syndrome; Progressive familial heart block type 1B; Progressive familial intrahepatic cholestasis 2 and 3; Progressive intrahepatic cholestasis; Progressive myoclonus epilepsy with ataxia; Progressive pseudorheumatoid dysplasia; Progressive sclerosing poliodystrophy; Prolidase deficiency; Proline dehydrogenase deficiency; Schizophrenia 4; Properdin deficiency, X-linked; Propionic academia; Proprotein convertase 1/3 deficiency; Prostate cancer, hereditary, 2; Protan defect; Proteinuria; Finnish congenital nephrotic syndrome; Proteus syndrome; Breast adenocarcinoma; Pseudoachondroplastic spondyloepiphyseal dysplasia syndrome; Pseudohypoaldosteronism type 1 autosomal dominant and recessive and type 2; Pseudohypoparathyroidism type 1A, Pseudopseudohypoparathyroidism; Pseudoneonatal adrenoleukodystrophy; Pseudoprimary hyperaldosteronism; Pseudoxanthoma elasticum; Generalized arterial calcification of infancy 2; Pseudoxanthoma elasticum-like disorder with multiple coagulation factor deficiency; Psoriasis susceptibility 2; PTEN hamartoma tumor syndrome; Pulmonary arterial hypertension related to hereditary hemorrhagic telangiectasia; Pulmonary Fibrosis And/Or Bone Marrow Failure, Telomere-Related, 1 and 3; Pulmonary hypertension, primary, 1, with hereditary hemorrhagic telangiectasia; Purine-nucleoside phosphorylase deficiency; Pyruvate carboxylase deficiency; Pyruvate dehydrogenase E1-alpha deficiency; Pyruvate kinase deficiency of red cells; Raine syndrome; Rasopathy; Recessive dystrophic epidermolysis bullosa; Nail disorder, nonsyndromic congenital, 8; Reifenstein syndrome; Renal adysplasia; Renal carnitine transport defect; Renal coloboma syndrome; Renal dysplasia; Renal dysplasia, retinal pigmentary dystrophy, cerebellar ataxia and skeletal dysplasia; Renal tubular acidosis, distal, autosomal recessive, with late-onset sensorineural hearing loss, or with hemolytic anemia; Renal tubular acidosis, proximal, with ocular abnormalities and mental retardation; Retinal cone dystrophy 3B; Retinitis pigmentosa; Retinitis pigmentosa 10, 11, 12, 14, 15, 17, and 19; Retinitis pigmentosa 2, 20, 25, 35, 36, 38, 39, 4, 40, 43, 45, 48, 66, 7, 70, 72; Retinoblastoma; Rett disorder; Rhabdoid tumor predisposition syndrome 2; Rhegmatogenous retinal detachment, autosomal dominant; Rhizomelic chondrodysplasia punctata type 2 and type 3; Roberts-SC phocomelia syndrome; Robinow Sorauf syndrome; Robinow syndrome, autosomal recessive, autosomal recessive, with brachy-syn-polydactyly; Rothmund-Thomson syndrome; Rapadilino syndrome; RRM2B-related mitochondrial disease; Rubinstein-Taybi syndrome; Salla disease; Sandhoff disease, adult and infantil types; Sarcoidosis, early-onset; Blau syndrome; Schindler disease, type 1; Schizencephaly; Schizophrenia 15; Schneckenbecken dysplasia; Schwannomatosis 2; Schwartz Jampel syndrome type 1; Sclerocornea, autosomal recessive; Sclerosteosis; Secondary hypothyroidism; Segawa syndrome, autosomal recessive; Senior-Loken syndrome 4 and 5; Sensory ataxic neuropathy, dysarthria, and ophthalmoparesis; Sepiapterin reductase deficiency; SeSAME syndrome; Severe combined immunodeficiency due to ADA deficiency, with microcephaly, growth retardation, and sensitivity to ionizing radiation, atypical, autosomal recessive, T cell-negative, B cell-positive, NK cell-negative of NK-positive; Severe congenital neutropenia; Severe congenital neutropenia 3, autosomal recessive or dominant; Severe congenital neutropenia and 6, autosomal recessive; Severe myoclonic epilepsy in infancy; Generalized epilepsy with febrile seizures plus, types 1 and 2; Severe X-linked myotubular myopathy; Short QT syndrome 3; Short stature with nonspecific skeletal abnormalities; Short stature, auditory canal atresia, mandibular hypoplasia, skeletal abnormalities; Short stature, onychodysplasia, facial dysmorphism, and hypotrichosis; Primordial dwarfism; Short-rib thoracic dysplasia 11 or 3 with or without polydactyly; Sialidosis type I and II; Silver spastic paraplegia syndrome; Slowed nerve conduction velocity, autosomal dominant; Smith-Lemli-Opitz syndrome; Snyder Robinson syndrome; Somatotroph adenoma; Prolactinoma; familial, Pituitary adenoma predisposition; Sotos syndrome 1 or 2; Spastic ataxia 5, autosomal recessive, Charlevoix-Saguenay type, 1,10, or 11, autosomal recessive; Amyotrophic lateral sclerosis type 5; Spastic paraplegia 15, 2, 3, 35, 39, 4, autosomal dominant, 55, autosomal recessive, and 5A; Bile acid synthesis defect, congenital, 3; Spermatogenic failure 11, 3, and 8; Spherocytosis types 4 and 5; Spheroid body myopathy; Spinal muscular atrophy, lower extremity predominant 2, autosomal dominant; Spinal muscular atrophy, type II; Spinocerebellar ataxia 14, 21, 35, 40, and 6; Spinocerebellar ataxia autosomal recessive 1 and 16; Splenic hypoplasia; Spondylocarpotarsal synostosis syndrome; Spondylocheirodysplasia, Ehlers-Danlos syndrome-like, with immune dysregulation, Aggrecan type, with congenital joint dislocations, short limb-hand type, Sedaghatian type, with cone-rod dystrophy, and Kozlowski type; Parastremmatic dwarfism; Stargardt disease 1; Cone-rod dystrophy 3; Stickler syndrome type 1; Kniest dysplasia; Stickler syndrome, types 1 (nonsyndromic ocular) and 4; Sting-associated vasculopathy, infantile-onset; Stormorken syndrome; Sturge-Weber syndrome, Capillary malformations, congenital, 1; Succinyl-CoA acetoacetate transferase deficiency; Sucrase-isomaltase deficiency; Sudden infant death syndrome; Sulfite oxidase deficiency, isolated; Supravalvar aortic stenosis; Surfactant metabolism dysfunction, pulmonary, 2 and 3; Symphalangism, proximal, 1b; Syndactyly Cenani Lenz type; Syndactyly type 3; Syndromic X-linked mental retardation 16; Talipes equinovarus; Tangier disease; TARP syndrome; Tay-Sachs disease, B1 variant, Gm2-gangliosidosis (adult), Gm2-gangliosidosis (adult-onset); Temtamy syndrome; Tenorio Syndrome; Terminal osseous dysplasia; Testosterone 17-beta-dehydrogenase deficiency; Tetraamelia, autosomal recessive; Tetralogy of Fallot; Hypoplastic left heart syndrome 2; Truncus arteriosus; Malformation of the heart and great vessels; Ventricular septal defect 1; Thiel-Behnke corneal dystrophy; Thoracic aortic aneurysms and aortic dissections; Marfanoid habitus; Three M syndrome 2; Thrombocytopenia, platelet dysfunction, hemolysis, and imbalanced globin synthesis; Thrombocytopenia, X-linked; Thrombophilia, hereditary, due to protein C deficiency, autosomal dominant and recessive; Thyroid agenesis; Thyroid cancer, follicular; Thyroid hormone metabolism, abnormal; Thyroid hormone resistance, generalized, autosomal dominant; Thyrotoxic periodic paralysis and Thyrotoxic periodic paralysis 2; Thyrotropin-releasing hormone resistance, generalized; Timothy syndrome; TNF receptor-associated periodic fever syndrome (TRAPS); Tooth agenesis, selective, 3 and 4; Torsades de pointes; Townes-Brocks-branchiootorenal-like syndrome; Transient bullous dermolysis of the newborn; Treacher collins syndrome 1; Trichomegaly with mental retardation, dwarfism and pigmentary degeneration of retina; Trichorhinophalangeal dysplasia type I; Trichorhinophalangeal syndrome type 3; Trimethylaminuria; Tuberous sclerosis syndrome; Lymphangiomyomatosis; Tuberous sclerosis 1 and 2; Tyrosinase-negative oculocutaneous albinism; Tyrosinase-positive oculocutaneous albinism; Tyrosinemia type I; UDPglucose-4-epimerase deficiency; Ullrich congenital muscular dystrophy; Ulna and fibula absence of with severe limb deficiency; Upshaw-Schulman syndrome; Urocanate hydratase deficiency; Usher syndrome, types 1, 1B, 1D, 1G, 2A, 2C, and 2D; Retinitis pigmentosa 39; UV-sensitive syndrome; Van der Woude syndrome; Van Maldergem syndrome 2; Hennekam lymphangiectasia-lymphedema syndrome 2; Variegate porphyria; Ventriculomegaly with cystic kidney disease; Verheij syndrome; Very long chain acyl-CoA dehydrogenase deficiency; Vesicoureteral reflux 8; Visceral heterotaxy 5, autosomal; Visceral myopathy; Vitamin D-dependent rickets, types land 2; Vitelliform dystrophy; von Willebrand disease type 2M and type 3; Waardenburg syndrome type 1, 4C, and 2E (with neurologic involvement); Klein-Waardenberg syndrome; Walker-Warburg congenital muscular dystrophy; Warburg micro syndrome 2 and 4; Warts, hypogammaglobulinemia, infections, and myelokathexis; Weaver syndrome; Weill-Marchesani syndrome 1 and 3; Weill-Marchesani-like syndrome; Weissenbacher-Zweymuller syndrome; Werdnig-Hoffmann disease; Charcot-Marie-Tooth disease; Werner syndrome; WFS1-Related Disorders; Wiedemann-Steiner syndrome; Wilson disease; Wolfram-like syndrome, autosomal dominant; Worth disease; Van Buchem disease type 2; Xeroderma pigmentosum, complementation group b, group D, group E, and group G; X-linked agammaglobulinemia; X-linked hereditary motor and sensory neuropathy; X-linked ichthyosis with steryl-sulfatase deficiency; X-linked periventricular heterotopia; Oto-palato-digital syndrome, type I; X-linked severe combined immunodeficiency; Zimmermann-Laband syndrome and Zimmermann-Laband syndrome 2; and Zonular pulverulent cataract 3. Reference is made to PCT Publication No. WO2020/191249A1, the entirety of which is incorporated by reference herein.

TABLE 1
Non-limiting Fanzor polypeptides associated
with the present disclosure

[symbol]

[symbol]

indicates data missing or illegible when filed

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

Both prokaryotic and eukaryotic genomes are replete with diverse transposons, a broad class of mobile genetic elements (MGE), that widely differ in abundance. Transposons of the highly abundant IS200/605 family encode the TnpA protein, which is a DDE class transposase responsible for the single-strand ‘peel and paste’ transposition mechanism of these MGEs, and TnpB protein the role of which in transposition remains unclear. Numerous non-autonomous transposons encode TnpB alone, requiring a transposase to be supplied in trans. RNA-programmable DNA nucleases serve multiple roles in prokaryotes, including in mobile element defense and spread. These nucleases include argonaut, CRISPR, and the obligate mobile element-guided activity (OMEGA) systems, the latter of which include the TnpB, IscB, IsrB, and IshB nucleases. TnpB contains a RuvC-like nuclease domain (RNase H fold) that is specifically related to the homologous nuclease domain of Cas12, the effector nuclease of type V CRISPR-Cas systems, specifically, CAS12F, suggesting that TnpB is the evolutionary ancestor of Cas12. Phylogenetic analysis of the RuvC-like domains, indeed, supports independent origins of Cas12s of different type V subtypes from distinct groups of TnpBs. Recently, it has been demonstrated experimentally (through biochemical and cellular experiments) that these TnpBs are components of OMEGA (obligate mobile element-guided activity) systems that encode the ωRNA next to the nuclease gene (often overlapping with the 3′-end or the coding region of the latter). The ωRNA-TnpB complex is a RNA-guided DNA endonuclease. The ωRNA resembles a ωRNA structurally but is larger and contains a spacer-like, target recognition sequence that lies immediately outside the transposon end suggesting that these nuclease are involved in RNA-guided transposition although other roles in the transposon life cycle cannot be ruled out. The OMEGA nucleases are programmable, that is, cleavage can be directed to any genomic region by replacing the spacer-like region by an arbitrary sequence. Hence these OMEGA nucleases have considerable potential as genome editing tools, and first attempts in this direction have been reported.

While TnpBs are highly abundant in bacteria and archaea, TnpB homologs (denoted Fanzors) have also been identified in diverse eukaryotes, including metazoans, fungi and many unicellular organisms, and some double-stranded (ds) DNA viruses with large genomes infecting unicellular eukaryotes. Two major groups of Fanzors have been identified: 1) Fanzor1 that are associated with eukaryotic transposons, including Mariners, IS4-like elements, Sola, Helitron, and MuDr, and 2) Fanzor2 systems that are found in IS607-like transposons and are present in dsDNA viral genomes. Despite the similarities between TnpB and Fanzors, Fanzors have not been surveyed comprehensively throughout eukaryotic diversity and, unlike the OMEGA nucleases, neither the biochemical activity of Fanzors nor their role in transposons have been studied experimentally.

The Examples herein report a comprehensive census of Fanzors in eukaryotic and viral genomes, phylogenetic analysis clarifying their prokaryotic origins and tracing their evolution, and RNA sequencing (RNA-seq) and biochemical experiments demonstrating the programmable RNA-guided endonuclease activity of the Fanzors, showcasing their utility as new genome editing tools.

### Example 1: RuvC Containing TnpB Homologs are Widespread in Eukaryotes and Giant Viruses

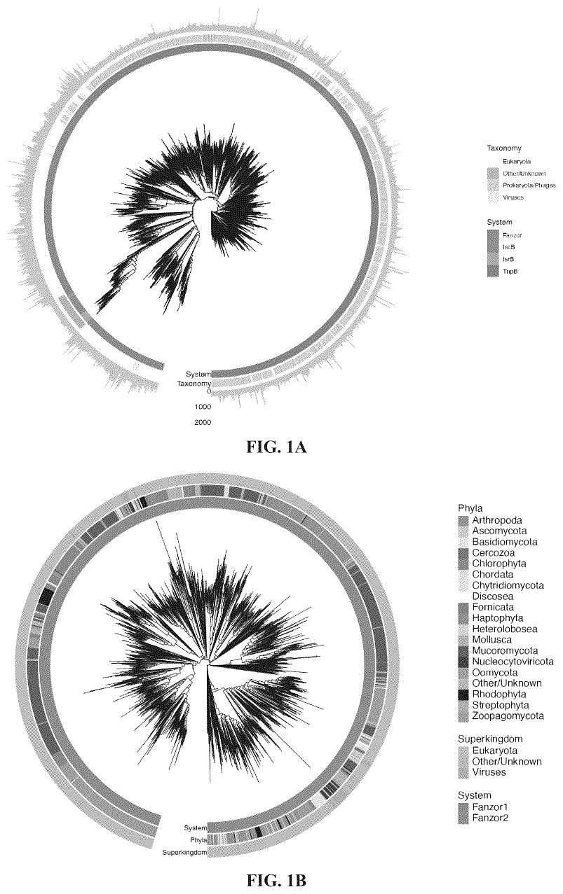

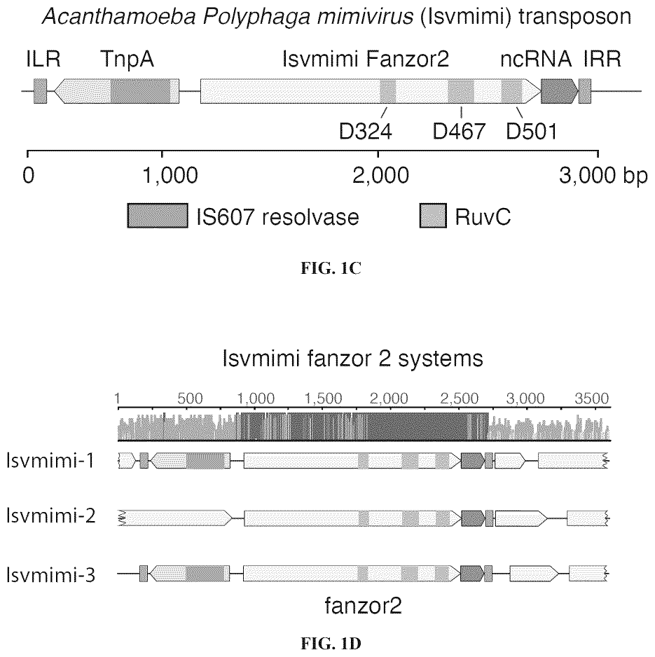

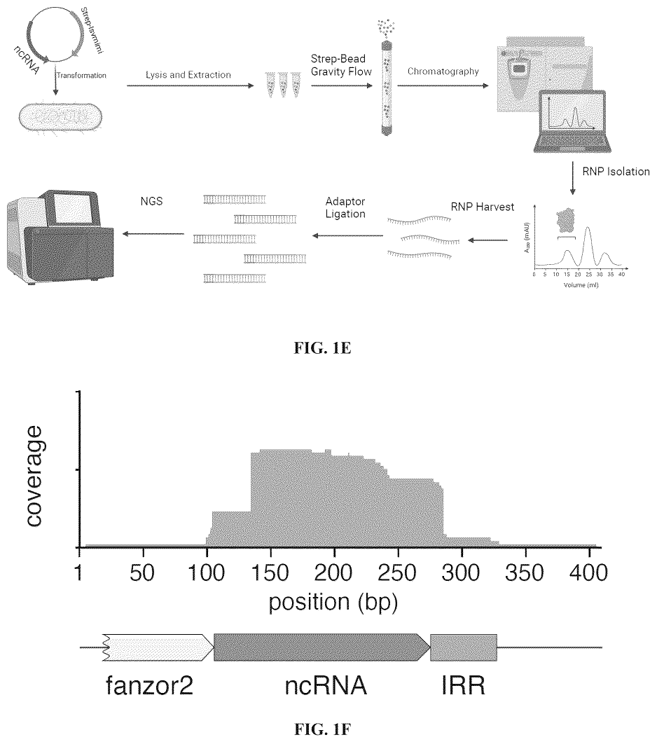

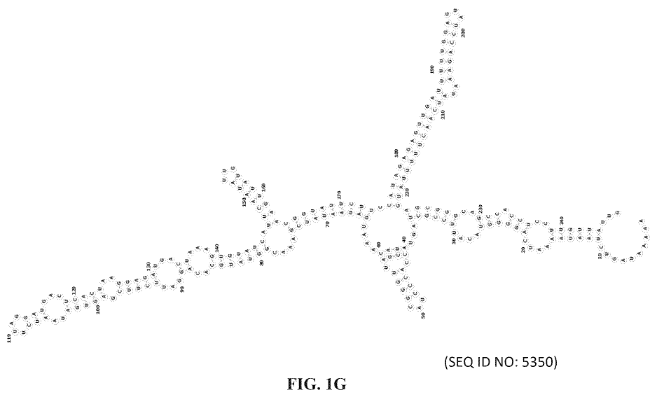

To identify putative RuvC nucleases in eukaryotic and viral genomes, a comprehensive search was performed across eukaryotic and viral genomes using a profile derived from the multiple alignment of the RuvC domains from bacterial TnpB, IscB and IsrB, and the previously identified Fanzor 1 and Fanzor 2 proteins. This search yielded Fanzor proteins occurring across metazoans, fungi, plants, and diverse unicellular eukaryotesas well as giant viruses of the family Mimiviridae (FIGS. 1A-1B). Clustering these putative nucleases with selected representatives of TnpB, IscB, and IsrB revealed several distinct families of eukaryotic RuvC containing nucleases. One Family, which contains the previously discovered Fanzor1 proteins, occurred in diverse eukaryotes, including fungi, plants, various protists, and animals (FIGS. 1A-1B). In contrast, another Family contains a subset of Fanzor2 proteins with similarity to TnpB and was identified primarily in giant dsDNA viruses of the family Mimiviridae, with most family members occurring at multiple locations within their host genome. Given that giant dsDNA viruses likely acquired bacterial MGEs like TnpBs in amoeba melting pots where viruses, bacteria, and bacteriophages could interact (Boyer et al. 2009), it suggests a potential evolutionary path via horizontal gene transfer. (FIG. 1B) Because of the sequence conservation and these relationships to bacterial TnpB systems, the Fanzor 2 family was selected for further analysis.

### Example 2: Fanzor2 is Associated with Conserved and Structured Non-Coding RNAs

To characterize the Fanzor2 family, the Fanzor2 from Acanthamoeba Polyphaga mimivirus (IsvMimi Fanzor2) was selected. Leveraging the fact that IsvMimi, is present multiple times in the mimivirus genome, all copies of this Fanzor 2 were aligned to find conserved elements both in the ORF and in the surrounding neighborhood. Similar to bacterial TnpB and IscB systems, a strong conservation both within protein-coding regions and in the non-coding region at the 3′ end of the IS607 MGE was found. This non-coding sequence conservation extended 200 base pairs past the end of IsvMimi ORF before reaching the right inverted repeat element IRR, in contrast to the more ORF-proximal IRR found in TnpB MGE. (FIG. 1C) Using in silico RNA secondary structure prediction, a stable fold was found (FIG. 1D), suggesting that it could serve as a nuclease-associated RNA, which is referred to herein as “fRNA”, that could complex with Fanzor2 and program its nuclease activity towards a specific sequence. Expanding this analysis beyond IsvMimi, fRNA conservation across the Fanzor2 family was analyzed by comparing similarities within clusters based on ORF alignments, and surprisingly found that all Fanzor2 clusters had strong conservation on the 3 end (FIG. 1B).

### Example 3: Fanzor Forms an Ribonucleoprotein Complex with fRNAs

To evaluate whether the strongly conserved fRNA was associating with the Fanzor2 protein, the Isvmimi locus containing the non-coding RNA region and E. coli codon-optimized Isvmimi Fanzor 2 protein in E. coli were co-expressed (FIG. 1E) Indicative of the functional importance of the fRNA, the Isvmimi Fanzor 2 protein was unstable when expressed alone, and required co-expression with the fRNA for stable expression. This contribution of the fRNA to the stability of small RuvC proteins has been similarly observed in TnpB systems (Altae-Tran et al. 2021; Karvelis et al. 2021). Purifying the co-complex of Fanzor2 with its fRNA, small RNA sequencing of the associated RNA component of the ribonucleoprotein (RNP) complex was performed, observing enrichment of reads between the 3′ end of the protein ORF and the IRR, in agreement with evolutionarily conserved regions. (FIG. 1F) The strong interaction of these fRNA species with the Fanzor protein suggests that the fRNA might serve as a guide RNA to direct targeting of Isvmimi Fanzor2, similar to the role of ωRNA for programming of TnpB (Karvelis et al. 2021; Altae-Tran et al. 2021). Within the Fanzor2 family, it was surprisingly found that there were multiple representative fRNA structures (FIG. 1G), each with features. This conservation of structure is reminiscent of the OMEGA families, where both the IscB and TnpB clades possess limited structural variation.

### Example 4: Fanzor2 is a Programmable RNA-Guided DNA Endonuclease

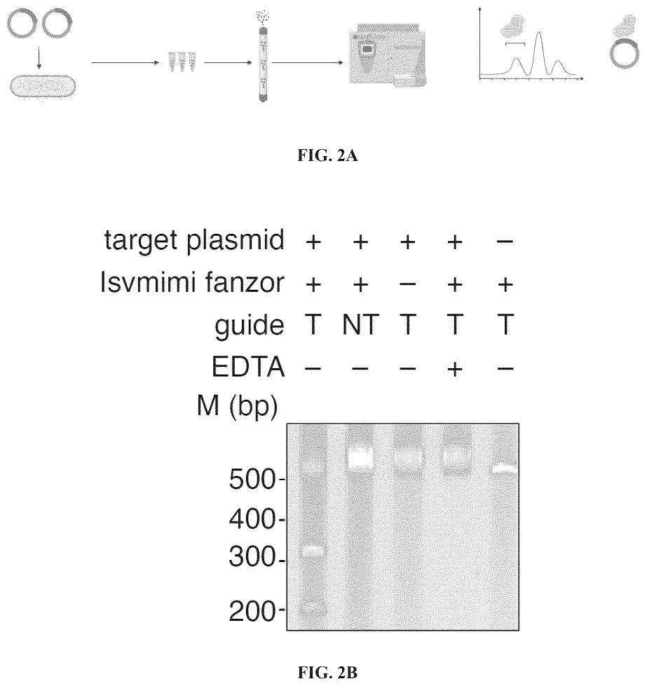

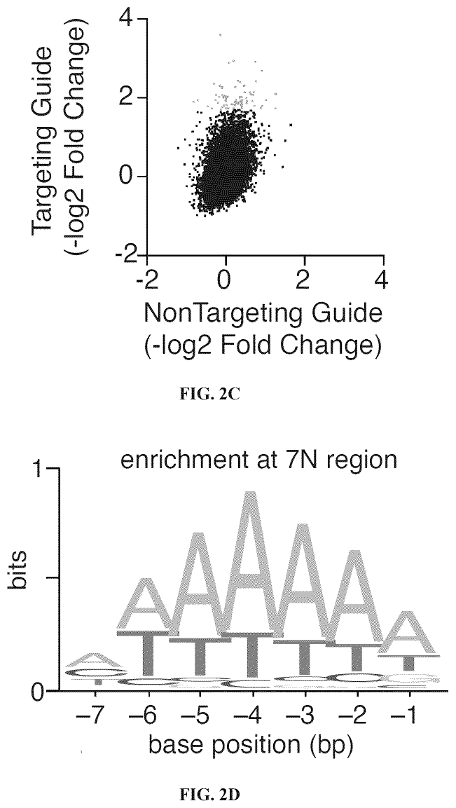

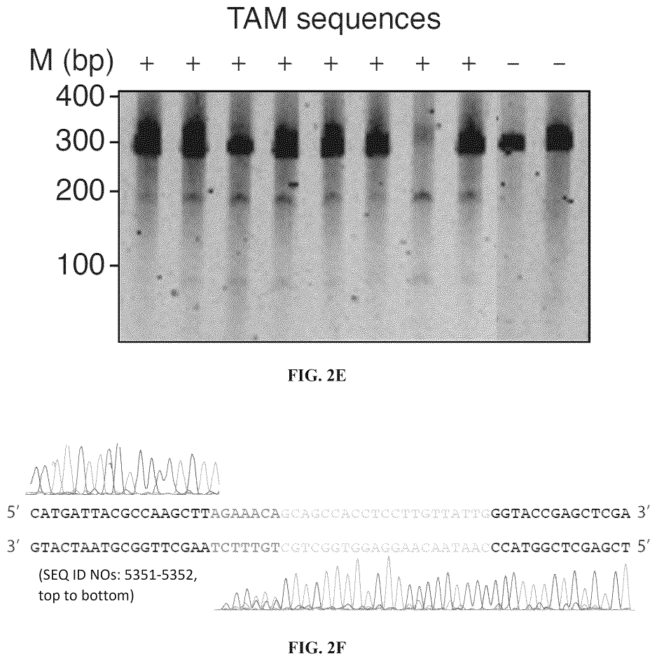

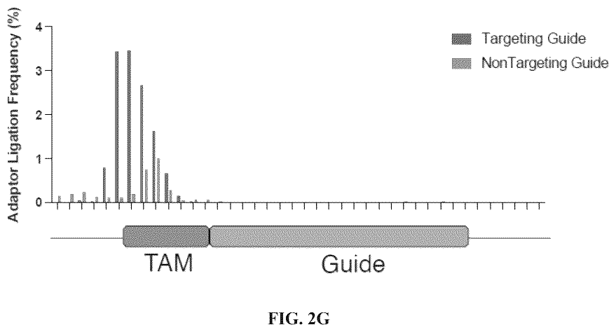

It was hypothesized herein that Isvmimi Fanzor2 is guided by the proximal fRNA to target and cleave DNA sequences. To reprogram this activity, an fRNA with the last 21 nucleotides targeting a novel 21 bp sequence was designed. Rosetta cells were co-transformed with both the fRNA with reprogrammed 3′ guide sequence and a Strep tagged Isvmimi to directly obtain the RNP in E. coli. (FIG. 2A) To account for any intrinsic sequence preferences of the Fanzor2 such as a target adjacent motif (TAM), cleavage on a target flanked by a randomized 7 nucleotide (TAM) at the 5′ ends of the 21 bp target spacer sequence was tested. Co-incubation of the Isvmimi RNP complex with this TAM library generated substantial cleavage of the TAM library, as visualized by gel electrophoresis, and cleavage was target dependent with no activity when either the guide or TAM library was changed to eliminate complementarity. (FIG. 2B) To understand the sequence restrictions on RNA-programmed DNA cleavage by Ismimi, the band corresponding to the uncleaved TAM library for next-generation sequencing was prepared and determined depleted TAMs due to Isvmimi cleavage. Significant depletion in the targeting guide condition of specific TAMs was found compared to a non-targeting guide condition with the consensus sequence of depleted sequences showing enrichment of A and T in positions 4 and 5 with semi-relaxed bases at positions 1-3 with the exception of G. (FIG. 2C) To confirm these preferences, the top 8 depleted TAMs were cloned and validated individually via biochemical cleavage assays, where it was found that all putative TAMs were robustly cut in vitro. (FIG. 2D) To confirm that conserved residues of the Isvmimi RuvC domain were responsible for cleavage, the catalytic asparagine (D) residue in the RuvC I domain of Isvmimi to alanine was mutated. The mutant was incapable of either dsDNA cleavage or ssDNA nicking. As prokaryotic RuvC-containing nucleases such as TnpB can demonstrate substantial thermophilic temperature preferences (Altae-Tran et al. 2021), Isvmimi Fanzor2 cleavage was evaluated over a range of temperatures, determining that optimal activity between 30 and 40 degree Celsius.

### Example 5: Fanzor2 Cuts within its TAM and Lacks Collateral Activity

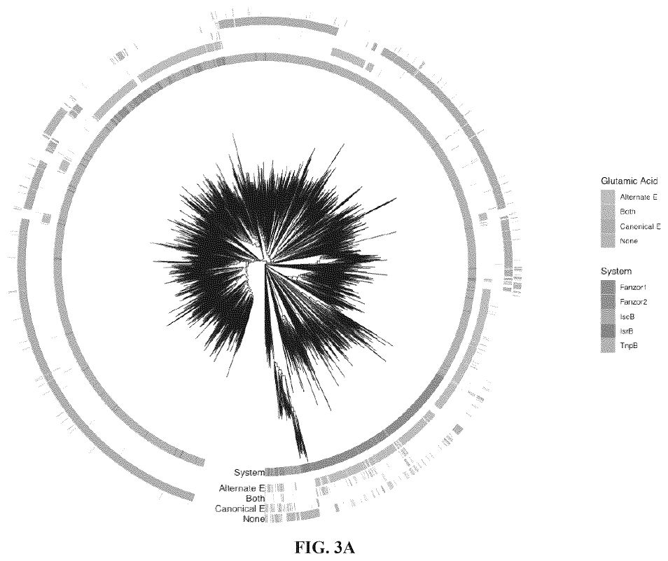

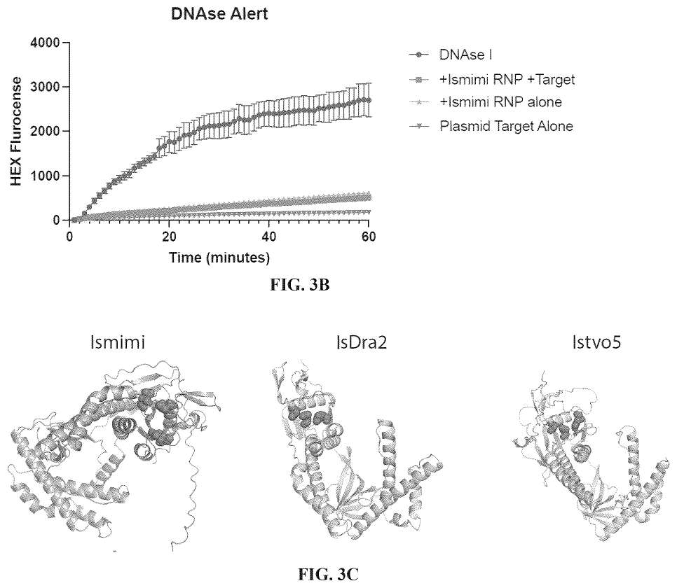

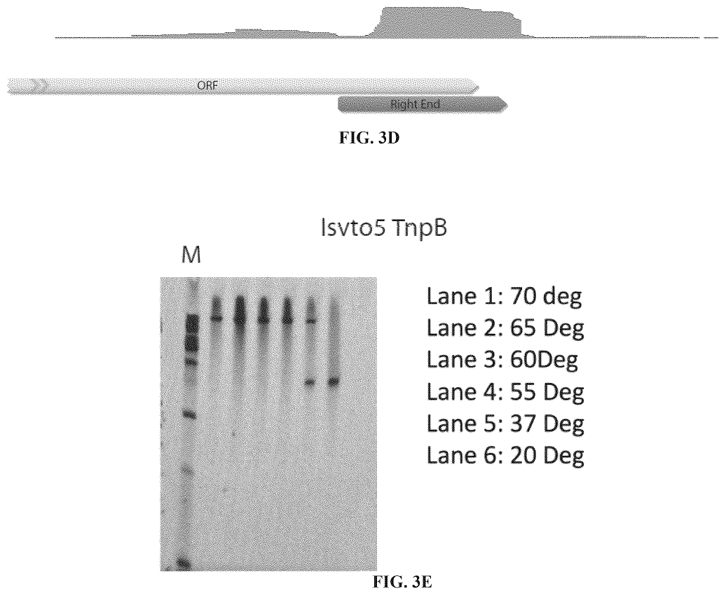

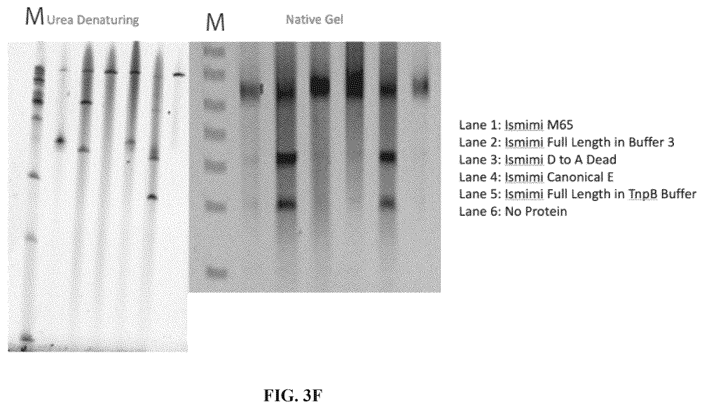

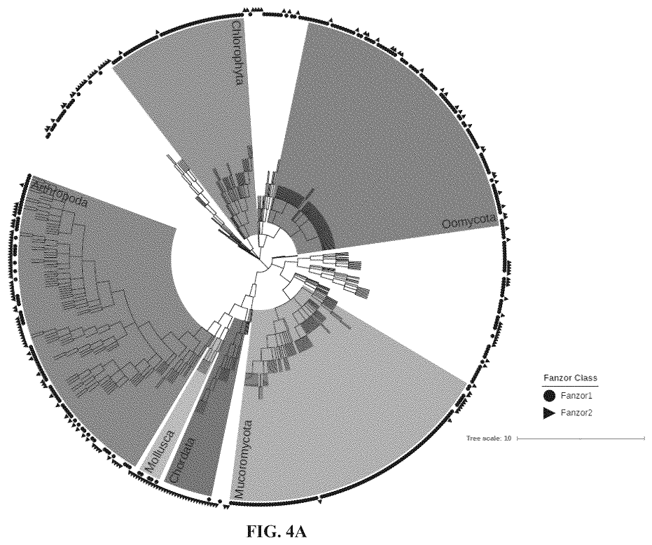

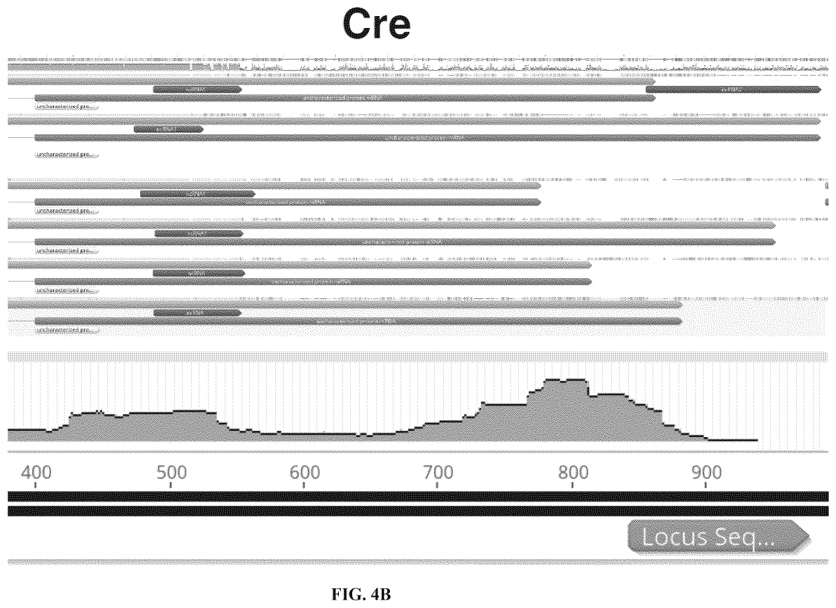

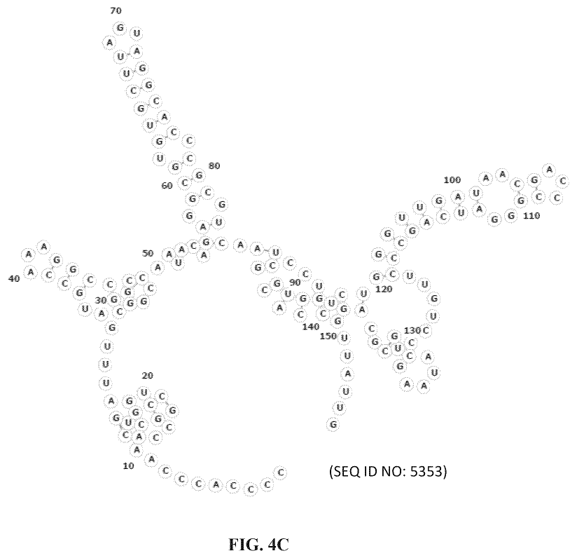

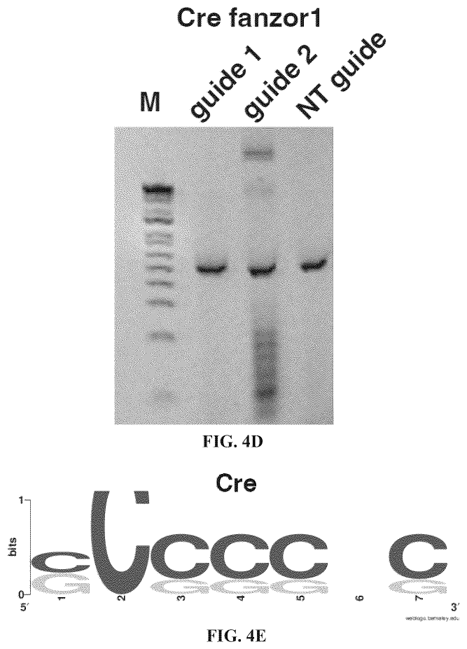

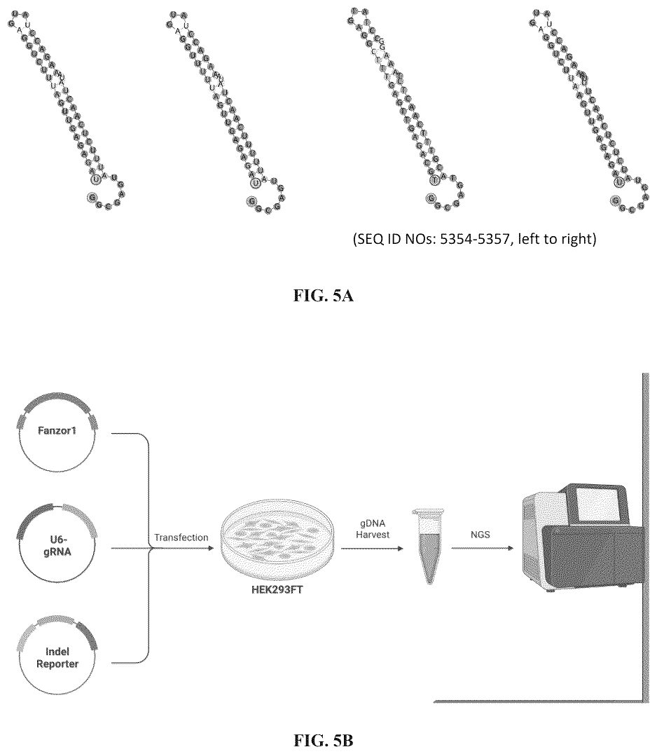

Having determined the constraints on Isvmimi Fanzor2 cleavage, the location of this cleavage within the target was then mapped. The products from Isvmimi Fanzor2 cleavage were isolated and the locations of the ends were mapped using Sanger sequencing, finding that cleavage occurred in the TAM, with multiple nicks within the non-target strand (NTS) and a single nick in the targeted strand (TS). (FIGS. 2E-2F). The 5′ cleavage location of Fanzor2 is in contrast to the observed cleavage location of Cas12 or TnpB nucleases, which cleave a specific distance away from the PAM or TAM, respectively, on the 3′ sides of the protospacer sequence. In comparison to canonical TnpB families, all observed Fanzor2 nucleases show a substitution of the catalytic RuvC site from a glutamate residue to an aspartate. (FIG. 3A). To find similar catalytic site substitutions among TnpB proteins, glutamate-containing RuvC domains were searched across both prokaryotic and eukaryotic genomes and a distinct arrangement of the RuvC II domain present in all Fanzor proteins was found, with a substantial subfamily of bacterial TnpB proteins also sharing this rearrangement (FIG. 3A). Without wishing to be bound by theory, it was hypothesized the observed cleavage pattern of Fanzor2 might be due to the unique re-arrangement of RuvC II domain glutamic acid residue. Comparing the orientation of the RuvC catalytic residues between Isvmimi Fanzor2, TnpB, Cas12f, and the TnpB (FIG. 3B) it was observed that, even with a glutamic acid in RuvC II, the three catalytic residues D324, E467, and D501 of Isvmimi Fanzor2 maintained the close contact of other RuvC pocket, explaining the cleavage activity in light of the rearranged site (FIG. 3B). Furthermore, without wishing to be bound by theory, it was hypothesized that if the distinct RuvC site was responsible for cleavage within in the TAM rather than on the 3′ end, the catalytic pocket would be less solvent exposed, reducing acceptance of outside nucleic acids and the subsequent collateral activity of the enzyme (Chen et al. 2018; Abudayyeh et al. 2016). The Isvmimi Fanzor2 was profiled for either RNA or DNA collateral cleavage activity, by co-incubating an Isvmimi or TnpB RNP complex with a cognate target along with either DNase alert or RNAse alert, single-stranded substrates that become fluorescent upon nucleolytic cleavage. In contrast to TnpB, Isvmimi nuclease was found to lack DNA collateral cleavage activity (FIG. 3C), with neither enzyme having collateral activity on RNA.

To understand if the glutamate rearrangement drives the unique cleavage properties, including cutting inside of the TAM and lack of collateral, the TnpB (Istvo5 TnpB) was purified, which also processes this glutamate rearrangement.

### Example 6: Diversity of Fanzor1 and Fanzor 2 Proteins Across the Eukaryotic Kingdom of Life

Having demonstrated that the Fanzor2 family had RNA programmable cleavage, the characterization shown herein was expanded to the additional families spanning viruses, plants, metazoans, fungi, and protists. Unlike the Fanzor2 systems, many of these broader family members are associated with diverse transposable element associations and sometimes lack readily identifiable MGE scars, complicating fRNA determination. To characterize an additional family member from plants, the Fanzor1 systems from the green algae Chlamydomonas reinhardtii (Cre Fanzor1) were selected, which contains multiple Fanzor1 copies. Cre Fanzor1 is associated with the eukaryotic Helitron 2 transposons, which do have identifiable short asymmetrical terminal inverted repeats (ATIRs) flanking the MGE insertion ends. The homologous Cre Fanzor1 was aligned to determine the putative conserved fRNA, and, similar to the Fanzor2 families, a strong conservation of fRNA regions was found.

To determine the relevant fRNA species, the region containing the putative Cre-1 Fanzor1 fRNA and a codon optimized Cre-1 Fanzor1 protein in E. coli were co-expressed. Similar to the Fanzor 2 family, the Fanzor 1 protein required fRNA co-expression for production of stable protein and RNA sequencing on purified RNP revealed a precise fRNA species processed near the 3′ end of the Fanzor1 protein, overlapping the 3′ ATIR of the MGE. This fRNA had strong predicted secondary structure, but was distinct from the Fanzor 2 clade. The conservation of this non-coding RNA was further studied with the closest systems to the Cre systems in terms of protein sequence similarity and found that the non-coding RNA was conserved in both sequence and structure.

To reprogram Cre Fanzor1 protein cleavage using the putative fRNA, a Cre Fanzor1 RNP containing a guide against the previously used TAM library was purified. As with Isvmimi Fanzor2, Cre Fanzor1 stability was fRNA dependent. Co-incubation of this complex with the TAM library generated two significant bands in a guide and magnesium dependent fashion. Sequencing the uncleaved TAM targets determined a specific TAM preference that validated upon testing individual TAM targets enriched in the screen. The in vitro activity of Cre Fanzor1 showcases that active Fanzor proteins are evolutionarily widespread across diverse lineages.

### Example 7: Fanzor Nucleases can be Adapted for Mammalian Genome Editing

To test whether programmable Fanzor nuclease could be applied for genome editing given their mesophilic operating temperature, the fRNA guide was engineered for expression in mammalian cells. Because there are two poly U stretches (>5 U) in the putative guide scaffolds for Isvmimi that can block U6 promoter expression, the fifth U inside the guide stem-loop region to interrupt the poly U stretch was mutated. 21 nt guides were designed using this redesigned scaffold against several positions inside the human EMX1 gene and tested for its indel activity in HEK293FT cells.

### Example 8: Widespread Fanzor ORFs Contain Spliced Introns

As Fanzors extend programmable nucleases into eukaryotes, the emergence of introns across Fanzor diversity was explored. Among the Fanzor families, a wide range of intron numbers was found. Using RNA sequencing data, the presence of three to four introns within the Cre Fanzor1 genes that are removed from the mature mRNA transcript was confirmed. Analyzing the conservation of the locus, it was surprisingly found that the introns are substantially less conserved than exonic sequences, implying that ancestral Fanzors inserted into host genomes via horizontal transfer and acquired introns overtime. It is unclear how splicing plays into the regulation of Fanzor expression and transposition activity.

### Example 9: Transposase Proteins are Associated with Fanzor Systems

Notably, Fanzor 2 proteins occur within the IS607 transposon, which is similar to the TnpA family of proteins, suggesting Fanzor2 might serve as the eukaryotic TnpB counterpart for the known bacterial IS200/605 superfamily. Because of these associations, the full extent of Fanzor 2 association with transposase domains was analyzed first, finding primarily an association with IS607 element transposases. These proteins are closely associated and can be found within readily identifiable inverted repeat element ends. By analyzing the host genome junctions with the IRL and IRR, it was found that the Fanzor 2 transposons primarily insert in A/T rich target sequences. Many of these target motifs appear similar to the Isvmimi TAM preference, suggesting that Fanzor 2 cleavage may be directly related to the insertion site preference for the transposon.

### Example 10: Characterization of Fanzor 1

Unlike Fanzor 2 systems, many previously found Fanzor 1 proteins are associated with eukaryotic transposons, including DNA transposons from different superfamilies including Helitron, Mariner, IS4-like, Sola and MuDr, however, the full extent of transposons acquiring Fanzor 1 into their MGE by analyzing nearby ORFs with transposon domains has not been previously characterized. While helitron and MuDr transposase ORFs do not directly associate with Fanzor 1 inside the transposon, the other transposases do strictly associate within the transposon, motivating our guilt by association approach for finding additional transposase associations.

RNA-guided nucleases serve vital roles in horizontal gene transfer in prokaryotic hosts and mobile elements, allowing for both adaptive immunity a programmable gene flow. RNA programmable DNA nucleases shown herein are similarly abundant in eukaryotic nuclear genomes and viruses, including plant, fungal, and metazoan groups. These Fanzor nucleases, which contain the previously discovered Fanzor 1 and Fanzor 2 systems (Bao and Jurka 2013), are evolutionarily similar to the TnpB nucleases associated with IS200/IS605 family transposons. This transfer of these nucleases from a prokaryotic to eukaryotic context may have occurred through large DNA viruses acquiring TnpBs via horizontal gene transfer from bacteria and phages in amoebae, serving as “melting pots” of HGT between prokaryotes and eukaryotes (Boyer et al. 2009). As Fanzor systems spread throughout eukaryotic diversity, introns were acquired within the Fanzor nucleases, likely driven by the improved fitness of spliced genes from enhanced nucleocytoplasmic transport (Dimaano and Ullman 2004). The co-evolution of Fanzors with the nuclear genomes of their eukaryotic hosts is supported by the intron density of Fanzor genes matching the intron density of their host genomes (Basu et al. 2008; Csuros et al. 2011). The co-evolution of Fanzor systems with their hosts nuclear genomes reported herein suggests preferential movement within hosts compared to HGT. The Fanzor family persistence and spread within eukaryotic genomes implies Fanzor systems spread within host genomes with minimal fitness cost or potential fitness gain to the host. Without wishing to be bound by theory, one possible mechanism of positive fitness of Fanzors could be maintenance of genome stability, as is the case with non-LTR retrotransposons that insert in repetitive regions and help maintain repetitive genes (Nelson et al. 2021).

Fanzor families are associated with diverse transposases, strongly suggesting multiple events capturing Fanzor proteins by these transposons during evolution and a putative role of RNA guided nuclease activity of Fanzors in transposition. This role could be through a variety of mechanisms, including: 1) precise excision of the transposon from the genome via self-homing, 2) passive homing of the transposon to new alleles via leveraging nuclease-induced DSBs and DNA repair mechanisms, such as homologous recombination, and 3) active homing of the transposon using RNA guided DNA binding or cleavage for direct targeting of transposase activity. The latter mechanism would be analogous to the CRISPR-associated Tn7-like transposons that possess RNA-guided transposition via acquisition of RNA-guided DNA binding CRISPR effectors in conjunction with transposase components (Strecker et al. 2019; Klompe et al. 2019). Moreover, as Fanzor-containing transposons harbor associated genes of diverse putative functions and multiple Fanzor families possess N-terminal domains of varying predicted functions, Fanzor families may have additional undetermined roles.

The biochemical characterization of Fanzor nucleases shown herein revealed both similarities with the related TnpB and CRISPR-Cas12f nuclease, as well as several important distinctions. Similar to the Cas12 and TnpB nucleases, Fanzors generate double stranded breaks through a single RuvC domain; however, unliked the Cas12 and TnpBs, which cut DNA targets distal from the 5′ PAM/TAM on the 3′ end of the guide, Fanzor proteins unexepectedly cut within the 5′ TAM region. Potentially related to the unique cleavage position is the surprising apparent loss of collateral activity from the Fanzor family. Without wishing to be bound by theory, it is hypothesized that because the TAM is more internal to the RNP: DNA complex, it is possible that the activated RuvC domain is not solvent exposed, preventing trans DNA cleavage upon target recognition. As opposed to the more T rich sequence constraints of Cas12 and TnpB families, the Fanzor TAM preference is surprisingly diverse, with AT rich preference for the Fanzor 2 family and a GC-rich preference for Fanzor 1 proteins. Lastly, while the non-coding RNA of Fanzor 2 overlaps with the transposon IRR, much like TnpB's ωRNA, it is further downstream of the Fanzor ORF, whereas the ωRNAs are contained within the 3′ of the TnpB ORF. Therefore, the Fanzors are a unique family of eukaryotic programmable nucleases distantly related to TnpBs and Cas12f systems.

It is surprisingly shown herein that Fanzors can be applied for genome editing with detectable cleavage and indel generation activity in human cells. The Fanzor enzymes provide multiple advantages including precise nuclease activity, a small size, and eukaryotic origins, which may reduce the immunogenicity of these nucleases in humans. The broad distribution of Fanzor proteins across the multiple eukaryotic kingdoms and associated viruses suggests a further, as yet-discovered abundance of RNA-guided systems. The evolution of these nucleases expands the field's understanding of horizontal gene transfer, transposition systems in eukaryotes, the evolution of programmable nucleases, and the spread of mobile genetic elements from prokaryotes to eukaryotes. Future studies utilizing improved abilities to infer spliced genes from eukaryotic diversity will likely uncover more RNA-guided enzymatic systems that might have broad biotechnological promise. Taken together, the Fanzor diversity leaves many systems and associated proteins to be explored and will expand the nuclease toolbox for new human therapeutics.

### Example 11: Fanzor Preliminary Analysis

Fanzors are predicted to be programmable nucleases. Fanzors (Fanzor 1 and Fanzor 2) are proteins that were found to contain RuvC nuclease domains in eukaryotic genomes. They are predicted to be programmable nucleases based on RuvC domain and similarity to bacterial TnpBs. Computational analyses conducted herein show how the presence of a conserved non-coding region near the Fanzor genes that is likely the guide RNA for the protein. In this example, a number of these proteins were tested and verified that they are programmable nucleases. The impact of these are that they can be new enzymes for genome editing and they come from eukaryotic systems making them safer and potentially better for human therapeutics.

### Example 12: Fanzor Nucleases are TnpB Homologs Widespread in Eukaryotes and Viruses

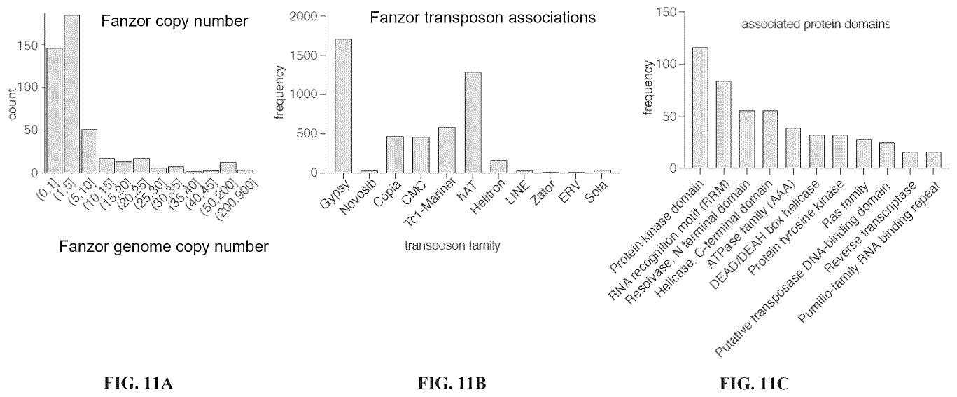

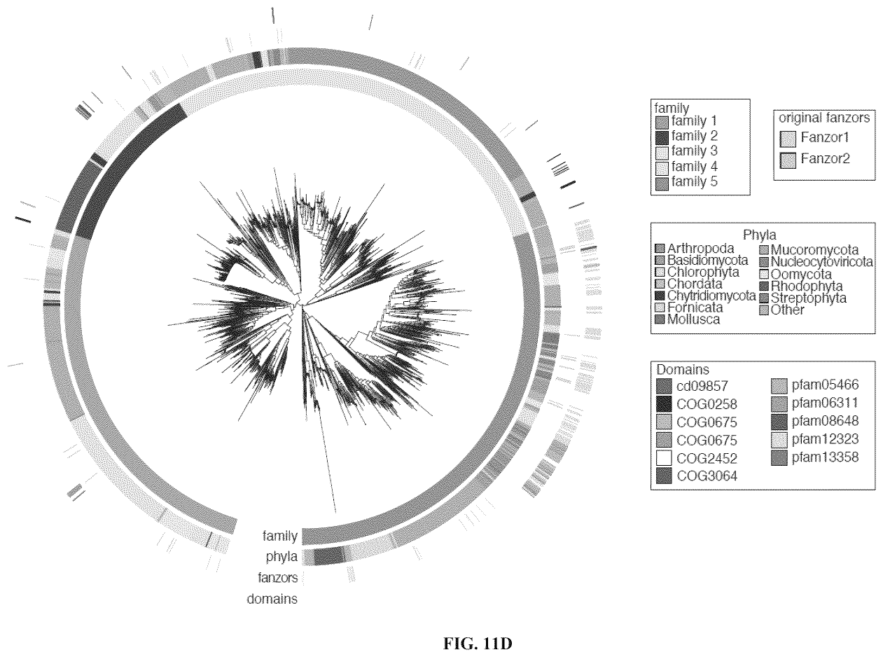

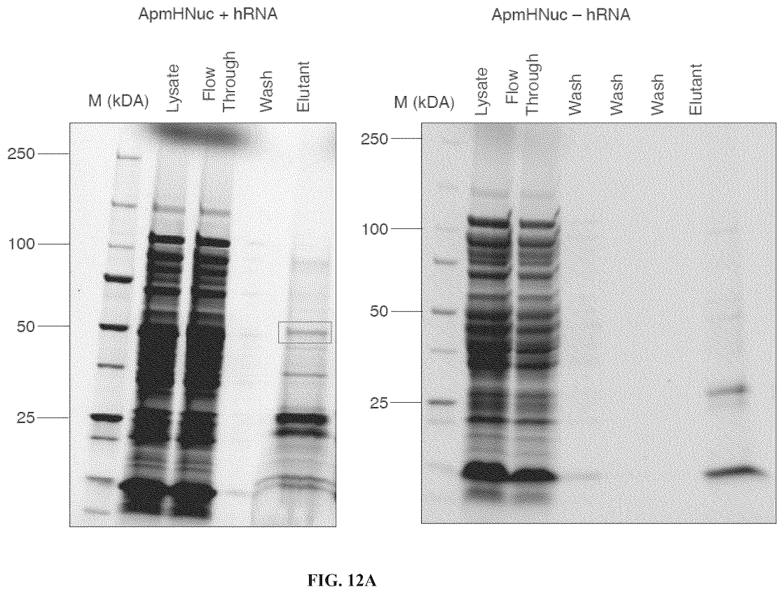

Putative RNA-guided nucleases were identified throughout eukaryotic genomes and their viral genomes by comprehensively mining 22,497 eukaryotic and viral assemblies from NCBI GenBank. This present search, seeded with a multiple alignment of RuvC domains from the previously identified Fanzor1 and Fanzor2 proteins (Bao et al. 2013), yielded 3,655 putative nucleases occurring across metazoans, fungi, algae, choanoflagellida, rhodophyta, unicellular eukaryotes, and multiple viral families (FIG. 6A), expanding on existing eukaryotic RuvC diversity by 100-fold. These nucleases contain existing Fanzor proteins that show similarity to their prokaryotic counterpart TnpB families (FIG. 6A) and frequently occur multiple times within genomes, indicating movement via MGEs in a similar fashion to TnpBs (FIG. 11A). The enzymes were termed Horizontally-transferred Eukaryotic RNA-guided Mobile Element Systems (Fanzor), owing to their mobility. A phylogenetic tree built from a multiple sequence alignment of Fanzor nucleases revealed 5 families, with Fanzor2 systems contained in Fanzor family 5 and Fanzor1 systems contained in all Fanzor families (FIG. 6B). Fanzor families are represented in diverse eukaryotes, including fungi, plants, various protists, and animals, with family 5 systems enriched in viruses, including Phycodnaviridae, Ascoviridae, and Mimiviridae (FIG. 6A-6B). Profiles of each Fanzor family were used to find the closest TnpB orthologs in prokaryotes and built a combined tree of Fanzor and closest TnpBs to understand their evolution (FIG. 6A). The different clades of Fanzor families and their related branches of TnpBs suggest that TnpBs were captured by eukaryotes on at least two independent occasions to convergently evolve the Fanzor superfamily, although many more seeding events are likely based on the presence of similar TnpBs within each of the five Fanzor clades (FIG. 6A).

### Example 13: Fanzor Nucleases Associate with Diverse Transposons

Given the association of Fanzors with different transposons (Bao et al. 2013), a comprehensive eukaryotic transposon search was performed (Riehl et al, 2022) within 10 kb of all Fanzor MGE sequences (FIG. 6B). This prediction yielded both previously reported transposon families including Mariner, Helitron, and Sola, and new ones that include both retrotransposons like Gypsy and ERV systems and DNA transposons like hAT and CMC (FIG. 11B). Interestingly, the two most frequent associations are with the retrotransposon Gypsy and the DNA transposon hAT, showing the potential acquisition of these Fanzor systems by eukaryotic transposons, potentially to help with retention of transposons inside the eukaryotic genome (FIG. 11B). Transposon association also clustered with Fanzor families: families 1, 3, and 5 commonly occurred with Gypsy domains, while families 2 and 4 associated with hAT, CMC, and Tc1-mariner systems (FIG. 6B).

Analyzing associations of Fanzor nucleases with surrounding proteins revealed numerous instances of transposase domains, including the serine resolvase found in IS607 elements, further demonstrating the inclusion of Fanzor in transposons (FIG. 11C). Fanzor proteins often contain additional domains beyond the characteristic RuvC-like domain (FIG. 11D), with family 5 containing profiles hits to the helix-turn-helix (HTH) domain and TnpB cluster COG0675, suggesting close evolutionary distance to their ancestor TnpBs.

### Example 14: Fanzor Loci are Associated with Conserved and Structured Non-Coding RNAs

Since TnpB and IscB systems are known to process either the 3′ end or 5′ end of the MGE RNA into ωRNA and subsequently bind to ωRNA for guided dsDNA cleavage activity (Karvelis et al., 2021; Altae-Train et al. 2021; Nety et al. 2023) a comprehensive noncoding RNA alignment search was performed on all Fanzor loci. The search revealed significantly longer Fanzor noncoding conservation on both the 3′ and 5′ ends of the MGEs compared to TnpB and IscB systems (FIG. 6C-6D). This strong conservation prompted a thorough investigation for specific structural hallmarks. The Fanzor family 5, containing Fanzor2 systems, are most closely related to TnpB, with Fanzor and TnpBs interspersed in the respective clade (FIG. 6A). Given the close relationship between TnpBs and Fanzor family 5, Fanzor family 5 was initially focused on as a likely source for RNA-guided DNA endonucleases. The Fanzor nuclease from the Acanthamoeba polyphaga mimivirus (ApmHNuc) within the IS607 MGE inside the mimivirus genome was selected (FIG. 6E). ApmHNuc co-clusters with an IS607 TnpA transposase inside the MGE flanked by defined inverted repeats elements (FIG. 6E). Copies of the ApmHNuc protein throughout the A. polyphaga mimivirus genome were searched for and three loci were found. Aligning these with the surrounding Fanzor loci to identify conservation throughout the locus (FIG. 6F), a strong conservation was found within the protein-coding regions of the ApmHNuc ORF and in the non-coding region at the 3′ ends of the IS607 MGE (FIG. 6E-6F), similar to bacterial TnpB systems. This non-coding sequence conservation extended 200 base pairs past the end of ApmHNuc ORF, ending at the right inverted repeat (IRR) of the MGE (FIG. 6F). In silico RNA secondary structure analysis of the region between the end of the ApmHNuc ORF and the IRR predicted a stable fold (FIG. 6G), suggesting that the transcript of this conserved region could function as a nuclease-associated RNA, which was termed a Fanzor RNA (fRNA). It was hypothesized that the fRNA could complex with ApmHNuc, potentially directing binding and cleavage activity to a specific sequence. Within the ApmHNuc cluster of systems, a consensus representative fRNA structure had high conservation (FIG. 6G). Interestingly, conservation of the consensus fRNA structure extended upstream into the coding region of the ApmHNuc ORF, indicating possible co-folding with the upstream region (FIG. 6G) and a potential RNA processing site (FIG. 6G triangle). This conservation of structure is reminiscent of the OMEGA families, where both the IscB and TnpB clades possess limited structural variation (Altae-Train et al. 2021) and where processing of the upstream region of the co-transcribed mRNA-ωRNA can release functional guide RNAs (Nety et al. 2023).

### Example 15: ApmHNuc is a fRNA-Guided DNA Endonuclease

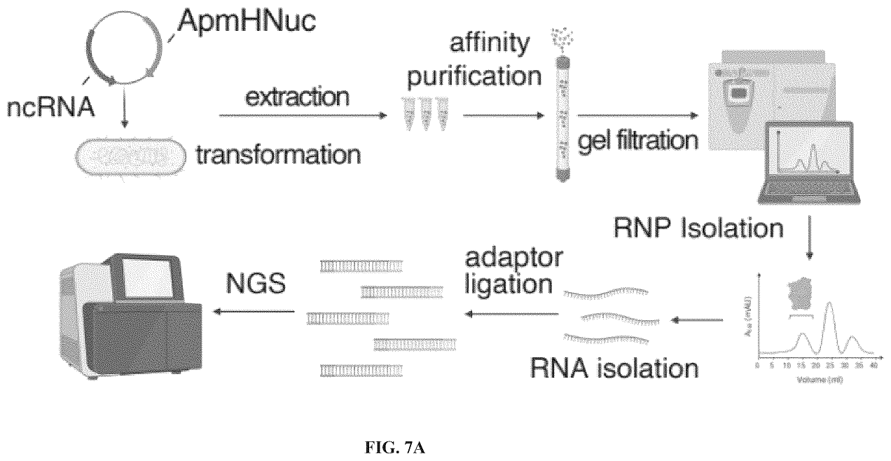

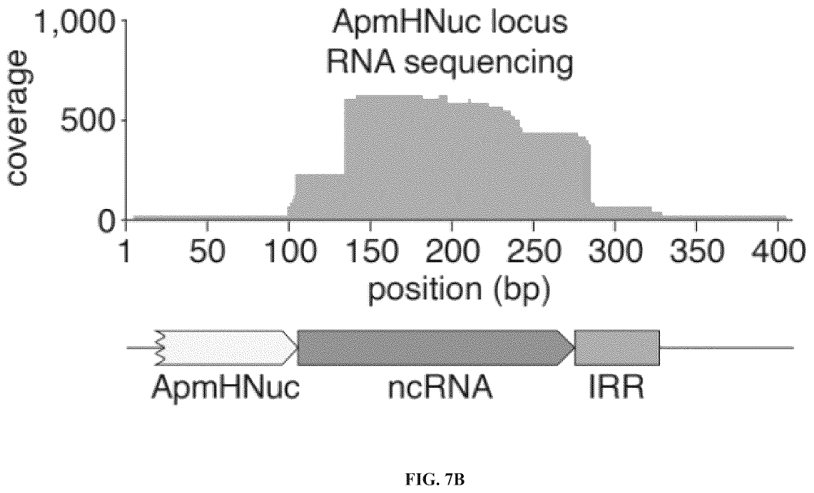

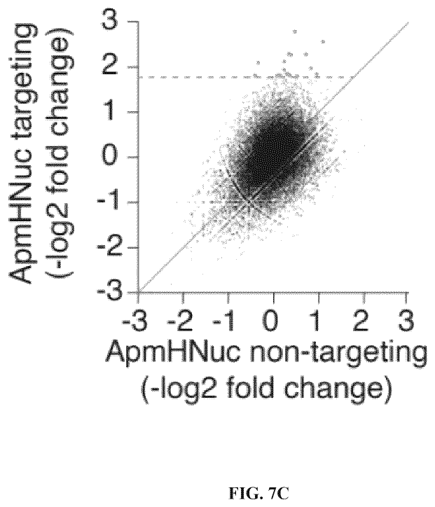

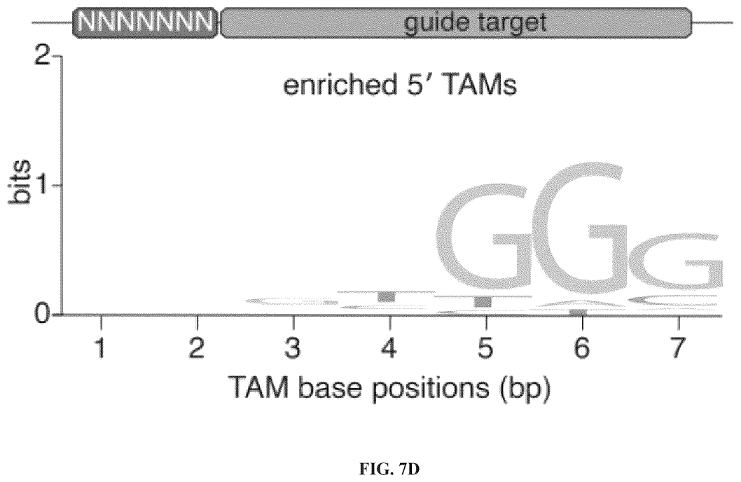

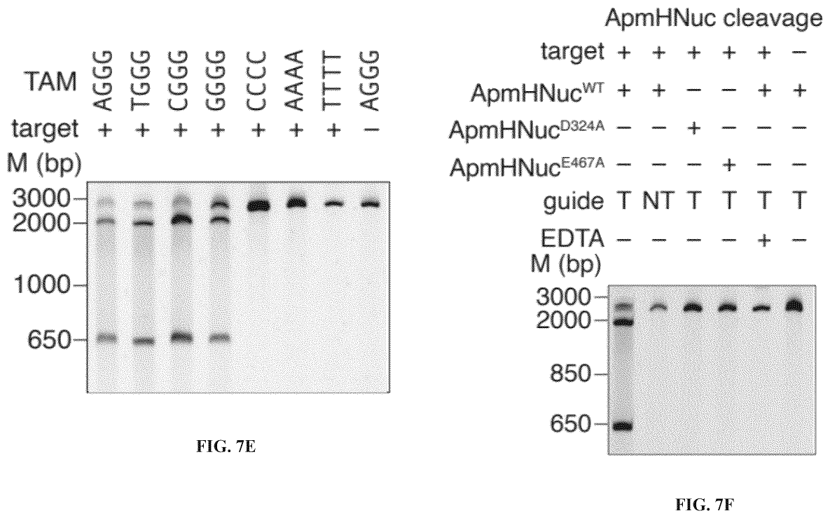

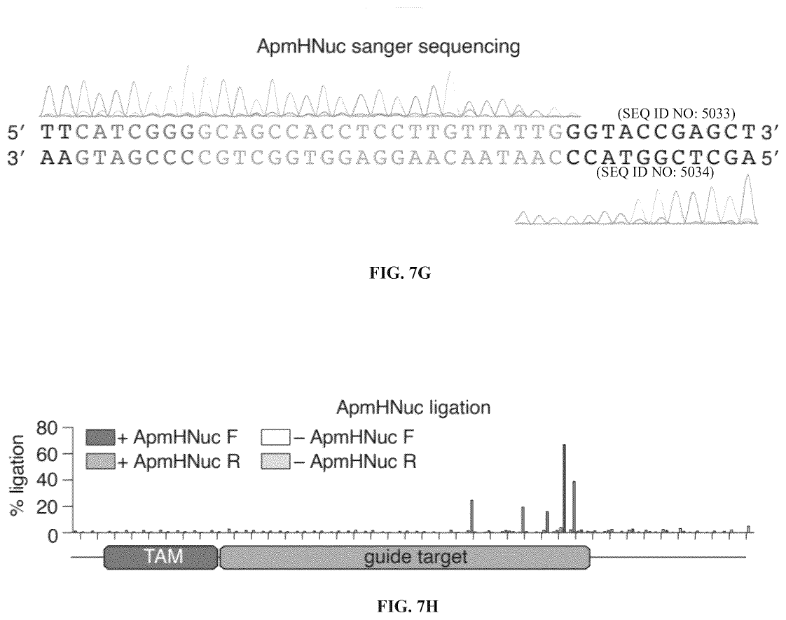

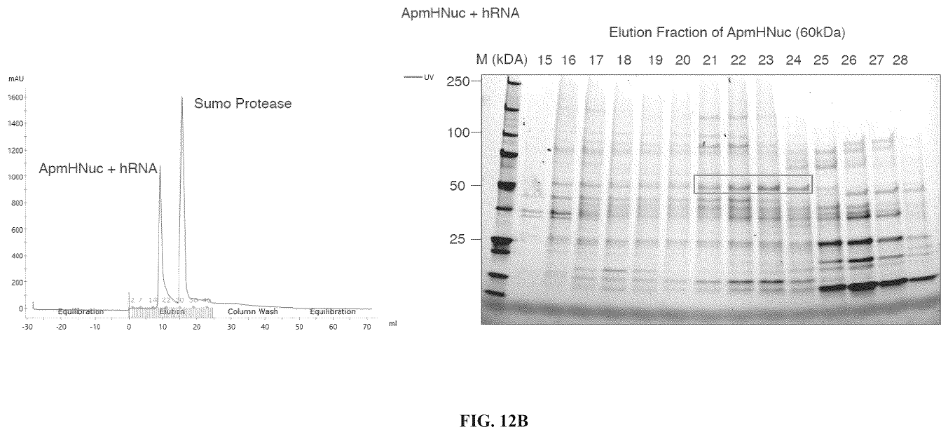

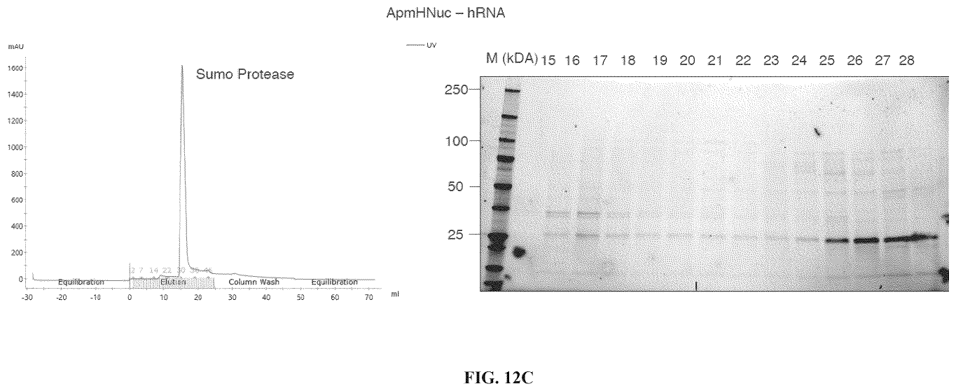

The conservation of the fRNA and similarity of Fanzor nucleases to prokaryotic RNA-guided nucleases suggested that the fRNA could associate with ApmHNuc and program DNA cleavage through ApmHNuc's conserved RuvC domains. To investigate potential fRNA-ApmHNuc binding, the A. polyphaga mimivirus Fanzor locus, containing the non-coding RNA region, and E. coli codon-optimized ApmHNuc, were co-expressed in E. coli (FIG. 7A, Table 2). Notably, ApmHNuc was unstable when expressed alone and required co-expression with the fRNA for protein stabilization and accumulation (FIGS. 12A-12C), similar to the instability of TnpB in the absence of @RN (Karvelis et a. 2021; Altae-Train et al. 2021). The ApmHNuc-bound fRNA species was profiled by purifying the fRNA-ApmHNuc ribonucleoprotein (RNP) and sequencing the RNA component of the complex. Small RNA sequencing revealed enriched coverage between the 3′ ends of the protein ORF and the IRR, in agreement with the evolutionary conservation across the region (FIG. 7B).

TABLE 2
Sequences associated with the present disclosure

| Fanzor/ | | | | | |
|---|---|---|---|---|---|
| TnpB | Genome | | SEQ | Associated fRNA Scaffold | SEQ |
| System | Accession | | ID | Sequence (neglecting | ID |
| names | Number | Protein Sequence | NO: | guide) | NO: |
| ApmHNuc | AY653733 | MKEAVKNVKPKVPAKKRIITGSKTKKKVFVK | 1 | AAAAATAGTCTAATAAA | 5 |
| | | KKPPDKKPLKKPVKKTVKTYKLKSIYVSNKD | | ATCAGGGGTACATTCCG | |
| | | LKMSKWIPTPKKEFTEIETNSWYEHRKFENP | | CTAGTACTCCACCCTAC | |
| | | NGSPIQSYNKIVPVVPPESIKQQNLANKRKKT | | GGGTTAAGCAAATGAG | |
| | | NRPIVFISSEKIRIYPTKEQQKILQTWFRLFAC | | AATATCGAAACGGTATG | |
| | | MYNSSIDYINSKKVVLESGRINVAATRKVCNK | | CACAGGATTCTTCGAGT | |
| | | ISVRKALKTIRDNLIKSTNPSIMTHIMDEAIGL | | GATAATCTTAGGATGAC | |
| | | ACSNYKTCLTNYIEGQIKKFDIKPWSISKRRKI | | TCACTAAGGAGATGACT | |
| | | IVIEPGYFKGNSFCPTVFPKMKSSKPLIMIDKT | | AAAGTGTATCATTCAAT | |
| | | VTLQYDSDTRKYILFVPRVTPKYSVNKEKNS | | ATTGTATTGAACGGTAT | |
| | | CGIDPGLRDFLTVYSENETQSICPIEIVVNTTK | | TCTTCCATAGAGAGTTG | |
| | | NEYKKIDKINEIIKTKPNLNSKRKKKLNRGLR | | ATTTTGGAGTATCCAGA | |
| | | KYHRRVTNKMKDMHYKVSHELVNTFDKICI | | AATATCAACTtTTTATGA | |
| | | GKLNVKSILSKANTVLKSALKRKLATLSFYRF | | GCGG | |
| | | TQRLTHMGYKYGTEVVNVNEYLTTKTCSNC | | | |
| | | GKIKDLGASKIYECESCGMYADRDENAAKNI | | | |
| | | LKVGLKPWYKQK | | | |
| CRE | NC057016 | MAPKRRRDEAEKAEEEKDHTTSTKCGLAGL | 2 | GCCGCCATGGCCGCCG | 6 |
| | | LSEKIEADGVAVTREESLAAVDFLVAALTRLRF | | GCGGCGGCGGGGCCGG | |
| | | EALCLLGLVAVRMCEDARREGQGLQPHCATC | | GCTGAGAGCCTGAACG | |
| | | RRLRKTELVEDDMYAAICAVSVCDLTEQGRK | | GCGCTAGCAGGGCGTG | |
| | | RGRPSKRDQHPEDDLFRHVCEEHFPRDEEAA | | GGGCTGAGGGTGCACG | |
| | | GARVNRSGLTPFLPPLSKGVFTNVKNHYAAN | | TGTTGATTGGCGGCGAG | |
| | | FAAWLARSFRCRIDDELRELRTPATKKLDKLA | | TGACGTGACTAGTTTGT | |
| | | WSMAHAVLYDGELEQPRWWVGWAQGAAG | | TAGCTGCGGGTTAGCAC | |
| | | AAAAAAAQGAGPAGGAAAAQAWTALVDYV | | GGACTGTGCACCCCAC | |
| | | NAQRASKRAAELLLREVKGAQATYKKASTR | | CCCACCGGCCACGTTCC | |
| | | HMEWAAEILAGLEARRDOLGAQVQQLTQAQ | | GGATTTGCGGGGATGCA | |
| | | PLTREDTQRLASLRRELHRARPFTLTPSPSFAPI | | AAGGCCCCCAACATAG | |
| | | YVPLDNTSMARLPGLLPTLARRHGEVFAGAG | | AGGCGTGTGCTTAGTAG | |
| | | AGAVAPSSFVQAAFGGGGMQSSATLNAVGW | | GCGCCCGCGTCAAGGT | |
| | | GLFQLGGVTSRNAPFANYITTDGVACSVARE | | GGCTGGGTTGATAACGA | |
| | | AHNKPLANLKPATAPADAEELCTLEEMKATQI | | CCCGGGAGGGGAGGGC | |
| | | IGVDPCGGGNWFMAARSPLYQPGPWAWEGV | | TCAGCCCTTTTCCTGCC | |
| | | GPAQRYLLELHDKQLDEELFPGQLPPEPRRRR | | TCCCTAAGGCAGCCACC | |
| | | KGVHRRKQSKHWQPRARTARRRRQKRGRFH | | TCCTTGT | |
| | | MSMGHWRHMSGLERLQPNRPQLAPALQAYV | | | |
| | | GGIPTAATASAARFEERLRYLFASGAAGQAAG | | | |
| | | GPAEAGPRGAVHVLWHYHFSAFRRKRWAAFI | | | |
| | | QRDRALHRVAKQLTGGRPKEEVVVGWGSWA | | | |
| | | FQGGKGGSPISVRGGRAPTGRLIKLLRERYAK | | | |
| | | HVFIIDEYKTSKTCYNCGCQEMAIKRLGGLK | | | |
| | | EGQRPWSVKVCNDCLTTWNRDVSAANVIRV | | | |
| | | LLLLKLMGFERPTKLQRPPWPPAAAGPG* | | | |
| TvoTnpB | NC_002689 | MKRANAVKLIVGKETHEKLKELAIVAAKCW | 3 | gggaagcccatgatgatgggcgtatt | 7 |
| | | NEVNWLRMQQFKEGERVDFSKTEKEVYEKY | | aagcgtggtctctataggtgtctccgc | |
| | | KQILKVNTQQVARKNAESWRSFFSLIEEKKG | | atagggaaggtaataaacgcagacct | |
| | | KLPKWFKPRPPGYWKDKSGKYKMLIIIRNDR | | gaatggtgcaataaatatcctacatatc | |
| | | YEIDEEKRIIYLKDFKLSLSFNGKLKWRGKQG | | cccgagtccctaggagctgggagca | |
| | | RLEIIYNEARRSWYAYIPVEVQNDVKAEDKL | | gagggcaactcacagtgagggatag | |
| | | KASIDLGIINLATVYVEDGSWYIFKGGSVLSQ | | gggtaatgggctgaagacccagccc | |
| | | YEYYSKRISVAQKTLARHKQGRSREMKLLHE | | gcggtctaccgctggacgaatggagc | |
| | | KRKRFLKHALNSMVRKIMEEFKNKGVGEIAI | | ggggtgggtgtcctcacccactagcta | |
| | | GYPKEISKDHGNKLTVNFWNYGYIIRRFEGV | | tgaagtgatgaaaatgaaggcggtaa | |
| | | GEELGVKVVKVDEAWTSKTCSLCGEAHDDG | | actgcaaaccaatgaatcgccacaag | |
| | | RIKRGLYRCLRIGKVINADLNGAINILHIPESL | | ggaaccttcaccctttagg | |
| | | GAGSRGQLTVRDRGNGLKTQPAVYRWTNGA | | | |
| | | GWVSSPTSYEVMKMKAVNCKPMNRHKGTFT | | | |
| | | L | | | |
| Isdra2TnpB | AE000513 | MIRNKAFVVRLYPNAAQTELINRTLGSARFV | 4 | GATTCAAGAATCCCGAA | 8 |
| | | YNHFLARRIAAYKESGKGLTYGQTSSELTLLK | | GTGAAGAATCTTGCCGT | |
| | | QAEETSWLSEVDKFALQNSLKNLETAYKNFF | | CCGTACATGGACTTGCC | |
| | | RTVKQSGKKVGFPRFRKKRTGESYRTQFTNN | | CGAACTGTGGGGAAAC | |
| | | NIQIGEGRLKLPKLGWVKTKGQQDIQGKILN | | CCATGACCGAGACGAG | |
| | | VTVRRIHEGHYEASVLCEVEIPYLPAAPKFAA | | AACGCTGCGCTGAACA | |
| | | GVDVGIKDFAIVTDGVRFKHEQNPKYYRSTL | | TTCGGCGTGAAGCGTT | |
| | | KRLRKAQQTLSRRKKGSARYGKAKTKLARI | | GGTGGCTGCGGGAATC | |
| | | HKRIVNKRQDFLHKLTTSLVREYEIIGTGHLK | | TCAGACACCTTAAACGC | |
| | | PDNMRKNRRLALSISDAGWGEFIRQLEYKAA | | TCATGGAGGCTATGTCA | |
| | | WYGRLVSKVSEYFPSSQLCHDCGFKNPEVKN | | GACCTGCTTCGGCGGG | |
| | | LAVRTWTCPNCGETHDRDENAALNIRREALV | | CAATGGTCTGCGAAGT | |
| | | AAGISDTLNAHGGYVRPASAGNGLRSENHAT | | GAGAATCACGCGACTTT | |
| | | LVV | | AGTCGTGTGAGGTTCA | |
| | | | | A | |

It was hypothesized that ApmHNuc is guided by its associated fRNA to target and cleave DNA sequences. Testing this activity required both the engineering of a reprogrammed fRNA and the determination of sequence preferences, akin to a target adjacent motif (TAM) (Karvelis et al. 2021; Altae-Tran et al. 2021). A synthetic fRNA was generated by combining a 3′-terminal 21-nt targeting sequence with the fRNA scaffold (ending at the IRR) determined through RNA profiling. Rosetta cells were co-transformed with plasmids coding for both the synthetic fRNA and ApmHNuc, and isolated the RNP complex from E. coli. To determine potential sequence preferences of ApmHNuc, cleavage on a DNA target containing a randomized 7 nucleotide TAM 5′ of a 21 bp target region complementary to the fRNA targeting sequence was tested. The TAM library was co-incubated with purified ApmHNuc RNPs containing either targeting or scrambled synthetic fRNA guide sequences, and the relative depletion of sequences was profiled with next-generation sequencing (NGS). TAM depletion analysis revealed a strong 5′ GGG motif adjacent to the target site (FIGS. 7C-7D). This TAM was validated on all four possible NGGG sequences, finding robust ApmHNuc cleavage on all four sequences, with no detectable cleavage on sequences lacking the TAM (FIG. 7E). This G rich ApmHNuc TAM is in contrast to the closely related TnpB homologs which universally prefer an A/T rich 5′ TAM similar to CRISPR Cas12 effectors (Nety et al. 2023). Without wishing to be bound by any theory, this change in TAM preference is likely attributed to the nearby IS607 transposase which starts with a recognition sequence of GGG at the 5′ end inverted left repeat element (ILR). Recently, TnpB has been reported to bias their nearby IS element's retention in the genome by targeting the donor joint of IS200/605 transposon for cleavage (Meers et al. 2023). It is likely that Fanzor family 5 members play a similar role in helping their host transposons to retain in the eukaryotic genome and their viruses.

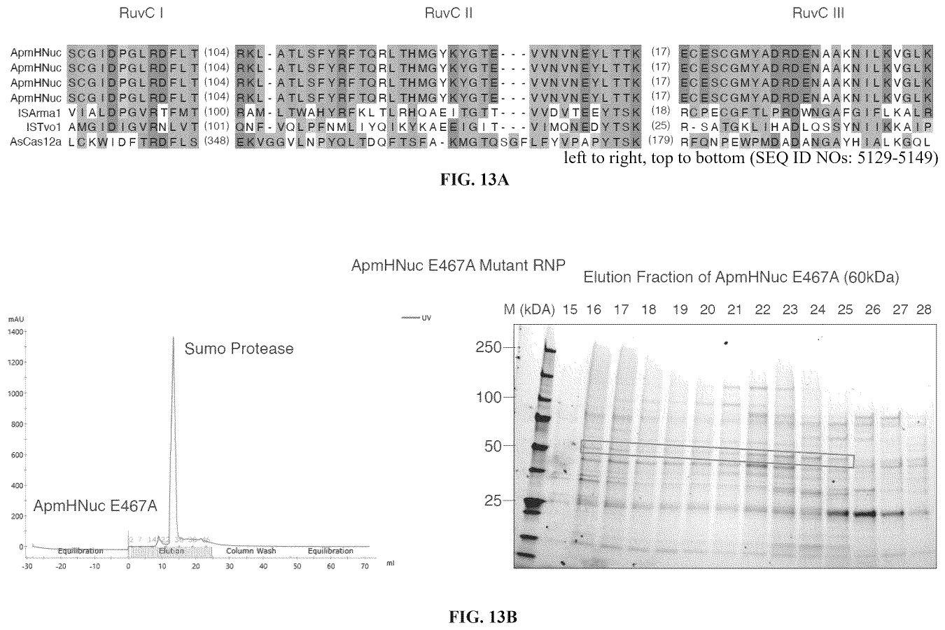

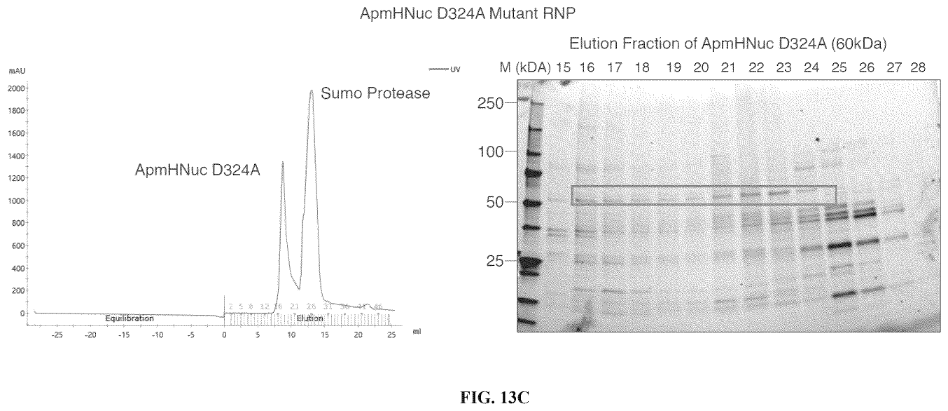

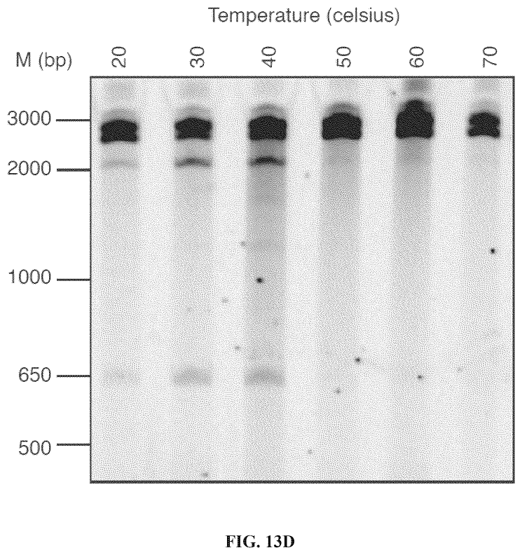

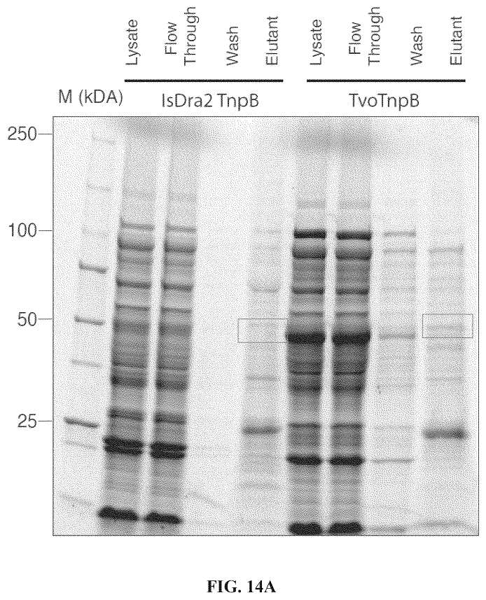

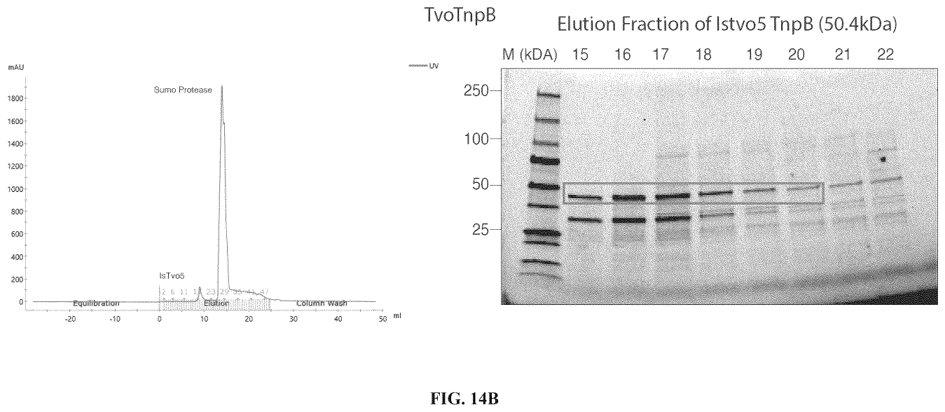

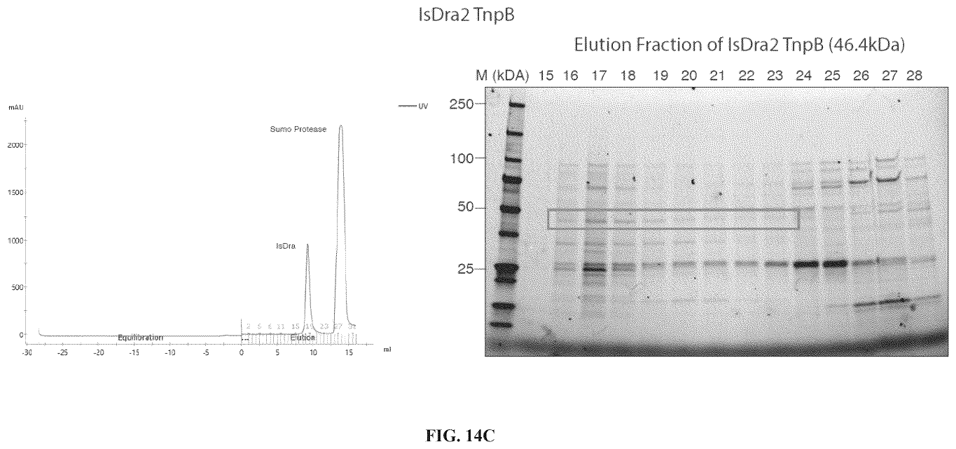

Similar to TnpB (Nakagawa et al. 2023; Sasnauskas et al. 2021), cleavage by ApmHNuc is likely mediated by conserved acidic residues in the RuvC domain (FIG. 13A). To confirm that the observed cleavage was dependent on the RuvC catalytic mechanism, two ApmHNuc RNP mutants at putative catalytic sites in either RuvC-I (D324A) or RuvC-II (E467A) were purified (FIGS. 13B-13C). While the D324A mutant had no change in RNP stability during protein purification, a significant decrease in expression of the E467A mutant relative to the wild type protein was noticed (FIG. 13B). The cleavage efficiency of these mutants was compared with the wild-type ApmHNuc and, in agreement with the nuclease mechanism, it was found that both RuvC-I and RuvC-II mutants abolished ApmHNuc cleavage activity (FIG. 7F). ApmHNuc cleavage requires magnesium (FIG. 7F), similar to other RuvC nucleases, and optimal activity is between 30 and 40 degree Celsius (FIG. 13D).

Cleavage locations of RNA-guided nucleases vary substantially, with cleavage sites both up and downstream from the target location. To profile ApmHNuc cleavage patterns, ApmHNuc reaction products were purified and the locations of the cleavage ends were mapped using Sanger sequencing. Cleavage occurred in the 3′ regions of the target sequence, with multiple nicks in both the target strand (TS) and the non-target strand (NTS) (FIG. 7G). The cleavage behavior of ApmHNuc at the 3′ end of the target is similar to the cleavage patterns of Cas12 or TnpB nucleases and in general agreement with programmable RuvC domains. The relative preference for these different nicking sites was sensitively quantified with an NGS-based assay, finding that during dsDNA cleavage by ApmHNuc the enzyme generates nicks on the NTS at positions 19 and 20 and on the TS at positions 15, 18, and 21 with all cleavage occurring inside the target spacer region, suggesting a slightly different cleavage pattern than TnpB nucleases (FIG. 7H).

### Example 16: Fanzor Nucleases Contain a Conserved Rearranged Catalytic Site and Lack Collateral Activity

Compared to a majority of TnpB families, Fanzor nucleases contain a substitution in the canonical catalytic RuvC-II site from a glutamate residue to a catalytically inert residue (proline, glycine) (FIG. 8A). To find if a subset of TnpBs similar to Fanzor nucleases might also display this substitution, a similarly modified RuvC nuclease domains among the TnpB families was searched for. A similar apparent catalytic inactivation of RuvC-II in a subset of TnpBs was found, in both the clade most related to Fanzor and one clade more distant to Fanzor nucleases (FIGS. 8A-8B). Anticipating the evolution of compensatory mutations in the RuvC-II domain to retain Fanzor activity, nearby conserved acidic residues that could serve as potential catalytic sites were searched for. Notably, all nucleases with a loss of the canonical glutamic acid in the RuvC-II, including all Fanzor members and the rearranged TnpB orthologs, contained an alternative conserved glutamate approximately 45 residues away (FIG. 8A-8I). it was hypothesized that this glutamic acid substituted the role of canonical one in the RuvC-II, to allow for effective cleavage activity.

To compare the structural conformations of the canonical and alternative catalytic sites, a TnpB from Thermoplasma volcanium GSS1 (TvoTnpB) harboring a rearranged site was selected, and compared experimentally determined or computationally predicted structures between ApmHNuc, TvoTnpB (re-arranged RuvC-II), TnpB from Deinococcus radiodurans R1 (Isdra2; canonical RuvC domain), and Cas12f from uncultured archaeon (UnCas12f) and compared the spatial configurations of the canonical and alternative catalytic glutamic acids (FIG. 8C). Notably, the alternative conserved glutamate of Fanzor nucleases and rearranged TnpBs (E467 of ApmHNuc and E323 of TvoTnpB) were in close proximity with catalytic residues in the RuvC-I and RuvC-III domains, suggesting that these alternatively conserved glutamates compensate for the mutation in the canonical RuvC-II residue (FIG. 8C). In addition, during structural analysis, we found that ApmHNuc is the only protein that has a long disordered stretch in the N-terminus (FIG. 8C). This disordered region is unseen in other TnpBs and CRISPR/Cas12 family members, suggesting that this N-terminal flexible region is an unique feature of Fanzor that likely plays a role in their activity.

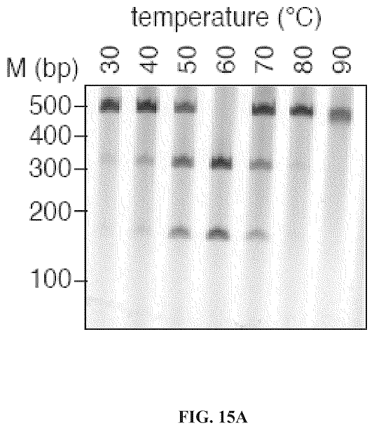

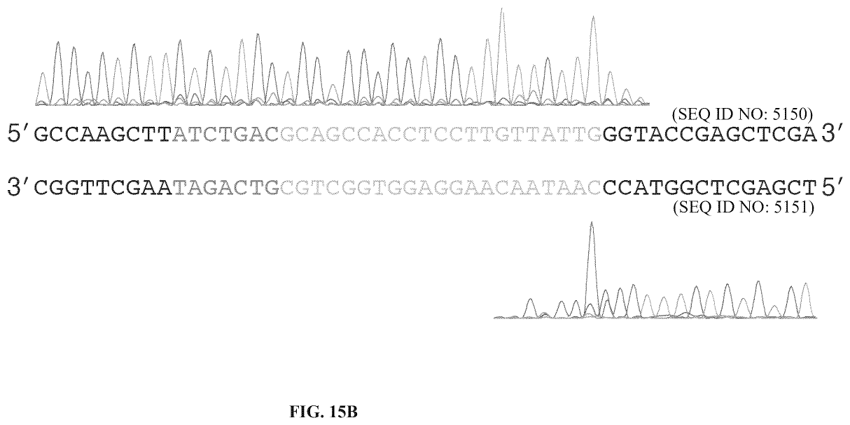

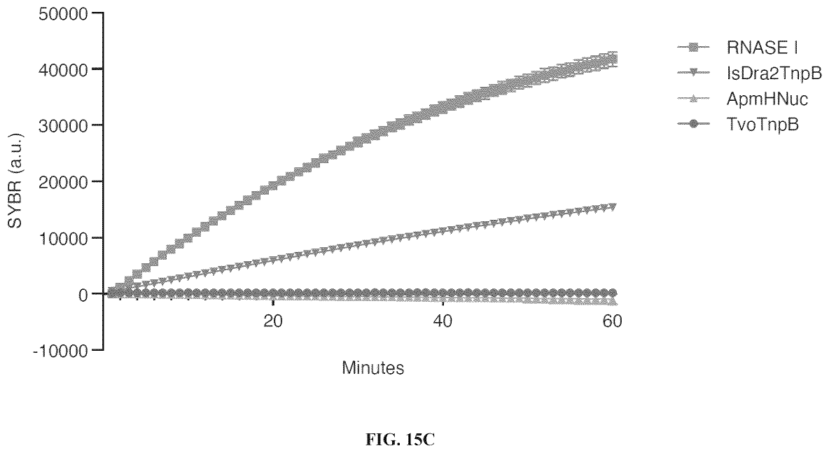

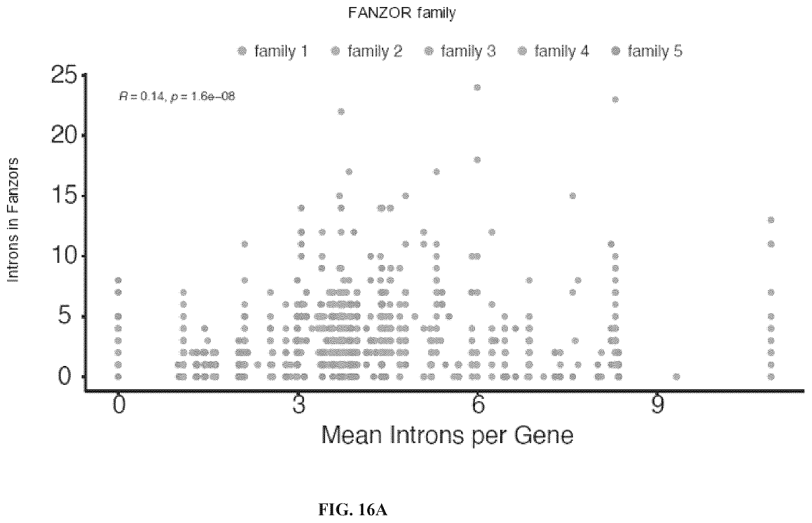

To generalize the activity of the rearranged RuvC domain beyond ApmHNuc, the nuclease activity of TvoTnpB was evaluated, which contains the alternative glutamic acid catalytic residue. TvoTnpB RNPs were generated by co-expressing the TvoTnpB protein with its native locus in E. coli, and these RNP were isolated to profile the associated noncoding RNA by NGS. A significant enrichment of noncoding RNA expression was found near the right end (RE) element, similar to other TnpB systems (FIG. 8E). Applying the TAM assay by coexpressing TvoTnpB with a synthetic ωRNA containing a reprogrammed 21 nt spacer, incubating the RNP with a 7N TAM library plasmid, and sequenced the cleavage products, a significant enrichment of a TGAC motif near the 5′ target spacer sequence (FIG. 8F). Notably, this TGAC motif is also present at the 5′ end of the left end (LE) element, marking the start of the Tvo mobile genetic element. As T. volcanium is a thermophile, the in vitro cleavage efficiency was optimized over a range of temperatures, determining an optimal temperature for cleavage of the TGAC TAM at 60° C. (FIG. 15A). All four possible NTGAC TAM sequences along with four negative TAM sequences were validated and TAM-specific cleavage was found, similar to other Fanzor and TnpB proteins (FIG. 8G). The ends of the cleavage products were profiled with NGS, mapping the cleavage position to position 22 in the non-targeting strand and positions 21 and 22 in the targeting strand (FIG. 8H), with a similar cleavage pattern found by Sanger sequencing (FIG. 15B).

Lastly, it was hypothesized that the rearranged RuvC catalytic site of the Fanzor might be less solvent exposed, as suggested by the structural analysis (FIG. 8C), reducing acceptance of outside nucleic acids and thus affecting the collateral cleavage activity of the enzyme (Chen et al. 2018; Abudayyeh et al. 2016) Both ApmHINuc and TvoTnpB were profiled for either RNA or DNA collateral cleavage activity by co-incubating the RNP complexes with their cognate targets along with either ssRNA or ssDNA cleavage reporters, single-stranded nucleic acid substrates functionalized with a quencher and fluorophore that become fluorescent upon nucleolytic cleavage. It was found that both ApmHNuc and TvoTnpB nucleases lacked collateral DNA and RNA cleavage activity in contrast to the strong collateral cleavage activity of the canonical TnpB Isdra2TnpB (FIG. 8I and FIG. 15C), suggesting that the rearranged RuvC domain has distinct biochemical properties compared to canonical RuvC domains in other TnpB systems and Cas12.

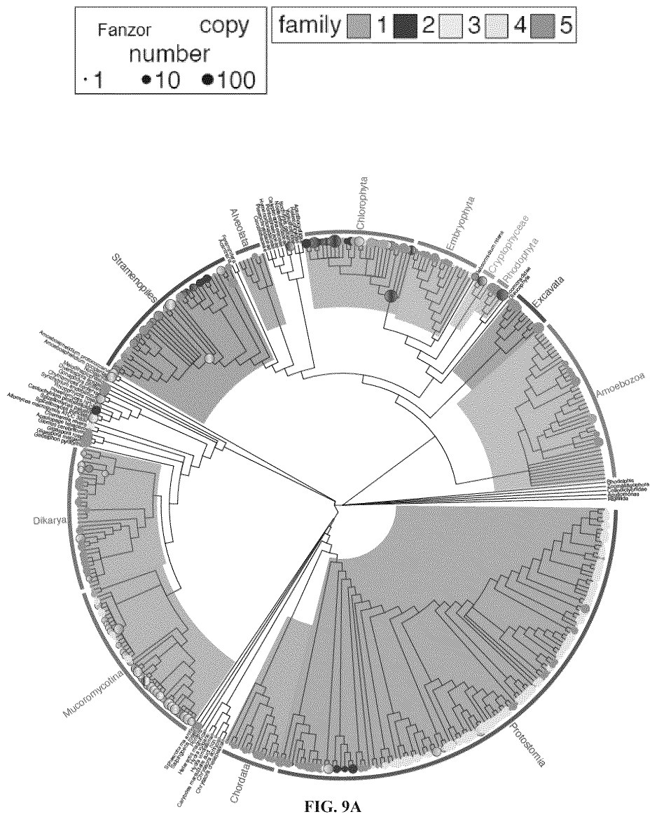

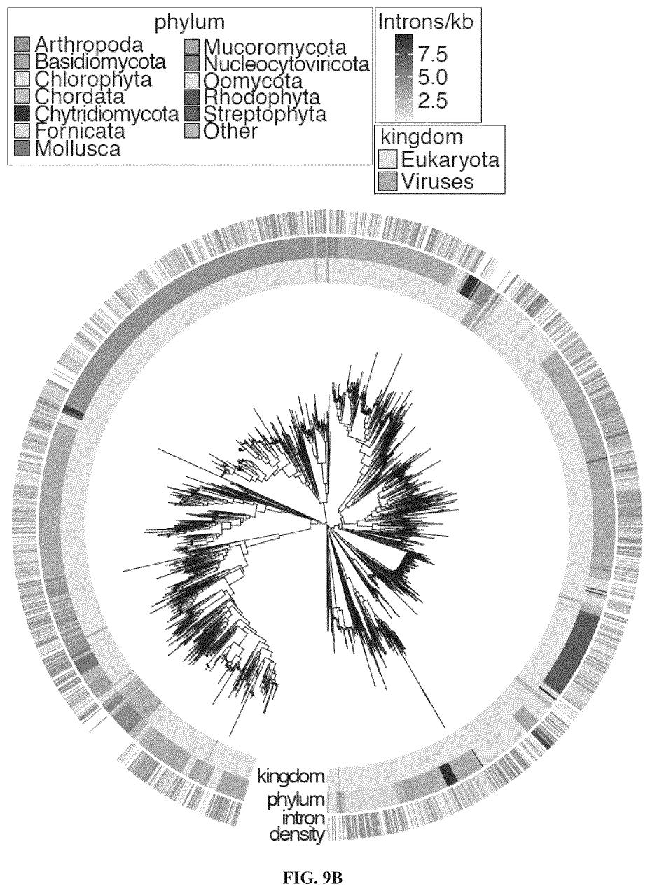

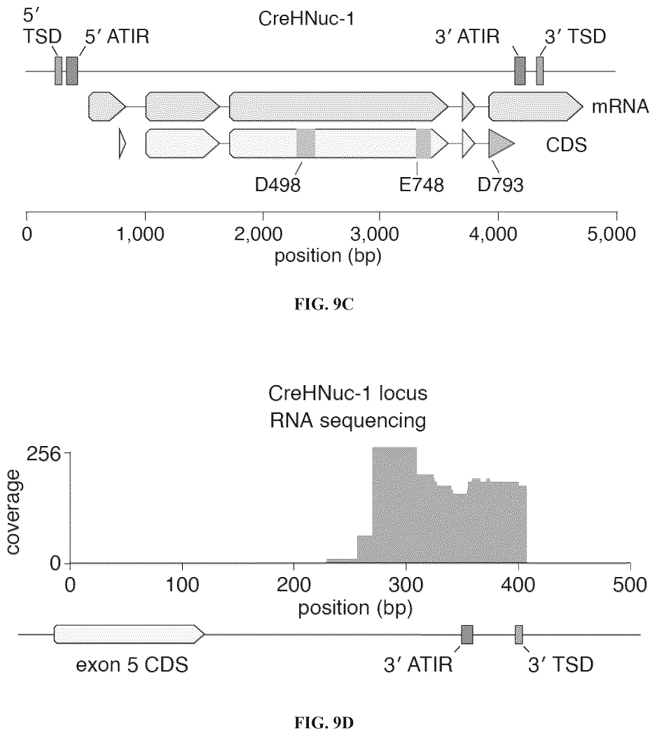

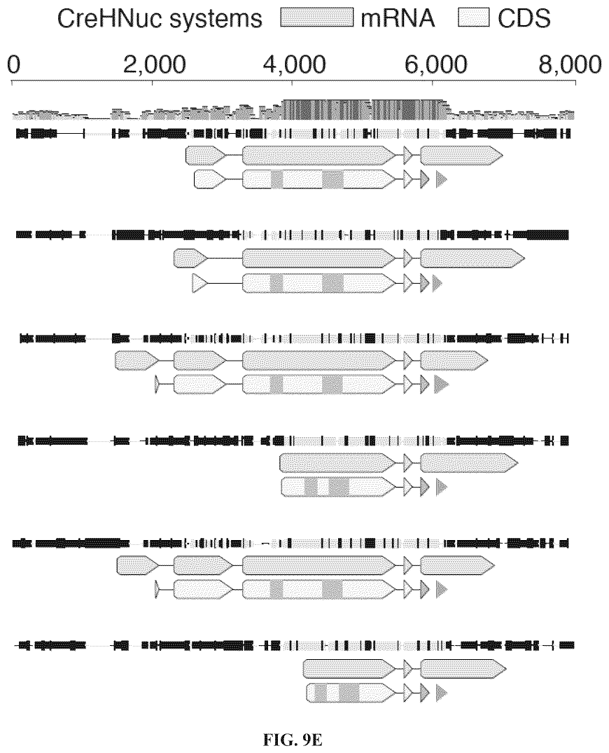

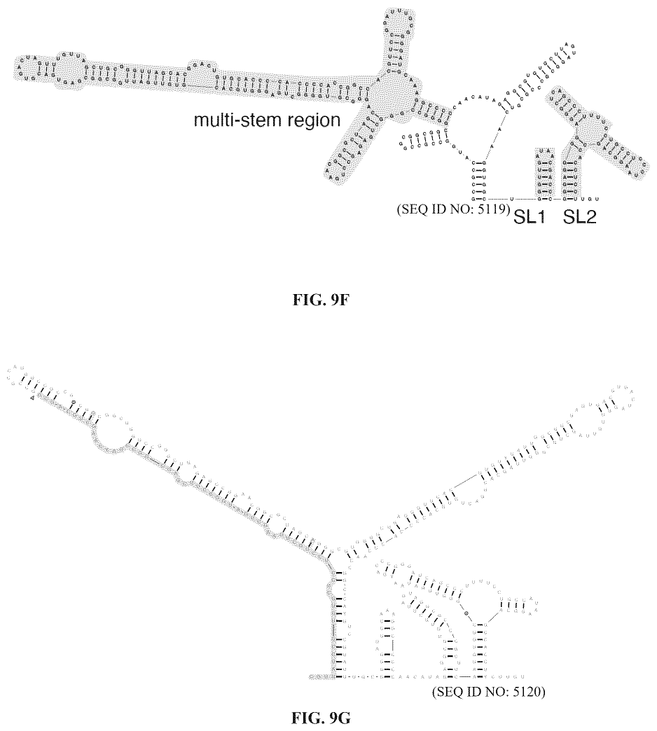

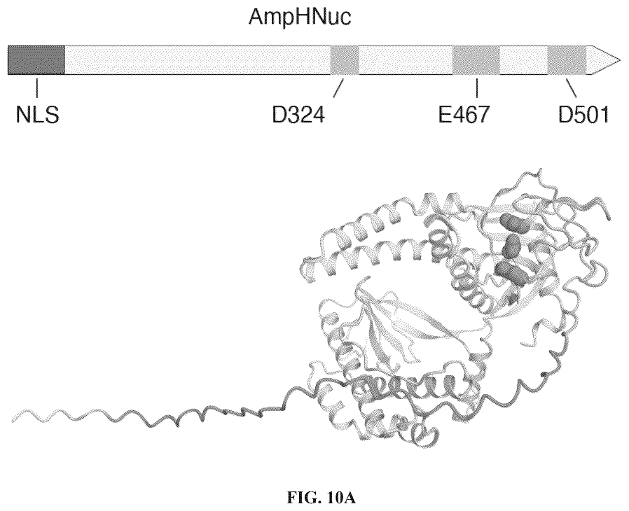

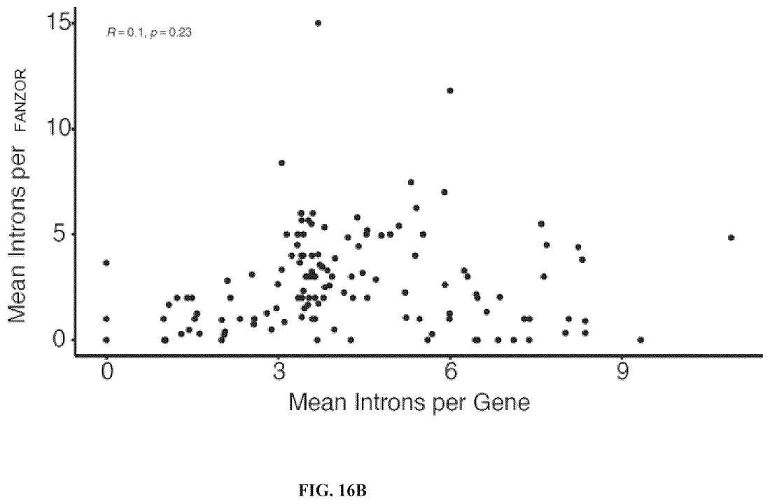

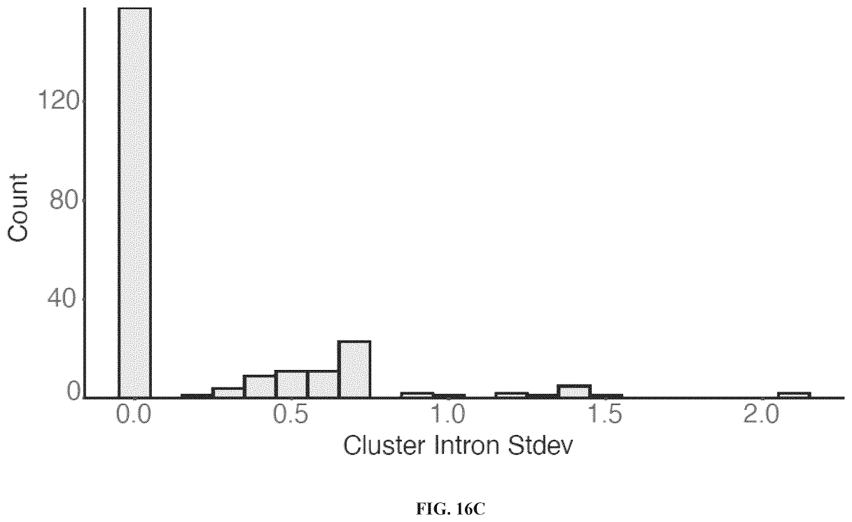

### Example 17: Fanzor Systems have Spread Throughout Diverse Eukaryotic Branches and Associate with their fRNAs

Whereas the Family 5 Fanzor systems are closely related to TnpB systems, it was found that most Fanzor orthologs, including Fanzor1 nucleases, are distantly related and have radiated throughout all eukaryotic branches of life, including amoeba, fungi, plates, and animals (FIG. 9A). Interestingly, Fanzor systems have even spread to certain higher-order phyla, such as Chordata and Arthopoda, suggesting extensive spread and evolution of these systems. Moreover, whereas many Fanzor systems contain no introns, as might be expected of TnpB-derived mobile genetic elements, we observed many Fanzor systems with extensive intron development of up to ~9.6 introns/kb (FIG. 9B). Intron acquisition further supports the notion that Fanzor systems have evolved in eukaryotes for significant evolutionary time. Intron densities of Fanzor systems have a weak but significant correlation with host genome intron densities, suggesting co-acquisition of introns with Fanzor systems and hosts after the first acquisition event (FIG. 16A-16B). While a majority of Fanzor clusters have similar numbers of introns, there are a number of clusters that show divergent numbers of introns, suggesting that closely related Fanzor systems are undergoing intron acquisition (FIG. 16C).

To demonstrate that these expanded Fanzor family members actively process and associate with their cognate fRNAs, family 1 Fanzor from the unicellular green alga Chlamydomonas reinhardtii (CreHNuc) was focused on (FIG. 9C). Notably, the multiple CreHINuc genes encoded in the algae genome transcribe pre-mRNAs with multiple introns. Interestingly, the RuvC domain is coded across multiple exons, with the RuvC-III aspartic acid encoded in the last exon away from the other two catalytic residues. Using RNA sequencing data, we confirmed the presence of four introns within the CreHNuc-1 pre-mRNA that are processed away from the mature mRNA transcript.

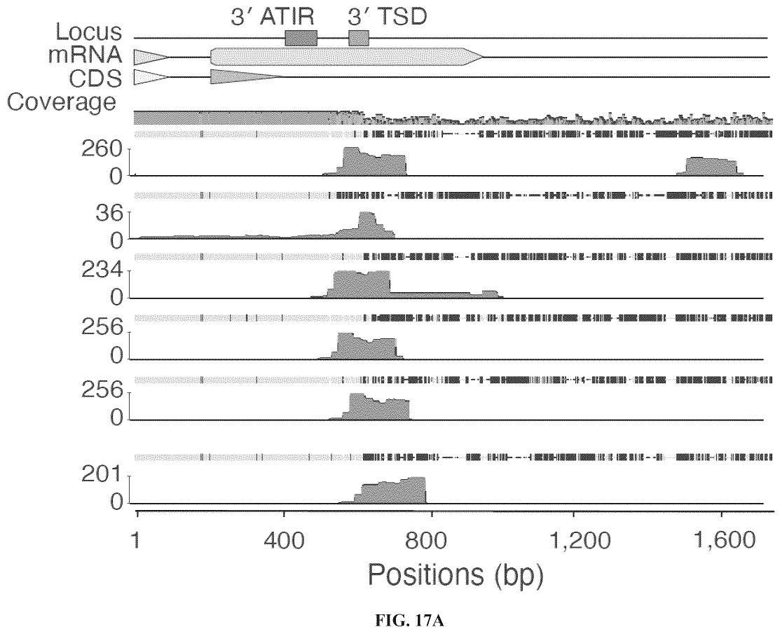

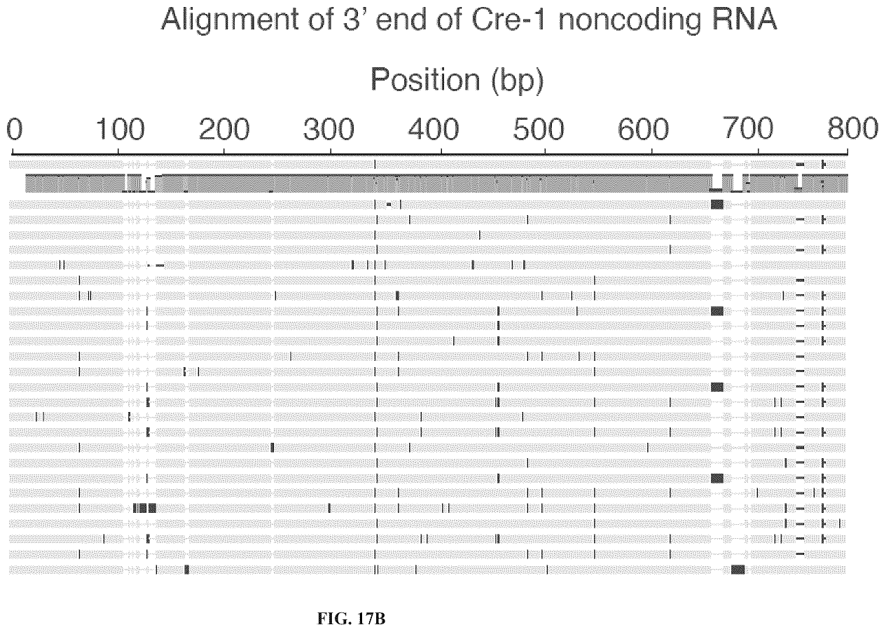

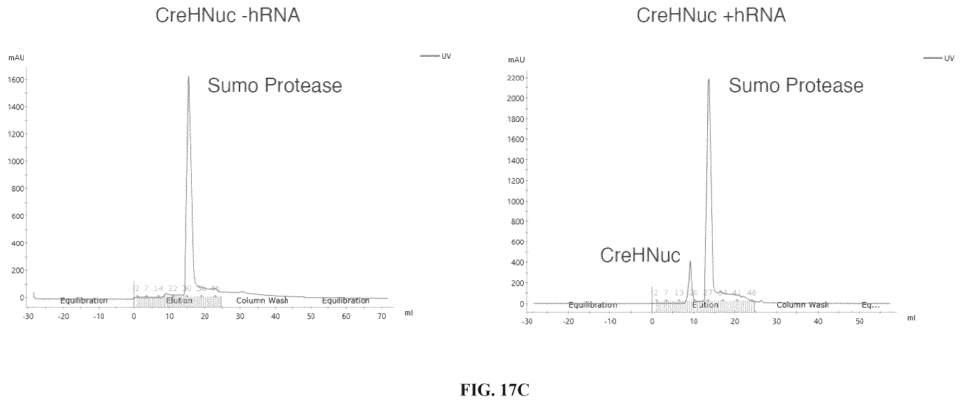

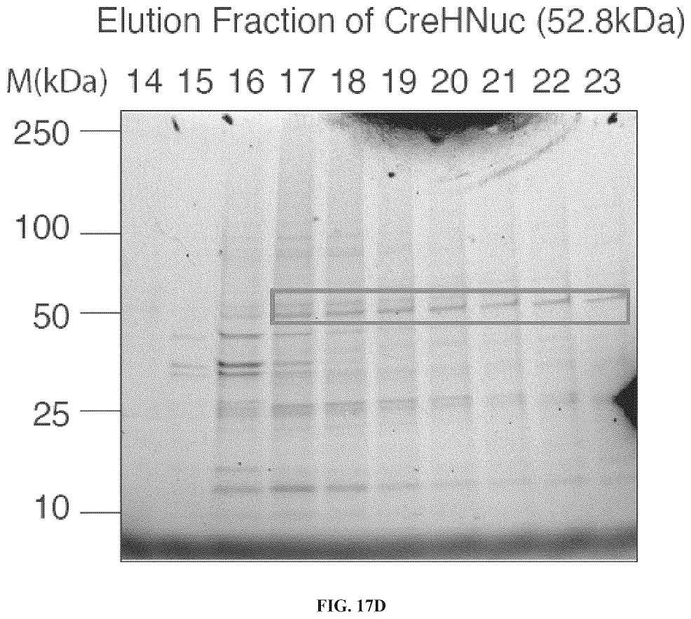

The CreHNuc systems are associated with Helitron 2 transposons, which contain identifiable short target site duplications (TSDs) and asymmetrical terminal inverted repeats (ATIRs). In the CreHNuc-1 system, we found defined TSD and ATIR sequences flanking 5′ and 3′ of the CreHNuc MGE. The CreHNuc-1 system lacks the RepHel domain, indicating that it is an non-autonomous Helitron. It was hypothesized that either the 3′ TSD or the 3′ ATIR sequence indicates the end of the fRNA of CreHNuc-1 and performed small RNA sequencing directly from the native green algae organism, finding significant enrichment of small non-coding RNAs aligning to the 3′ UTR of the CreHNuc-1 mRNA (FIG. 9D). Interestingly, fRNA traces at the CreHNuc-1 locus begin around 100 bp downstream of the end of the last exon and extend across the 3′ ATIR into the TSD (FIG. 9D), suggesting that CreHNuc-1 is likely involved in host Helitron transposition. We hypothesized that the fRNA for these CreHNuc systems are generally marked by the TSD produced by their native transposon upon insertion. Small RNA-sequencing traces were mapped onto all 6 functional copies of CreHNuc and found that all 6 instances of Cre-Hnuc fRNA lie inside the 3′ UTR of their mRNAs and are strongly conserved between the copies (FIG. 9E and FIG. 17A). Moreover, the conservation of this non-coding RNA was studied by searching for this sequence across the Chlamydomonas reinhardtii genome, finding that the non-coding RNA was highly conserved in sequence across 27 different instances (FIG. 17B). The observed fRNA for CreHNuc-1 was computationally folded and strong secondary structures were found, further supporting its potential role in serving as a guide RNA for CreHNuc-1 (FIG. 9F). To generalize these findings beyond the Cre Fanzor locus, fRNA conservation was analyzed by comparing similarities within the Cre Fanzor clusters. It was found that, within the CreHNuc cluster of systems, three representative fRNA structures had high conservation (FIG. 9G), with a conserved upstream region (FIG. 9G) and a putative cleavage site (FIG. 9G, triangle).

All 6 full-length copies of CreHNuc systems inside the genome were aligned and strong alignment was found near the C-terminal coding region of the Fanzor nuclease, which contains the RuvC domain, and variable N-terminal compositions (FIG. 9E). While unclear why the coding regions of CreHNuc are not conserved like ApmHNuc systems, one possible explanation, without wishing to be bound by any theory, is that the Helitron transposon undergoes rolling circle replication that starts at the 3′ end of the MGE, resulting in variable length replicons and truncations. The C-terminal RuvC domain is likely beneficial for this transposition process and thus is evolutionarily conserved.

To evaluate the functional role of the CreHNuc-1 fRNA, the CreHNuc-1 protein was co-expressed either with its native fRNA on the 3′ end of the MGE or a scramble RNA sequences. It was found that CreHNuc is only stable when coexpressed with its fRNA, suggesting that CreHNuc actively associates with its fRNA for stability (FIGS. 17C-17D) When the RNP was co-incubated with the 7N randomized TAM library plasmids, no cleavage was observed. This suggests that the CreHNuc and its associated Fanzor clusters might possess functions other than DNA endonuclease, which has been reported for some clades of TnpBs that actively process their own omegaRNA, but fail to cleave dsDNA (Nety et al. 2023)

### Example 18: Fanzor Nucleases Evolved Nuclear Localization Signals and can be Adapted for Mammalian Genome Editing

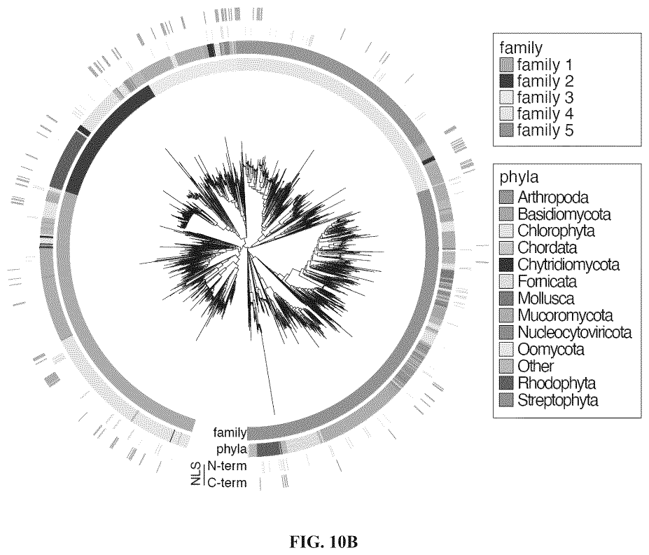

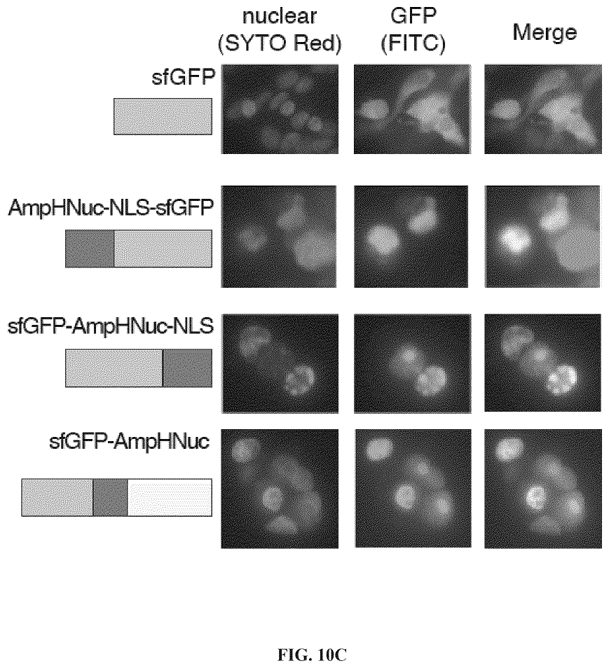

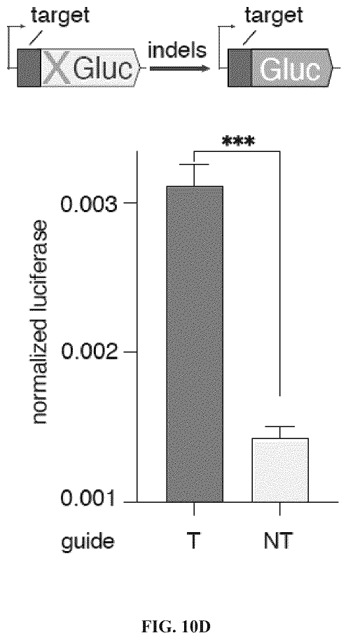

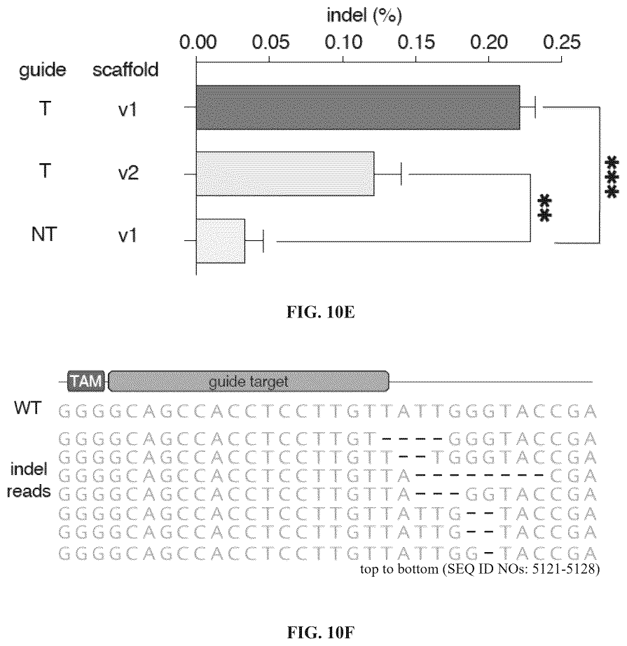

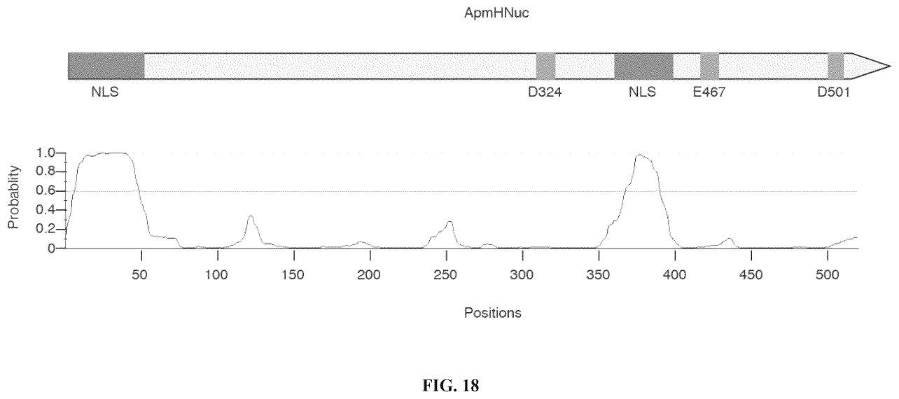

Since eukaryotic nucleases would need to invade nuclear membranes for genomic activity, unlike their prokaryotic counterpart TnpB, IscB, and CRISPR family proteins, it was hypothesized that Fanzor systems might have evolved nuclear localization signals to actively cross the nuclear membrane. Using Alphafold2 predicted structures of ApmHNuc, a disordered region of 64 amino acids on the N-terminus of ApmHNuc was identified, which was unique to ApmHNuc, but not its TnpB and CRISPR/Cas12 counter parts (FIG. 8C and FIG. 10A). The first 64 amino acids of ApmHNuc were analyzed with an NLS determination program and a strong similarity to canonical nuclear localization signal peptides that are rich in positively charged residues was found (FIG. 18). Given the evolutionary pressure to enter the nucleus, it was predicted that the N-terminal short peptide is likely acquired during evolution to aid entry into the nucleus. To understand how widespread this phenomenon is across Fanzor systems, the end termini of all Fanzor nucleases were analyzed and 8.6% of nucleases were found to have a readily identifiable NLS (FIG. 10B).

To evaluate the functional activity of the identified ApmHNuc NLS, the N-terminus NLS tag of ApmHNuc was fused to either the N-terminus or C-terminus of super-folded GFP (sfGFP). The sfGFP was also attached onto the N-terminus of wild-type ApmHNuc and visualized its location via fluorescent microscopy. It was found that compared to a wild-type sfGFP, the N-terminus NLS tag of ApmHNuc fused to either terminus of sfGFP resulted in a strong nuclear localization of sfGFP (FIG. 10C). Fusion of sfGFP with ApmHNuc also caused strong nuclear localization of sfGFP (FIG. 10C). These data suggest that the N-terminal NLS tag of ApmHNuc is a natural NLS peptide that likely evolved during the transition from prokaryotes to eukaryotes.

Next, to test whether Fanzor systems could be applied for mammalian genome editing given their mesophilic operating temperature and eukaryotic nature, ApmHNuc was codon-optimized for mammalian expression and engineered its fRNA guide for expression in mammalian cells. Since the fRNA is longer in length than typical ωRNAs (>350 nt), HEK293T cells were co-transfected with a T7 promoter-driven guide expression plasmid along with human codon-optimized T7 polymerase and wild-type ApmHNuc protein. A reporter plasmid that carries the 21 nt target matching the T7-driven guide was designed in front of a Gaussia luciferase (Gluc) out of frame from the start codon along with a cypridina luciferase (Cluc) driven by a constitutive promoter on the same plasmid to normalize for transfection efficiency. Indel activity would knock the Gluc into frame, allowing for detectable Gluc luciferase activity. Using this reporter system, a significant increase in normalized luciferase was found in the targeting guide condition compared to a non-targeting guide control, suggesting that indel were generated by the ApmHNuc protein (FIG. 10D). Indels were checked for by next-generation sequencing and indel editing in the targeting guide condition for ApmHNuc was found (FIG. 10E). Lastly, the indel pattern was analyzed and 2-5 bp deletions were found near the 3′ end of the target site (FIG. 10F), similar to the indel cleavage patterns of other programmable RuvC containing nucleases like Cas12 or TnpB systems.

### Example 19: Fanzor Nucleases are TnpB Homologs Widespread in Eukaryotes and Viruses

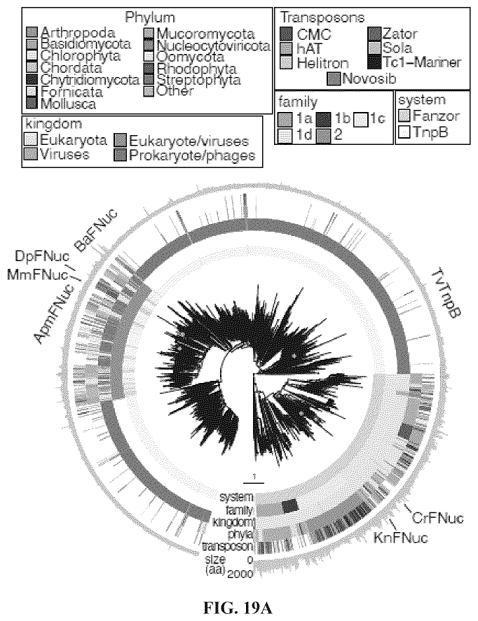

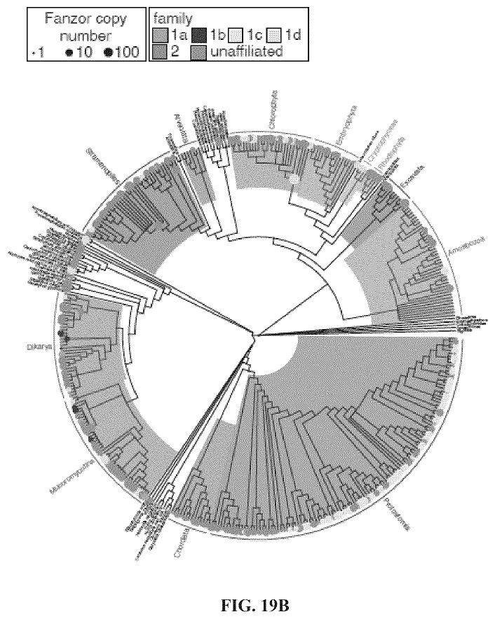

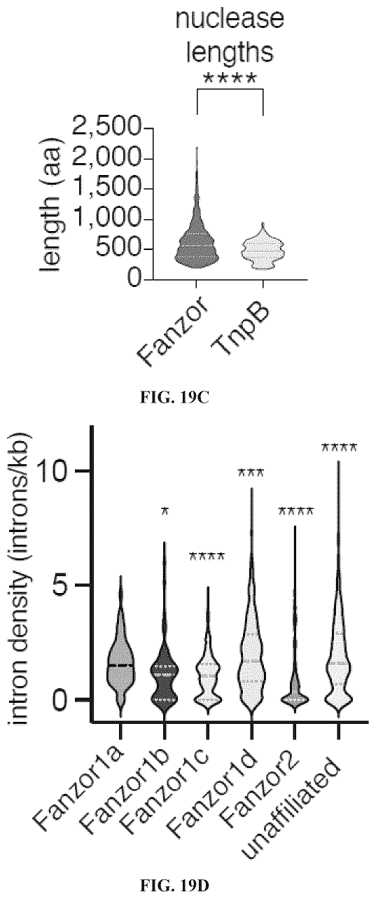

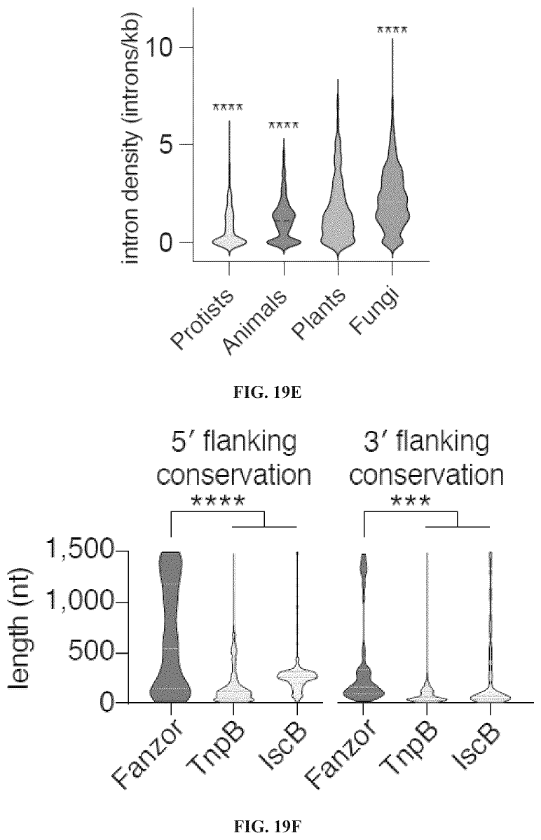

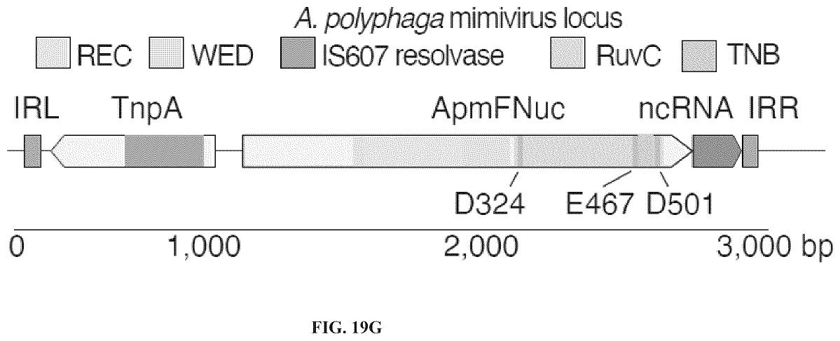

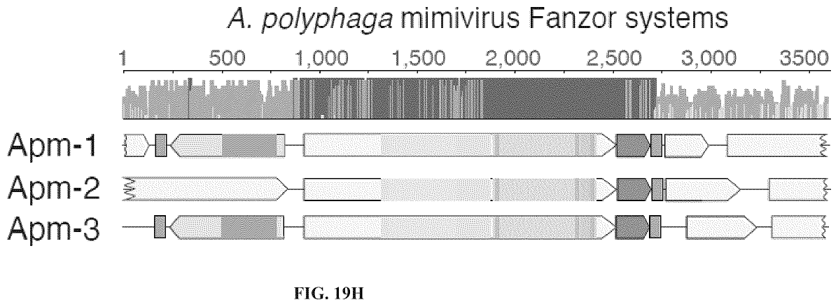

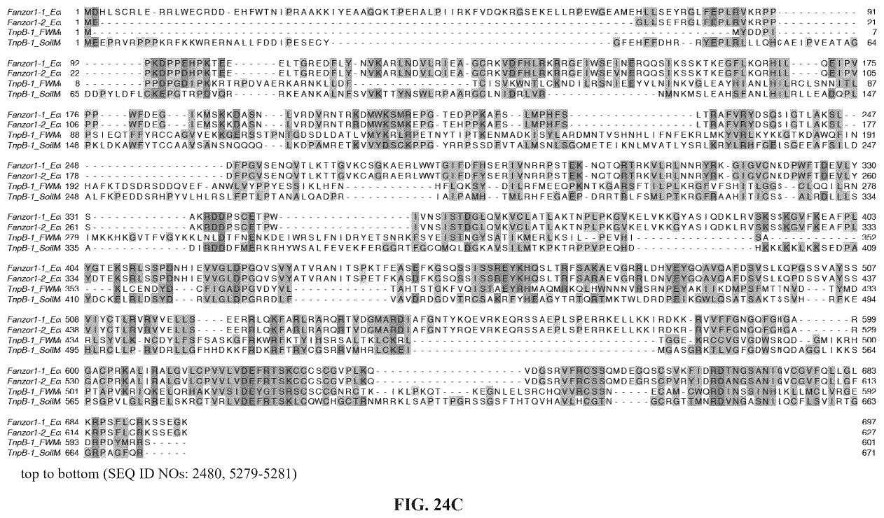

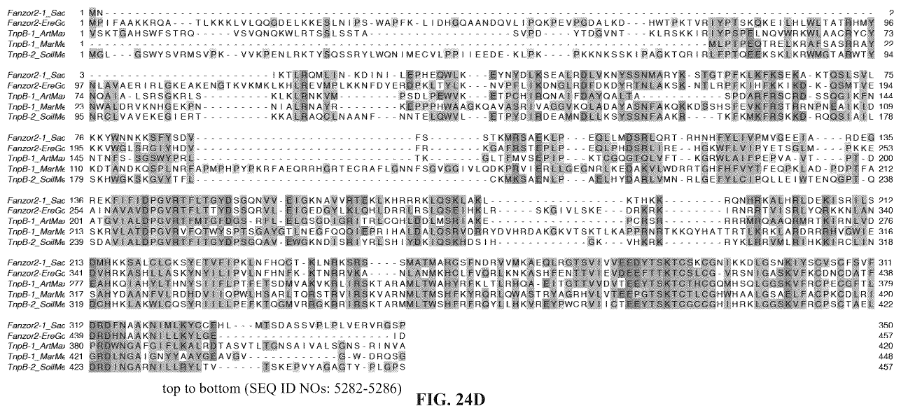

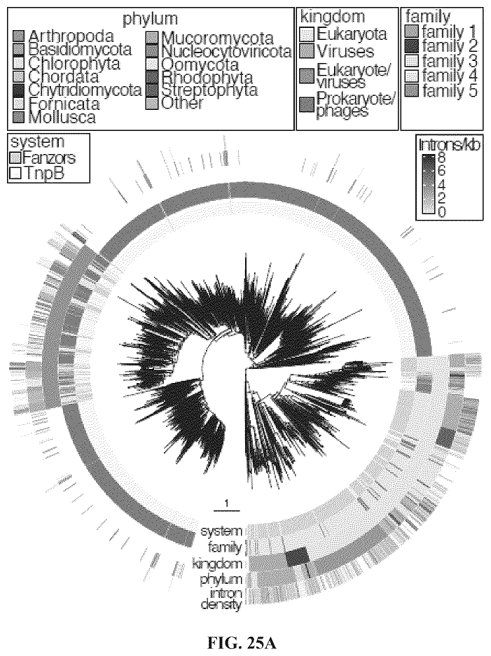

Putative RNA-guided nucleases were identified across 22,497 eukaryotic and viral assemblies from NCBI GenBank by searching for similarity to a multiple alignment of RuvC domains from known Fanzor1 and Fanzor2 proteins (Bao et al. 2013). There were 3,655 putative nucleases with unique sequences (using a 70% similarity clustering threshold) that occurred across metazoans, fungi, choanoflagellates, algae, rhodophyta, diverse unicellular eukaryotes, and multiple viral families (FIG. 19A and FIG. 19B), expanding the known diversity of eukaryotic RuvC homologs over 100-fold (FIG. 19A). These Fanzor homologs frequently occur in multiple copies across eukaryotic genomes, with some genomes carrying up to 122 copies. This wide spread of the Fanzors is strongly suggestive of intragenomic mobility, similar to TnpBs (FIG. 24A). Fanzor proteins also are typically substantially larger than TnpB, with a mean size of 620 residues, compared to 480 residues for TnpB proteins (FIG. 19C).

Phylogenetic analysis of the expanded set of Fanzor nucleases and a selection of closely related TnpBs revealed 5 distinct Fanzor clades supported by bootstrap analysis, with four Fanzor1 families (Fanzorla-1d) and a single Fanzor2 clade (FIG. 19A). In addition, there are a number of unaffiliated Fanzor systems that could not confidently be assigned to any Fanzor family based on phylogeny. Fanzors are each broadly represented in diverse eukaryotes, and Fanzor2 shows a pronounced enrichment of virus-encoded Fanzors (18.4%, p<1017), including Phycodnaviridae, Ascoviridae, and Mimiviridae (FIG. 19A). Fanzor proteins often contain various domains, in addition to the RuvC-like nuclease domain; in particular, Fanzor2 members contain a helix-turn-helix (HTH) domain, mimicking the domain architecture of the TnpBs (FIG. 24B). Furthermore, direct comparison of specific Fanzors and their closest TnpBs further supports the close evolutionary relationship between these enzymes (FIG. 24C and FIG. 24D). In all families, Fanzors are interspersed with TnpBs, suggesting multiple acquisitions of TnpB during the evolution of eukaryotes. Moreover, TnpB-containing clades that include sparse Fanzors might reflect direct acquisitions from symbiotic bacteria (FIG. 19A).

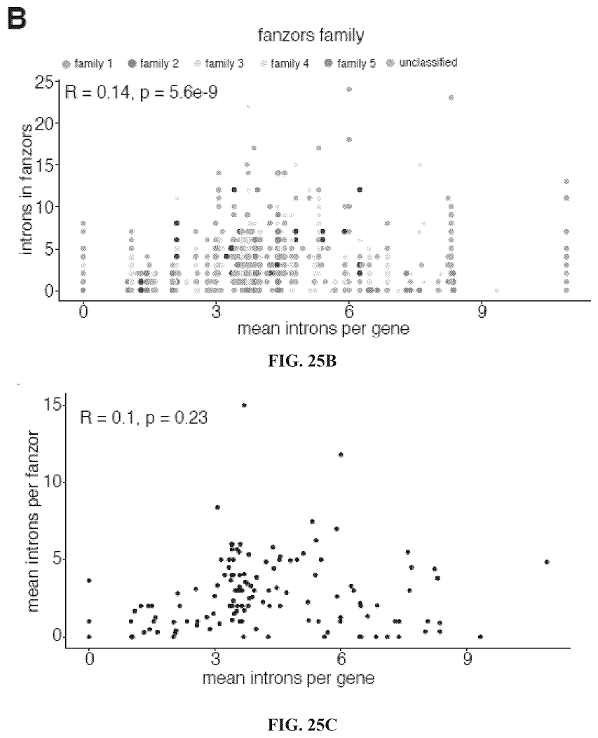

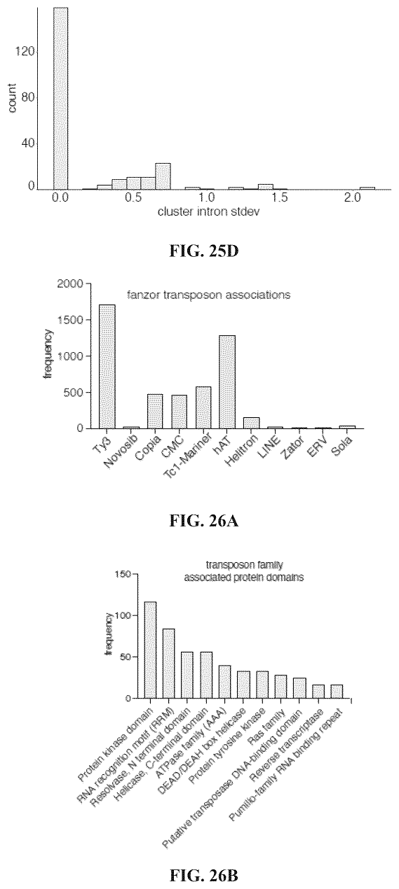

Projecting Fanzor hosts onto the eukaryotic tree of life shows broad spread into amoebozoa, several other groups of unicellular eukaryotes, plants, fungi, and animals, including Chordata and Arthopoda (FIG. 19B). Notably, assimilation of Fanzors in eukaryotic genomes was accompanied by intron acquisition: numerous Fanzor loci have intron densities similar to those in host genes, up to ~9.6 introns/kb (FIG. 19D, FIG. 19E, and FIG. 25).

### Example 20: Fanzor Nucleases Associate with Diverse Transposons

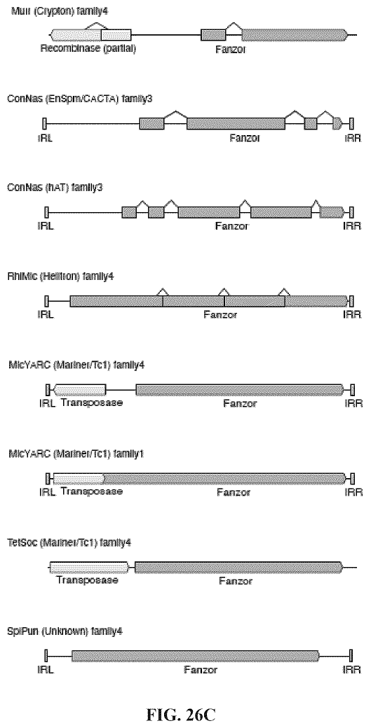

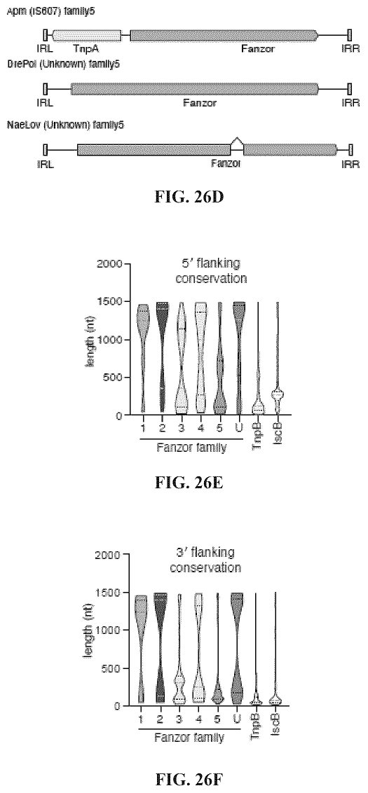

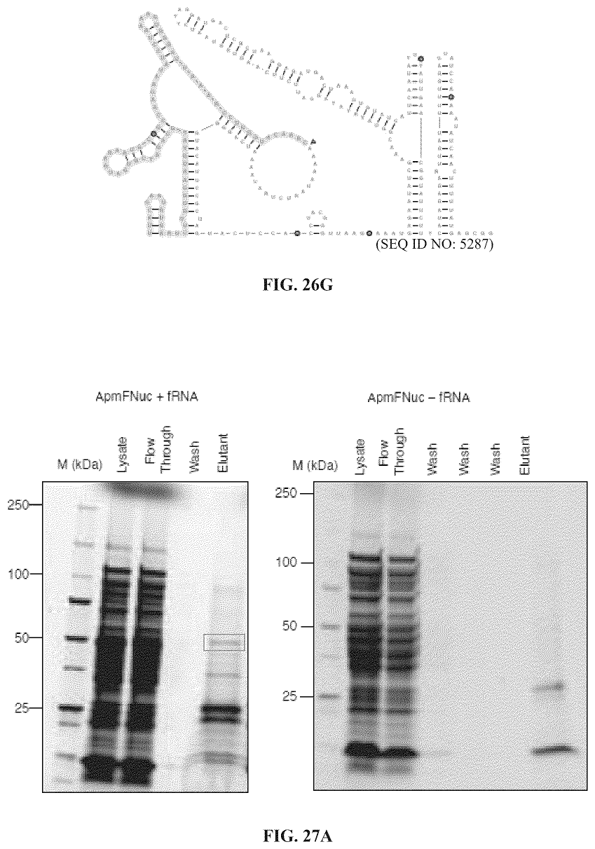

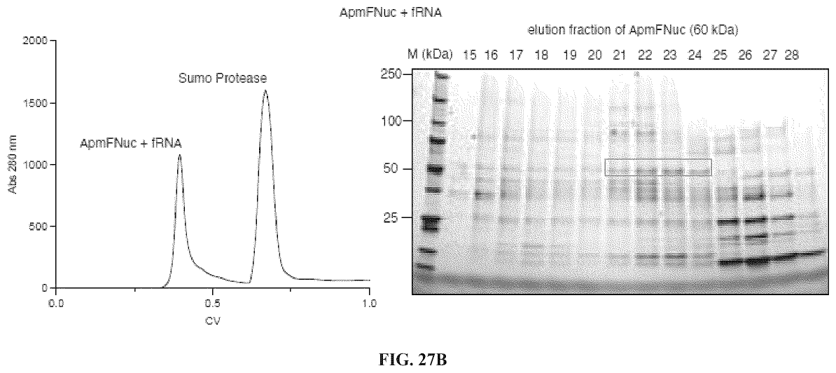

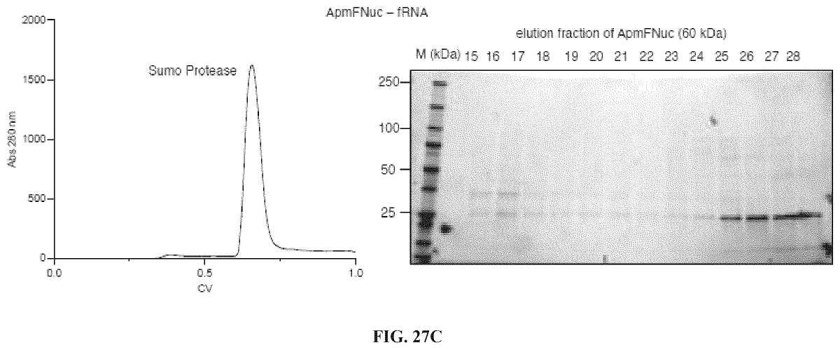

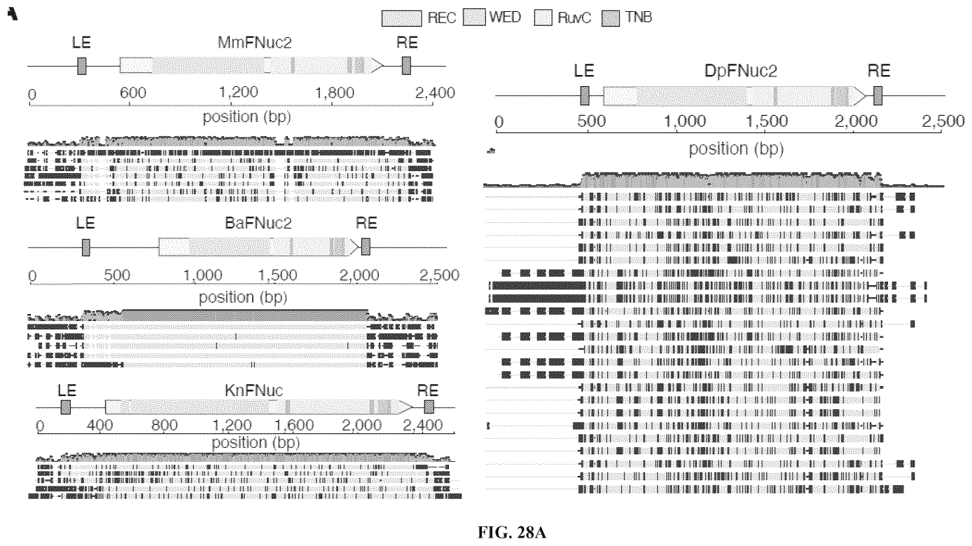

Fanzors commonly associate with different transposons (Bao et al. 2013). A comprehensive transposon search was performed (Chen et al. 2018) within 10 kb of Fanzors, analyzing the identity of the associated ORFs by domain search (FIG. 19, FIG. 26A, and FIG. 26B; Table 3). Amongst eukaryotic transposons, both previously reported transposon families, including Mariner/Tc1, Helitron, and Sola, and families not previously known to associate with Fanzors, including hAT and CMC DNA transposons were found (FIG. 26A and Table 3). Fanzor-transposon associations included autonomous transposons encoding a transposase, such as in the Crypton and Mariner/Tc1 families, as well as non-autonomous transposons including only transposon ends, such as hAT, EnSpm, and Helitron families (FIGS. 26A-26D and Table 3). Notably, the most frequent associations were with the DNA transposon hAT, suggesting that Fanzors might have some role with these transposons in the respective eukaryotic genomes. Fanzor1a,b, and d clades are most commonly associated with hAT, whereas Fanzor1c preferentially associated with LINE, CMC, and Mariner/Tc1 transposons (FIG. 19A and FIGS. 26A-26D). Fanzor2s associated with diverse transposons, including, Helitron, hAT, and IS607 (FIG. 19A and FIGS. 26B-FIG. 26D). The IS607 transposons encode a TnpA-like transposase, further cementing the close relationship between Fanzor2 and TnpBs.

TABLE 3
Fanzor families in eukaryotics genomes and their identified transposon associations.

| | Copy | | TIR | TSD | | Fanzor protein | | Tpase # | |
|---|---|---|---|---|---|---|---|---|---|
| Family (bp) | No. | Termini | (bp) | (bp) | | (aa) &(No. Exons) | | (Superfamily) | Comments |
| MDe-1 | 2 | | | | | 815 | (3) | | |
| MDe-2 | 2 | | | | | 698 | (4) | | |
| MDe-3 | 1 | | | | | 620 | (4) | | |
| MDe-4 | 4 | L.R. | N | n.a. | | 731 | (4) | | |
| MDe-5 | 4 | L.R. | N | n.a. | | 656 | (4) | | |
| MDe-6 (3852) | 10 | L.R. | N | n.a. | | 661 | (4) | | |
| MDe-7 (3937) | 8 | L.R. | 24 | 2 | (TA) | 772 | (3) | | |
| MDe-8 | 4 | R. | | | | 745 | (3) | | |
| MDe-9 | 3 | R. | | | | 764 | (5) | | |
| MDe-10 | 1 | | | | | 779 | (3) | | |
| MDe-11 | 3 | R. | | | | 713 | (4) | | |
| MDe-12 (3875) | 5 | L.R. | N | n.a. | | 677 | (4) | | |
| MDe-13 | 3 | R. | | | | 680 | (2) | | |
| HMa-1 | 1 | | | | | i.c. | | Mariner | Probably |
| | | | | | | | | | from virus |
| SAl-1* | 3 | R. | | | | 400 | (1) | | |
| SAl-2* | 3 | R. | | | | 498 | (4) | | |
| SPu-1 (2149) | 25 | L.R. | 33 | 2 | (TA) | 633 | (1) | | |
| SPu-2 | 2 | | | | | 663 | (1) | | |
| SPu-3 (2288) | 2 | L.R. | 25 | 2 | (TA) | 626 | (1) | | |
| ROr-1 (5190) | 10 | L.R. | 90 | 2 | (TA) | 928 | (3) | Mariner | |
| ROr-2 (4073) | 18 | L.R. | 46 | 2 | (TA) | 690 | (2) | Mariner | |
| ROr-3 (2862) | 16 | L.R. | 133 | 2 | (TA) | 720 | (2) | | |
| ROr-4 (5244) | 9 | L.R. | 38 | 9 | | 1165 | (3) | (MuDr) | |
| AMa-1 | 1 | | | | | 871 | (4) | | |
| AMa-2 | 1 | | | | | 645 | (3) | | |
| AMa-3 | 1 | | | | | 789 | (7) | | |
| PBl-1 (3938) | 4 | L.R. | 12 | 3 | (TAN) | 683 | (4) | | |
| PBl-2 | 3 | | | | | 677 | (2) | | |
| PBl-3 (4614) | 6 | L.R. | 42 | 9 | | 1186 | (3) | (MuDr) | |
| MCi-1 (4036) | 4 | L.R. | 20 | 2 | (TA) | 686 | (2) | Mariner | |
| MCi-2A (10235) | 3 | L.R. | N | 11 | | 1375 | (4) | Crypton | |
| MCi-2B | 2 | R. | — | — | | 1375 | (4) | | |
| MCi-2C | 3 | R. | — | — | | 1375 | (4) | | |
| MCi-2D (9295) | 2 | L.R. | N | 12 | | 1375 | (4) | | |
| MCi-3 (5305) | 2 | L.R. | 39 | 4? | (TTAA) | 1304 | (2) | | |
| MCi-4 (4508) | 6 | L.R. | 31 | 9 | | 1245 | (3) | (MuDr) | |
| MCi-5 (7323) | 5 | L.R. | N | n.a. | | 1212 | (3) | Harbinger | |
| MCi-6 | 2 | | | | | 1231 | (2) | | |
| MCi-7 | 1 | R. | | | | 1153 | (3) | | |
| MCi-8 | 1 | | | | | 1067 | (2) | | |
| MCi-9 | 1 | | | | | 1149 | (3) | | |
| MCi-10 | 1 | | | | | 1135 | (4) | | |
| AGo-1* | 1 | | | | | 457 | (1) | | |
| ECy-1* | 1 | | | | | 455 | (1) | | |
| SCe-1* | 1 | | | | | 350 | (1) | | |
| TDe-1* (1785) | 7 | L.R. | | | | 486 | (1) | | |
| DFa-1 (11949) | 12 | L.R. | 12 | 4 | | 1241 | (10) | (Sola2) | |
| DFa-2 (12887) | 7 | L.R. | 12 | 4 | | 1010 | (9) | Sola2 | |
| DFa-3 (10254) | 2 | L.R. | 13 | 4 | | 1084 | (10) | (Sola2) | |
| DFa-4 | 1 | | | | | 1020 | (13) | — | |
| PPa-1 (13566) | 3 | L.R. | 22 | 4 | | 1699 | (7) | Sola2 | |
| PPa-2 | 1 | | | | | 945 | (8) | | |
| PPa-3 | 1 | | | | | 970 | (9) | | |
| PPa-4 (14423) | 3 | L.R. | 16 | 4 | | 1827 | (14) | Sola2 | |
| PPa-5 (15292) | 3 | L.R. | 16 | 4 | | 1388 | (12) | Sola2 | |
| PPa-6 | 2 | R. | 16 | 4 | | 1218 | (13) | | |
| PPa-7 | 1 | | | | | 1756 | (16) | | |
| ACa-1* (2675) | 2 | L.R. | N | 0 | | 603 | (1) | TnpA_IS607 | |
| ACa-2* | 1 | | | | | 653 | (1) | TnpA_IS607 | |
| VCa-1 | 1 | | | | | 768 | (1) | | |
| VCa-2 | 1 | | | | | i.c. | | | |
| CRe-1 (3992) | >100 | L.R. | N | 0 or n | | 830 | (5) | (Helitron) | Expressed |
| CRe-2 (4882) | >100 | L.R. | N | 0 or n | | 906 | (10) | (Helitron) | Expressed |
| CRe-3 (4688) | >100 | L.R. | N | 0 or n | | 967 | (10) | (Helitron) | Expressed |
| CRe-4 | 3 | R. | | | | 944 | (6) | | |
| CRe-5 | 3 | R. | | | | i.c. | | | |
| CVu-1 | n.a | | | | | i.c. | | | |
| CMe-1A (3169) | 150 | L.R. | N | n.a. | | 734 | (1) | | |
| PUl-1 (3620) | 8 | L.R. | 24 | 2 | (TA) | 802 | (1) | Mariner | |
| PUl-2 (3820) | 1 | L.R. | 33 | 2 | (TA) | 643 | (3) | Mariner | |
| PUl-3 | 1 | | | | | 799 | (1) | | |
| PUl-4 (3356) | 3 | L.R. | 26 | 2 | (TA) | 809 | (1) | | |
| PUl-5 | 1 | R. | | | | 617 | (1) | | |
| PUl-6 | 5 | R. | | | | 642 | (1) | | |
| NOc-1 | 4 | | | | | i.c. | | | |
| PSo-1 | 2 | R. | | | | 660 | (1) | | |
| PSo-2 | 4 | R | | | | 726 | (1) | | |
| PSo-3 | 3 | | | | | 716 | (1) | | |
| PSo-4 | 3 | | | | | 785 | | | |
| PSo-5* | 1 | | | | | i.c. | | | |
| PCa-1, | 2 | R. | | | | 788 | (1) | | |
| PCa-2 (2107) | 2 | L.R. | N | N | | 611 | (1) | | |
| PCa-3* | 2 | R. | | | | 483 | | | |
| PRa-1 | 1 | | | | | i.c. | | | |
| PRa-2* | 2 | R. | | | | i.c. | | | |
| ALa-1 | 1 | | | | | i.c. | | | |
| ALa-2 | 1 | | | | | i.c. | | | |
| ESvi-1A (3180) | 1 | L.R. | 59 | | | 890 | (1) | | |
| ESvi-1B (4052) | 1 | L.R. | 25 | 8 | | 890 | (1) | IS4 | |
| ESv-1 (2639) | 2 | L.R. | 40 | 2 | (TA) | 693 | (1) | | |
| ESv-2 (3603) | 2 | L.R. | 18 | | | 757 | (1) | IS4 | |
| SWv-1 (2633) | 1 | L.R. | 21 | 6 | | 779 | (1) | | |
| HAgv-1 (1963) | 2 | L.R. | 13 | 4 | (TTAT) | 572 | (1) | | |
| HAmv-1 (1925) | 1 | L.R. | 13 | 4 | (TTAA) | 592 | (1) | | |
| PUgv-1 (1961) | 2 | L.R. | 13 | 4 | (TTAT) | 571 | (1) | | |
| SFav-1 (1954) | 2 | L.R. | 13 | 4 | (TTAN) | 606 | (1) | | |
| HVav-1 (1955) | 5 | L.R. | 13 | 4 | (TTAN) | 608 | (1) | | |
| MCnv-1 | 1 | R. | | | | i.c. | | | |
| PGv-1 (4442) | 1 | L.R. | 29 | 2 | (TA) | 625 | (1) | Mariner | |

| EHv88-1 | 1 | | 650 | (1) | | |
|---|---|---|---|---|---|---|
| EHv99B1-1* (2126) | 1 | L.R. | 640 | (1) | | |
| ISvMimi_1* (2549) | 3 | L.R. | 520 | (1) | TnpA_IS607 | =APmv-2, |
| | | | | | | =ACmv-2 |
| ISvMimi_2* | 1 | | 545 | (1) | TnpA_IS607 | =APmv-1, |
| | | | | | | =ACmv-1 |
| APmv-3* | 1 | | 482 | (1) | | =ACmv-3 |
| MGvc-1*, | 1 | | 526 | (1) | | |
| MGvc-2* | 1 | | 493 | (1) | | |
| ISvAR158_1* | 1 | | 351 | (1) | TnpA_IS607 | |
| ISvNY2A_1* (2164) | 3 | L.R. | 395 | (1) | TnpA_IS607 | |
| ISvNY2A_2* (1443) | 2 | L.R. | 432 | (1) | | |
| CRv-1* | 1 | | 416 | (1) | TnpA_IS607 | |
| FEsv-1* | 1 | | 408 | (1) | | |

| Fanzor1-1_{—}SitMos | >12 | L.R. | 11-bp | 2-bp | (NN) | (3) | EnSpm? | |
|---|---|---|---|---|---|---|---|---|
| Fanzor1-2_{—}SitMos | >8 | L.R. | 74 | 8 | | (5) | hAT | |
| | | | | (ATGTANNN) | | | | |
| Fanzor1-3_{—}SitMos | >14 | L.R. | 12 | 8 | | (5) | hAT | |
| Fanzor1-4_{—}SitMos | 1 | — | — | | | | | fragmental |
| Fanzor1-5_{—}SitMos | 6 | R. | — | — | | | Helitron? | |
| Fanzor1-6_{—}SitMos | >10 | L.R. | 21 | 2-bp | (NN) | | EnSpm? | |
| Fanzor1-7_{—}SitMos | 6 | L.R. | 127 | 8 | | (4) | hAT | |
| | | | | GT(GTGNNNNN) | | | | |
| Fanzor1-8_{—}SitMos | >7 | L.R. | 12 | 2-bp | (NN) | (3) | EnSpm? | |
| Fanzor1-9_{—}SitMos | >16 | R. | — | — | | (4) | | fragmental |
| Fanzor1-10_{—}SitMos | >9 | R. | — | — | | | | fragmental |
| Fanzor1-11_{—}SitMos | 1 | — | — | | | | | |
| Fanzor1-1_{—}ConNas | >20 | L.R. | 12 | 2 | | | EnSpm? | |
| Fanzor1-2_{—}ConNas | >6 | L.R. | 12 | 2 | | | EnSpm? | |
| Fanzor1-3_{—}ConNas | >50 | L.R. | 12 | 2 | | (2) | EnSpm? | |
| Fanzor1-4_{—}ConNas | >20 | L.R. | 11 | 2 | | (3) | EnSpm? | |
| Fanzor1-5_{—}ConNas | >7 | L.R. | 11 | 2 | | (3) | EnSpm? | |
| Fanzor1-6_{—}ConNas | >10 | L.R. | 133 | 8 | | (5) | hAT | |
| | | | | (ATGTANNN) | | | | |
| Fanzor1-7_{—}ConNas | >3 | L.R. | 126 | 8 | | (3) | hAT | |
| | | | | (GTGNNNNN) | | | | |
| Fanzor1-8_{—}ConNas | >8 | L.R. | 12 | 2 | | (4) | EnSpm? | |
| Fanzor1-9_{—}ConNas | >13 | L.R. | 126 | 8 | | (3) | hAT | |
| | | | | (ATGTANNN) | | | | |
| Fanzor1-10_{—}ConNas | >6 | L.R. | none | 8 | | (4) | hAT? | |
| | | | | (GCANNNNN) | | | | |
| Fanzor1-11_{—}ConNas | >10 | L.R. | 133 | 8 | | (5) | hAT | |
| | | | | (ATGTANNN) | | | | |
| Fanzor1-12_{—}ConNas | >10 | L.R. | 130 | 8 | | (3) | hAT | |
| | | | | (ATGTANNN) | | | | |
| Fanzor1-13_{—}ConNas | >11 | L.R. | 72 | 2 | (TA) | (4) | EnSpm? | |
| Fanzor1-14_{—}ConNas | >4 | L.R. | 12 | 2 | (TA) | (3) | EnSpm? | |
| Fanzor1-15_{—}ConNas | >3 | L.R. | 16 | 8 | | (1) | hAT? | |
| | | | | (GGTANNNN) | | | | |
| Fanzor1-16_{—}ConNas | >3 | L.R. | none | 8 | | (6) | hAT? | |
| | | | | (GGTANNNN) | | | | |
| Fanzor1-17_{—}ConNas | >2 | L.R. | 15 | 8 | | (3) | hAT? | |
| | | | | (GGTANNNN) | | | | |
| Fanzor1-18_{—}ConNas | >20 | R. | | | | | | |
| Fanzor1-19_{—}ConNas | >4 | L.R. | 121 | 8 | | (4) | hAT | |
| | | | | (ATGTANNN) | | | | |
| Fanzor1-1_{—}ApoVar | >16 | L.R. | none | 0 | | (7) | Crypton | |
| Fanzor1-2_{—}ApoVar | 12 | L.R. | none | 0 | | | Crypton? | |
| Fanzor1-3_{—}ApoVar | >4 | L.R. | none | | | | Helitron | |
| Fanzor1-4_{—}ApoVar | >11 | L.R. | none | | | | Crypton | |
| Fanzor1-5_{—}ApoVar | >6 | L.R. | none | | | | Helitron | |
| Fanzor1-6_{—}ApoVar | >6 | L?.R. | none | | | | Helitron? | |
| Fanzor1-7_{—}ApoVar | >5 | L.R. | none | | | | Helitron | |
| Fanzor1-8_{—}ApoVar | >5 | L.R. | | TA | | | Mariner | |
| Fanzor1-8B_{—}ApoVar | >5 | L.R. | | TA | | | Mariner | |
| Fanzor1-9_{—}ApoVar | =4 | L.R. | 19-bp | TA | | | Mariner? | |
| (3996) | | | | | | | | |
| Fanzor1-1_{—}RhiMic | 3 | L.R. | 90 | 2 | (TA) | (1) | Mariner? | |
| Fanzor1-2_{—}RhiMic | >3 | L.R. | none | 2 | (TA) | 3 | Mariner (+) | |
| Fanzor1-3_{—}RhiMic | >4 | L.R. | none | | | | Helitron | |
| Fanzor1-4_{—}RhiMic | ~4 | L.?R? | none | | | | | |
| Fanzor1-1_{—}MuIr | ~3 | R. | | 0 | | | Crypton | |
| Fanzor1-2_{—}Mulr | ~4 | L.R. | 36 | 9 | | (5) | MuDR? | |
| Fanzor1-3_{—}Mulr | >4 | R. | | | | | | |
| Fanzor1-4_{—}Mulr | >3 | L.R. | | 9 | | | MuDR? | |
| Fanzor1-5_{—}MuIr | >4 | L.R. | Weak | 9 | | | MuDR? | |
| | | | subterminal | | | | | |
| | | | TIRs | | | | | |
| Fanzor1-1_{—}ParPar | >10 | L.R. | none | | | | Crypton | |
| Fanzor1-2_{—}ParPar | >10 | L.R. | 142 | 2 | (TA) | | | |
| Fanzor1-3_{—}ParPar | >3 | L.R. | 24 | 3 | (TWA) | | | |
| Fanzor2-1_{—}ParPar | >40 | L.R. | 14 | 4 | (TTAA) | | | |
| (1660) | | | | | | | | |
| Fanzor1-1_{—}KleNit | >6 | L.R. | 27 | 2 | (TA) | (1) | Mariner | |
| Fanzor1-1_{—}KleNit | >5 | L.R. | 27 | ? | | | | |
| Fanzor1-1_{—}ChlPri | >4 | L.?R? | | | | | | |
| Fanzor2-1_{—}ChlPri | >23 | L.R. | 13 | 5 | | | | |
| (2654) | | | | | | | | |
| Fanzor1-1_{—}CarMem | =6 | L.R. | none | 5 | | 1 | | |
| Fanzor1-2_{—}CarMem | >6 | L.R. | none | 5 | | 1 | | |
| Fanzor1-3_{—}CarMem | =3 | L.R. | | 5 | | | | |
| Fanzor1-1_{—}MicYARC | >100 | L.R. | 27 | 2 | (TA) | (1) | Mariner (+) | Target CTA |
| (3453) | | | | | | | | |
| Fanzor1-1N1_{—}MicYARC | >14 | L.R. | 27 | 2 | (TA) | (1) | Mariner | |
| Fanzor1-2_{—}MicYARC | | L.R. | 27 | 2 | (TA) | (1) | Mariner | Target CATA |
| Fanzor1-3_{—}MicYARC | >16 | L.R. | | 2 | (TA) | | Mariner | |
| Fanzor1-4_{—}MicYARC | >50 | L.R. | 32 | 2 | (TA) | | Mariner (+strand) | Target GTTA, |
| | | | | | | | | specific |
| Fanzor1-5_{—}MicYARC | >2 | L.R. | | 2 | (TA) | (1) | Mariner (−strand) | Target CATA, |
| | | | | | | | | specific |
| IS607EU-1_{—}MicYARC | >20 | L.R. | none | none | | | IS607, S- | |
| | | | | | | | recombinase | |
| IS607EU-1_{—}MicYARC | | L.R. | | | | | IS607, S- | |
| (2163) | | | | | | | recombinase | |
| Fanzor1-1_{—}XesXan | >4 | L.R. | | TTAA | | | piggyBac | |
| | | | | | | | (by TIR) | |
| Fanzor1-1_{—}CycCry | >9 | L.R? | none | | | >3 | | 88% |
| Fanzor1-1_{—}EreLig | =3 | L.R. | 17 | 4-bp | | 1 | piggyBac? | |
| Fanzor1-1_{—}AbrTri | =7 | L.R. | 13-bp | 4-bp | | | ? | |
| (1873) | | | | | | | | |
| Fanzor1-1_{—}CydSpl | =5 | L.R. | 12-bp | 4-bp | | 1 | | |
| (1931) | | | | | | | | |
| Fanzor1-1_{—}NeHa | >6 | L.R. | | none | | 1 | Crypton?? | 14642-bp |
| Fanzor1-2_{—}NeHa | >3 | L.R. | | none | | | | |
| Fanzor1-1_{—}HypPro | >4 | L.R. | 9 | TTAA | | 1 | piggyBac? | Inserted with |
| | | | | | | | | I-element. |
| Fanzor1-1_{—}LysCor | =3 | L.R. | 10 | TTAA | | 1 | piggyBac? | |
| (2202) | | | | | | | | |
| Fanzor1-1_{—}NeYa | >40 | R. | | | | | | |
| IS607EU-h1_{—}PhySoj | >2 | L.? R. | | | | | | |
| Fanzor1-6_{—}PhySoj | >2 | R. | | | | | | |
| (2476) | | | | | | | | |
| IS607EU-1_{—}UndPin | >3(*) | indeterminate | | | | | IS607 | Integrated |
| | | | | | | | | insideMuDR |
| Fanzor1-1_{—}LepBou | >3 | L.R. | 24-bp | TA | | 1 | Mariner (byTIR) | Target TGTA |
| Fanzor1-2_{—}LepBou | 2 | L.R. | 33-bp | Mostly TA | | 1 | EnSpm (byTIR) | |
| Fanzor1-3_{—}LepBou | 2 | L.R. | | 2-bp | | 1 | EnSpm (byTIR) | |
| IS607EU-1_{—}GiMa | | | | | | | IS607 | |
| IS607EU-2_{—}GiMa | >60 | L.R. | none | none | | | IS607 | TnpB degraded. |
| IS607EU-3_{—}GiMa | >14 | L.R. | none | none | | | IS607 | |
| Fanzor1-1_{—}PilApi | >40 | L.R. | 18-bp | 4-bp | | | | |
| Fanzor1-2_{—}PilApi | 8 | L.R. | none | none | | | | |
| Fanzor1-3_{—}PilApi | >6 | L.R. | 169-bp | TA, likely | | | | Old repeat, |
| | | | | | | | | 86% identity |
| IS607EU-1_{—}SchTIO01 | >20 | L.R. | none | none | | | IS607 | |
| Fanzor1-1_{—}VerVer | >28 | L.R. | 20 | 2-bp | | | | |
| Fanzor1-1_{—}EuLap | >7 | R. | | | | | | |
| Fanzor1-1_{—}GuiThe | 9 | L.R. | 15-bp | 4-bp | (ATAN) | | | |
| (2751) | | | | | | | | |
| Fanzor1-2_{—}GuiThe | >10 | L.R. | none | 4-bp | (TTAW) | | | TnpB |
| (2714) | | | | | | | | truncated at |
| | | | | | | | | the C- |
| | | | | | | | | terminal |
| Fanzor1-3_{—}GuiThe | 1 | L.R. | 18-bp | 4-bp | (predicted) | | | |
| (2261) | | | | | | | | |
| Fanzor1-1_{—}ApoBC | ~4 | R. | uncertain | uncertain | | | | |
| Fanzor1-1_{—}AphGif | 8 | R. | uncertain | Uncertain | | | | 5′-end is |
| | | | | | | | | flexible. |
| Fanzor1-1_{—}MucSat | >9 | L.R. | 27-bp | 2-bp | (TA) | | | |
| Fanzor1-1_{—}BomMaj | >4 | R. | uncertain | uncertain | | | | |
| Fanzor1-2_{—}BomMaj | =3 | R. | uncertain | uncertain | | | | |
| Fanzor1-1_{—}RhiDel | =3 | L.R. | 78-bp | TA? | | | Mariner? | TGTA |
| Fanzor2-1_{—}MerMer | =4 | R. | | | | | IS607? | |
| Fanzor1-1_{—}MucSat | >9 | L.R. | 27-bp | 2-bp | (TA) | | | |

| | | | | | | |
|---|---|---|---|---|---|---|
| Fanzor1-1_{—}BomMaj | >4 | R. | uncertain | uncertain | | |
| Fanzor1-2_{—}BomMaj | =3 | R. | uncertain | uncertain | | |
| Fanzor1-1_{—}RhiDel | =3 | L.R. | 78-bp | TA? | Mariner? | TGTA |
| Fanzor2-1_{—}MerMer | =4 | R. | | | IS607? | |

Fanzor elements are named after the host species.
Fanzor2 elements are indicated by(*).
The left and right termini are indicated by L. and R. respectively, in the orientation of the encoded Fanzor protein.
N: none;
n.a.: not available;
i.c.: incomplete.
#: The encoded Tpase (or coding sequences).
If a given Fanzor element does not encode Tpase, but the superfamily it belongs can be determined, the superfamily name is parenthesized.
Rows highlighted in white correspond to Fanzor-Transposon associations previously identified (Bao et al. 2013).
Bold rows correspond to new transposon associations identified in this study.

### Example 21: Fanzors are Associated with Conserved, Structured Non-Coding RNAs

TnpB and IscB nucleases process the ends of the transposon-encoded RNA transcript into ωRNA, which complex with the respective nucleases to form a RNA-guided dsDNA endonuclease ribonucleoprotein (RNP) (Karvel et al. 2021; Altae-Tran et al. 202; Nety et al. 2023). Fanzor loci were searched for putative regions encoding OMEGA-like RNAs, based on conservation of non-coding sequence. There was conservation extending beyond the detectable Fanzor ORF on both 5′ and 3′ ends of the ORF, with the conserved regions significantly longer for some Fanzor families than those in TnpB and IscB loci, although some families like the viral-enriched Fanzor2 have non-coding lengths similar to those of TnpB systems (FIG. 19F and FIGS. 26E-26F). These conserved regions indicate either strong conservation within the transposon boundaries, or longer guide RNAs associated with Fanzor enzymes.

The Fanzor2 from the Acanthamoeba polyphaga mimivirus (ApmFNuc) that is encoded within a IS607 transposon and contains a TnpA transposase and defined inverted terminal repeats was further investigated to explore the potential activity and expression of these conserved regions (FIG. 19E). The A. polyphaga mimivirus genome contains three IS607 copies which show strong sequence conservation, both within the protein-coding regions but also in the non-coding region at the 3′ ends of the IS607 MGE (FIGS. 19E-19F). This non-coding sequence conservation extended 200 base nucleotides (nt) past the end of ApmFNuc ORF, ending upstream of the right inverted repeat (IRR), designating the right end (RE) of the MGE (FIG. 19G). In silico RNA secondary structure analysis predicted a stable fold (FIG. 19H and FIG. 26E), suggesting that the transcript of this conserved region could function as a Fanzor-associated guide RNA (fRNA). In the alignment of ApmFNuc loci, the predicted fRNA structure was highly conserved, with the conservation extending upstream into the coding region of ApmFNuc, indicating possible co-folding with this portion of the coding region and potential RNA processing site (FIG. 19I and FIG. 26G). This apparent RNA structure conservation is reminiscent of the OMEGA families, where both the IscB and TnpB families show limited structural variation (Altae-Tran et al. 2021), and processing of the upstream region of the mRNA releases functional guide RNAs (Nety et al. 2023).

### Example 22: Viral-Encoded ApmFNuc is a fRNA-Guided DNA Endonuclease

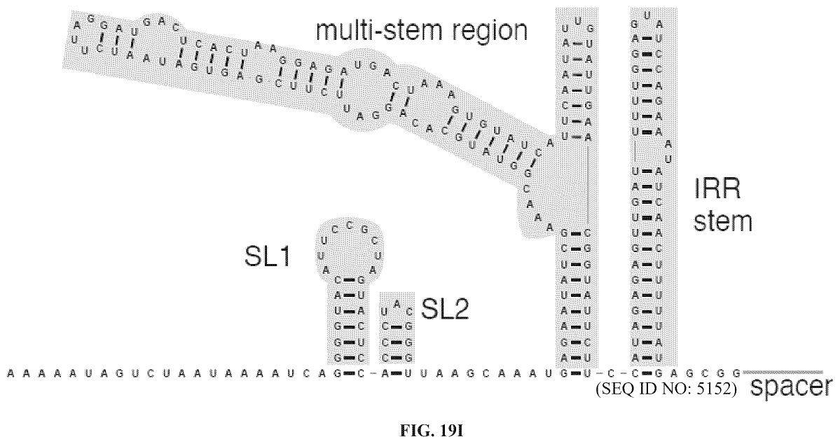

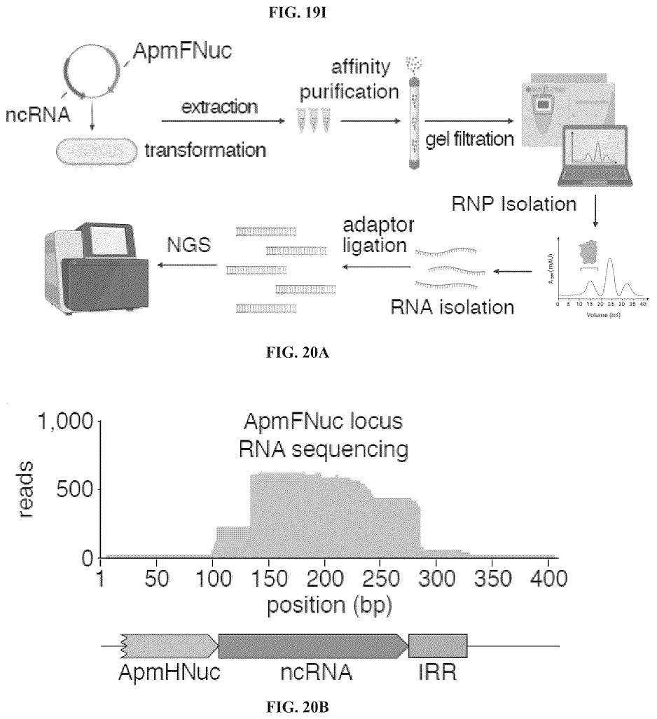

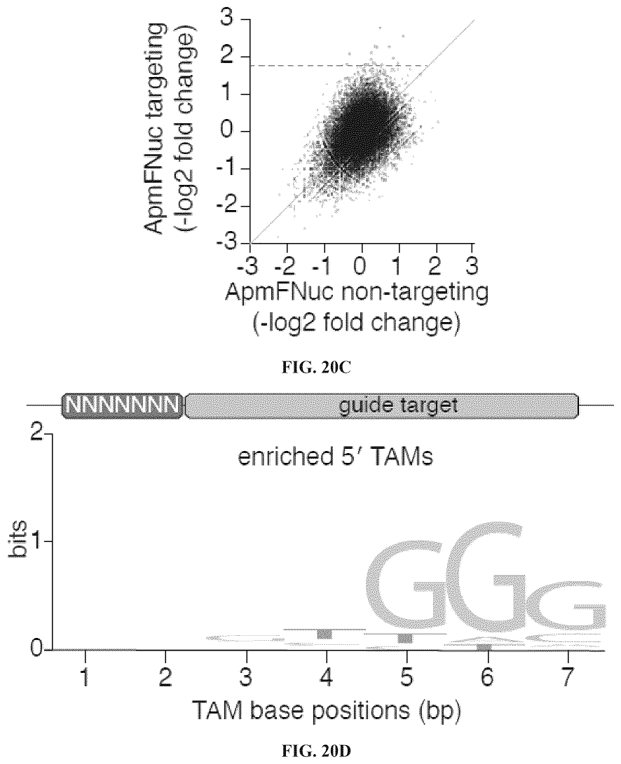

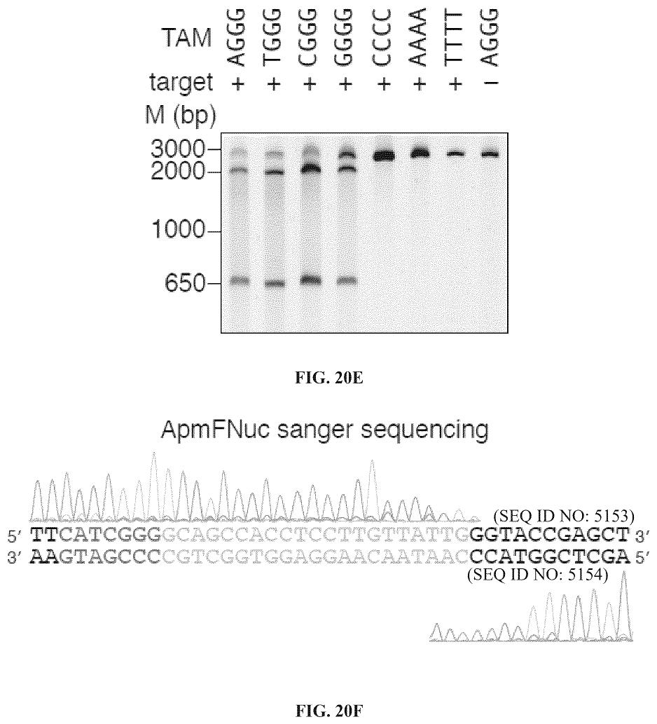

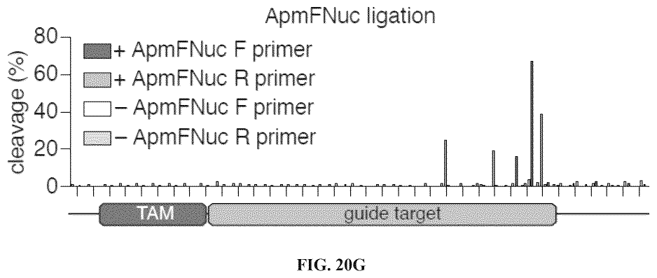

It was hypothesized that the fRNA forms a complex with ApmFNuc and directs binding and DNA cleavage to a specific sequence in the target. To investigate potential fRNA-ApmFNuc binding, the A. polyphaga mimivirus Fanzor locus, containing the non-coding RNA region, and an E. coli codon-optimized ApmFNuc was co-expressed in E. coli (FIG. 20A, Table 4). Notably, ApmFNuc protein was unstable when expressed alone and required co-expression with its fRNA for protein stabilization and accumulation (FIG. 27), similar to the instability of TnpB in the absence of ωRNA (Karvelis et al. 2021, Altae-Train et al. 2021). The fRNA-ApmfNuc RNP was purified and the RNA component of the complex was sequenced. Small RNA sequencing revealed enriched coverage between the 3′ ends of the protein ORF and the IRR, in agreement with the evolutionary conservation across the region (FIG. 20B).

Testing RNP cleavage activity required both the engineering of a reprogrammed fRNA and the determination of any sequence preferences, akin to the target adjacent motif (TAM) in the case of TnpB and IscB (Karvelis et al. 2021, Altae-Train et al. 2021). A 3′-terminal 21-nt targeting sequence was combined with the fRNA scaffold determined through RNA profiling to engineer a synthetic fRNA, co-expressed the synthetic fRNA and ApmFNuc in E. coli, and isolated the reprogrammed RNP complex. Cleavage on a DNA target containing a randomized 7 nucleotide TAM 5′ of a 21 nt target region complementary to the fRNA targeting sequence was tested to determine potential sequence preferences of ApmFNuc. This TAM library was co-incubated with purified ApmFNuc RNPs containing either targeting or scrambled synthetic fRNA guide sequences. The relative depletion of sequences was profiled with next-generation sequencing (NGS). TAM depletion analysis revealed a strong 5′ GGG motif adjacent to the target site (FIGS. 20C-20D). Robust ApmFNuc activity was validated on all possible NGGG TAMs, with no detectable cleavage of sequences lacking the TAM (FIG. 20E). In contrast to the G-rich ApmFNuc TAM, TnpB homologs of ApmFNuc universally prefer an A/T rich 5′ TAM (Nety et al. 2023). Interestingly, the GGG motif is present at the start of ApmFNuc MGE sequence and likely contributed to the TAM preference of ApmFNuc.

Cleavage locations of RNA-guided nucleases vary substantially, with cleavage sites located either upstream or downstream of the target sequence. To profile ApmFNuc cleavage patterns, ApmFNuc reaction products were purified and mapped the locations of the cleavage ends using Sanger sequencing. Cleavage occurred in the 3′ regions of the target sequence, with multiple nicks in both the target strand (TS) and the non-target strand (NTS) (FIG. 20F). The cleavage behavior of ApmFNuc at the 3′ end of the target is similar to the cleavage patterns of Cas12 or TnpB nucleases and in general agreement with the properties of programmable RuvC domains (Zetsche et al. 2015, Karvelis et al. 2021, Altae-Tran et al. 2021). The relative preference was quantified for these different nicking sites using an NGS-based assay, finding that during dsDNA cleavage by ApmFNuc the enzyme generated nicks in the NTS at positions 19 and 20, and in the TS at positions 15, 18, and 21 with all cleavage occurring inside the target region, indicating a slightly different cleavage pattern compared to TnpB nucleases (FIG. 20G).

TABLE 4
Fanzor Protein and fRNA sequences relevant for the present disclosure.

| Fanzor/ | Fanzor/ | | SEQ | Associated fRNA Scaffold | SEQ |
|---|---|---|---|---|---|
| TnpB | Tnp B | | ID | Sequence for biochemistry | ID |
| systems | types | Protein Sequence | NO: | (neglecting guide) | NO: |
| ApmFNuc | Fanzor | MKEAVKNVKPKVPAKKRIITGSKTKK | 1 | AAAAATAGTCTAATAAAATCA | 5 |
| | 2 | KVFVKKKPPDKKPLKKPVKKTVKTY | | GGGGTACATTCCGCTAGTACTC | |
| | | KLKSIYVSNKDLKMSKWIPTPKKEFT | | CACCCTACGGGTTAAGCAAATG | |
| | | EIETNSWYEHRKFENPNGSPIQSYNKI | | AGAATATCGAAACGGTATGCA | |
| | | VPVVPPESIKQQNLANKRKKTNRPIVF | | CAGGATTCTTCGAGTGATAATC | |
| | | ISSEKIRIYPTKEQQKILQTWFRLFAC | | TTAGGATGACTCACTAAGGAG | |
| | | MYNSSIDYINSKKVVLESGRINVAAT | | ATGACTAAAGTGTATCATTCAA | |
| | | RKVCNKISVRKALKTIRDNLIKSTNPS | | TATTGTATTGAACGGTATTCTT | |
| | | IMTHIMDEAIGLACSNYKTCLTNYIEG | | CCATAGAGAGTTGATTTtTGGA | |
| | | QIKKFDIKPWSISKRRKIIVIEPGYFKG | | GTATCCAGAAATATCAACTtTTT | |
| | | NSFCPTVFPKMKSSKPLIMIDKTVTLQ | | ATGAGCGG | |
| | | YDSDTRKYILFVPRVTPKYSVNKEKN | | | |
| | | SCGIDPGLRDFLTVYSENETQSICPIEI | | | |
| | | VVNTTKNEYKKIDKINEIIKTKPNLNS | | | |
| | | KRKKKLNRGLRKYHRRVTNKMKDM | | | |
| | | HYKVSHELVNTFDKICIGKLNVKSILS | | | |
| | | KANTVLKSALKRKLATLSFYRFTQRL | | | |
| | | THMGYKYGTEVVNVNEYLTTKTCSN | | | |
| | | CGKIKDLGASKIYECESCGMYADRDE | | | |
| | | NAAKNILKVGLKPWYKQK | | | |
| DpFNuc | Fanzor | MKRKREDLTLWDAANVHKHKSMW | 9 | ATTGGATGTTCAAAATGAAGCA | 13 |
| | 2 | YWWEYIRRKDMVNHEKTDCDVIQLL | | TACACTTCGAAGACGTGTGGAG | |
| | | QSASVKKQKTQSDKFLTSFSVGIRPTK | | TGTGTGGAACAATAAACAAAA | |
| | | HQKRVLNEMLRVSNYTYNWCLWLV | | ATCTAGAAAAGAGTGAAACAT | |
| | | NEKGLKPHQFELQKIVCKTNANDVDP | | TTTATTGCGATAACTGCAAATA | |
| | | QYRMENDDWFFNNKMTSVKLTSCK | | TAACACACACAGAGACGTTAA | |
| | | NFCTSYKSAKSLKSKLKRPMSVSNIIQ | | TGGTGCTAGAAATATtTTGCTA | |
| | | GSFCVPKLFIRHLSSKDVSTDNTNMQ | | AAATCGTTGCGCATGTTTCCAT | |
| | | NRYICMMPDNFEKRSNPKERFLKLAK | | TTGTCAATTCGCAGTTATAATT | |
| | | PITKIPPIDHDVKIVKRADGMFIMNIPC | | ACTCTGTAACAATTAGGTCGAT | |
| | | DPKYTRRNASNDTIEKRVCGIDPGGR | | CCATCCTAAATTCGAAAGTCCA | |
| | | TFATVYDPIDCCVFQVGIKEDKQYVIS | | TTGCTACGAGACTTTGCGTATG | |
| | | KLHNKIDHAHMHLTKAQNKKQQQA | | CTTAGTCCAGGGCAATtTTCTGC | |
| | | ARERIVSLKKTHLKLKTFVDDIHLKLS | | CGAATGAAATGGGTTA | |
| | | SHLVKEYQYVALGKINVAQLVKTDR | | | |
| | | PKPLSKRAKRDLLYWQHYRFRQRLT | | | |
| | | HRTTNTECILDVQNEAYTSKTCGVCG | | | |
| | | TINKNLEKSETFYCDQCKYNTHRDVN | | | |
| | | GARNILLKSLRMFPFEKQQQ* | | | |
| MmFNuc | Fanzor | MKRKREQMTLWKAAFVNGQETFKS | 10 | ACTTCCAAGACCTGTGGTAATT | 14 |
| | 2 | WIDKARMLELNCDVSSASSTHYSDLN | | GCGGTGTGAAGAACAACAAAC | |
| | | LKTKCAKMEDKFMCTFSVGIRPTSKQ | | TTGGTGGAAAGGAAACGTTTAC | |
| | | KRTLNQMLKVSNHAYNWCNYLVKE | | TTGTGAGTGTTGCAATTACAAA | |
| | | KDFKPKQFDLQRVVTKTNSTDVPAE | | ACTCATCGAGACGTCAACGGA | |
| | | YRLPGDDWFFDNKMSSIKLTACKNFC | | GCGAGAAACATTCTGTGCAAAT | |
| | | TMYKSAQTNQKKTKVDLRNKDIAML | | ACTTGAAACTTTTTCCATTCGC | |
| | | REGSFEVQKKYVRLLTEKDIPDERIRQ | | AGCATAACGAAAGAAACTGAC | |
| | | SRIALMADNFSKSKKDWKERFLRLSK | | AATCGATTTTTTCGGGTTCGAT | |
| | | NVSKIPPLSHDMKVCKRPNGKFVLQI | | TCTATCCCACTTGACTCAAAGA | |
| | | PCDPIYTRQIQVHTSDSICSIDPGGRTF | | GTCAGAGGGCTCGAATACATTT | |
| | | ATCYDPSNIKAFQIGPEADKKEVIHKY | | TCTGCACAGGTTTTGCTAATGC | |
| | | HEKIDYVHRLLAYAQKKKQTQAVQD | | AAGATCTGGGGCAAGAATGTG | |
| | | RIGQLKKLHLKLKTYVDDVHLKLCS | | TTCGGGTCAAATGAGTTA | |
| | | YLVKNYKLVVLGKISVSSIVRKDRPN | | | |
| | | HLAKKANRDLLCWQHYRFRQRLLHR | | | |
| | | VRGTDCEAIAQDERYTSKTCGNCGV | | | |
| | | KNNKLGGKETFICESCNYKTHRDVN | | | |
| | | GARNILCKYLGLFPFAA* | | | |
| BaFNuc | Fanzor | MKRTYSATKSSLTLWTAASVKTTSAP | 11 | CGCTCGAGTGGAGGGAACTGA | 15 |
| | 2 | KVVTTFSGWMKKILPTRAETSLTLINP | | CAACTGTAGAGTGGAGATCAC | |
| | | ADIADPSPPKKKAKKTTPATPKPTLRI | | CGACGAACGCTGGACCTCAAA | |
| | | YKIGLRPSPAQRKTLNACIVAANFAY | | GACGTGTGGCATGTGCAGATCC | |
| | | NQCVHLVQHKVCKPHLYDLQKIVAK | | ATCCACCGCGAACTTGGGGCA | |
| | | MKTPEDINHRYAPDRDGWFWKSSTI | | AAAGAACTATTCGAGTGCCCCA | |
| | | VRLLATKDFCAAYKAIVSNKKKDVA | | ACTGCCACTACACCTGCCACAG | |
| | | VIKYKTYDDPEAINPLSGLFGCQKQY | | AGACGTGCACGGAGCTCGAAA | |
| | | ATVTQAGLRLLPRLFGKDPIPLVKKK | | CATCTTGCTGAGAAGCTTCGGA | |
| | | LKVATIDHDFKIEKTSKGKFVLCLTVE | | CAGTTTCCAGTCTAGAAAAACA | |
| | | CSLLRRVKPPAPLFEDGYIHACGIDPG | | CGAACTTTTTCCTTGGCCCAAG | |
| | | VRSFVTVYDPTRQDCYQFGTSAQKA | | GATTGCCAAACACCACTCAAAT | |
| | | ERLDPITNAIDNWNSFVDQHRDKAPP | | CCTCGTTCAGGGGCCTCGAGTG | |
| | | TAIESWSRKTKKLWYKLKNQVRSLH | | GCTGGGCATATATGGGTTA | |
| | | DQVIAHLLGAYNFISLGKLDVSCFRR | | | |
| | | GTTAKSTNRWLRIYRHFEFRTKLLAR | | | |
| | | VEGTDNCRVEITDERWTSKTCGMCR | | | |
| | | SIHRELGAKELFECPNCHYTCHRDVH | | | |
| | | GARNILLRSFGQFPV | | | |
| KnFnuc | Fanzor | MDEGADDSEEAKRKRPDITLRRALRK | 12 | GGAGGAGGGAGGACAAGCTAC | 16 |
| | 1 | DKETSVVQTGWKFLCQELGIRDRIEEI | | AAGACGTGCCACAACGTGCGA | |
| | | IPEVTRIRVETCLLLNLHFIRLLDEGRP | | GCGTGTACGAACCCGCTCTGTC | |
| | | IPVIDQNLVGRAMQCTYSKNPQADPD | | GCATGGTGTGGAATAGAGACG | |
| | | LHETFVHHYLPLCPNRPNNSCLPRITN | | TCAACGCAGCTCGTAACATAGC | |
| | | VLLDLRNQLLSNIKNHVAVLFQSRHR | | TTGGATCTGTATGAGCATAGTC | |
| | | AFMKLLLREAAPDVPFFGDADEDLES | | AGAGGCGAGGGCAGGCCAGCG | |
| | | CTRLLTTATLWRPNESVRELLPEYPRI | | GAGTTCACGAGAGCAGGAATG | |
| | | YGRIPEAAIECLQDLVDSVRPEVGPLP | | TGAGGATGACTGAGAATTAGTC | |
| | | AAPQSRPHLYMPWMRIISEEFSDREL | | GAAAGACATAGCTGCCTAGAA | |
| | | RSFSLVPHASFSAPFIAITPTTWPELQP | | ACGAGTTCATCTAGGCACTTCG | |
| | | KSGKRKAPGELRDAFPSIGRLESGGK | | GTGAGAATCCGAGATACGGCT | |
| | | TFADRITTDGVSASVYFLVEKRTPPPE | | GGGTACTGTGGCGAGTGTGCCA | |
| | | DRVVHIHPKQRVVGLDPGKHPDFLTG | | TTTTACTCTGAACAGACTGTA | |
| | | IAVTGDWDGIERQEEIIGLGTRDFYHR | | | |
| | | AGFKKRTFLMHSWMSRDLDVAAFN | | | |
| | | KDAPSGNTVSLEDFGKRVTFVCANLY | | | |
| | | VLVRFHTARRVRKLRRRVTIKKQIEV | | | |
| | | DRACKRITAGKKTVVAFGAAQVWA | | | |
| | | GRTKRQCGPCESVKRRLSSHHKATV | | | |
| | | VMIDEFRTSQVCSTCHSDVGKFAVLK | | | |
| | | RQRVMEDGLPTVTEGGRREDEDEDG | | | |
| | | GGRTSYKTCHNVRACTNPLCRMVW | | | |
| | | NRDVNAARNIAWICMSIARGEGRPAE | | | |
| | | FTRAGVWG* | | | |
| CrFNuc | Fanzor | MAPKRRRDEAEKAEEEKDHTTSTKC | 2 | GCCGCCATGGCCGCCGGCGGC | 6 |
| | 1 | GLAGLLSEKIEADGVAVTREESLAAV | | GGCGGGGCCGGGCTGAGAGCC | |
| | | DFLVAALTRLRFEALCLLGLVAVRM | | TGAACGGCGCTAGCAGGGCGT | |
| | | CEDARREGQGLQPHCATCRRLRKTEL | | GGGGCTGAGGGTGCACGTGTT | |
| | | VEDDMYAAICAVSVCDLTEQGRKRG | | GATTGGCGGCGAGTGACGTGA | |
| | | RPSKRDQHPEDDLERHVCEEHFPRDE | | CTAGTTTGTTAGCTGCGGGTTA | |
| | | EAAGARVNRSGLTPFLPPLSKGVFTN | | GCACGGACTGTGCACCCCACCC | |
| | | VKNHYAANFAAWLARSFRCRIDDEL | | CACCGGCCACGTTCCGGATTTG | |
| | | RELRTPATKKLDKLAWSMAHAVLYD | | CGGGGATGCAAAGGCCCCCAA | |
| | | GELEQPRWWVGWAQGAAGAAAAA | | CATAGAGGCGTGTGCTTAGTAG | |
| | | AAQGAGPAGGAAAAQAWTALVDYV | | GCGCCCGCGTCAAGGTGGCTG | |
| | | NAQRASKRAAELLLREVKGAQATYK | | GGTTGATAACGACCCGGGAGG | |
| | | KASTRHMEWAAEILAGLEARRDQLG | | GGAGGGCTCAGCCCTTTTCCTG | |
| | | AQVQQLTQAQPLTREDTQRLASLRRE | | CCTCCCTAAGGCAGCCACCTCC | |
| | | LHRARPFTLTPSPSFAPIYVPLDNTSM | | TTGT | |
| | | ARLPGLLPTLARRHGEVFAGAGAGA | | | |
| | | VAPSSFVQAAFGGGGMQSSATLNAV | | | |
| | | GWGLFQLGGVTSRNAPFANYITTDG | | | |
| | | VACSVAREAHNKPLANLKPATAPAD | | | |
| | | AEELCTLEEMKATQIIGVDPCGGGNW | | | |
| | | FMAARSPLYQPGPWAWEGVGPAQR | | | |
| | | YLLELHDKQLDEELFPGQLPPEPRRR | | | |
| | | RKGVHRRKQSKHWQPRARTARRRR | | | |
| | | QKRGRFHMSMGHWRHMSGLERLQP | | | |
| | | NRPQLAPALQAYVGGIPTAATASAAR | | | |
| | | FEERLRYLFASGAAGQAAGGPAEAGP | | | |
| | | RGAVHVLWHYHFSAFRRKRWAAFIQ | | | |
| | | RDRALHRVAKQLTGGRPKEEVVVG | | | |
| | | WGSWAFQGGKGGSPISVRGGRAPTG | | | |
| | | RLIKLLRERYAKHVFIIDEYKTSKTCY | | | |
| | | NCGCQEMAIKRLGGLKEGQRPWSVK | | | |
| | | VCNDCLTTWNRDVSAANVIRVLLLL | | | |
| | | KLMGFERPTKLQRPPWPPAAAGPG* | | | |
| TvoTnpB | TnpB2 | MKRANAVKLIVGKETHEKLKELAIV | 3 | gggaagcccatgatgatgggcgtattaagcgt | 7 |
| | | AAKCWNEVNWLRMQQFKEGERVDF | | ggtctctataggtgtctccgcatagggaaggt | |
| | | SKTEKEVYEKYKQILKVNTQQVARK | | aataaacgcagacctgaatggtgcaataaata | |
| | | NAESWRSFFSLIEEKKGKLPKWFKPR | | tcctacatatccccgagtccctaggagctggg | |
| | | PPGYWKDKSGKYKMLIIIRNDRYEID | | agcagagggcaactcacagtgagggatagggg | |
| | | EEKRIIYLKDFKLSLSFNGKLKWRGK | | taatgggctgaagacccagcccgcggtctacc | |
| | | QGRLEIIYNEARRSWYAYIPVEVQND | | gctggacgaatggagcggggtgggtgtcctca | |
| | | VKAEDKLKASIDLGIINLATVYVEDG | | cccactagctatgaagtgatgaaaatgaaggc | |
| | | SWYIFKGGSVLSQYEYYSKRISVAQK | | ggtaaactgcaaaccaatgaatcgccacaagg | |
| | | TLARHKQGRSREMKLLHEKRKRFLK | | gaaccttcaccctttagg | |
| | | HALNSMVRKIMEEFKNKGVGEIAIGY | | | |
| | | PKEISKDHGNKLTVNFWNYGYIIRRF | | | |
| | | EGVGEELGVKVVKVDEAWTSKTCSL | | | |
| | | CGEAHDDGRIKRGLYRCLRIGKVINA | | | |
| | | DLNGAINILHIPESLGAGSRGQLTVRD | | | |
| | | RGNGLKTQPAVYRWTNGAGWVSSPT | | | |
| | | SYEVMKMKAVNCKPMNRHKGTFTL | | | |

### Example 23: Fanzor RNA-Guided DNA Endonucleases are Present in Diverse Eukaryotic Genomes

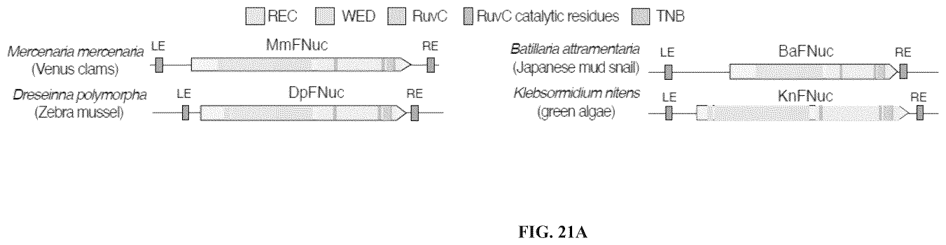

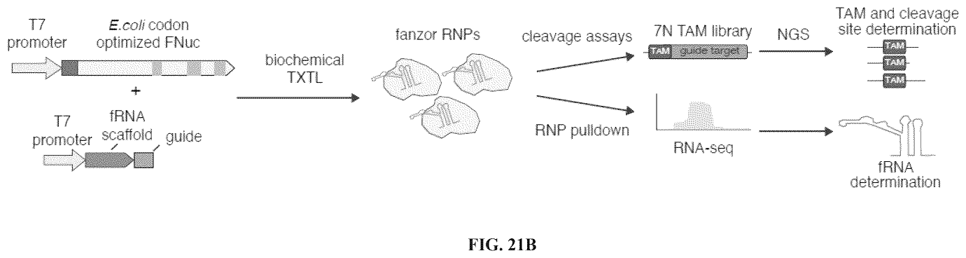

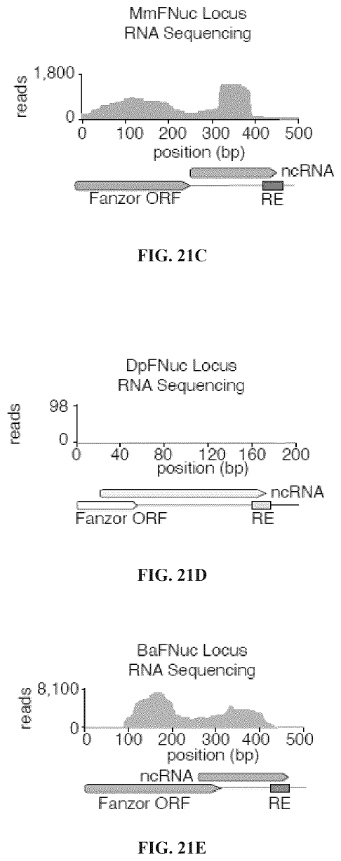

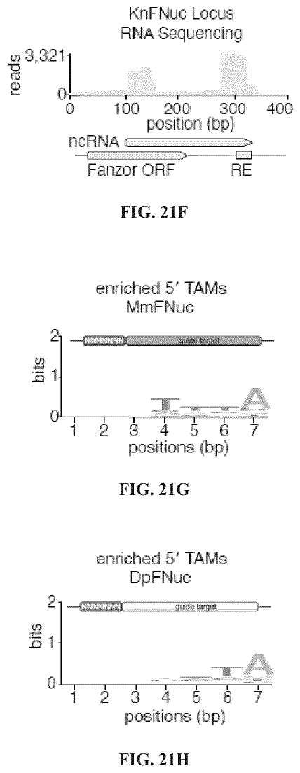

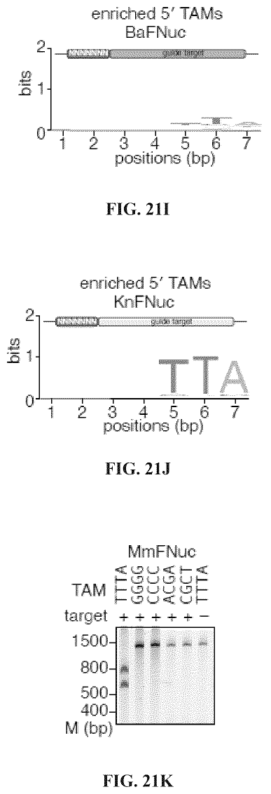

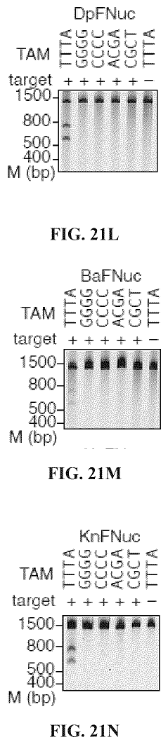

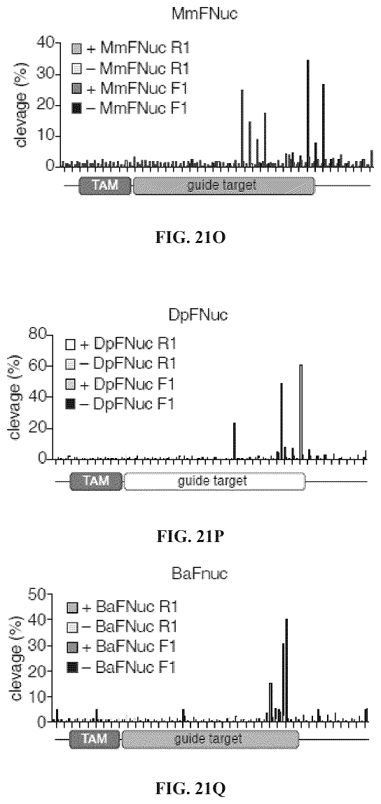

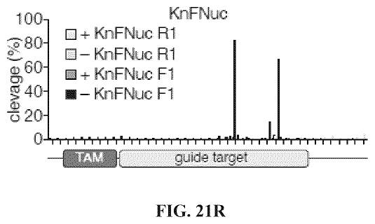

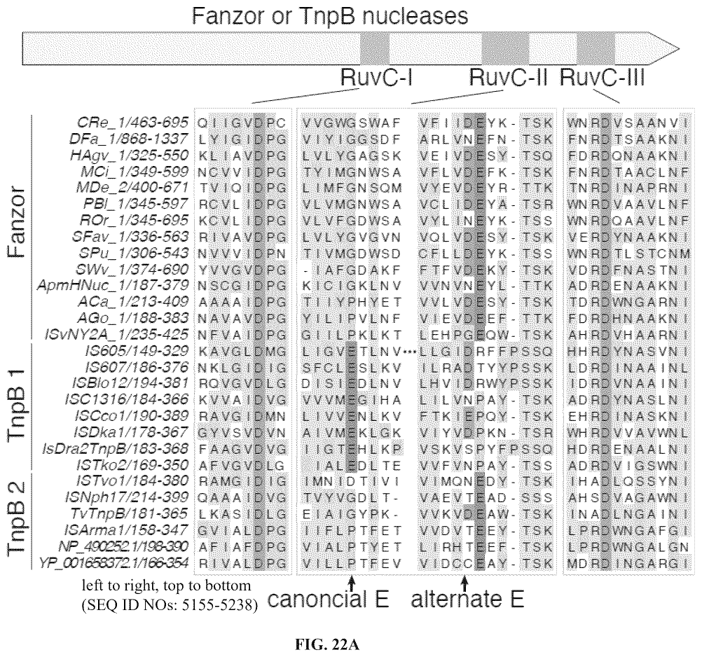

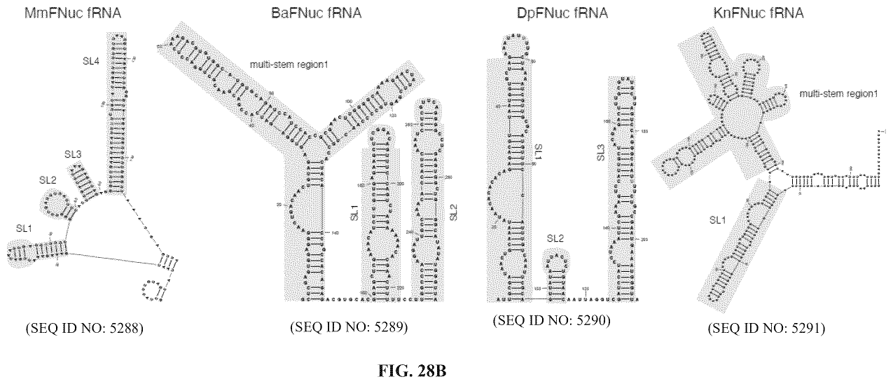

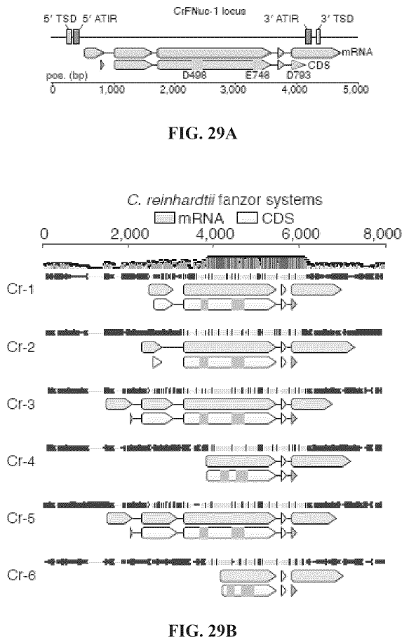

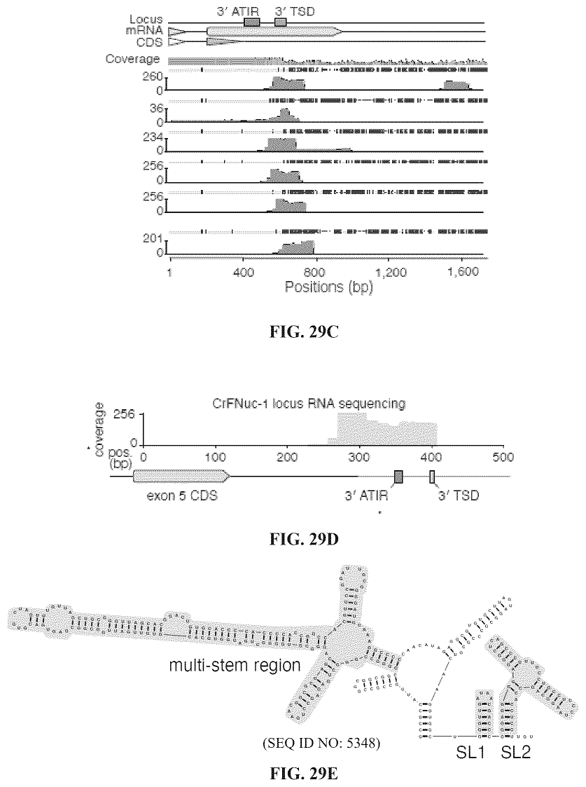

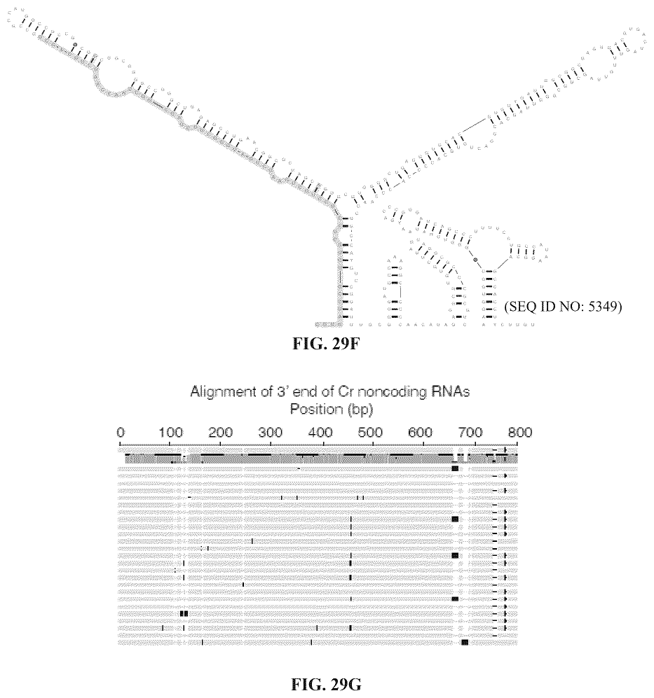

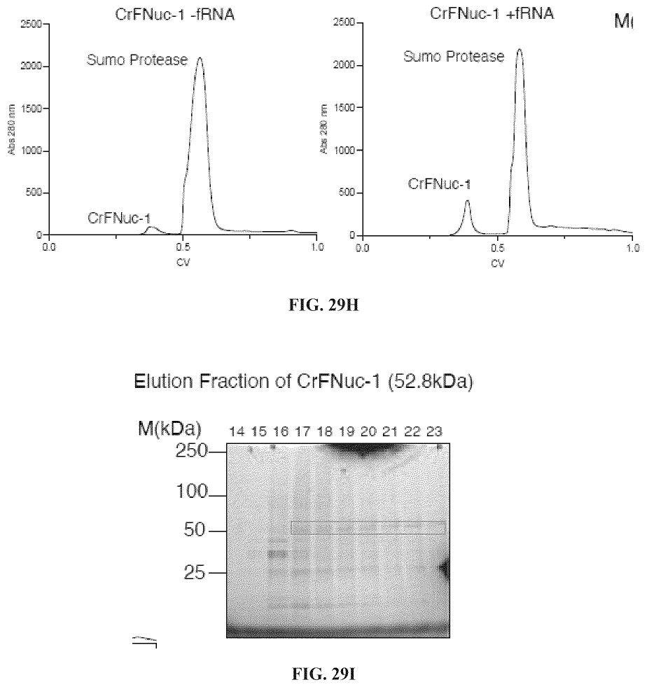

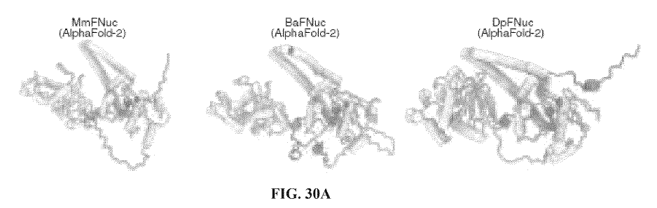

This study sought to explore whether Fanzor2 proteins from diverse eukaryotes also are active RNA-guided nucleases. Three Fanzor 2 representatives from three animals and a Fanzor 1 representative from a plant were chosen for this study: 1) Fanzor 2 from Mercenaria mercenaria (Venus clam; MmFNuc), 2) Fanzor 2 from Dreissena polymorpha (Zebra mussel; DpFNuc), 3) Fanzor 2 from Batillaria attramentaria (Japanese mud snail; BaFNuc), and 4) Fanzor 1 from Klebsormidium nitens (freshwater green algae; KnFNuc) (FIG. 21A). MmFNuc, DpFnuc, BaFnuc, and KnFNuc are all represented by multiple copies in the respective organisms, with 7, 24, 5, and 5 copies per genome, respectively (FIG. 21A and FIG. 28A), suggesting recent mobility of their associated transposons. Constructs for co-expression of the fRNA and Fanzor nuclease were cloned in a cell-free transcription/translation system, allowing for isolation of the resulting RNPs to study their fRNA sequences and cleavage activity (FIG. 21B). The RNPs were affinity purified and the bound fRNAs were sequenced, demonstrating that all four Fanzors co-purified with an RNA species derived from the 3′ non-coding region abutting the transposon RE (FIGS. 21C-21F). These fRNAs were highly structured with diverse structural motifs and domains (FIG. 28B).

Next, a 7N TAM library was challenged with MmFNuc, DpFNuc, BaFNuc, and KnFNuc RNPs with fRNA guide sequences complementary to the library target. There was strong TAM selection corresponding to TTTA, TA, TTA, and TTA TAMs for MmFNuc, DpFNuc, BaFNuc, and KnFNuc, respectively (FIGS. 21G-21J). Incubation of RNPs with individual preferred TAMs showed robust cleavage, validating all four eukaryotic Fanzor enzymes as RNA-guided nucleases (FIGS. 21K-21N). As with ApmFNuc, these Fanzors generated multiple nicks in the top and bottom DNA strands near the 3′ end of the target (FIGS. 21O-21R). Specific cleavage sites showed diversity, with MmFNuc and KnFNuc nicking more upstream and downstream within the guide target sequence than DpFNuc or BaFNuc (FIGS. 21O-21R). Interestingly, KnFNuc produced highly focused nicks in both the top and the bottom strands rather than multiple nicks, suggesting mechanistic differences between Fanzor1 and Fanzor2 nucleases.

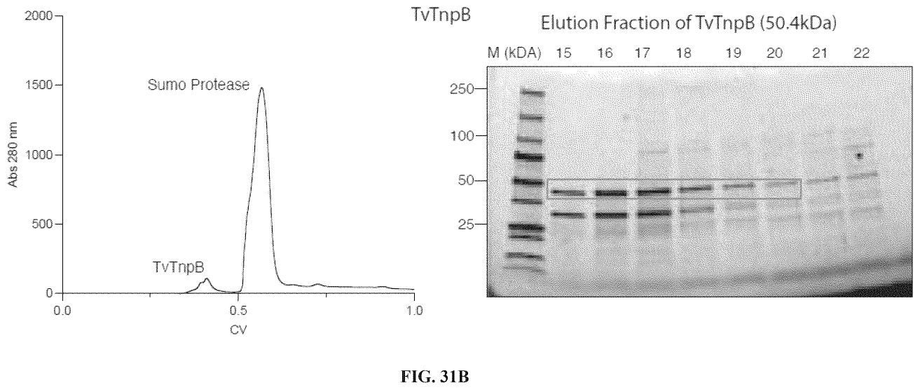

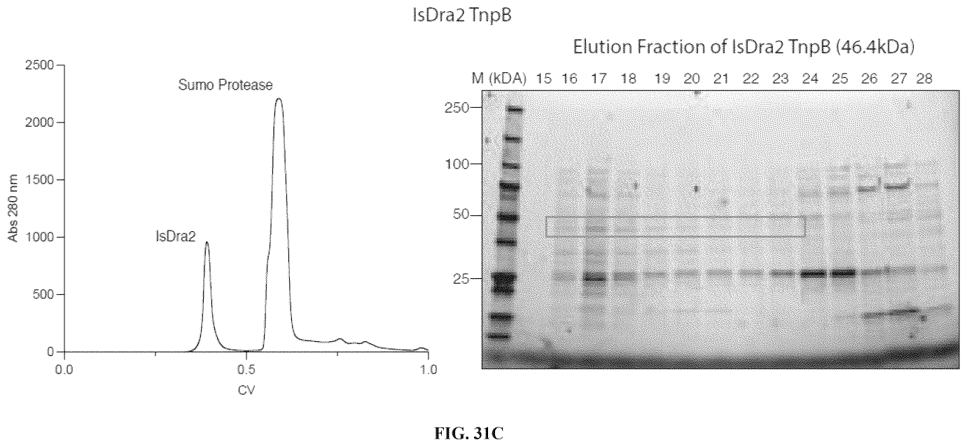

Given that ApmFNuc, MmFNuc, DpFNuc, BaFNuc, and KnFNuc all lack introns, an intron-containing Fanzor1c from the unicellular green alga Chlamydomonas reinhardtii (CrFNuc) was evaluated (FIGS. 29A-29C). There are six CrFNuc copies in the genome, and they are all associated with Helitron 2 transposons, which contain identifiable short target site duplications (TSDs) and asymmetrical terminal inverted repeats (ATIRs). Small RNA sequencing of a C. reinhardtii isolate showed strong enrichment of non-coding RNAs aligning to the 3′ UTR of the Cr-1 Fanzor mRNA (FIG. 29D), which was strongly conserved across all six copies CrFNuc-1 (FIGS. 31A-31B). Computational secondary structure prediction for the CrFNuc-1 fRNA with the fRNAs of the other five loci revealed a conserved stable secondary structure with a conserved upstream region not present in the RNA-sequencing trace, suggesting possible RNA processing of this region to serve as a guide RNA for CrFNuc-1 (FIGS. 29E-29F). Searches for similar sequences across the C. reinhardtii genome identified 20 additional distinct but highly conserved copies of the fRNA (FIG. 29G). Co-expression of CrFNuc-1 either with its native fRNA on the 3′ end of the MGE or a scrambled RNA sequence produced stable RNP only when coexpressed with its fRNA, similar to ApmFNuc (FIGS. 29H-29I). However, no cleavage was detected when the RNP was co-incubated with the 7N randomized TAM library plasmids, suggesting either failure to reconstitute the RNP activity under the experimental conditions or a lack of endonuclease activity of the native CrFNuc-1.

### Example 24: Fanzor Nucleases Contain a Conserved Rearranged Catalytic Site and Lack Collateral Activity

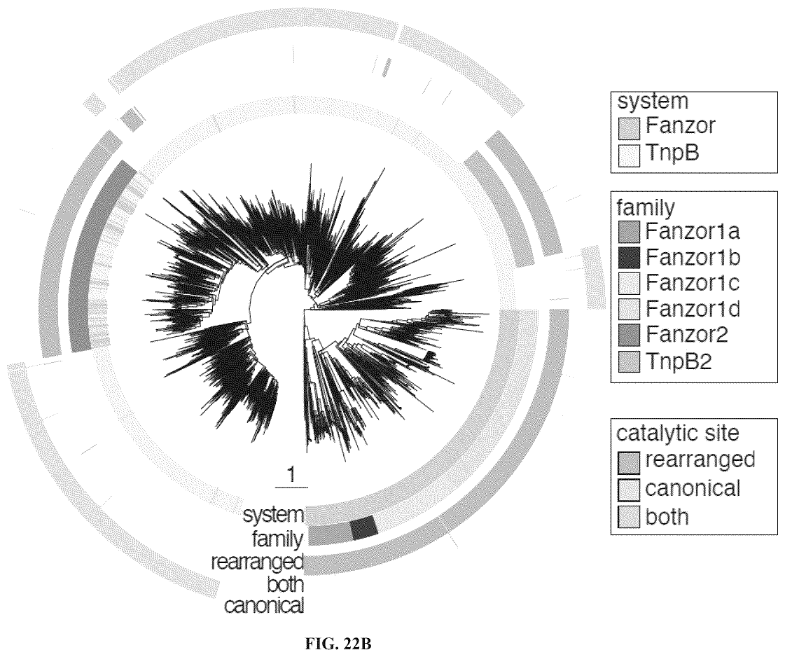

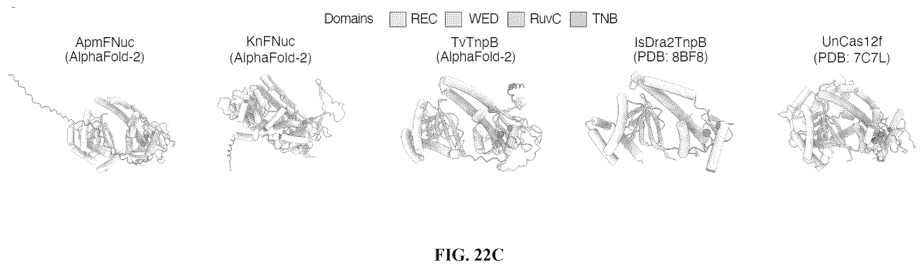

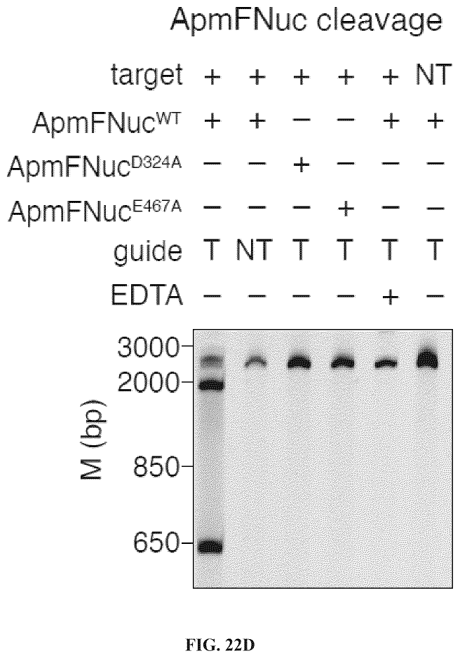

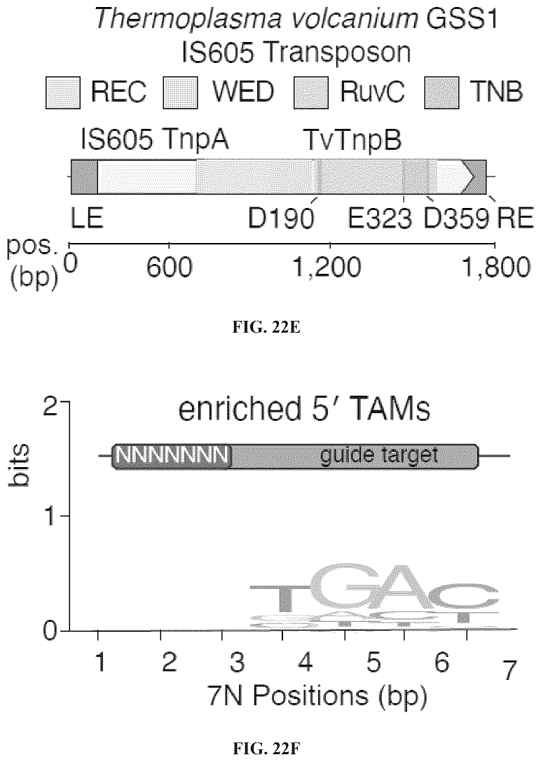

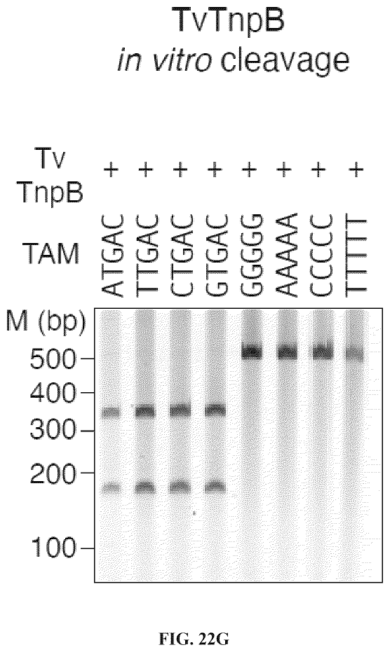

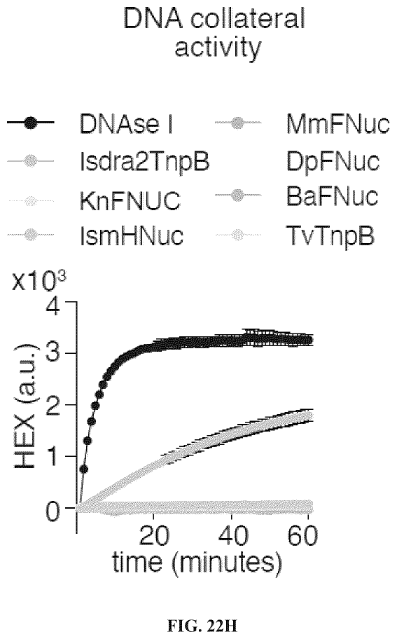

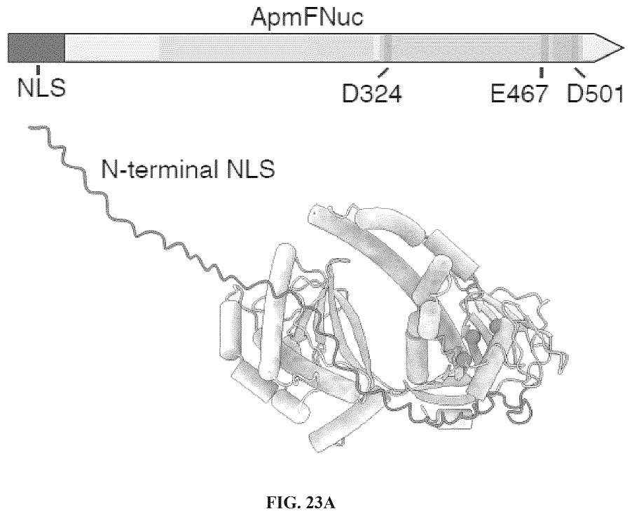

Alignment of Fanzor nucleases and TnpB members shows that, compared to the majority of TnpBs, Fanzor nucleases contain a substitution in the catalytic RuvC-II motif from a glutamate to a catalytically inert residue (proline or glycine) (FIG. 22A). To find TnpBs clades with this substitution, similarly modified RuvC nuclease domains were searched for among the TnpBs. There was similar apparent inactivation of RuvC-II in TnpBs across multiple clades, including a monophyletic group, which was termed TnpB2, in contrast to canonical TnpB1 (FIGS. 22A-22B). Given the demonstrated nuclease activity of ApmFNuc, a search for conserved acidic residues that could potentially compensate for the RuvC-II-inactivating mutations was performed. Indeed, all Fanzor proteins and TnpBs with a loss of the canonical glutamic acid in RuvC-II contained an alternative conserved glutamate approximately 45 residues away (FIGS. 22A-22B).

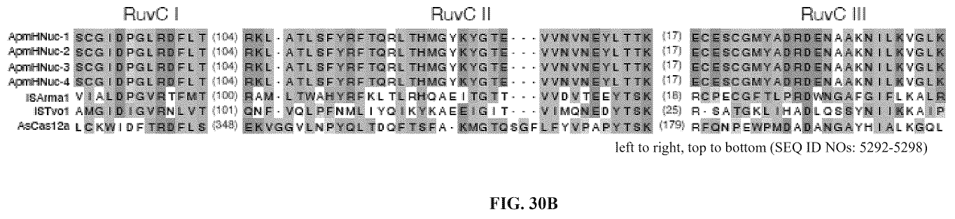

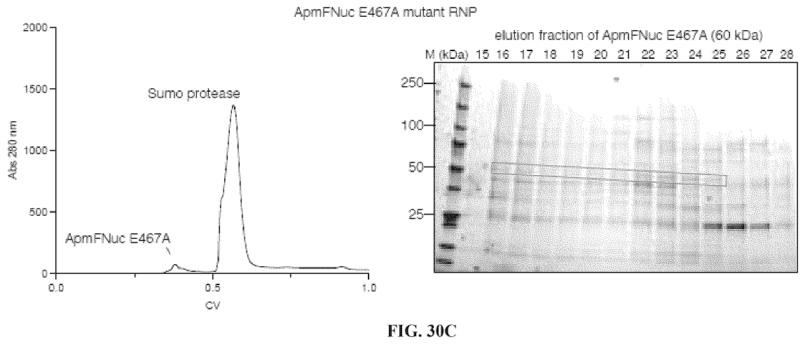

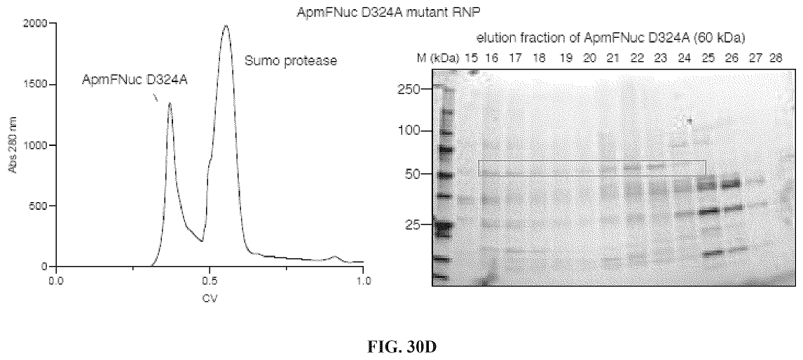

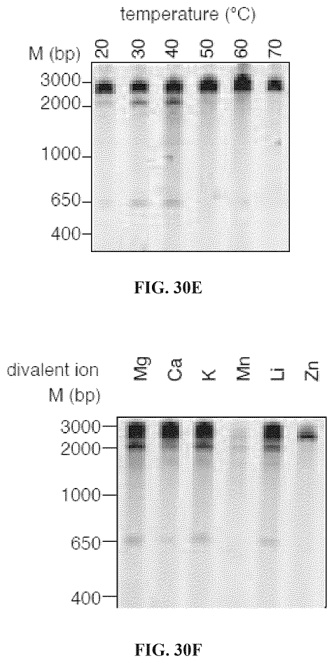

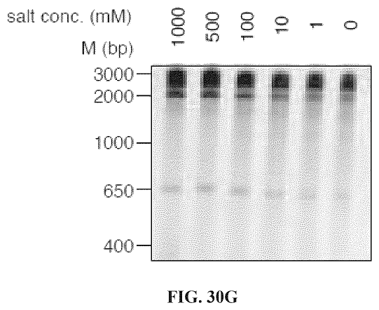

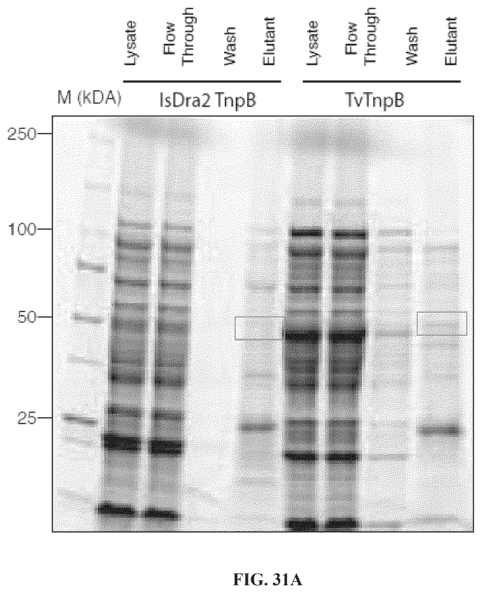

AlphaFold2-generated structural models of ApmFNuc, MmFNuc, DpFNuc, BaFNuc, KnFNuc, and a TnpB from Thermoplasma volcanium GSS1 (TvTnpB) that both contain a rearranged catalytic site with the Cryo-EM structures of TnpB from Deinococcus radiodurans R1 (Isdra2) and Cas12f from uncultured archaeon (UnCas12f) containing the canonical catalytic site were compared (FIG. 22C and FIG. 30A) (Takeda et al. 2021, Nakagawa et al. 2023). This comparison showed that the alternative conserved glutamate of Fanzor nucleases and rearranged TnpB (E467 of ApmFNuc and E323 of TvTnpB) were in close proximity with the catalytic residues in the RuvC-I and RuvC-III motifs, suggesting that these alternative, conserved glutamates compensate for the mutation in RuvC-II (FIG. 22C and FIG. 30A).

To test the predicted role of the conserved alternative glutamate in Fanzor activity, two ApmFNuc RNP with mutations at predicted catalytic sites in RuvC-I (D324A) or the alternative glutamate in RuvC-II (E467A) were purified (FIGS. 30B-30D). While the D324A mutant showed no change in the RNP stability during protein purification, there was a substantial decrease in the expression of the E467A mutant relative to the wild type protein (FIG. 26B). Comparison of the cleavage efficiencies of these mutants with that of the wild-type ApmFNuc showed, in agreement with the nuclease mechanism, that both RuvC-I and RuvC-II mutants abolished ApmFNuc cleavage activity (FIG. 22D). Thus, the alternative Fanzor glutamate is indeed essential for the nuclease activity. Activity required a temperature range of 30° C. and 40° C. for optimal activity, similar to other mesophilic RuvC nucleases, needed complexing with magnesium or a compensatory metal ion, and was robust across a range of salt concentrations (FIGS. 30E-30G).

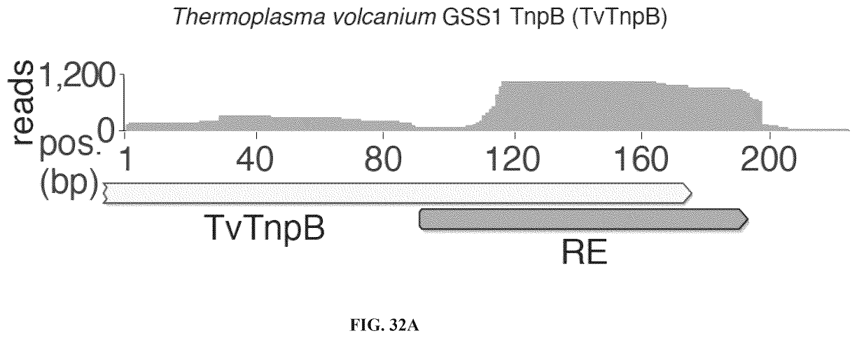

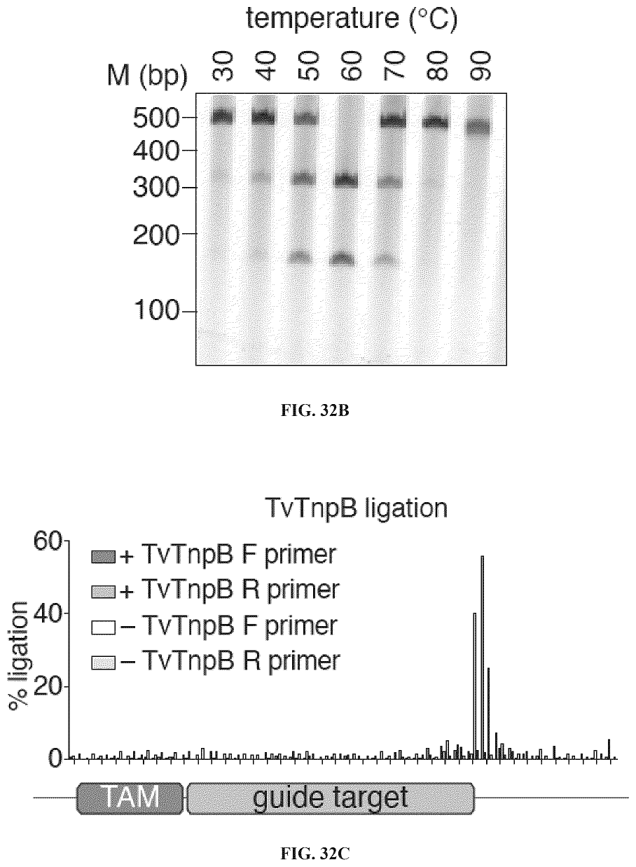

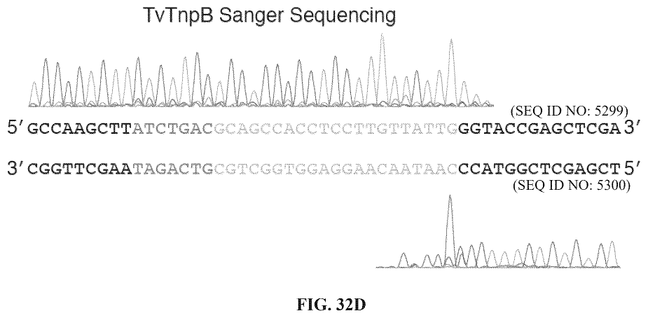

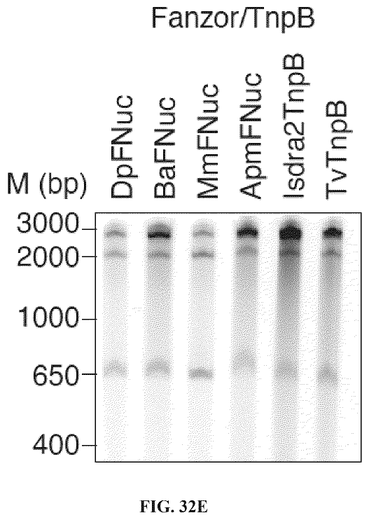

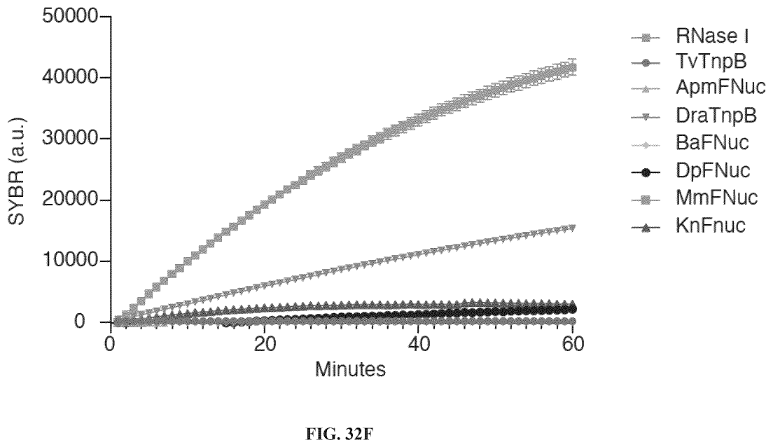

The activity of the TnpB2, TvTnpB, was then profiled to determine if these re-arranged TnpBs were similarly active. TvTnpB RNPs were isolated by co-expressing the enzyme with its native locus in E. coli and profiled associated noncoding RNA by NGS (FIG. 31). Expression of the noncoding RNA species mapped proximal to the RE element, similar to other TnpB systems (FIG. 22E and FIG. 32A). Applying the TAM assay by coexpressing TvTnpB with a synthetic ωRNA containing a reprogrammed 21 nt spacer, incubating the RNP with a 7N TAM library plasmid, and sequencing the cleavage products, showed strong enrichment of a TGAC motif near the 5′ target spacer sequence (FIG. 22F). Notably, this TGAC motif is also present at the 5′ end of the left end (LE), marking the beginning of the TvTnpB-encoding transposon. Because T. volcanium is a thermophile, in vitro cleavage efficiency was optimized over a range of temperatures. The optimal temperature for cleavage at the TGAC TAM at 60° C. (FIG. 32B). All four possible NTGAC TAM sequences were validated along with four negative TAM sequences and demonstrated TAM-specific cleavage, similar to other Fanzors and TnpB nucleases (FIG. 22G). The ends of the cleavage products were profiled with NGS, mapping the cleavage position to position 22 in the non-targeting strand and positions 21 and 22 in the targeting strand (FIG. 32C), with a similar cleavage pattern found by Sanger sequencing (FIG. 32D).

Although the rearranged RuvC catalytic site of the Fanzors and TnpB2 did not impact on target cleavage, it was hypothesized that it could affect the collateral cleavage activity of the enzyme (Chen et al. 2018, Abudayyeh et al. 2016). ApmFNuc, MmFNuc, DpFNuc, BaFNuc, TvTnpB, and the canonical TnpB Isdra2TnpB were profiled for either RNA or DNA collateral cleavage activity by co-incubating the RNP complexes with their cognate targets along with either RNA or DNA cleavage reporters, single-stranded nucleic acid substrates functionalized with a quencher and fluorophore that become fluorescent upon nucleolytic cleavage. While all nucleases had similar on-target cleavage efficiencies (FIG. 32E), the Fanzor orthologs and TvTnpB lacked detectable collateral DNA and RNA cleavage activity in contrast to the strong collateral cleavage activity Isdra2TnpB (Karvelis et al. 2021) (FIG. 22H and FIG. 32F).

### Example 25: Fanzor Nucleases Contain Nuclear Localization Signals and are Functional for Mammalian Genome Editing

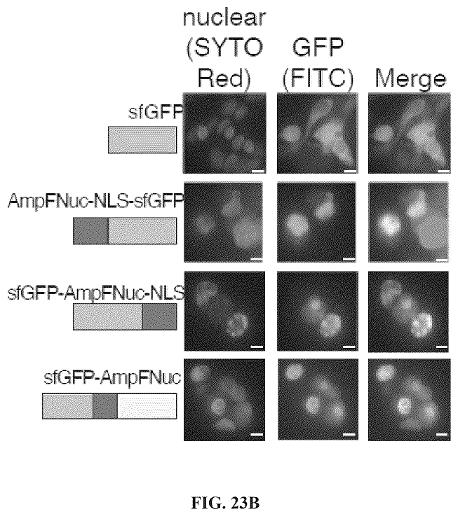

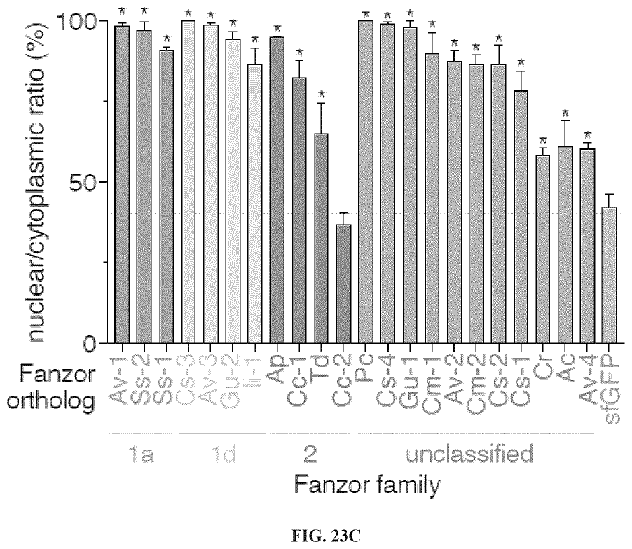

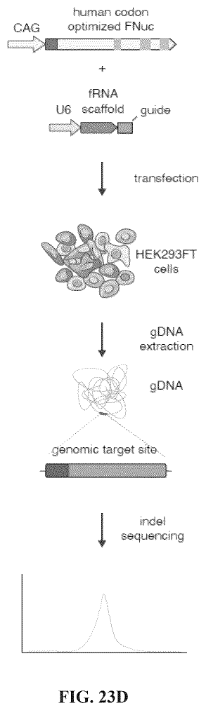

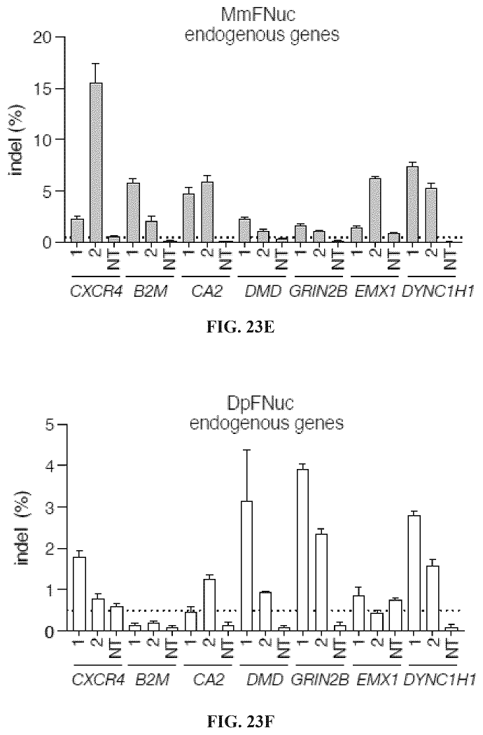

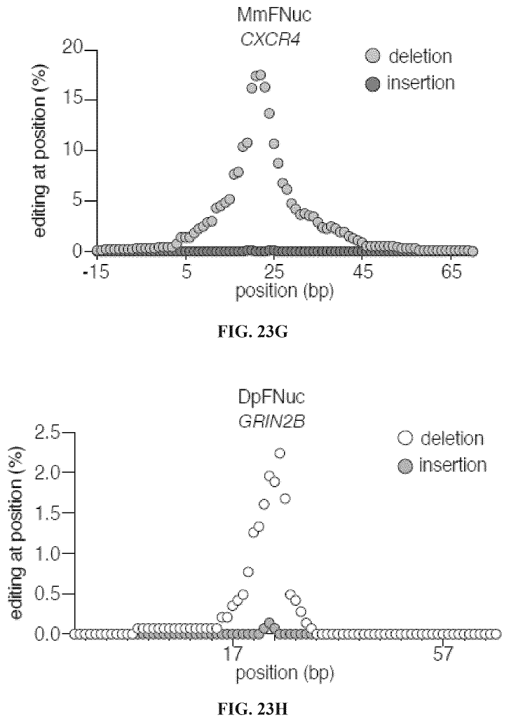

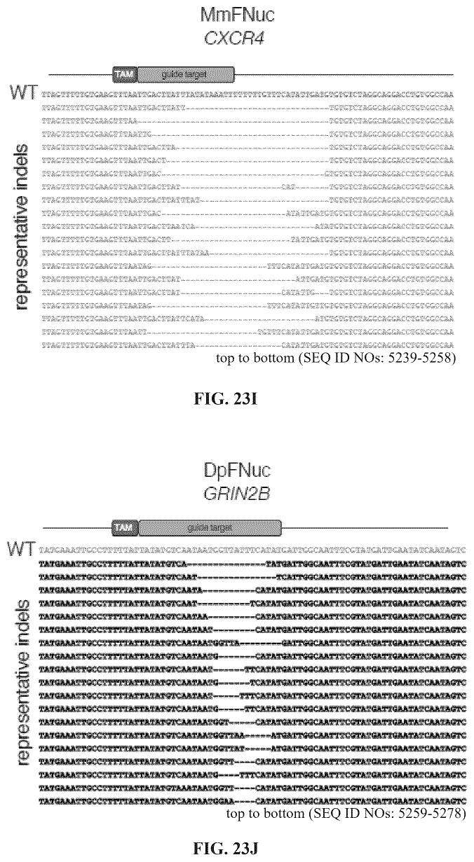

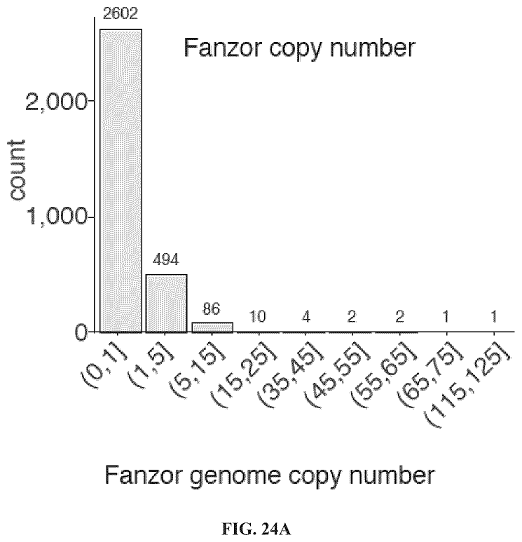

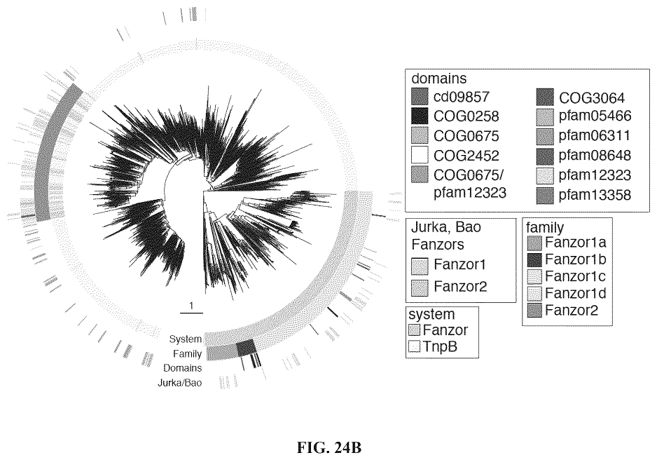

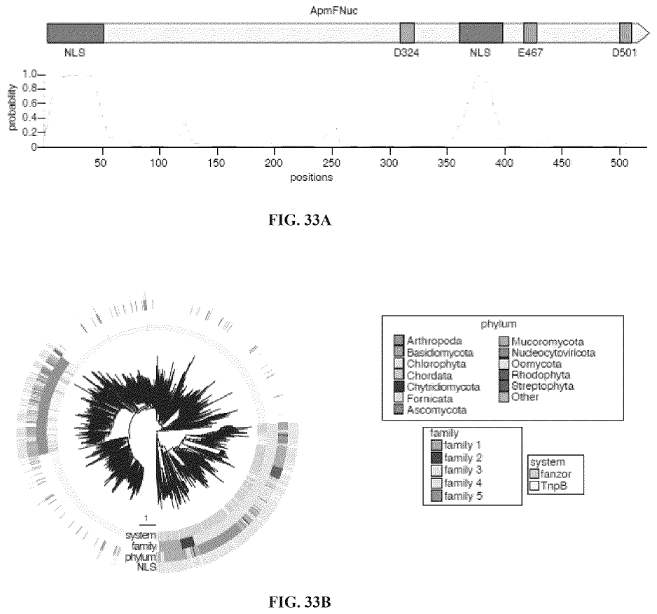

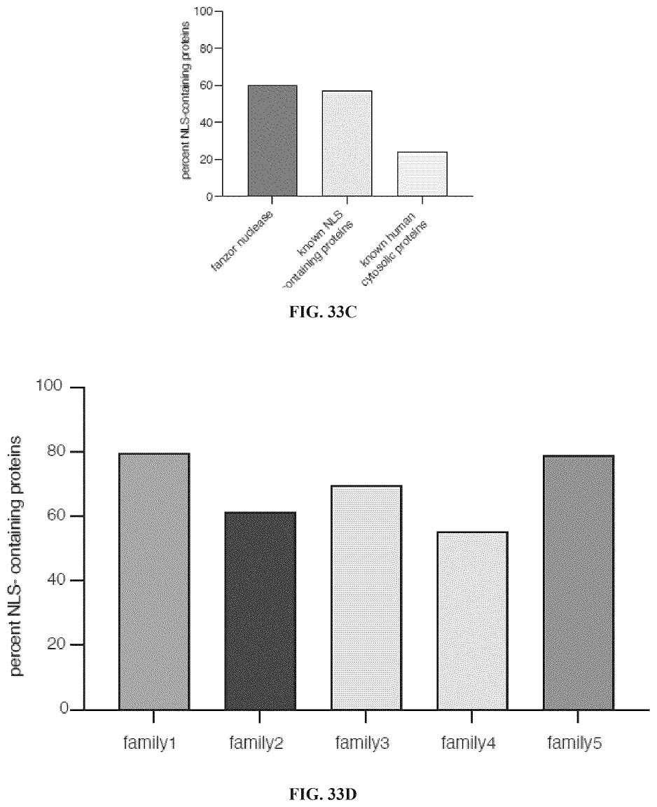

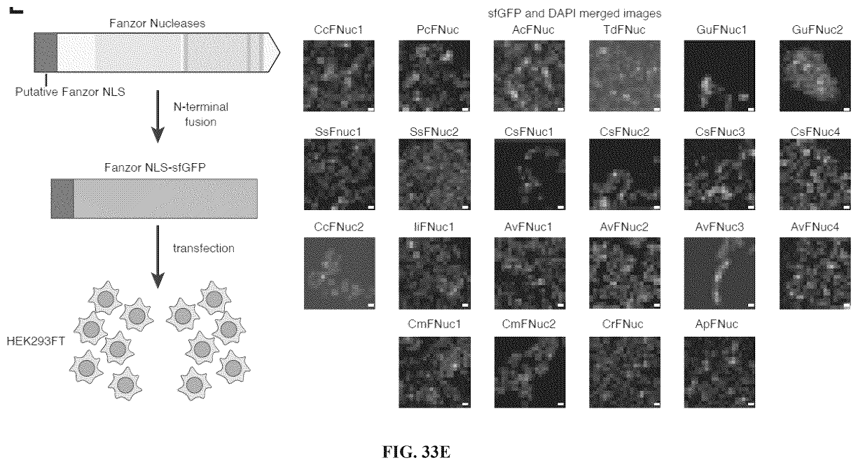

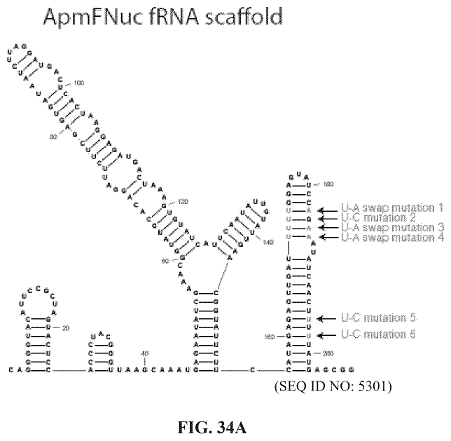

As eukaryotic RNA-guided endonucleases would need to enter the nucleus to access their genomic targets, it was hypothesized that Fanzor nucleases might have harbor nuclear localization signals to actively cross the nuclear membrane. In the Alphafold2 predicted structures of ApmFNuc, a disordered region of 64 amino acids was discovered at the N-terminus (FIG. 23A). Computational prediction of the nuclear localization signal (NLS) identified a strong, positively-charged NLS within the N-terminal region of ApmFNuc (FIG. 33A).

To evaluate the localization of ApmFNuc and its NLS, a super-folder GFP (sfGFP) was fused to the N-terminus of ApmFNuc and attached the N-terminal portion of ApmFNuc containing the NLS to either the N-terminus or C-terminus of sfGFP. sfGFP localization was visualized via fluorescent microscopy, finding that sfGFP with the NLS from ApmFNuc fused to either terminus had strong nuclear localization (FIG. 23B). Fusion of sfGFP with the complete ApmFNuc also caused strong nuclear localization of sfGFP (FIG. 23B). These results suggest that ApmFNuc indeed contains a functional NLS, likely acquired after the capture of TnpBs by eukaryotes.

Next, a broad search for Fanzor-encoded NLS sequences was performed by analyzing each Fanzor ORF for a predicted NLS. Across all Fanzor families, ~60% of ORFs had readily identifiable NLS sequences, on par with the prediction accuracy of a validated set of NLS-containing proteins (Nguyen et al. 2009) and substantially greater than the fraction of NLS sequences predicted for cytosolic human proteins (FIGS. 33B-33D). A subset of 22 Fanzors across Fanzor1 and Fanzor2 families with predicted N-terminal NLS sequences was selected and screened by fusing the N-terminal 100 amino acids of each Fanzor ortholog to sfGFP, transfecting this panel into HEK293FT cells and visualizing sfGFP distribution. 21 out of 22 predicted N-terminal NLS sequences were functional for nuclear localization in mammalian cells, with varying nuclear localization efficiencies (FIG. 23C, FIG. 33E). This experimental validation of the predicted NLS domains shows that Fanzor nucleases acquired mechanisms for nuclear import to access the genome and perform their genomic functions.

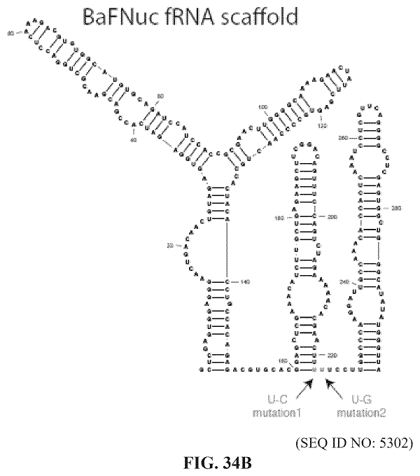

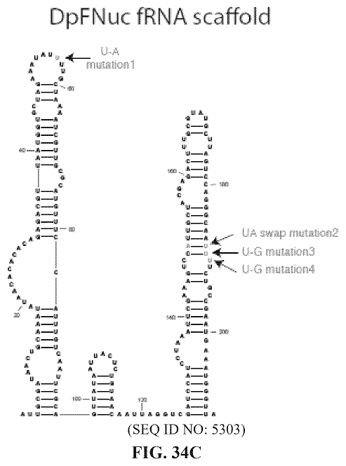

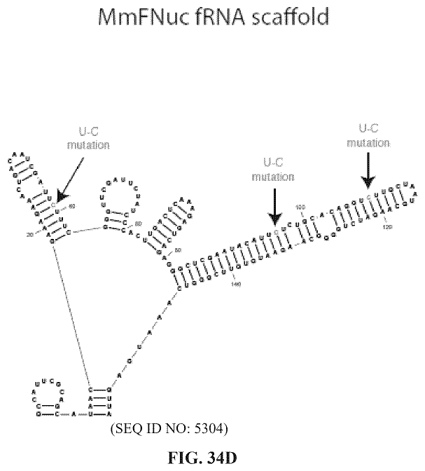

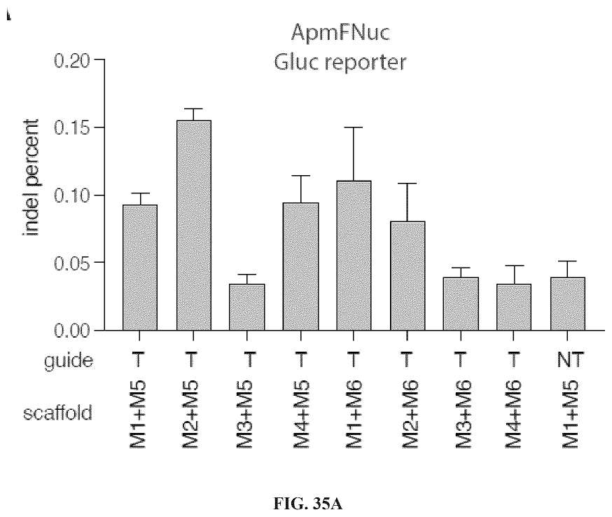

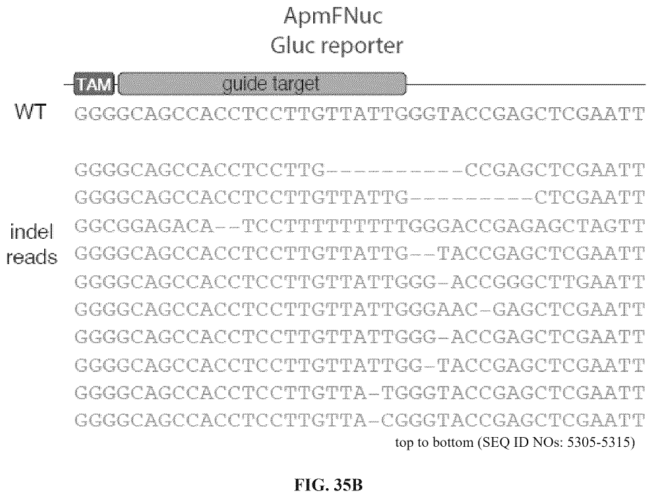

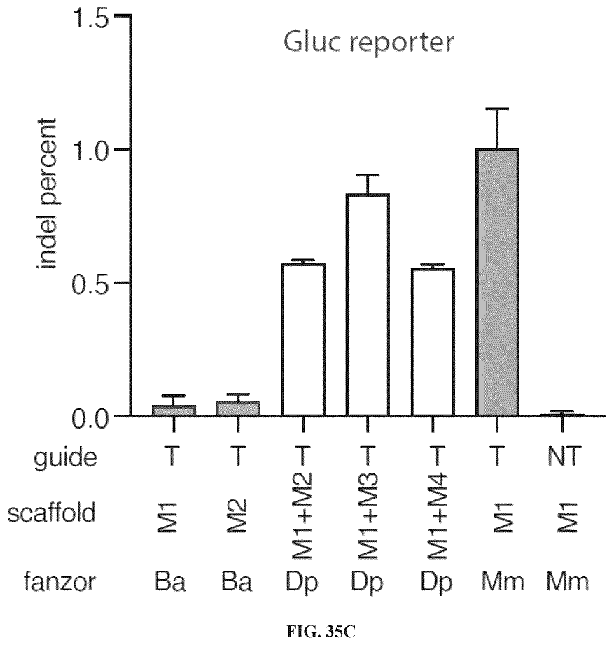

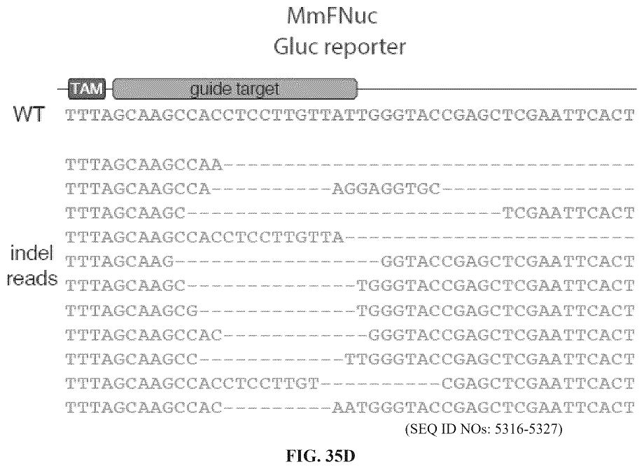

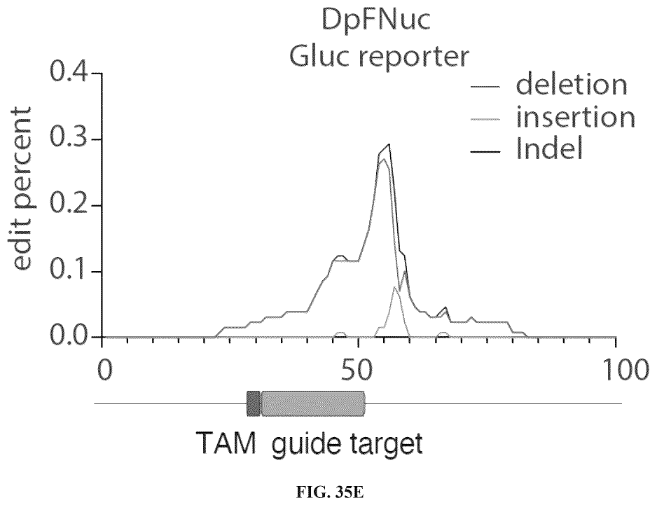

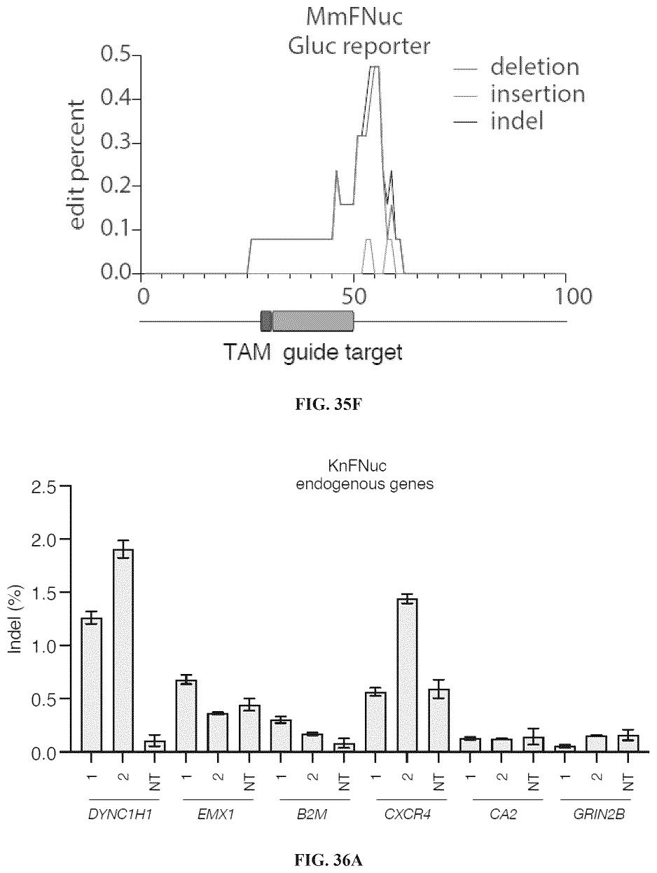

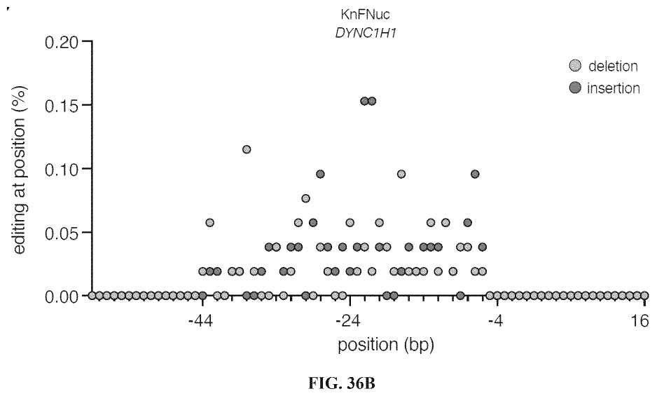

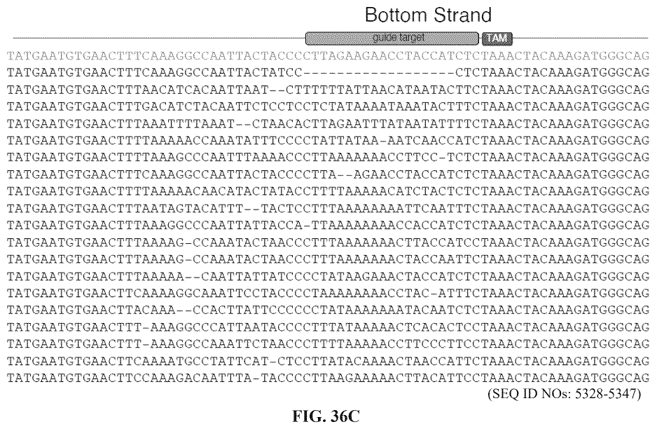

Next, codon-optimizing ApmFNuc, DpFNuc, MmFNuc, and BaFNuc were then tested for mammalian genome editing by engineering their fRNA guide scaffolds for optimal U6-based expression in mammalian cells by removing poly-U stretches (FIG. 34). A reporter plasmid carrying the 21 nt target matching the fRNA guide was designed and its editing was evaluated by next-generation sequencing of generated insertions and deletions (indels). DpFNuc, MmFNuc, and ApmFNuc with engineered fRNAs had detectable editing activity, with DpFNuc and MmFNuc, achieving ~0.5%-1% editing on plasmids inside human cells (FIGS. 35A-35D). The indel patterns of DpFnuc and MmFNuc showed 2-35 bp deletions near the 3′ end of the target site (FIGS. 35E-35F), similar to the indel cleavage patterns of other programmable RuvC containing nucleases, such as Cas12 or TnpB (Zetsche et al. 2015, Karvelis et al. 2021, Altae-Tran et al. 2021). Because DpFNuc and MmFNuc displayed the highest levels of plasmid editing, a panel of guides against 7 endogenous genomic targets was designed (FIG. 23D) and showed varying levels of editing, from ~0.5%-15% (FIGS. 23E-235F), validating Fanzors as RNA-guided nucleases with activity in mammalian cells. As with plasmid editing, editing outcomes were primarily large deletions, ranging in size from 1-25 bp (FIGS. 23G-23J). To evaluate if Fanzor1 orthologs are also functional for genome editing, the editing efficiency of KnFNuc was also tested and showed editing up to 2% across multiple endogenous genomic targets (FIG. 36), demonstrating that both Fanzor1 and Fanzor2 nucleases can be reprogrammed for human genome editing.

RNA-guided DNA endonucleases are prominent in prokaryotes including roles in innate immunity mediated by prokaryotic Argonautes (Swarts et al. 2014); adaptive immunity by CRISPR systems (Hsu et al. 2014, Hille et al. 2018, Doudna et al. 2014); RNA-guided transposition by CRISPR-associated transposases (Strecker et al. 2019, Klompe et al. 2019), and still uncharacterized functions of OMEGA nucleases in transposon life cycles (Karvelis et al. 2021, Altae-Tran et al. 2021). In eukaryotes, whereas RNA-guided cleavage of RNA is the cornerstone of the RNA-interference defense machinery and post-transcriptional regulation (Hannon et al. 2002, Hutvagner et al. 2008), RNA-guided cleavage of genomic DNA has not been demonstrated, to our knowledge. The examples show that the previously uncharacterized eukaryotic homologs (Bao et al. 2013) of the OMEGA effector nuclease TnpB are RNA-guided, programmable DNA nucleases. Extensive searching of diverse genomes of eukaryotes and their viruses enabled the discovery of thousands of RuvC-containing Fanzor nucleases. While this manuscript was in review, additional work characterized Fanzor nucleases biochemically and in mammalian cells, further confirming Fanzors as RNA-guided nucleases (Saito et al. 2023).

Phylogenetic analysis of the Fanzors together with their closest TnpB relatives revealed 5 major Fanzor families, which all contain Fanzor nucleases interspersed with prokaryotic TnpBs, suggesting that TnpBs entered the eukaryotic genomes on multiple, independent occasions. Considering the high abundance of TnpBs in bacteria and archaea, and their mobility, along with the exposure of unicellular eukaryotes to bacteria, this apparent history of multiple jumps into eukaryotic genomes does not appear surprising. Furthermore, given the wide spread of Fanzors in eukaryotes, together with the near ubiquity of TnpBs in bacteria and archaea, it appears likely that TnpBs were originally inherited from both archaeal and bacterial partners in the original endosymbiosis that triggered eukaryogenesis (Lopez-Garcia and Moreira 2023). Subsequent events of TnpB capture by eukaryotes could occur via additional endosymbioses as well as sporadic contacts with bacterial DNA. Notably, however, the high intron density in many Fanzors implies their long evolution in many groups of eukaryotes. The history of Fanzor2, however, is quite distinct from the four Fanzor1 families. This variety of Fanzors are enriched in viruses and in IS607 transposons and are far more closely similar to TnpB than members of other Fanzor families, suggesting likely origin from phagocytosis of TnpB-containing bacteria by amoeba and subsequent spread via amoeba-trophic giant viruses (Boyer et al. 2009).

Association of Fanzor nucleases with transposases suggests a role for their RNA-guided nuclease activity in transposition similarly to the case of TnpB. The exact nature of that role, however, remains unknown. TnpB has been reported to boost the persistence of the associated transposons in bacterial populations (Pasternak et al. 2013, Meers et al. 2023). TnpB and Fanzors potentially could perform different mechanistic roles in transposon maintenance. In particular, these RNA-guided nucleases could target sites from which a transposon was excised, initiating homology directed repair through a transposon-containing locus, restoring the transposon in the original site and thus serving as an alternate mechanism of transposon propagation (Meers et al. 2023). The association of TnpBs and Fanzors with diverse types of transposases suggests that the function(s) of the RNA-guided nucleases do not strictly depend on the transposition mechanism.

The biochemical characterization of both viral and eukaryotic Fanzor nucleases revealed both similarities with the homologous TnpB and Cas12 RNA-guided nucleases and several notable distinctions. Like TnpB and Cas12, Fanzor nucleases generate double-stranded breaks through a single RuvC domain and cleave the target DNA near the 3′ end of the target. However, unlike canonical TnpB and Cas12 enzymes, which possess strong collateral activity against free ssDNA, Fanzor nucleases and a subset of related TnpBs contain rearranged catalytic sites that are not conducive to collateral activity. In contrast to the T-rich TAMs of TnpB and PAMs of Cas12, the Fanzor TAM preference is diverse, with a GC preference observed for the viral ApmFNuc and A/T rich preferences for the eukaryotic MmFNuc, DpFNuc, and BaFNuc. In some cases, the TAM preference agrees with the insertion site sequence, which is compatible with the role of Fanzors in transposition. Finally, the fRNA of Fanzors overlaps with the transposon IRR and TIR, much like TnpB's ωRNA, but extends farther downstream of the Fanzor ORF, in contrast to the ωRNAs that ends near the 3′ regions of the TnpB ORF. Furthermore, although the Fanzor nucleases originated from TnpB, some features of these eukaryotic RNA-guided nucleases notably differ from those of the prokaryotic ones, reflecting their adaptation functioning in eukaryotic cells, such as the acquisition of introns and functional NLS sequences for nuclear localization.

The examples demonstrate that Fanzor nucleases can be applied for efficient genome editing with detectable cleavage and indel generation activity in human cells. While the Fanzor nucleases are compact (~600 amino acids), which could facilitate delivery, and their eukaryotic origins might help to mitigate the immunogenicity of these nucleases in humans, additional engineering is needed to further improve the activity of these systems in human cells, as has been accomplished for other miniature RNA-guided nucleases such as Cas12f (Bigelyte et al. 2021, Wu et al. 2021, Xu et al. 2021, Kin et al. 2021). The broad distribution of Fanzor nucleases among diverse eukaryotic lineages and associated viruses suggests many more currently unknown RNA-guided systems could exist in eukaryotes, serving as a rich resource for future characterization and development of new biotechnologies.

### Example 26: Phage-Assisted Continuous Evolution (PACE) Selection for Improving the Editing Efficiency of Fanzor Proteins (Prophetic)

Following protein purification and sequencing, variants of Fanzor proteins are evolved using PACE systems to form a large library of Fanzor mutants. Mutants are then subjected to selection based on the lack of DNA collateral activity using an antibiotic resistance selection system. Cells harboring Fanzor mutants that restore antibiotic resistance are isolated and subjected to additional successive rounds of mutation and selection under varying selection stringencies.

Those Fanzor mutants that conferred a survival advantage are tested for base editing activity in mammalian cells across >5 endogenous genomic loci to assess editing efficiency, product purity, the size of the editing window, and sequence context preferences. Successive rounds of directed evolution are then performed until the resulting Fanzors perform at a useful level (e.g., >20% editing, >50% product purity, <5% indels, and an editing window of 2-8 nucleotides).

### Example 27: Computational Structure Prediction (Prophetic)

For each position that is experimentally screened for single mutation effects on Fanzor activity, each residue is computationally mutated into other amino acid types. Single sequence structure prediction is performed using AlphaFold2. The model with the highest per-residue confidence score (pLDDT) is computationally evaluated for enzyme and substrate binding free energy. Candidate Fanzor proteins are physically synthesized and evaluated for their genome editing activity using methods described herein.

### Methods

### Computational Discovery of Fanzors

A profile of the Fanzor RuvC domain (Fanzor profile) was constructed by aligning the previously discovered Fanzor proteins (seed sequences) with MUSCLE v5 (-align), extracting the RuvC domain, and building a profile HMM with hmmbuild (default options) from the HMMER v3 suite of programs. An initial set of putative Fanzor proteins was gathered by searching all annotated proteins and translated ORFs (stop codon to stop codon) longer than 100 residues in NCBI eukaryotic and viral assemblies (one assembly per species) as well as all full length proteins annotated on eukaryotic and viral sequences in GenBank (hmmsearch-E 0.001-Z 61295632). To predict introns in Fanzor ORFs, AUGUSTUS v3.5.0 and Spaln v2.4.13f were applied to the genomic region containing the ORF (10 kb upstream/downstream). AUGUSTUS was used for ab initio gene prediction when there was an available parameter set of the same class as the target species. Tantan was used to soft-mask the genome prior to gene prediction using an “-r” parameter of 0.01 if the genome AT fraction was less than 0.8 and 0.02 otherwise (with the suggested scoring matrix for AT-rich genomes). Spaln was used to splice-align Fanzor proteins to the Fanzor ORFs (default options). The protein query set for Spaln was generated by searching UniClust90 and GenBank eukaryotic proteins with the Fanzor profile. The Fanzor profile was iteratively refined by repeatedly searching the initial set of proteins (hmmsearch-E 0.0001-domE 1000-Z 69000000), extracting the RuvC domain, clustering with Mmseq2 (--min-seq-id 0.5-c 0.9), aligning the cluster representatives with the profile seed sequences, manually refining the alignment, building a new profile, and using the new profile for the next round. Three rounds of refinement were completed. The refined profile was used for a final round of searches and clusters that would have been included in the profile were kept for the subsequent filtering steps. To reduce the likelihood of including genome assembly contaminants in downstream analysis, all Fanzor proteins from NCBI assemblies marked as contig level completeness or those originating from contigs shorter than 50 kb (only from assemblies) were discarded. The remaining sequences were clustered using a combination of Diamond v2.1.6 (--evalue 0.0001-id 70-query-cover 90-subject-cover 90-max-target-seqs 500-comp-based-stats 3) and MCL (-I 4.0). Each cluster was aligned with MUSCLE and a consensus sequence was computed using a custom python script. The RuvC domains were extracted from each consensus sequence and all aligned with MUSCLE. The alignment was manually inspected and filtered to yield a final set of Fanzor sequences.

### Computational Discovery of TnpBs

A profile HMM was constructed from a multiple sequence alignment of each Fanzor family and used to query a custom database of prokaryotic and metagenomic assemblies using HMMER (-E 0.0001-Z 61295632). Sequences identical to another sequence were discarded and the remaining were clustered with Mmseqs2 (--min-seq-id 0.7-c 0.9-s 7). Each TnpB sequence was assigned to a Fanzor family based on the profile that matched it with the highest domain bitscore. The split-RuvC domain was extracted from each cluster representative and further clustered with Mmseqs2 (--min-seq-id 0.5-c 0.9-s 7) for a two-step clustering process. These cluster representatives were aligned with MUSCLE and sequences without alignment to the conserved DED motif were discarded.

### Phylogenetic Analysis of Fanzor

To make a combined tree of TnpBs and Fanzor sequences, the split-RuvC domain was extracted from every Fanzor consensus sequence and clustered with Mmseqs2 (--min-seq-id 0.9-c 0.9). These cluster representatives were aligned, along with the TnpB split-RuvC domain cluster representatives, using MUSCLE. To make a tree of only Fanzor sequences, the extracted split-RuvC domains were aligned with MUSCLE without clustering. In both cases, a approximately-maximum-likelihood phylogenetic tree was constructed with FastTree2 (-lg-gamma) and visualized with R and the ggtree suite of packages.

### Phylogenetic Analysis of Fanzors and TnpBs

To make a phylogenetic tree of TnpB and Fanzor sequences, the split-RuvC domain was extracted from every Fanzor consensus sequence and aligned to the split-RuvC domain of a 3k random subset of the two-step clustered TnpB representatives using MUSCLE (-super5). Sequences appearing to be fragments were discarded from the alignment and the remaining sequences were realigned. An approximately-maximum-likelihood phylogenetic tree was constructed with FastTree2 (-lg-gamma). All branches with a local support value (as computed by FastTree) less than 0.7 were collapsed and the tree rooted at the midpoint. The subsequent tree was visualized with R and the ggtree suite of packages.

### Prediction of NLS in Fanzors

NLStradamus was used with default threshold at 0.6 and model option 2 (four-state bipartite model) to predict NLS domains. For background false positive rate determination, a comprehensive search on Uniprot is performed by looking for Homo sapiens cytosolic proteins (with reviewed status) and a total of 1126 proteins are pulled out for analysis. For on target false negative rate determination, the original set of training sequences that include known NLS containing proteins from NLStradamus is used (Nguyen Ba et al. 2009). NLS sequences cloned for experimental testing are listed in Table 5.

TABLE 5
NLS sequences relevant for the present disclosure.

| | | | SEQ |
|---|---|---|---|
| | | | ID |
| Organism | Family | NLS sequences | NO: |
| Catovirus CTV1 | Family 5 | ATGGACTGTTTTATCACTTGCTTGCAGTCTTGGGAGAGAATTTTG | 17 |
| | | AAACGAAAGCAACAGAAGAAAAGGCCGCGCTTGTTCTCTATTC | |
| | | TCCCTCGGAAGTCTGGATTCACTATAAGCTATGTCCCAAATCTT | |
| | | GTCTGACGGGAAA | |
| Prototheca cutis | Unclassified | ATGATGAGGGAAGTTTCTAAAAAAGGGAAAGGAAAGGAAAAG | 18 |
| | | TCCTCTGCTTCCACTTCAAGGAGTAGGAAGAGGAAGAGGAAAA | |
| | | GGCAAAAAAGGTCTTCACAAGCTGCCTCTTCTGCCAAAGCCAGA | |
| | | GCGTCCGCAGTTAATCAC | |
| Andricus curvator | Unclassified | ATGATGGCCTGTAAAATTGGCGCTCTGAAAAGGCGCAAGGGTA | 19 |
| | | AACACGGTAAGATTAATATAAGCTATGCGGAATACAAGGAAAA | |
| | | TCCGTTCAGTTGTTGGAACTATGTTTTTGACATGTATAAGATTAT | |
| | | GAAATAGGCATAGAT | |
| Torulaspora | Family 5 | ATGATGACGGAGATCAACTATTACTGGTTTAAAAAGAAAAAAA | 20 |
| delbrueckii | | AAAAAAACATTGAGTCTAACTCTTGGTTTAACATCAATAGCATA | |
| | | GAAAACAAGAAAAAAGAGTTTGAAGAGAATGATATACCTCGAA | |
| | | CAATGTGAAAGAC | |
| Globisporangim | Unclassified | ATGAAACGCAAACAGCAGAAGAAACGACCGAGACTCTTTTCCA | 21 |
| ultimum | | TCCTTCCGCGCAAGTCAGGATTCACCATTTCCTACGTCCCTATTT | |
| | | CTAGTATGACACTGATGAAACTGCTTTCTATGGGGGATACAGGC | |
| | | ATCAGAGGACGTG | |
| Globisporangim | Family 4 | ATGATGATTAAAGAAAAGTACTCTAGCAACAAGCGCAAAAGGT | 22 |
| ultimum | | ATCCTACCACACACCGAAAGAAACGCATGTCAGACGCCCAAAT | |
| | | CAGTACGAAAGCTACGACAATACACGGCAGAAGCATCCCTCCC | |
| | | GTTTTATGTGCGGAGGTCA | |
| Scenedesmus sp. | Family 1 | ATGATGAATGAAATCCAACTCCCTACCCCGAGGGGGTCCGCGA | 23 |
| PABB004 | | GGCGGAAACGAAAGAGACAAACCGAACCCCAAATAAGTTACGA | |
| | | TCAGGCCAAAAACACTTTGCTTGGTGTGCTTTTGCAGAAACTGA | |
| | | CCGCATCCCCCGGGGCAGT | |
| Scenedesmus sp. | Family 1 | ATGATGAGCTATGGGATTGAGATTGAGACGGTAGCAAAACGAA | 24 |
| PABB004 | | CGAGCAAAAGTAAAAAAAAACGGAAGTTCGCACAGCAACTGCA | |
| | | TTCAGATGGAGAAAGCGTTACCATCCTGTATGAGTCAGAGCTTG | |
| | | AAACTCAAATCTAAACAT | |
| Chlamydomonas | Unclassified | ATGATGAAAGAGGCAGTGAAGAATGTGAAACCCAAAGTGCCAG | 25 |
| sp. ICE-L | | CGAAGAAACGAATAATTACAGGTAGTAAAACTAAGAAGAAGGT | |
| | | TTTCGTGAAAAAGAAGCCGCCGGACAAAAAACCCTTGAAGACC | |
| | | CAACAAGAGCCCGGTCCAA | |
| Chlamydomonas | Unclassified | ATGCCTTTCCTCTGCACGACTCGATACTGTAGACGGCCAAGCAA | 26 |
| sp. ICE-L | | GAATGAGAAAAGAAAGCGCAAGACCTCTCACATTTTGGTGGTG | |
| | | GCACTCCAAATTTGGATTCATAGCGTCGCTCATAGTGATTACTG | |
| | | ATGGTTTCGCCGTC | |
| Chlamydomonas | Family 4 | ATGAAGCGAGCAGGCGGTCGAAAAGGAGGTCACCGGCGAAAG | 27 |
| sp. ICE-L | | CAGTCAAAGCATTGGCAACCGCGGGCACGAACCGCAAGAAGAA | |
| | | GACGCCAAAAAAGAGGAAGACTGCACATGTCCATGGGCCACTT | |
| | | GCGGGGCAGGCTGCCGGACAG | |
| Chlamydomonas | Unclassified | ATGATGCGGGAGGTCAAGGCGGGAACTAAGAGAGCGAGACAG | 28 |
| sp. ICE-L | | CCTGAGGTGAAGAGTGTAGCATTGAAAAAAGCTAAGAAGACAG | |
| | | GTAGGGCTTCCAAGCAGGCTTCTTCCTCTAACACGGCGTTTAGT | |
| | | CGTAGTCGAAGCACACAGA | |
| Catovirus CTV1 | Family 5 | ATGTACCTCTTGATGAAGAAGAAAAAAGAACCTGACAAAAACA | 29 |
| | | AAAGTGACAAAGAAAAAGAGTATGAAGAAAAGTATCGAAAGT | |
| | | ATATCACATCCTATAAGACACACAAGACATCACTCGAAAACAC | |
| | | CACGATGAAGTTGTTC | |
| Indivirus ILV1 | Family 4 | ATGATGAAAAAGCCTAAGGTGAAAGAGAAAGAGAAGGAAAAG | 30 |
| | | GAGAAAGAAAATTTCGATTTTATGAAGACTAATAAGGGGAATA | |
| | | TCCATAAGCTCATAAAGGATAAGATGGTACTCTCTATAATCGGG | |
| | | TAAAGGAGGTTATACT | |
| Apophysomyces | Family 1 | ATGATGGAGACTATCGTAAATAAAGAACCACCCGACAAGCGCA | 31 |
| variabilis | | CCCGCCCGGATCGGGCTGCAAAAATTAAAGACCGCAAAAATGG | |
| | | GGAAGAAAACGTCGTTAAATGTACTCTTTCCAGGATCATAGGTA | |
| | | CCTTCCGTTGACCAAAAT | |
| Apophysomyces | Unclassified | ATGAGCCCCGGATCATCTGCGGCGAGAAAGAAAAACGAGAAGC | 32 |
| variabilis | | AGTGTCGGGTGCAGAAGAAGCGAAAGAGACGCGGCCCGAAAG | |
| | | GTGGGGGTCCGGCCAGTAAAACCGCAAGAAAGACGACAGTAAT | |
| | | GTCTCAAGAAGGGATGCCCATGGG | |
| Apophysomyces | Family 4 | ATGATGGCAAGCCGAAACAAGCGGAAAAAAAAGCCGCAGGCG | 33 |
| variabilis | | AGCACGAGTGCCGACACCCAGAGCGACGACGATTTCCAACAAC | |
| | | TCCTTCCGCCGAAGGGTAAATTGAATATGAAAATGCAGATGAC | |
| | | GAAAACAACCTCTGCAGGTT | |
| Apophysomyces | Unclassified | ATGGTTCACCTTATACTCATTCTTATGACGAAGAAAAAGAAAAA | 34 |
| variabilis | | ATTCAAGAAAAAGAAGATTTTTTACAAAAAATACCACAAATTC | |
| | | AACTGGCTCTCCAGGCTCTTCAATGATAATCAGTTTAGTGAAGA | |
| | | CAAAATTT | |
| Cyanidischyzon | Unclassified | ATGCCACTGACGCGAAGGCGACGACAAAAATCCCGGAGAGGGC | 35 |
| merolae | | TTCACCGGAGACATAGGACGAGGCGGGCGCGACGCAAAGAGCG | |
| | | AGTCATCGAAATCTCCACCCCCAAGTATCGACATCTCGCCCGGT | |
| | | GTTTAGTGCGGAACAG | |
| Cyanidischyzon | Unclassified | ATGTCTCCACGGCCGCAGCCGGCTGCGCCTCCTGCAGCGCAGGG | 36 |
| merolae | | AAGAGCCCGCGGGGGCGCCCCGGCACCCGCTGGCAGACGAGGG | |
| | | GGGGCTGCAGCACCTAGACCGGGGGCGAGGAGACGGGCAGGG | |
| | | CGCAACATCACGAACGGGACGCC | |
| Chlamydomonas | unclassified | ATGTGTAGGAGGTGCCGCATCACGCCACTTTGGCTGGCTGGTCG | 37 |
| reinhardtii | | GAGGATGAAAAAGAGACGACGACGGGTCCTCCGACCCAAAAAG | |
| | | TGCATGATAACAACCCTGTCTCTGGCCAGAACACGGGGTAGGG | |
| | | ATGCGGGCATGAAGGACs | |
| Contarinia | Family 3 | ATGATGTATTGTATGCATGAGGATTCTAGTCATAAAAAGGGTCG | 38 |
| nasturtii | | GCGGCGGACGATGCGGATCAGCTCAAGGGAGTGGGCTTTTCTG | |
| | | ACTCGATCTCGCAAATTTCGACGCCTGTTGAGAAGGCTTAGAAA | |
| | | ACTTAGGCTGTGGACG | |

### Prediction of NLS in Fanzor

NLStradamus was used with default options to predict NLS domains.

### Prediction of NLS in Fanzors

NLStradamus was used with default threshold at 0.6 and model option 2 (four-state bipartite model) to predict NLS domains. For background false positive rate determination, a comprehensive search on Uniprot is performed by looking for Homo sapiens cytosolic proteins (with reviewed status) and a total of 1126 proteins are pulled out for analysis. For on target false negative rate determination, the original set of training sequences that include known NLS containing proteins from NLStradamus is used (Nguyen Ba et al 2009). NLS sequences cloned for experimental testing are listed in Table 5.

Prediction of Transposon Associations with Fanzor Systems

RFSB transposon classifier (Riehl et al. 2022) is used to classify Fanzor-transposon associations by inputting the surrounding 10 kb genomic sequence around the Fanzor protein. The classify mode is used with default parameters to make the prediction. Afterward, all predicted DNA transposon is mapped back to the phylogenetic tree. For all Fanzor nucleases that were classified with transposons, cd-hit is used to cluster these sets of Fanzor proteins with default parameters to find any clusters with two or more sequences for multiple sequence alignments. Then these clusters containing (>2 Fanzor systems) were blasted against all Repbase documented transposons (Bao et al. 2015). Left and right end elements, terminal inverted repeats (TIR), and their associated transposons are then determined by either protein homology to known transposons in Repbase or high similarity of TIR/LE/RE element to known transposon profiles.

Prediction of Fanzor-Associated ncRNA

Fanzor that were not simply ORF translations were clustered along their entire length at 70% sequence identity and 95% coverage with Mmseqs2 (--min-seq-id 0.7-c 0.95). Each cluster with at least two sequences was subject to ncRNA prediction. For each cluster, the 5′ region of the first exon plus 1.5 kb upstream bases and 3′ region of the last exon plus 1.5 kb downstream bases were cut from sequence. The 5′ and 3′ regions were aligned separately with MAFFT (default options). Each column of the alignment was scored for conservation and the change point in conservation scores was predicted with the R changepoint package to detect a drop in conservation. If the predicted change point was found to be at least 13 bases outside of the exon boundary of every sequence in the alignment, the conserved portion of the exon, plus 11 bases past the change point, were folded with RNAalifold from the ViennaRNA software suite.

### Fanzor and TnpB Protein Purification

To purify Fanzor or TnpB protein, Rosetta2 DE3 pLys cells were transformed with a twin-strep-sumo tag fused to the N-term of a Fanzor or TnpB construct along with the predicted fRNA/wRNA driven by a separate vector. Following transformation, single colonies were picked from the agar plate containing antibiotics and picked into a starter culture of 10 mL for overnight incubation at 37 degree Celsius. The starter culture was transferred to 2L of TB with the designated antibiotics and grown until the OD reached between 0.6-0.8. The culture was moved to 4C for 30 minutes prior to induction with 0.5 mM IPTG induction. The cultures were then grown at 16 degree Celsius overnight and harvested by centrifugation the next day. The pellet is then flash frozen at −80C and subsequently homogenized in lysis buffer (0.02M Tris-HCl pH8.0, 0.5M NaCl, 1 mM DTT, and 0.1M Complete™, EDTA-free Protease Inhibitor Cocktail (Merck Millipore) with high-pressure sonication for 15 minutes. The homogenized lysates are then centrifuged at 14,000 RPM for 30 minutes at 4C. The clarified supernatant is isolated from the subsequent bacterial pellet and incubated with Strep-Tactin®XT 4Flow® high capacity resin (Cat. No. 2-5030-010) for 1 hour. Following incubation, the crude solution is loaded onto a Glass Econo-Column® Column for gravity flow chromatography and washed three times with the previously described lysis buffer. To elute tagged protein, 10 units of sumo protease is then added onto the column for on-column cleavage overnight at 4C. The next day, the eluent is collected and concentrated through an Amicon® Ultra-15 Centrifugal Filter (Cat. No. UFC9030) before continuing to FPLC. To purify desired protein from added sumo protease, the concentrated eluent is loaded onto a Superdex® 200 Increase 10/300 GL gel filtration column (GE Healthcare). The column was equilibrated with running buffer (10 mM HEPES (pH 7.0 at 25C), 1M NaCl, 5 mM MgCl2, 2 mM DTT). The Peak fractions containing RNP are pulled and analyzed by SDS-PAGE. Correct fractions are concentrated again with amicon filter tubes and subsequently buffer is exchanged into storage buffer (0.02M Tris HCL PH8, 0.25M NaCl, 50% glycerol, 2 mM DTT) and stored at −20 for further use. TnpB proteins follow the same purification procedure with the following modifications: T7 express (NEB) pLys strain is used for transformation and subsequent culture.

### Cell-Free Transcription/Translation TAM Screen

Fanzor protein sequences were E. coli codon optimized using the IDT codon optimization tool, and fRNA scaffolds were synthesized by IDT eBlock gene fragments. Cell-free transcription/translation reactions were carried out using a PURExpress In Vitro Protein Synthesis Kit (NEB) as per the manufacturer's protocol with half-volume reactions, using 75 ng of template for the protein of interest, 125 ng of template for the corresponding fRNA or ωRNA with a guide targeting the TAM library and 30 ng of TAM library plasmid. Reactions were incubated at 37° C. for 4 hours, then quenched by heating up to 95 degree Celcius for 15 minutes and cooling down to 4° C. 10 μg RNase A (Qiagen) is added followed by a 15 min incubation at 50° C. DNA was extracted by PCR purification and adaptors were ligated using an NEBNext Ultra II DNA Library Prep Kit for Illumina (NEB) using the NEBNext Adaptor for Illumina (NEB) as per the manufacturer's protocol. Following adaptor ligation, cleaved products were amplified specifically using one primer specific to the TAM library backbone and one primer specific to the NEBNext adaptor with a 10-cycle PCR using NEBNext High Fidelity 2× PCR Master Mix (NEB) with an annealing temperature of 65° C., followed by a second 12-cycle round of PCR to further add the Illumina i5 adaptor. Amplified libraries were gel extracted, quantified by qubit (Invitrogen) and subjected to paired-end sequencing on an Illumina MiSeq with Read 1 200 cycles, Index 1 8 cycles, Index 2 8 cycles and Read 2 80 cycles. TAMs were extracted and position weight matrix based on the enrichment score was generated and Weblogos were visualized based on this position weight matrix using a custom Python script. All sequencing primers used are listed in Table 6.

TABLE 6
NGS primers relevant for the present disclosure.

| | | SEQ ID |
|---|---|---|
| Name | NGS Primers | NO |
| TAM_NGS_F1 | ACACTCTTTCCCTACACGACGCTCTTCCGATCTCtggaattgtgagcggataacaattt | 39 |
| | cacacagg | |
| TAM_NGS_R | GTGACTGGAGTTCAGACGTGTGCTCTTCCGATCTctgcaaggcgattaagttgggta | 40 |
| | acgcc | |
| Luciferase_Indel_ | ACACTCTTTCCCTACACGACGCTCTTCCGATCTCacgtggagtccaaccctggacc | 41 |
| NGS_F1 | | |
| Luciferase_Indel_ | GTGACTGGAGTTCAGACGTGTGCTCTTCCGATCTtcagcatcgagatccgtggtcgc | 42 |
| NGS_R1 | | |
| EMX1_Fanzor2_ | ACACTCTTTCCCTACACGACGCTCTTCCGATCTCtttgttggagttcgttttcttccttga | 43 |
| NGS_F | aatttgttgg | |
| EMX1_Fanzor2_ | GTGACTGGAGTTCAGACGTGTGCTCTTCCGATCTattgactgtagacctagactacag | 44 |
| NGS_R | accgtcac | |
| HPRT1_Fanzor2 | ACACTCTTTCCCTACACGACGCTCTTCCGATCTCgggtcacagggcaagactttgtct | 45 |
| NGS_F | C | |
| HPRT1_Fanzor2_ | GTGACTGGAGTTCAGACGTGTGCTCTTCCGATCTtgccaccacgcctggctaatt | 46 |
| NGS_R | | |
| dync1h1_NGS_F | ACACTCTTTCCCTACACGACGCTCTTCCGATCTCatcattccaccaatcaggactcgg | 47 |
| | C | |
| dync1h1_NGS_R | GTGACTGGAGTTCAGACGTGTGCTCTTCCGATCTccagcctggtcaacctagcgag | 48 |
| | a | |
| b2m_NGS_F | ACACTCTTTCCCTACACGACGCTCTTCCGATCTCccttetccccacagcctccc | 49 |
| b2m_NGS_R | GTGACTGGAGTTCAGACGTGTGCTCTTCCGATCTgctgtaaactagccaggttggga | 50 |
| | atatattgcc | |
| cxcr4_NGS_F | ACACTCTTTCCCTACACGACGCTCTTCCGATCTCgtctgagtcttcaagttttcactcca | 51 |
| | gctaacac | |
| cxcr4_NGS_R | GTGACTGGAGTTCAGACGTGTGCTCTTCCGATCTacagtcctaccacgagacataca | 52 |
| | gcaac | |
| CA2_NGS_F | ACACTCTTTCCCTACACGACGCTCTTCCGATCTCagagactcagagtccaagaggg | 53 |
| | aagcc | |
| CA2_NGS_R | GTGACTGGAGTTCAGACGTGTGCTCTTCCGATCTactagggagtggcttatgcacag | 54 |
| | gtatattatgtg | |
| DMD_NGS_F | ACACTCTTTCCCTACACGACGCTCTTCCGATCTCTCCTTCAGTTCTATC | 55 |
| | CATGTTGTTGCAAATGGTAAG | |
| DMD_NGS_R | GTGACTGGAGTTCAGACGTGTGCTCTTCCGATCTTCTTTAATCAATGCT | 56 |
| | TTGTAGTTTTCACTGTATAAATATTTCACC | |
| Grin2b_NGS_F | ACACTCTTTCCCTACACGACGCTCTTCCGATCTCATGTCTGGAATTGAG | 57 |
| | CCAGGTACTGGG | |
| Grin2b_NGS_R | GTGACTGGAGTTCAGACGTGTGCTCTTCCGATCTCATTAACCAGGTAC | 58 |
| | TGGCCCACTATAGGG | |

### Cell-Free Transcription/Translation TAM Screen

Fanzor protein sequences were E. Coli codon optimized using the IDT codon optimization tool, and fRNA scaffolds were synthesized by IDT eBlock gene fragments. Cell-free transcription/translation reactions were carried out using a PURExpress In Vitro Protein Synthesis Kit (NEB) as per the manufacturer's protocol with half-volume reactions, using 75 ng of template for the protein of interest, 125 ng of template for the corresponding fRNA or ωRNA with a guide targeting the TAM library and 30 ng of TAM library plasmid. Reactions were incubated at 37° C. for 4 hours, then quenched by heating up to 95 degree Celcius for 15 minutes and cooling down to 4° C. 10 μg RNase A (Qiagen) is added followed by a 15 min incubation at 50° C. DNA was extracted by PCR purification and adaptors were ligated using an NEBNext Ultra II DNA Library Prep Kit for Illumina (NEB) using the NEBNext Adaptor for Illumina (NEB) as per the manufacturer's protocol. Following adaptor ligation, cleaved products were amplified specifically using one primer specific to the TAM library backbone and one primer specific to the NEBNext adaptor with a 10-cycle PCR using NEBNext High Fidelity 2× PCR Master Mix (NEB) with an annealing temperature of 65° C., followed by a second 12-cycle round of PCR to further add the Illumina i5 adaptor. Amplified libraries were gel extracted, quantified by qubit (Invitrogen) and subjected to paired-end sequencing on an Illumina MiSeq with Read 1 200 cycles, Index 1 8 cycles, Index 2 8 cycles and Read 2 80 cycles. TAMs were extracted and position weight matrix based on the enrichment score was generated and Weblogos were visualized based on this position weight matrix using a custom Python script. All sequencing primers used are listed in table S4.

### In Vitro Biochemical TAM Screen

1 μM of purified RNP and 100 ng of the 7N TAM library is incubated at 37 degree Celsius in NEB buffer 3 for 3 hours. Subsequently, reaction is purified and analyzed following the same procedure as cell-free transcription/translation TAM screen. TAM library sequence and guides used are listed in table S5.

### Cell-Free Transcription/Translation Cleavage Assays

Cell-free transcription/translation reactions were carried out using a PURExpress In Vitro Protein Synthesis Kit (NEB) as per the manufacturer's protocol with half-volume reactions using 75 ng of template for the protein of interest and a 100 ng of fRNA or ωRNA. Reactions were incubated at 37° C. for 4 hours to allow for RNP formation, then placed on ice to quench in vitro transcription/translation. 50-100 ng of target substrate was then added, and the reactions were incubated at the specified temperature for 1 additional hour. Reactions were then quenched by heating up to 95 degrees for 15 minutes and cooling back down to 50-degrees Celcius for addition of 10 μg RNase A (Qiagen) for 10 minutes incubation. DNA was extracted by PCR purification using minElute columns (Qiagen) and run on 6% Novex TBE gels (Thermo Fisher Scientific) as per the manufacturer's protocols, as specified in figures. Gels were stained with 1× SYBR Gold (Thermo Fisher Scientific) for 10-15 min and imaged on a ChemiDoc imager (BioRad) with optimal exposure settings. Each condition was performed twice for replicability.

TABLE 7
TAM library and spacer sequences relevant for the present disclosure.

| | | SEQ ID |
|---|---|---|
| Name | NGS Primers | NO |
| TAM | gatcaaaggatcttcttgagatcctttttttctgcgcgtaatctgctgcttgcaaacaaaaaaaccaccgctaccagcggt | 59 |
| Library | ggtttgtttgccggatcaagagctaccaactctttttccgaaggtaactggcttcagcagagcgcagataccaaatact | |
| Plasmid | gttcttctagtgtagccgtagttaggccaccacttcaagaactctgtagcaccgcctacatacctcgctctgctaatcctg | |
| | ttaccagtggctgctgccagtggcgataagtcgtgtcttaccgggttggactcaagacgatagttaccggataaggcg | |
| | cagcggtcgggctgaacggggggttcgtgcacacagcccagcttggagcgaacgacctacaccgaactgagatac | |
| | ctacagcgtgagctatgagaaagcgccacgcttcccgaagggagaaaggcggacaggtatccggtaagcggcag | |
| | ggtcggaacaggagagcgcacgagggagcttccagggggaaacgcctggtatctttatagtcctgtcgggtttcgc | |
| | cacctctgacttgagcgtcgatttttgtgatgctcgtcaggggggcggagcctatggaaaaacgccagcaacgcggc | |
| | ctttttacggttcctggccttttgctggccttttgctcacatgttctttcctgcgttatcccctgattctgtggataaccgtatta | |
| | ccgcctttgagtgagctgataccgctcgccgcagccgaacgaccgagcgcagcgagtcagtgagcgaggaagcg | |
| | gaagagcgcccaatacgcaaaccgcctctccccgcgcgttggccgattcattaatgcagctggcacgacaggtttcc | |
| | cgactggaaagcgggcagtgagcgcaacgcaattaatgtgagttagctcactcattaggcaccccaggctttacactt | |
| | tatgcttccggctcgtatgttgtgTGGAATTGTGAGCGGATAACAATTTCACACAGGA | |
| | AACAGCTATGACCATGATTACGCCAAGCTTNNNNNNNGCAGCCACCTC | |
| | CTTGTTATTGGGTACCGAGCTCGAATTCACTGGCCGTCGTTTTACAACG | |
| | TCGTGACTGGGAAAACCCTGGCGTTACCCAACTTAATCGCCTTGCAGca | |
| | catccccctttcgccagctggcgtaatagcgaagaggcccgcaccgatcgcccttcccaacagttgcgcagcctgaa | |
| | tggcgaatggcgcctgatgcggtattttctccttacgcaTCTGTGCGGTATTTCACACCGCATA | |
| | TGGTGCACTCTCAGTACAATCTGCTCTGATGCCGCATAGTTAAGCCAG | |
| | CCCCGACACCCGCCAACACCCGCTGACGCGCCCTGACGGGCTTGTCTG | |
| | CTCCCGGCATCCGCTTACAGACAAGCTGTGACCGTCTCcgggagctgcatgtgtc | |
| | agaggttttcaccgtcatcaccgaaacgcgcgagacgaaagggcctcgtgatacgcctatttttataggttaatgtcat | |
| | gataataatggtttcttagacgtcaggtggcacttttcggggaaatgtgcgcggaacccctatttgtttatttttctaaatac | |
| | attcaaatatgtatccgctcatgagacaataaccctgataaatgcttcaataatattgaaaaaggaagagtatgagtattc | |
| | aacatttccgtgtcgcccttattcccttttttgcggcattttgccttcctgtttttgctcacccagaaacgctggtgaaagtaa | |
| | aagatgctgaagatcagttgggtgcacgagtgggttacatcgaactggatctcaacagcggtaagatccttgagagtt | |
| | ttcgccccgaagaacgttttccaatgatgagcacttttaaagttctgctatgtggcgcggtattatcccgtattgacgccg | |
| | ggcaagagcaactcggtcgccgcatacactattctcagaatgacttggttgagtactcaccagtcacagaaaagcatc | |
| | ttacggatggcatgacagtaagagaattatgcagtgctgccataaccatgagtgataacactgcggccaacttacttct | |
| | gacaacgatcggaggaccgaaggagctaaccgcttttttgcacaacatgggggatcatgtaactcgccttgatcgttg | |
| | ggaaccggagctgaatgaagccataccaaacgacgagcgtgacaccacgatgcctgtagcaatggcaacaacgtt | |
| | gcgcaaactattaactggcgaactacttactctagcttcccggcaacaattaatagactggatggaggcggataaagtt | |
| | gcaggaccacttctgcgctcggcccttccggctggctggtttattgctgataaatctggagccggtgagcgtgggtctc | |
| | gcggtatcattgcagcactggggccagatggtaagccctcccgtatcgtagttatctacacgacggggagtcaggca | |
| | actatggatgaacgaaatagacagatcgctgagataggtgcctcactgattaagcattggtaactgtcagaccaagttt | |
| | actcatatatactttagattgatttaaaacttcatttttaatttaaaaggatctaggtgaagatcctttttgataatctcatgacc | |
| | aaaatcccttaacgtgagttttcgttccactgagcgtcagaccccgtagaaaa | |
| 21ntre- | GCAGCCACCTCCTTGTTATTG | 60 |
| port | | |
| EMX1-1 | aaaaaaaagaaaagaaaaaa | 61 |
| EMX1-2 | aagagtggccttgatttgta | 62 |
| EMX1-3 | aaataaaatttaaaaaaaaa | 63 |
| EMX1-4 | gtttccagttttattttgtta | 64 |
| EMX1-5 | gagaaacaaatgaaagggac | 65 |
| DYNC1h1_ | gagatggtaggttcttctaa | 66 |
| G1 | | |
| DYNC1h1_ | aatacacatagatatagggtc | 67 |
| G2 | | |
| DYNC1h1_ | aaaaaaacaaaaaaaccaaaa | 68 |
| G3 | | |
| DYNC1h1_ | aacatcaaagtgcactgtcag | 69 |
| G4 | | |
| DYNC | caaaattcttaattt | 70 |
| B2m_G1 | gtgatcatgtaccctgaata | 71 |
| B2m_G2 | aaagaattttatacacata | 72 |
| B2m_G3 | tacacatatatttagtgtca | 73 |
| B2m_G4 | gtagcactaacacttctctt | 74 |
| B2m_G5 | aatacacttatattcagggt | 75 |
| cxcr4_G1 | tatctgaaaaatgtgtaact | 76 |
| cxcr4_G4 | tatctgaaaaatgtgtaact | 76 |
| cxcr4_G2 | tacgataaataactttt | 77 |
| cxcr4_G3 | agttacacatttttcagata | 78 |
| cxcr4_G5 | attgacttatttatataaat | 79 |
| CA2_G1 | tagtcagaagaagaagtttg | 80 |
| CA2_G2 | cagaaagatccaaacttctt | 81 |
| CA2_G3 | ttcatctgacaacttccttt | 82 |
| CA2_G4 | tagatgaggagacttgtaga | 83 |
| CA2_G5 | attctacaatgatatattgt | 84 |
| DMD_G1 | TATAAATGAATATTCCGTTGT | 85 |
| DMD_G2 | TCCATTTATCTGTTAATGGC | 86 |
| DMD_G3 | CAGTATCATCAGGAAGAATAA | 87 |
| DMD_G4 | TTCTTCCTGATGATACTGTA | 88 |
| DMD_G5 | GTTAAATTTATTCCTCTTTT | 89 |
| GRIN2b_ | GCTCCCTAAGGGGACAGACC | 90 |
| G1 | | |
| GRIN2b_ | AGTTTAACTTTATGAAATTGC | 91 |
| G2 | | |
| GRIN2b_ | ACTTTATGAAATTGCCTTTT | 92 |
| G3 | | |
| GRIN2b_ | TTATATGTCAATAATGGTTA | 93 |
| G4 | | |
| GRIN2b_ | TATGTCAATAATGGTTATTTC | 94 |
| G5 | | |

### Small RNA Sequencing

Heterologous expression in E. coli: Rosetta2 chemically competent E. coli were transformed with plasmids containing the locus of interest. A single colony was used to seed a 5 mL overnight culture. Following overnight growth, cultures were spun down, resuspended in 750 μL TRI reagent (Zymo) and incubated for 5 min at room temperature. 0.5 mm zirconia/silica beads (BioSpec Products) were added and the culture was vortexed for approximately 1 minute to mechanically lyse cells. 200 μL chloroform (Sigma Aldrich) was then added, culture was inverted gently to mix and incubated at room temperature for 3 min, followed by spinning at 12000×g at 4° C. for 15 min. The aqueous phase was used as input for RNA extraction using a Direct-zol RNA miniprep plus kit (Zymo). Extracted RNA was treated with 10 units of DNase I (NEB) for 30 min at 37° C. to remove residual DNA and purified again with an RNA Clean & Concentrator-25 kit (Zymo). Ribosomal RNA was removed using a RiboMinus Transcriptome Isolation Kit for bacteria (Thermo Fisher Scientific) as per the manufacturer's protocol using half-volume reactions. The purified sample was then treated with 20 units of T4 polynucleotide kinase (NEB) for 6 h at 37° C. and purified again with an RNA Clean & Concentrator-25 (Zymo) kit. The purified RNA was treated with 20 units of 5′ RNA phosphatase (Lucigen) for 30 min at 37° C. and purified again using an RNA Clean & Concentrator-5 kit (Zymo). Purified RNA was used as input to an NEBNext Small RNA Library Prep for Illumina (NEB) as per the manufacturer's protocol with an extension time of 60 s and 16 cycles in the final PCR. Amplified libraries were gel extracted, quantified by qPCR using a KAPA Library Quantification Kit for Illumina (Roche) on a StepOne Plus machine (Applied Biosystems/Thermo Fisher Scientific) and sequenced on an Illumina NextSeq with Read 1 42 cycles, Read 2 42 cycles and Index 1 6 cycles. Adapters were trimmed using CutAdapt and mapped to loci of interest using BWA-align. Reads were visualized using Genious.

Ribonucleoprotein: RNPs were purified as described. 100 μL concentrated RNP was used as input. The above protocol was followed with the following modifications: 300 μL TRI reagent (Zymo) and 60 μL chloroform (Sigma Aldrich) were used for RNA extraction.

PureExpress RNPs: 75 ng of plasmid encoding the Fanzor ORF and 125 ng of the plasmid containing the locus were incubated in 1 unit of pureexpress reactions for 4 hours at 37 degrees Celcius. Afterward, the RNP is affinity purified using the protocol described above for heterologous Rosetta cell protein production and subjected to the same pipeline for small RNA sequencing.

Chlamydomonas reinhardtii was obtained from the University of Minnesota (CRC). The algae was lysed in trizol with glass beads vigorously shaken for 2 hours at room temperature. Then the above protocol was followed with the following modifications: Ribosomal RNA was removed using a plant specific ribominus rRNA depletion kits as per the manufacturer's protocol and the rRNA-depleted sample was purified using Agencourt RNAClean XP beads (Beckman Coulter) prior to T4 PNK treatment. T4 PNK treatment was performed for 1.5 h and purified with an RNA Clean & Concentrator-5 kit (Zymo). Final PCR in the small RNA library prep contained 10 cycles.

### Collateral Activity Testing

DNase alert and Rnase alert were purchased from IDT. 1 μM of RNP or 10 μL of PureExpress generated RNP and 10 nM of DNA target containing either the target spacer or a scramble spacer are diluted in 1× DNase/Rnase alert reaction buffer into 50 μL reactions. The solution is mixed well in the reaction test tube and subsequently aliquoted into 384 well plates. The plates are loaded onto applied biosystems qPCR machines and reactions were ran at 37 degree Celsius for ApmHNuc, AmpFNuc2, DrpFNuc2, BaaFNuc2, MemFNuc2, and Isdra2 TnpB, and 60 degree Celsius for TvoTnpB. The SYBR and HEX channel fluorescence intensity is recorded every minute for a duration of 60 minutes. The intensity is normalized by subtracting the non-target DNA sequence from the target DNA sequence group. A positive control DNase (2 μL) and RNAse (2 μL) is ran along with the Fanzor/TnpB group as a positive control to monitor the assay.

### Cloning PAM/TAM Libraries

Target sequences with 7N degenerate flanking sequences were synthesized by IDT and amplified by PCR with NEBNext High Fidelity 2× Master Mix (NEB). Backbone plasmid was digested with restriction enzymes (pUC19: KPNI and HindIII, Thermo Fisher Scientific) and treated with FastAP alkaline phosphatase (Thermo Fisher Scientific). The amplified library fragment was inserted into the backbone plasmid by Gibson assembly at 50° C. for 1 hour using 2× Gibson Assembly Master Mix (NEB) with an 8:1 molar ratio of insert:vector. The Gibson assembly reaction was then isopropanol precipitated by the addition of an equal volume of isopropanol (Sigma Aldrich), the final concentration of 50 mM NaCl, and 1 μL of GlycoBlue nucleic acid co-precipitant (Thermo Fisher Scientific). After a 15 min incubation at room temperature, the solution was spun down at max speed at 4° C. for 15 min, then the supernatant was pipetted off and the pelleted DNA has resuspended in 12 μL TE and incubated at 50° C. for 10 minutes to dissolve. 2 μL were then transformed by electroporation into Endura Electrocompetent E. coli (Lucigen) as per the manufacturer's instructions, recovered by shaking at 37° C. for 1 h, then plated across 5 22.7 cm×22.7 cm BioAssay plates with the appropriate antibiotic resistance. After 12-16 hours of growth at 37 C, cells were scraped from the plates and midi- or maxi-prepped using a NucleoBond Midi- or Maxi-prep kit (Machery Nagel). The sequence is provided in Table 7.

### In Vitro TAM Discovery

1 μM of RNP and 25 ng of TAM library plasmid were incubated at 37 degree for 2 hours in NEB Buffer 3. Reactions were quenched by placing at 4° C. or on ice and adding 10 μg Rnase A (Qiagen) and 8 units Proteinase K (NEB) each followed by a 5 min incubation at 37° C. DNA was extracted by PCR purification and adaptors were ligated using an NEBNext Ultra II DNA Library Prep Kit for Illumina (NEB) using the NEBNext Adaptor for Illumina (NEB) as per the manufacturer's protocol. Following adaptor ligation, cleaved products were amplified specifically using one primer specific to the TAM library backbone and one primer specific to the NEBNext adaptor with a 12-cycle PCR using NEBNext High Fidelity 2× PCR Master Mix (NEB) with an annealing temperature of 63° C., followed by a second 20-cycle round of PCR to further add the Illumina i5 adaptor. Amplified libraries were gel extracted, quantified by qubit dsDNA kit (Invitrogen) and subject to single-end sequencing on an Illumina MiSeq with Read 1 200 cycles, Index 1 8 cycles and Index 2 8 cycles. TAMs were extracted and visualized by Weblogo3. Alternatively, a primer set targeting the TAM library plasmid is used to amplify the uncleaved product for 12 cycle and followed by a second 20 cycle rounds of PCR to add the Illumina i5 adaptor. Amplified libraries were gel extracted and subjected to single end sequencing on an Illumina MiSeq with Read 1 200 cycles, Index 1 8 cycles and Index 2 8 cycles. Depletion of TAMs were calculated by comparing to a non-targeting RNP as control and normalized to the original plasmid library distribution. Primers used are listed in Table 8.

TABLE 8
Additional sequences relevant for the present disclosure

| SEQ ID | | |
|---|---|---|
| NO: | NGS Primers | Name |
| 39 | ACACTCTTTCCCTACACGACGCTCTTCCGATCTCtggaattgtga | TAM_NGS_F1 |
| | gcggataacaatttcacacagg | |
| 40 | GTGACTGGAGTTCAGACGTGTGCTCTTCCGATCTctgcaaggc | TAM_NGS_R |
| | gattaagttgggtaacgcc | |
| 41 | ACACTCTTTCCCTACACGACGCTCTTCCGATCTCacgtggagtc | Luciferase_Indel_ |
| | caaccctggacc | NGS_F1 |
| 42 | GTGACTGGAGTTCAGACGTGTGCTCTTCCGATCTtcagcatcg | Luciferase_Indel_ |
| | agatccgtggtcgc | NGS_R1 |

### In Vitro Cleavage Assays

Double-stranded DNA (dsDNA) substrates were produced by PCR amplification of pUC19 plasmids containing the target sites and the TAM sequences. All ωRNA and fRNA used in the biochemical assays was in vitro transcribed using the HiScribe T7 Quick High Yield RNA Synthesis kit (NEB) from the DNA templates purchased from IDT. Target cleavage assays performed with ApmHNuc contained 10 nM of DNA substrate, 1 μM of protein, and 4 μM of fRNA in a final 1× reaction buffer of NEB Buffer 3. Assays were allowed to proceed at 37° C. for 2 hour, then briefly shifted to 50° C. for 5 min, and immediately placed on ice to help relax the RNA structure prior to RNA digestion. Reactions were then treated with Rnase A (Qiagen), and Proteinase K (NEB), and purified using a PCR cleanup kit (Qiagen). DNA was resolved by gel electrophoresis on Novex 6% TBE polyacrylamide gels (Thermo Fisher Scientific). 1 μM of purified RNP and 100 ng of the 7N TAM library is incubated at 37 degree Celsius in NEB buffer 3 for 3 hours. Subsequently, reaction is purified and analyzed following the same procedure as cell-free transcription/translation TAM screen. TAM library sequence and guides used are listed in Table 7.

### Cleavage Position Mapping by Next Generation Sequencing

1 μM of RNP and 100 ng of the target plasmid were incubated at 37 degree for 3 hours in NEB Buffer 3. Reactions were quenched by placing at 4° C. or on ice and adding 10 μg RNase A (Qiagen) and 8 units Proteinase K (NEB) each followed by a 5 min incubation at 37° C. DNA was extracted by PCR purification and adaptors were ligated using an NEBNext Ultra II DNA Library Prep Kit for Illumina (NEB) using the NEBNext Adaptor for Illumina (NEB) as per the manufacturer's protocol. Following adaptor ligation, cleaved products were amplified specifically using one primer specific to the target plasmid (one on 5′ site of the cleavage and one on 3′ side of the cleavage) and one primer specific to the NEBNext adaptor with a 12-cycle PCR using NEBNext High Fidelity 2× PCR Master Mix (NEB) with an annealing temperature of 63° C., followed by a second 20-cycle round of PCR to further add the Illumina i5 adaptor. Amplified libraries were gel extracted, quantified by qubit dsDNA kit (Invitrogen) and subject to single-end sequencing on an Illumina MiSeq with Read 1 100 cycles, Index 1 8 cycles and Index 2 8 cycles. All sequencing primers are listed in Table 6.

### Confocal Images of Nuclear Localization

The N-terminal predicted NLS sequences of Fanzor is cloned onto N-terminal of sfGFP by Gibson assembly into a pCMV promoter backbone (NLS sequences cloned are listed in Table 5). 24 hours before transfection, 15,000 HEK293FT cells were plated onto a glass bottom 96 well plates pre-coated with poly-D lysine. 100 ng of NLS-sfGFP construct is transfected into HEK293FT cells using lipofectamine 3000 and 24 hours after transfection, cells were fixed and permeabilized using Fix and Perm kit (Thermofisher) and subsequently stained by either DAPI or SYTO-Red nuclear stain (Thermofisher). All wells were measured via confocal microscopy at room temperature. Cells were focused in the 488 nm channel on the basis of the sfGFP protein. For each well, a 2×2 field of view image at 20× magnification was collected under the following settings and stitched around the center point. Images were collected in 488 nm (32.8% power, 100 ms exposure), 359 nm (35.2% power, 100 ms exposure), and 633 nm (80% power, 100 ms exposure).

### Mammalian Cell Culture and Transfection

Mammalian cell culture experiments were performed in the HEK293FT line (Thermo Fisher) grown in Dulbecco's Modified Eagle Medium with high glucose, sodium pyruvate, and GlutaMAX (Thermo Fisher), additionally supplemented with 1× penicillin-streptomycin (Thermo Fisher), 10 mM HEPES (Thermo Fisher), and 10% fetal bovine serum (VWR Seradigm). All cells were maintained at confluency below 80%.

All transfections were performed with Lipofectamine 3000 (Thermo Fisher). Cells were plated 16-20 hours prior to transfection to ensure 90% confluency at the time of transfection. For 96-well plates, cells were plated at 20,000 cells/well. For each well on the plate, transfection plasmids were combined with OptiMEM I Reduced Serum Medium (Thermo Fisher) to a total of 10 μL.

### Mammalian Genome Editing

fRNA scaffold backbones were cloned into a pUC19-based human U6 expression backbone and human codon-optimized Fanzor proteins were cloned into pCMV-based or pCAG-based destination vector by Gibson Assembly. Then 50 ng of protein expression construct, 50 ng of the corresponding guide construct and an optionally 20 ng of luciferase reporter were transfected in one well of a 96-well plate using lipofectamine 3000 transfection reagent. After 48 hours, reporter DNA was harvested by washing the cells once in 1×DPBS (Sigma Aldrich) and resuspended in 50 μL QuickExtract DNA Extraction Solution (Lucigen) and cycled at 65° C. for 15 min, 68° C. for 15 min then 95° C. for 10 min to lyse cells. 2.5 μL of lysed cells were used as input into each PCR reaction. For library amplification, target reporter regions were amplified with a 12-cycle PCR using NEBNext High Fidelity 2× PCR Master Mix (NEB) with an annealing temperature of 63° C. for 15 s, followed by a second 18-cycle round of PCR to add Illumina adapters and barcodes. The libraries were gel extracted and subject to single-end sequencing on an Illumina MiSeq with Read 1 220 cycles, Index 1 8 cycles, Index 2 8 cycles and Read 2 80 cycles. Insertion/deletion (indel) frequency was analyzed using CRISPResso2. All sequencing primers are listed in Table 6. Guides used for genomic target are listed in Table 7.

### REFERENCES

1. V. V. Kapitonov, K. S. Makarova, E. V. Koonin, ISC, a Novel Group of Bacterial and Archaeal DNA Transposons That Encode Cas9 Homologs. J. Bacteriol. 198, 797-807 (2015).
2. S. He, C. Guynet, P. Siguier, A. B. Hickman, F. Dyda, M. Chandler, B. Ton-Hoang, IS200/IS605 family single-strand transposition: mechanism of IS608 strand transfer. Nucleic Acids Res. 41, 3302-3313 (2013).
3. B. Zetsche, J. S. Gootenberg, O. O. Abudayyeh, I. M. Slaymaker, K. S. Makarova, P. Essletzbichler, S. E. Volz, J. Joung, J. van der Oost, A. Regev, E. V. Koonin, F. Zhang, Cpf1 is a single RNA-guided endonuclease of a class 2 CRISPR-Cas system. Cell. 163, 759-771 (2015).
4. I. Fonfara, H. Richter, M. Bratovič, A. Le Rhun, E. Charpentier, The CRISPR-associated DNA-cleaving enzyme Cpf1 also processes precursor CRISPR RNA. Nature. 532, 517-521 (2016).
5. L. B. Harrington, D. Burstein, J. S. Chen, D. Paez-Espino, E. Ma, I. P. Witte, J. C. Cofsky, N. C. Kyrpides, J. F. Banfield, J. A. Doudna, Programmed DNA destruction by miniature CRISPR-Cas14 enzymes. Science (2018), doi: 10.1126/science.aav4294.
6. T. Karvelis, G. Druteika, G. Bigelyte, K. Budre, R. Zedaveinyte, A. Silanskas, D. Kazlauskas, Č. Venclovas, V. Siksnys, Transposon-associated TnpB is a programmable RNA-guided DNA endonuclease. Nature. 599, 692-696 (2021).
7. W. Bao, J. Jurka, Homologues of bacterial TnpB_IS605 are widespread in diverse eukaryotic transposable elements. Mob. DNA. 4, 12 (2013).
8. H. Altae-Tran, S. Kannan, F. E. Demircioglu, R. Oshiro, S. P. Nety, L. J. McKay, M. Dlakić, W. P. Inskeep, K. S. Makarova, R. K. Macrae, E. V. Koonin, F. Zhang, The widespread IS200/IS605 transposon family encodes diverse programmable RNA-guided endonucleases. Science. 374, 57-65 (2021).
9. K. Riehl, C. Riccio, E. A. Miska, M. Hemberg, TransposonUltimate: software for transposon classification, annotation and detection. Nucleic Acids Res. 50, e64 (2022).
10. S. P. Nety, H. Altae-Tran, S. Kannan, F. E. Demircioglu, G. Faure, S. Hirano, K. Mears, Y. Zhang, R. K. Macrae, F. Zhang, The Transposon-Encoded Protein TnpB Processes Its Own mRNA into ωRNA for Guided Nuclease Activity. CRISPR J. 6, 232-242 (2023).
11. C. Meers, H. Le, S. R. Pesari, F. T. Hoffmann, M. W. G. Walker, J. Gezelle, S. H. Sternberg, Transposon-encoded nucleases use guide RNAs to selfishly bias their inheritance. bioRxiv (2023), p. 2023.03.14.532601.
12. R. Nakagawa, H. Hirano, S. N. Omura, S. Nety, S. Kannan, H. Altae-Tran, X. Yao, Y. Sakaguchi, T. Ohira, W. Y. Wu, H. Nakayama, Y. Shuto, T. Tanaka, F. K. Sano, T. Kusakizako, Y. Kise, Y. Itoh, N. Dohmae, J. van der Oost, T. Suzuki, F. Zhang, O. Nureki, Cryo-EM structure of the transposon-associated TnpB enzyme. Nature. 616, 390-397 (2023).
13. G. Sasnauskas, G. Tamulaitiene, G. Druteika, A. Carabias, A. Silanskas, D. Kazlauskas, Č. Venclovas, G. Montoya, T. Karvelis, V. Siksnys, TnpB structure reveals minimal functional core of Cas12 nuclease family. Nature. 616, 384-389 (2023).
14. J. S. Chen, E. Ma, L. B. Harrington, M. Da Costa, X. Tian, J. M. Palefsky, J. A. Doudna, CRISPR-Cas12a target binding unleashes indiscriminate single-stranded DNase activity. Science. 360, 436-439 (2018).
15. O. O. Abudayyeh, J. S. Gootenberg, S. Konermann, J. Joung, I. M. Slaymaker, D. B. T. Cox, S. Shmakov, K. S. Makarova, E. Semenova, L. Minakhin, K. Severinov, A. Regev, E. S. Lander, E. V. Koonin, F. Zhang, C2c2 is a single-component programmable RNA-guided RNA-targeting CRISPR effector. Science. 353, aaf5573 (2016).
16. M. Boyer, N. Yutin, I. Pagnier, L. Barrassi, G. Fournous, L. Espinosa, C. Robert, S. Azza, S. Sun, M. G. Rossmann, M. Suzan-Monti, B. La Scola, E. V. Koonin, D. Raoult, Giant Marseillevirus highlights the role of amoebae as a melting pot in emergence of chimeric microorganisms. Proc. Natl. Acad. Sci. U.S.A 106, 21848-21853 (2009).
17. G. Bigelyte, J. K. Young, T. Karvelis, K. Budre, R. Zedaveinyte, V. Djukanovic, E. Van Ginkel, S. Paulraj, S. Gasior, S. Jones, L. Feigenbutz, G. S. Clair, P. Barone, J. Bohn, A. Acharya, G. Zastrow-Hayes, S. Henkel-Heinecke, A. Silanskas, R. Seidel, V. Siksnys, Miniature type V-F CRISPR-Cas nucleases enable targeted DNA modification in cells. Nat. Commun. 12, 6191 (2021).
18. Z. Wu, Y. Zhang, H. Yu, D. Pan, Y. Wang, Y. Wang, F. Li, C. Liu, H. Nan, W. Chen, Q. Ji, Programmed genome editing by a miniature CRISPR-Cas12f nuclease. Nat. Chem. Biol. 17, 1132-1138 (2021).
19. X. Xu, A. Chemparathy, L. Zeng, H. R. Kempton, S. Shang, M. Nakamura, L. S. Qi, Engineered miniature CRISPR-Cas system for mammalian genome regulation and editing. Mol. Cell. 0 (2021), doi: 10.1016/j.molcel.2021.08.008.
20. D. Y. Kim, J. M. Lee, S. B. Moon, H. J. Chin, S. Park, Y. Lim, D. Kim, T. Koo, J.-H. Ko, Y.-S. Kim, Efficient CRISPR editing with a hypercompact Cas12f1 and engineered guide RNAs delivered by adeno-associated virus. Nat. Biotechnol., 1-9 (2021).
21. S. Shmakov, A. Smargon, D. Scott, D. Cox, N. Pyzocha, W. Yan, O. O. Abudayyeh, J. S. Gootenberg, K. S. Makarova, Y. I. Wolf, K. Severinov, F. Zhang, E. V. Koonin, Diversity and evolution of class 2 CRISPR-Cas systems. Nat. Rev. Microbiol. 15, 169-182 (2017).
22. S. N. Takeda, R. Nakagawa, S. Okazaki, H. Hirano, K. Kobayashi, T. Kusakizako, T. Nishizawa, K. Yamashita, H. Nishimasu, O. Nureki, Structure of the miniature type V-F CRISPR-Cas effector enzyme. Mol. Cell. 81, 558-570.e3 (2021).
23. A. N. Nguyen Ba, A. Pogoutse, N. Provart, A. M. Moses, NLStradamus: a simple Hidden Markov Model for nuclear localization signal prediction. BMC Bioinformatics. 10, 202 (2009).
24. D. C. Swarts, K. Makarova, Y. Wang, K. Nakanishi, R. F. Ketting, E. V. Koonin, D. J. Patel, J. van der Oost, The evolutionary journey of Argonaute proteins. Nat. Struct. Mol. Biol. 21, 743-753 (2014).
25. P. D. Hsu, E. S. Lander, F. Zhang, Development and applications of CRISPR-Cas9 for genome engineering. Cell. 157, 1262-1278 (2014).
26. F. Hille, H. Richter, S. P. Wong, M. Bratovič, S. Ressel, E. Charpentier, The Biology of CRISPR-Cas: Backward and Forward. Cell. 172, 1239-1259 (2018).
27. J. A. Doudna, E. Charpentier, The new frontier of genome engineering with CRISPR-Cas9. Science. 346, 1258096 (2014).
28. J. Strecker, A. Ladha, Z. Gardner, J. L. Schmid-Burgk, K. S. Makarova, E. V. Koonin, F. Zhang, RNA-guided DNA insertion with CRISPR-associated transposases. Science (2019), doi: 10.1126/science.aax9181.
29. S. E. Klompe, P. L. H. Vo, T. S. Halpin-Healy, S. H. Sternberg, Transposon-encoded CRISPR-Cas systems direct RNA-guided DNA integration. Nature. 571, 219-225 (2019).
30. G. J. Hannon, RNA interference. Nature. 418, 244-251 (2002).
31. G. Hutvagner, M. J. Simard, Argonaute proteins: key players in RNA silencing. Nat. Rev. Mol. Cell Biol. 9, 22-32 (2008).
32. M. Saito, P. Xu, G. Faure, S. Maguire, S. Kannan, H. Altae-Tran, S. Vo, A. Desimone, R. K. Macrae, F. Zhang, Fanzor is a eukaryotic programmable RNA-guided endonuclease. Nature (2023), doi: 10.1038/s41586-023-06356-2.
33. P. López-García, D. Moreira, The symbiotic origin of the eukaryotic cell. C. R. Biol. 346, 55-73 (2023).
34. M. Boyer, N. Yutin, I. Pagnier, L. Barrassi, G. Fournous, L. Espinosa, C. Robert, S. Azza, S. Sun, M. G. Rossmann, M. Suzan-Monti, B. La Scola, E. V. Koonin, D. Raoult, Giant Marseillevirus highlights the role of amoebae as a melting pot in emergence of chimeric microorganisms. Proc. Natl. Acad. Sci. U.S.A 106, 21848-21853 (2009).
35. C. Pasternak, R. Dulermo, B. Ton-Hoang, R. Debuchy, P. Siguier, G. Coste, M. Chandler, S. Sommer, ISDra2 transposition in Deinococcus radiodurans is downregulated by TnpB. Mol. Microbiol. 88, 443-455 (2013).
36. C. Meers, H. Le, S. R. Pesari, F. T. Hoffmann, M. W. G. Walker, J. Gezelle, S. H. Sternberg, Transposon-encoded nucleases use guide RNAs to selfishly bias their inheritance. bioRxiv (2023), doi: 10.1101/2023.03.14.532601.
37. W. Bao, K. K. Kojima, O. Kohany, Repbase Update, a database of repetitive elements in eukaryotic genomes. Mob. DNA. 6, 11 (2015).
38. J. A. Rees, K. Cranston, Automated assembly of a reference taxonomy for phylogenetic data synthesis. Biodivers Data J, e12581 (2017).

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

1-77. (canceled)

78. A method of modifying a target polynucleotide sequence in a cell, comprising delivering to the cell
(a) a nucleic acid encoding a Fanzor polypeptide comprising an RuvC domain; and
(b) a nucleic acid encoding a fRNA molecule comprising a scaffold and a reprogrammable target spacer sequence,
wherein the fRNA molecule is capable of forming a complex with the Fanzor polypeptide and directing the Fanzor polypeptide to a target polynucleotide sequence.

79. The method of claim 78, wherein the modifying comprises cleavage of the target polynucleotide sequence, optionally wherein the target polynucleotide sequence is DNA.

80. The method of claim 79, wherein the cleavage occurs within the target polynucleotide near the 3′ end of the target polynucleotide sequence, about −6 to about +3 nucleotides relative to the 3′ end of the target polynucleotide sequence, or within a TAM sequence.

81. The method of claim 78, wherein one or more mutations comprising substitutions, deletions, and insertion are introduced into the target polynucleotide sequence.

82. The method of claim 78, wherein (a) and (b) are delivered to the cell together.

83. The method of claim 78, wherein (a) and (b) are delivered to the cell separately.

84. The method of claim 78, wherein the delivering to a cell occurs
(a) in vivo;
(b) ex vivo; or
(c) in vitro.

85. The method of claim 78, wherein the cell is a mammalian cell, a human cell, a eukaryotic cell, a prokaryotic cell, a plant cell, a bacterial cell, a fungal cell, a yeast cell, a rodent cell, or a primate cell.

86. A composition comprising a stabilized Fanzor polypeptide comprising an RuvC domain, comprising one or more mutations relative to wildtype Fanzor polypeptide, wherein the mutations stabilize the Fanzor polypeptide.

87. A method of modifying a target polynucleotide sequence in a cell, comprising:
(a) delivering to the cell the composition of claim 86; and
(b) separately delivering to the cell a fRNA molecule.
